(19) United States
(12) Patent Application Publication
(10) Pub. No.: US 2013/0244966 A1
(43) Pub. Date: Sep. 19, 2013

(54) FATTY ACID ANTIVIRAL CONJUGATES AND THEIR USES

(71) Applicant: CATABASIS PHARMACEUTICALS, INC., Cambridge, MA (US)

(72) Inventors: Jill C. Milne, Brookline, MA (US); Michael R. Jirousek, Cambridge, MA (US); Chi B. Vu, Arlington, MA (US); Allison Wensley, Cambridge, MA (US); Amal Ting, Newton, MA (US)

(73) Assignee: CATABASIS PHARMACEUTICALS, INC., Cambridge, MA (US)

(21) Appl. No.: 13/712,544

(22) Filed: Dec. 12, 2012

Related U.S. Application Data
(60) Provisional application No. 61/569,592, filed on Dec. 12, 2011.

(51) Int. Cl. C07D 487/04; C07D 405/04

(52) U.S. Cl. CPC C07D 487/04; C07D 405/04
USPC 514/48; 536/26.8; 514/51; 536/26.7

(57) ABSTRACT

The invention relates to fatty acid antiviral conjugates; compositions comprising an effective amount of a fatty acid antiviral conjugate; and methods for treating or preventing a viral infection comprising the administration of an effective amount of a fatty acid antiviral conjugate.

### PRIORITY

This application claims the benefit of U.S. Provisional Application No. 61/569,592, filed Dec. 12, 2011, the entire disclosure of which is relied on and incorporated into this application by reference.

### FIELD OF THE INVENTION

The invention relates to fatty acid antiviral conjugates; compositions comprising an effective amount of a fatty acid antiviral conjugate; and methods for treating or preventing a viral infection comprising the administration of an effective amount of a fatty acid antiviral conjugate. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in patients at risk of a cardiovascular event. Omega-3 fatty acids have also been taken as part of the dietary supplement portion of therapy used to treat dyslipidemia. Last, but not least, omega-3 fatty acids have been known to have a number of anti-inflammatory properties. For instance, a higher intake of omega-3 fatty acids lower levels of circulating TNF-α and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has been shown to increase levels of the well-characterized anti-inflammatory cytokine IL-10 (Bradley et al, Obesity (Silver Spring) 2008, 16, p. 938-944).

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

Viruses are basically small infectious agents that can replicate inside living cells of human, animals or plants. Viruses consist of two or three parts: the genetic material made from either DNA or RNA; a protein coat that protects these genes; and in some cases an envelope of lipids that surrounds the protein coat when they are outside of cells. Viruses come in all kinds of shapes and sizes and are grouped according to the Baltimore classification: Group I, double-stranded DNA viruses; Group II, single-stranded DNA viruses; Group III, double-stranded RNA viruses; Group IV, (+)-single stranded RNA viruses; Group V, (−)-single-stranded RNA viruses; Group VI, single stranded RNA reverse-transcribing viruses; Group VII, double-stranded DNA reverse-transcribing viruses. A few examples of human diseases caused by viruses include the common cold, influenza, chicken pox, AIDS, and hepatitis. Viral infection provokes an immune response that can eventually help to eliminate the infecting virus. However, some viruses, including those causing AIDS and viral hepatitis, can manage to evade these immune responses and result in chronic infections. In these cases, treatment with an appropriate antiviral agent becomes necessary.

A fatty acid antiviral conjugate represents a covalently linked antiviral agent and an omega-3 fatty acid such as DHA or EPA or a fatty acid that can be metabolized in vivo to an omega-3 fatty acid. A fatty acid antiviral conjugate is designed to be stable in the plasma; and once inside target cells can undergo hydrolysis to safely release the individual components (i.e. antiviral agent and omega-3 fatty acid as defined herein). Because the antiviral agent is released only inside target cells, the fatty acid antiviral conjugate exhibits less side effects than the corresponding unconjugated antiviral agents. Furthermore, since omega-3 fatty acids have been shown to have anti-inflammatory properties, the corresponding fatty acid antiviral conjugates display greater anti-inflammatory properties than the corresponding unconjugated antiviral agents. This property is useful in certain cases of viral infection where the harmful inflammation hinders the efficacy of the antiviral agent. Because the overall physical properties of the fatty acid antiviral conjugates are different than the corresponding free antiviral agents, the fatty acid antiviral conjugates can be designed to target certain tissue types such as lymph nodes or liver. Selective targeting to certain tissue types can enhance the overall efficacy, as well as reduced the side effects. Therefore, fatty acid antiviral conjugates that are described herein offer new treatment options for virus-associated diseases.

### SUMMARY OF THE INVENTION

The invention is based in part on the discovery of fatty acid antiviral conjugates and their demonstrated effects in achieving improved treatment that cannot be achieved by administering fatty acids or antiviral, alone, or in simple (non-covalently linked) combination. These novel compounds are useful to treat or prevent a viral infection.

Accordingly in one aspect, a molecular conjugate is described which comprises an antiviral agent and a fatty acid directly or indirectly covalently linked, wherein the fatty acid is selected from the group consisting of omega-3 fatty acids, fatty acids that are metabolized in vivo to omega-3 fatty acids, and lipoic acid, and the conjugate is stable in the plasma and capable of hydrolysis to produce free antiviral and free fatty acid, with the proviso that the molecular conjugate is not

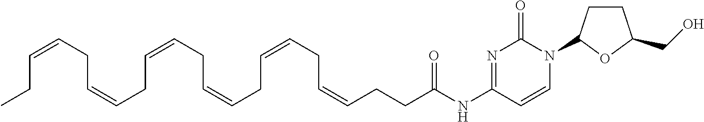

(4Z,7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-4,7,10,13,16,19-hexaenamide,

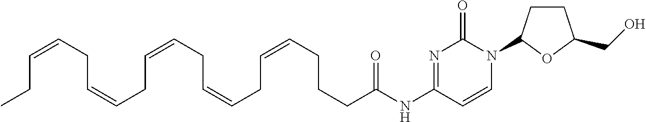

(5Z,8Z,11Z,14Z,17Z)—N-(1-((2S,5S)-5-(hydroxymethyl)tetrahydrofuran-2-yl)-2-oxo-1,2-dihydropyrimidin-4-yl)icosa-5,8,11,14,17-pentaenamide,

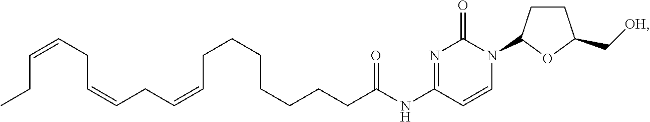

(9Z,12Z,15Z)—N-(1-((2S,5S)-5-(hydroxymethyl)tetrahydrofuran-2-yl)-2-oxo-1,2-dihydropyrimidin-4-yl)octadeca-9,12,15-trienamide,

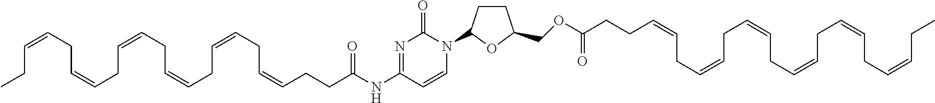

(4Z,7Z,10Z,13Z,16Z,19Z)-((2S,5S)-5-(4-((4Z,7Z,10Z,13Z,16Z,19Z)-docosa-4,7,10,13,16,19-hexaenamido)-2-oxopyrimidin-1(2H)-yl)tetrahydrofuran-2-yl)methyl docosa-4,7,10,13,16,19-hexaenoate,

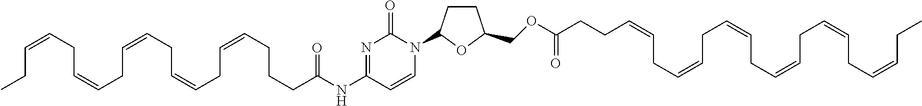

(5Z,8Z,11Z,14Z,17Z)-((2S,5S)-5-(4-((5Z,8Z,11Z,14Z,17Z)-icosa-5,8,11,14,17-pentaenamido)-2-oxopyrimidin-1(2H)-yl)tetrahydrofuran-2-yl)methyl icosa-5,8,11,14,17-pentaenoate,

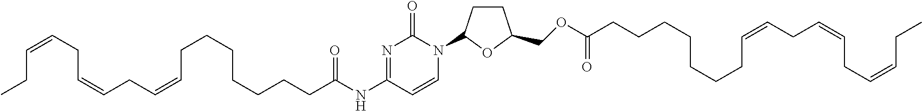

(9Z,12Z,15Z)-((2S,5S)-5-(4-((9Z,12Z,15Z)-octadeca-9,12,15-trienamido)-2-oxopyrimidin-1(2H)-yl)tetrahydrofuran-2-yl)methyl octadeca-9,12,15-trienoate,

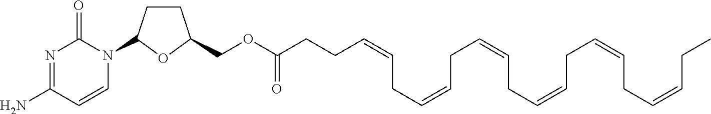

(4Z,7Z,10Z,13Z,16Z,19Z)-((2S,5S)-5-(4-amino-2-oxopyrimidin-1(2H)-yl)tetrahydrofuran-2-yl)methyl docosa-4,7,10,13,16,19-hexaenoate,

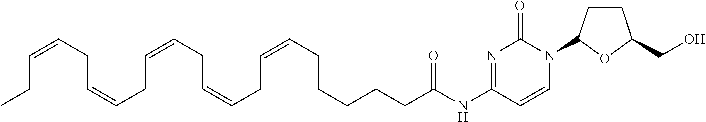

(7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-7,10,13,16,19-pentaenamide,

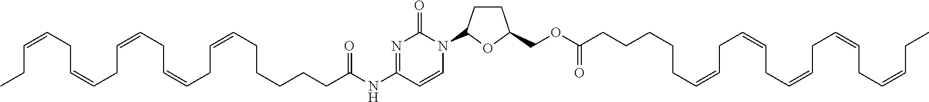

(7Z,10Z,13Z,16Z,19Z)-((2S,5S)-5-(4-((7Z,10Z,13Z,16Z,19Z)-docosa-7,10,13,16,19-pentaenamido)-2-oxopyrimidin-1(2H)-yl)tetrahydrofuran-2-yl)methyl docosa-7,10,13,16,19-pentaenoate,

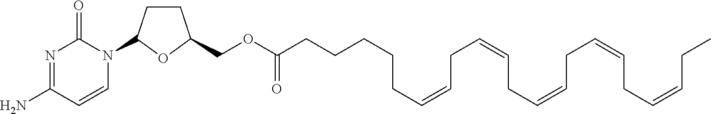

(7Z,10Z,13Z,16Z,19Z)-((2S,5S)-5-(4-amino-2-oxopyrimidin-1(2H)-yl)tetrahydrofuran-2-yl)methyl docosa-7,10,13,16,19-pentaenoate,

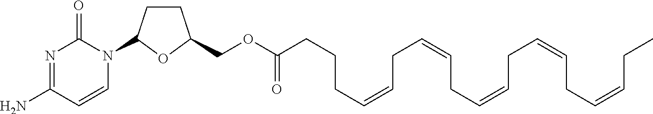

(5Z,8Z,11Z,14Z,17Z)-((2S,5S)-5-(4-amino-2-oxopyrimidin-1(2H)-yl)tetrahydrofuran-2-yl)methyl icosa-5,8,11,14,17-pentaenoate,

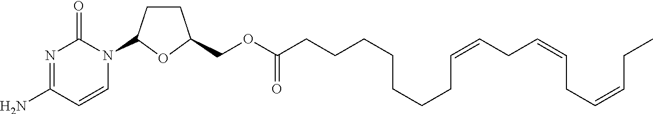

(9Z,12Z,15Z)-((2S,5S)-5-(4-amino-2-oxopyrimidin-1(2H)-yl)tetrahydrofuran-2-yl)methyl octadeca-9,12,15-trienoate, or

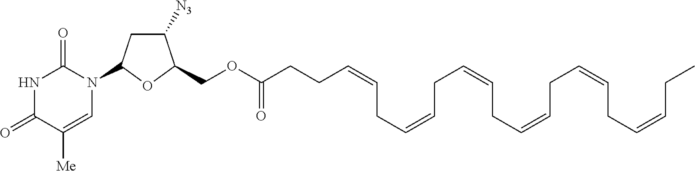

(4Z,7Z,10Z,13Z,16Z,19Z)-((2S,3S,5S)-3-azido-5-(5-methyl-2,4-dioxo-3,4-dihydropyrimidin-1(2H)-yl)tetrahydrofuran-2-yl)methyl docosa-4,7,10,13,16,19-hexaenoate.

In another aspect, a molecular conjugate is described which comprises a nucleoside antiviral agent and a fatty acid covalently linked via a phosphoramidate moiety, wherein the fatty acid is selected from the group consisting of omega-3 fatty acids, fatty acids that are metabolized in vivo to omega-3 fatty acids, and lipoic acid, and the conjugate is stable in the plasma and capable of hydrolysis to produce free phosphorylated antiviral and free fatty acid.

In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid. In other embodiments, the fatty acid is selected from eicosapentaenoic acid, docosahexaenoic acid and lipoic acid. In some embodiments, the antiviral agent is selected from the group consisting of non-nucleoside antiviral agents that include, but are not limited to, atazanavir, amprenavir, indinavir, imiquimod, lopinavir, nelfinavir, oseltamivir, ritonavir, saquinavir, rimantadine, darunavir, boceprevir, telaprevir, zanamivir, laninamivir, peramivir, VX-222, TMC 435, asunaprvir, danoprevir, daclatasvir, MK 5172, ABT-450, and GS 9190. In some embodiments, the antiviral agent is selected from the group consisting of nucleoside antiviral agents that include, but are not limited to, abacavir, aciclovir, adefovir dipivoxil, carbovir, cidofovir, didanosine, emtricitabine, entecavir, lamivudine, famciclovir, ganciclovir, penciclovir, ribarivin, sorivudine, tenofovir, zalcitabine, stavudine, zidovudine (AZT), clevudine, telbivudine, INX-189, IDX-184, GS 6620, RG 7128, RG 7432 and PSI-7977.

Nucleoside antiviral agents undergo phosphorylation in cells and targeted tissues to generate the corresponding monophosphate, diphosphate and triphosphate species. For many of these nucleoside antiviral agents, the triphosphate species is the more active metabolite. In some embodiments, the fatty acid antiviral conjugates are created by covalently joining the nucleoside moiety to the omega-3 fatty acid portion via a phosphoramidate functionality at the 5′ position of the nucleoside. With this type of phosphoramidate functionality, enzymatic degradation in targeted tissues can generate the corresponding nucleoside monophosphate and the omega-3 fatty acid. The nucleoside monophosphate, in turn, can be phosphorylated further to the corresponding triphosphate species.

In some embodiments, the hydrolysis is enzymatic. Fatty acid antiviral conjugates are inactive until they enter the cell and are hydrolyzed into the individual components to produce free antiviral agent and free fatty acid. Thus, the side effects of many antiviral agents are minimized. In some embodiments, the fatty acid antiviral conjugates are targeted preferentially to certain tissues such as liver. In Hepatitis B (HBV) or Hepatitis C (HCV) where the viral infection takes place in the liver, fatty acid antiviral conjugates that accumulate preferentially in the liver have greater efficacy. Fatty acid antiviral agents that are targeted to the liver include, but are not limited to, those conjugates having lamivudine, adefovir, entecavir, boceprevir, and telaprevir. In some embodiments, the fatty acid antiviral conjugates are targeted preferentially to certain tissues such as lymph nodes. Fatty acid antiviral agents that are targeted to the lymph nodes include, but are not limited to, those conjugates having oseltamivir, peramivir, laninamivir, zanamivir, amprenavir, indinavir, and zidovudine.

In another aspect, compounds of the Formula I are described:

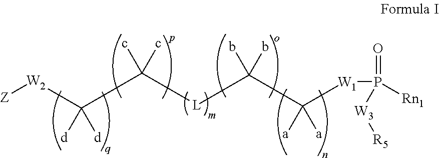

and pharmaceutically acceptable salts, hydrates, solvates, prodrugs, enantiomers, and stereoisomers thereof;

wherein

Rₙ₁ is a nucleoside antiviral agent;

W₁ and W₂ are each independently null, O, S, NH, NR, or W₁ and W₂ can be taken together can form an imidazolidine or piperazine group, with the proviso that W₁ and W₂ can not be O simultaneously;

W₃ is each independently O or NR.

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

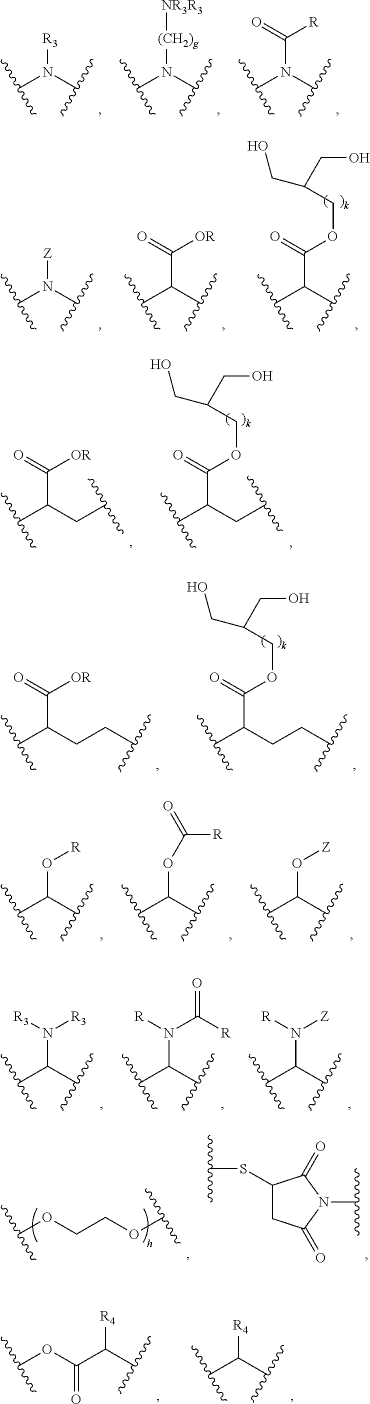

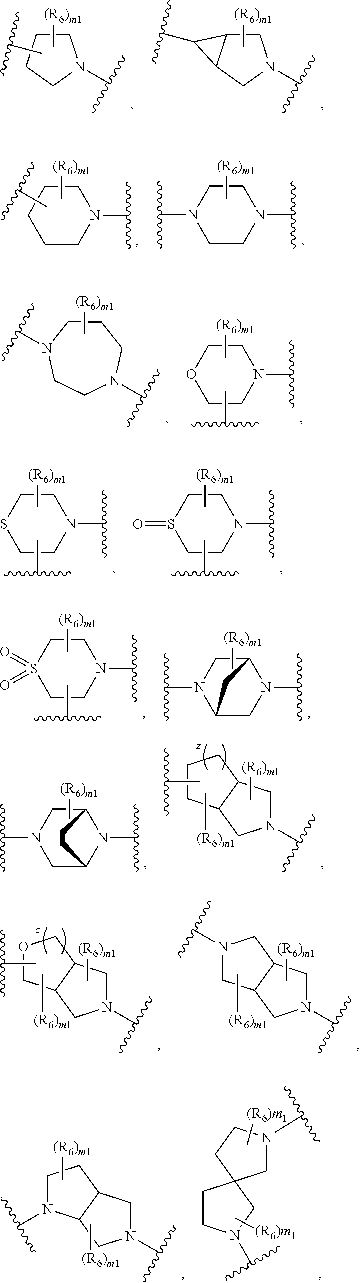

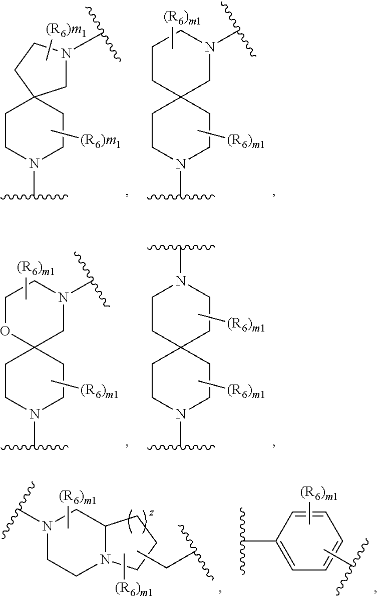

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each R₅ is independently H, aryl, heteroaryl, heterocyclic, straight or branched C₁-C₁₀ alkyl which can be optionally substituted with one or two groups selected from halogen, e, OH, NH₂, CO₂R, CONH₂, CONR₂, phenyl, C₆H₄OH, imidazole or arginine;

each Z is independently —H,

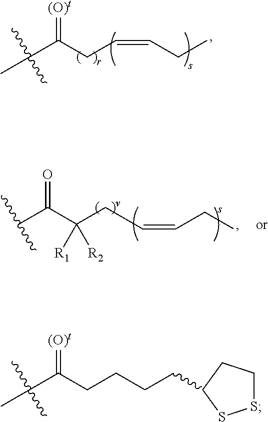

with the proviso that there is at least one

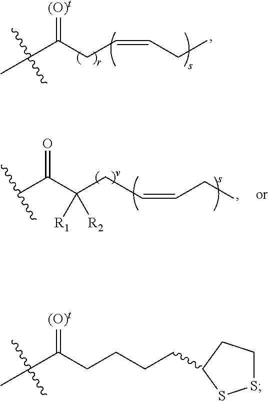

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

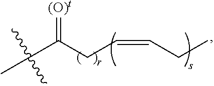

then t must be 0; and
when m, n, o, p, and q are each 0, and W₁ and W₂ are each null, then Z must not be

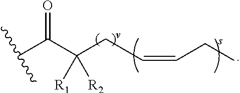

In another aspect, compounds of the Formula II are described:

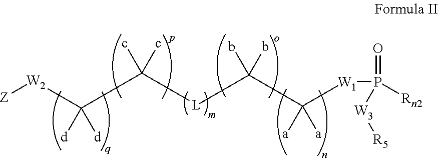

and pharmaceutically acceptable salts, hydrates, solvates, prodrugs, enantiomers, and stereoisomers thereof;

wherein Rₙ₂ is independently

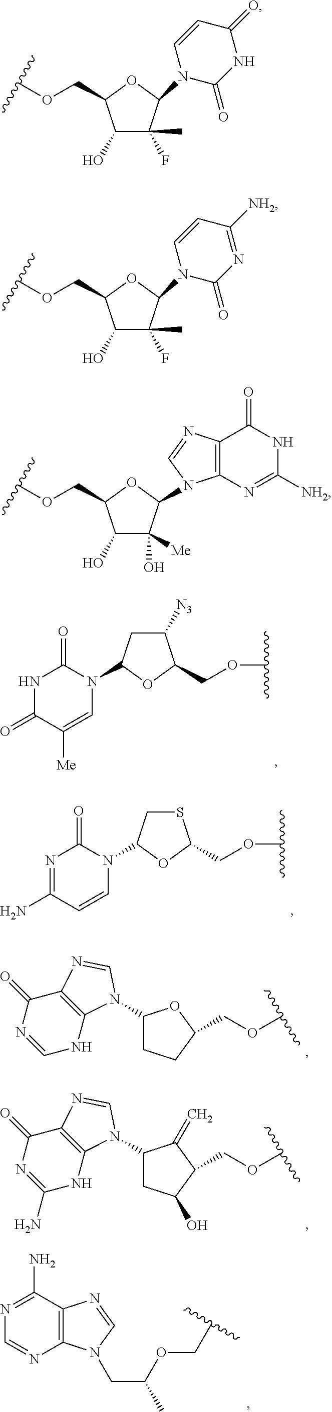

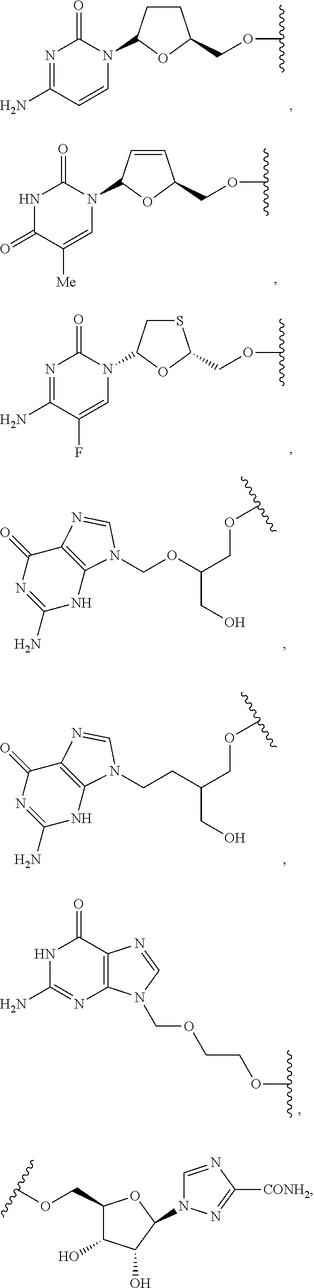

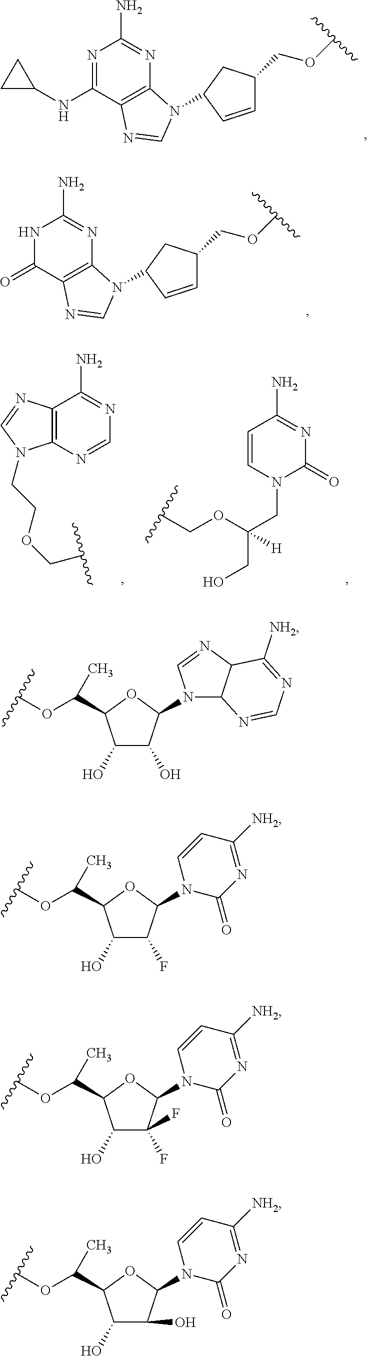

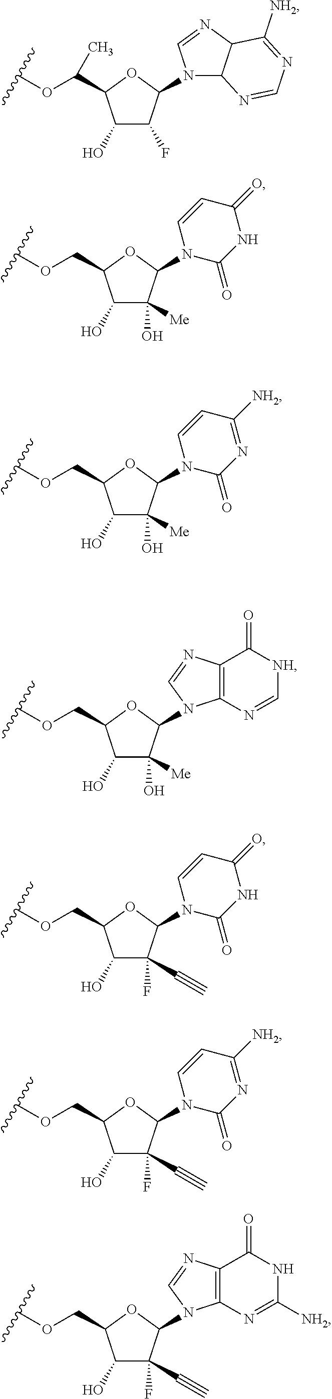

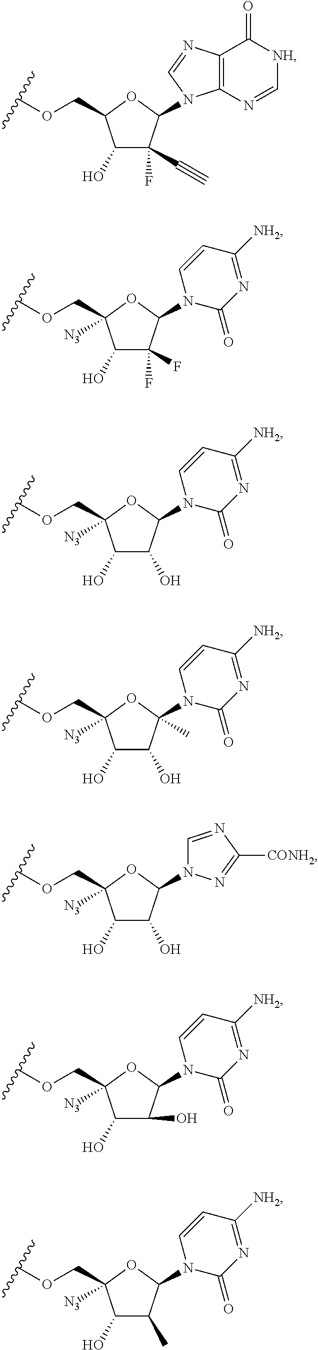

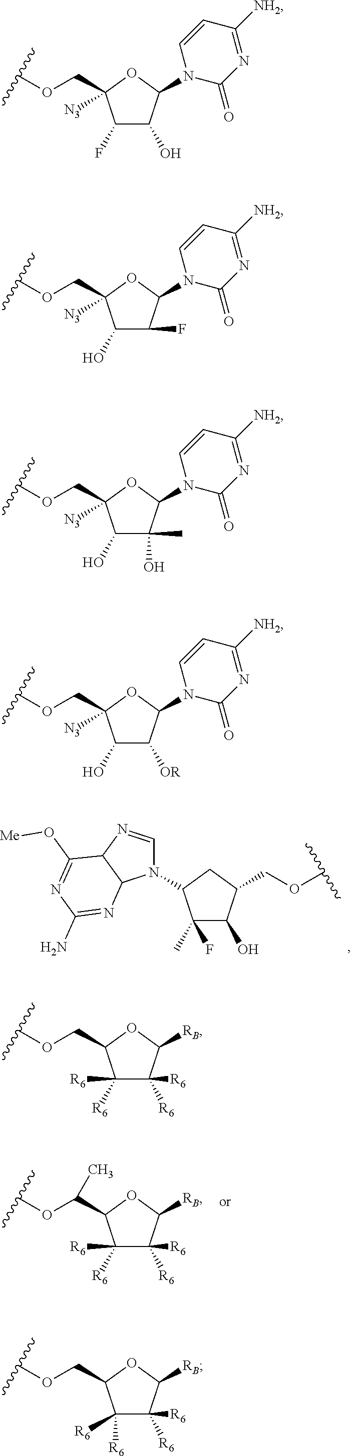

W₁ and W₂ are each independently null, O, S, NH, NR, or W₁ and W₂ can be taken together can form an imidazolidine or piperazine group, with the proviso that W₁ and W₂ can not be O simultaneously;

W₃ is each independently O or NR.

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

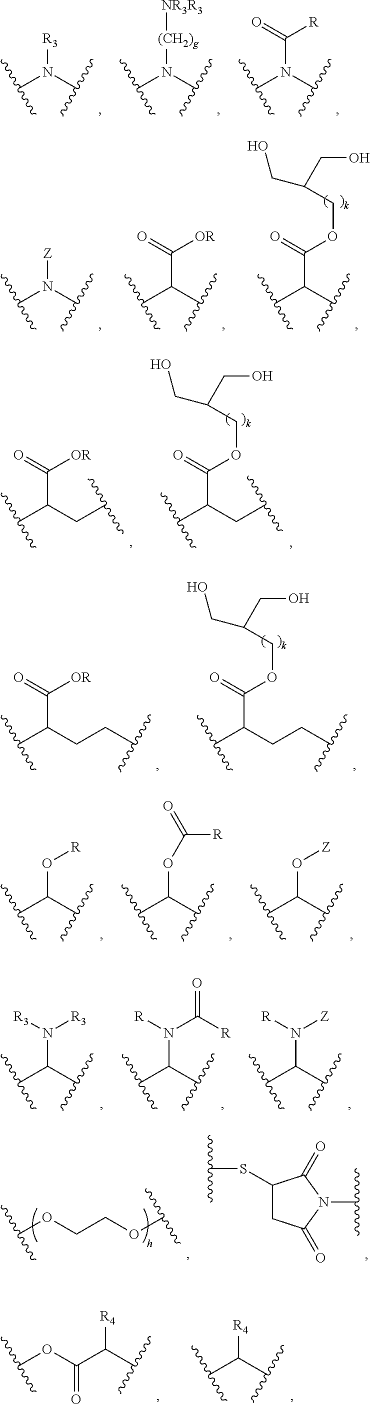

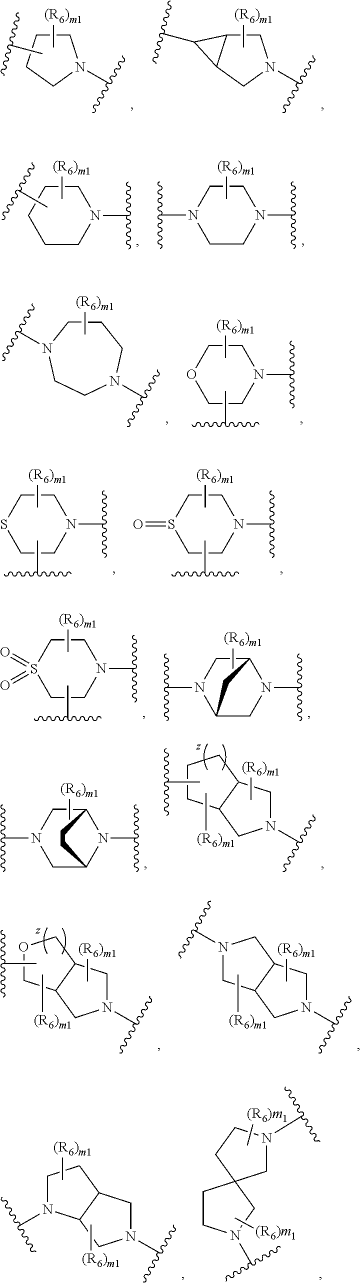

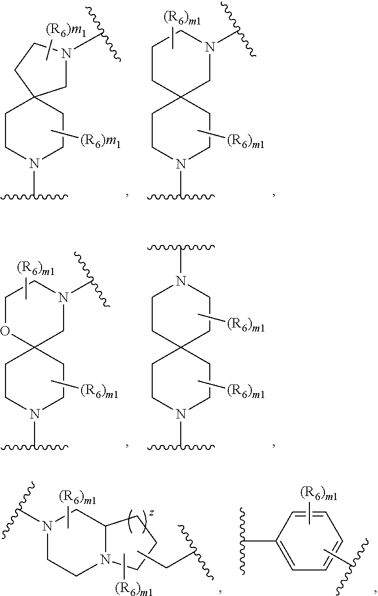

wherein the representation of L is not limited directionally left to right as is depicted, rather either the left side or the right side of L can be bound to the W₁ side of the compound of Formula II;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

R_{B} is independently

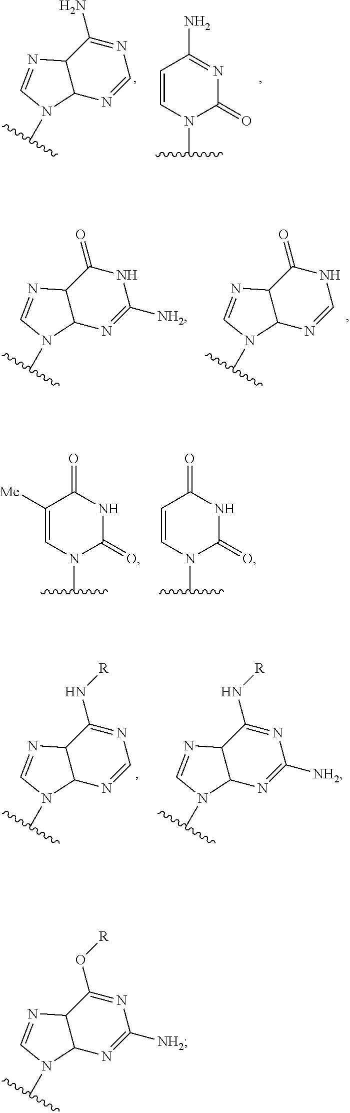

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each R₅ is independently H, aryl, heteroaryl, heterocyclic, straight or branched C₁-C₁₀ alkyl which can be optionally substituted with one or two groups selected from halogen, e, OH, NH₂, CO₂R, CONH₂, CONR₂, phenyl, C₆H₄OH, imidazole or arginine;

each Z is independently —H,

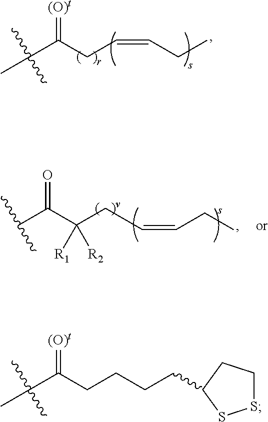

with the proviso that there is at least one

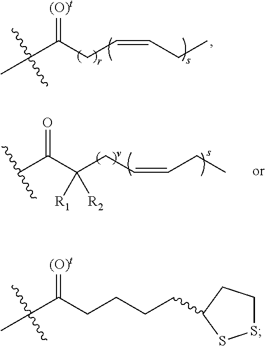

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

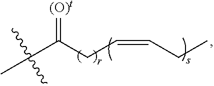

then t must be 0; and

when m, n, o, p, and q are each 0, and W₁ and W₂ are each null, then Z must not be

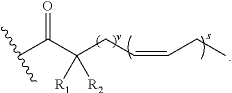

In one aspect, compounds of the Formula III are described:

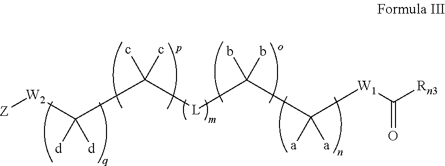

and pharmaceutically acceptable salts, hydrates, solvates, prodrugs, enantiomers, and stereoisomers thereof;

wherein

Rₙ₃ is an antiviral agent;

W₁ and W₂ are each independently null, O, S, NH, NR, or W₁ and W₂ can be taken together can form an imidazolidine or piperazine group, with the proviso that W₁ and W₂ can not be O simultaneously;

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

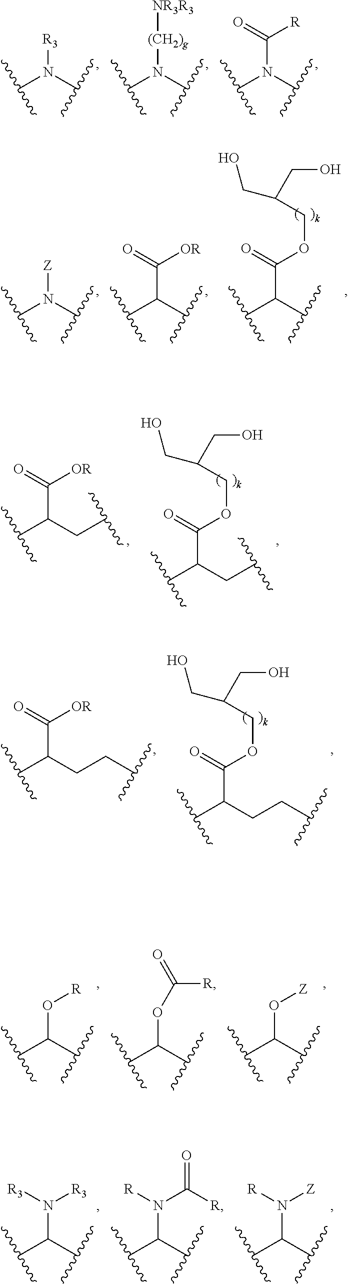

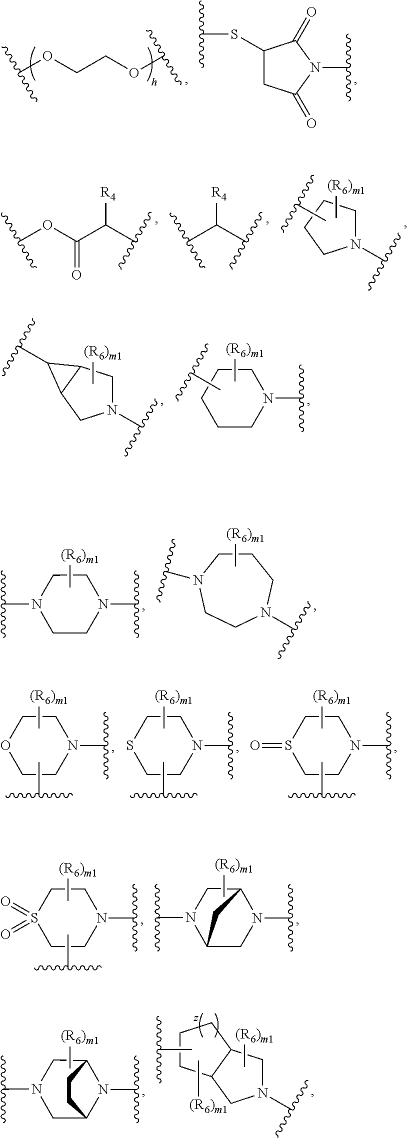

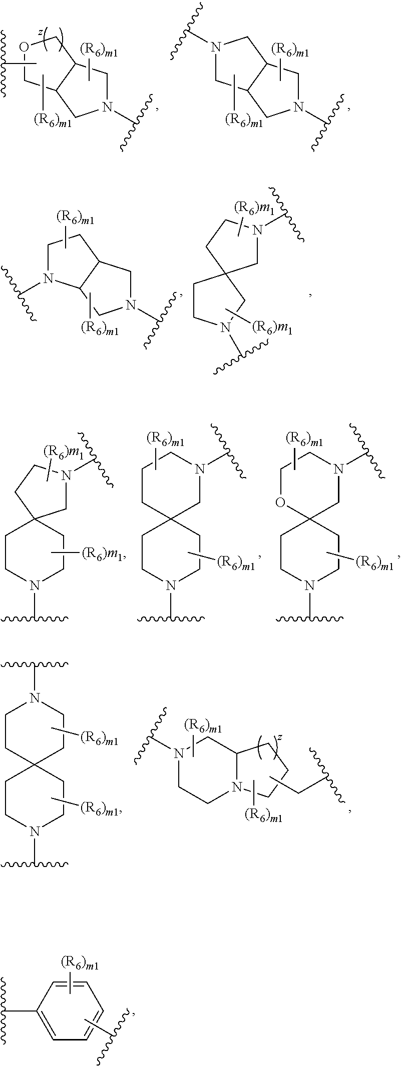

wherein the representation of L is not limited directionally left to right as is depicted, rather either the left side or the right side of L can be bound to the W₁ side of the compound of Formula III;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

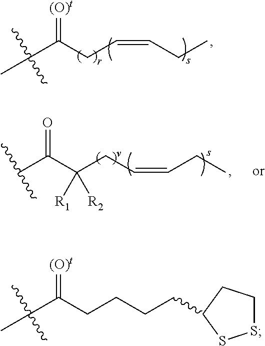

with the proviso that there is at least one

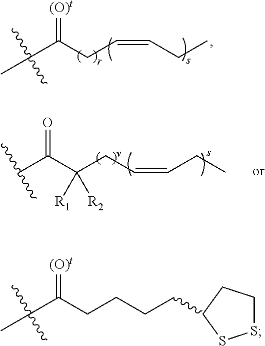

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

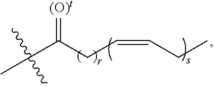

then t must be 0; and
when m, n, o, p, and q are each 0, and W₁ and W₂ are each null, then Z must not be

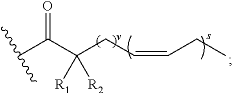

with the further proviso that the compound is not

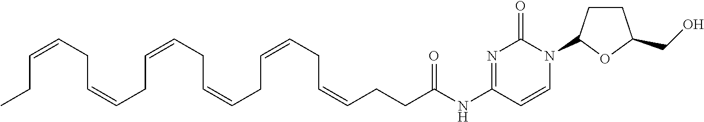

(4Z,7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-4,7,10,13,16,19-hexaenamide,

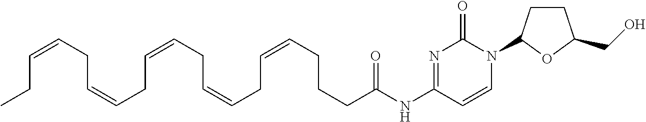

(5Z,8Z,11Z,14Z,17Z)—N-(1-((2S,5S)-5-(hydroxymethyl)tetrahydrofuran-2-yl)-2-oxo-1,2-dihydropyrimidin-4-yl)icosa-5,8,11,14,17-pentaenamide,

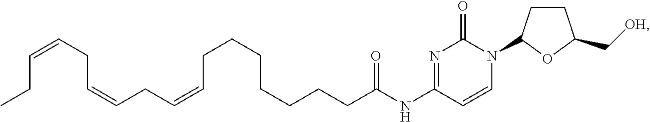

(9Z,12Z,15Z)—N-(1-((2S,5S)-5-(hydroxymethyl)tetrahydrofuran-2-yl)-2-oxo-1,2-dihydropyrimidin-4-yl)octadeca-9,12,15-trienamide,

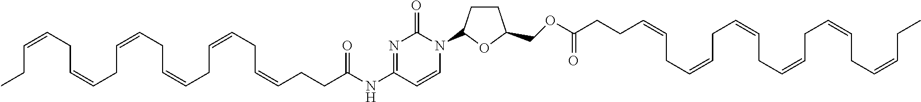

(4Z,7Z,10Z,13Z,16Z,19Z)-((2S,5S)-5-(4-((4Z,7Z,10Z,13Z,16Z,19Z)-docosa-4,7,10,13,16,19-hexaenamido)-2-oxopyrimidin-1(2H)-yl)tetrahydrofuran-2-yl)methyl docosa-4,7,10,13,16,19-hexaenoate,

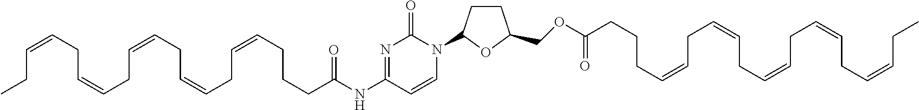

(5Z,8Z,11Z,14Z,17Z)-((2S,5S)-5-(4-((5Z,8Z,11Z,14Z,17Z)-icosa-5,8,11,14,17-pentaenamido)-2-oxopyrimidin-1(2H)-yl)tetrahydrofuran-2-yl)methyl icosa-5,8,11,14,17-pentaenoate,

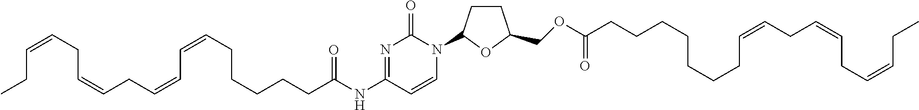

(9Z,12Z,15Z)-((2S,5S)-5-(4-((9Z,12Z,15Z)-octadeca-9,12,15-trienamido)-2-oxopyrimidin-1(2H)-yl)tetrahydrofuran-2-yl)methyl octadeca-9,12,15-trienoate,

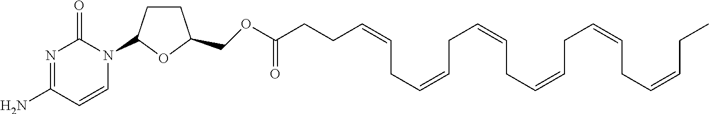

(4Z,7Z,10Z,13Z,16Z,19Z)-((2S,5S)-5-(4-amino-2-oxopyrimidin-1(2H)-yl)tetrahydrofuran-2-yl)methyl docosa-4,7,10,13,16,19-hexaenoate,

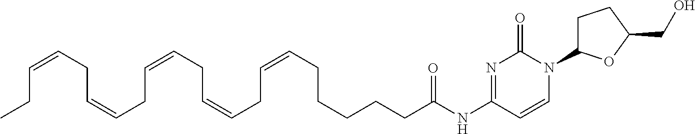

(7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-7,10,13,16,19-pentaenamide,

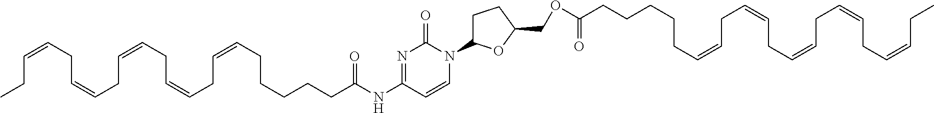

(7Z,10Z,13Z,16Z,19Z)-((2S,5S)-5-(4-((7Z,10Z,13Z,16Z,19Z)-docosa-7,10,13,16,19-pentaenamido)-2-oxopyrimidin-1(2H)-yl)tetrahydrofuran-2-yl)methyl docosa-7,10,13,16,19-pentaenoate,

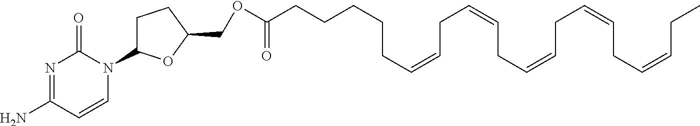

(7Z,10Z,13Z,16Z,19Z)-((2S,5S)-5-(4-amino-2-oxopyrimidin-1(2H)-yl)tetrahydrofuran-2-yl)methyl docosa-7,10,13,16,19-pentaenoate,

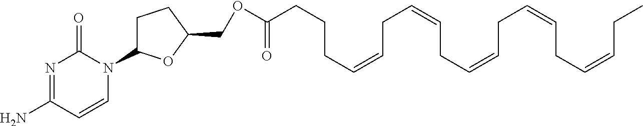

(5Z,8Z,11Z,14Z,17Z)-((2S,5S)-5-(4-amino-2-oxopyrimidin-1(2H)-yl)tetrahydrofuran-2-yl)methyl icosa-5,8,11,14,17-pentaenoate,

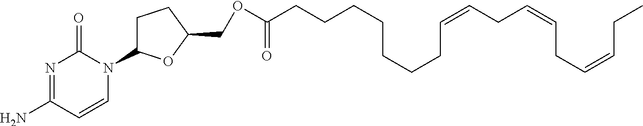

(9Z,12Z,15Z)-((2S,5S)-5-(4-amino-2-oxopyrimidin-1(2H)-yl)tetrahydrofuran-2-yl)methyl octadeca-9,12,15-trienoate, or

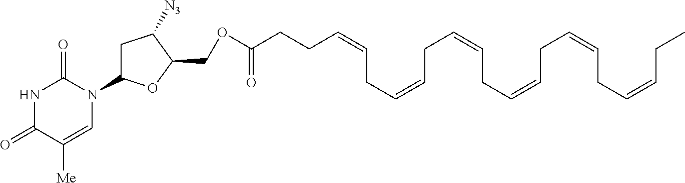

(4Z,7Z,10Z,13Z,16Z,19Z)-((2S,3S,5S)-3-azido-5-(5-methyl-2,4-dioxo-3,4-dihydropyrimidin-1(2H)-yl)tetrahydrofuran-2-yl)methyl docosa-4,7,10,13,16,19-hexaenoate.

In another aspect, fatty acid antiviral conjugates of the Formula IV are described:

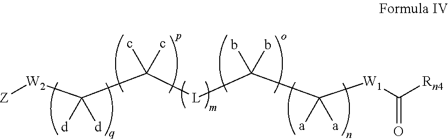

and pharmaceutically acceptable salts, hydrates, solvates, prodrugs, enantiomers, and stereoisomers thereof;

wherein

Rₙ₄ is

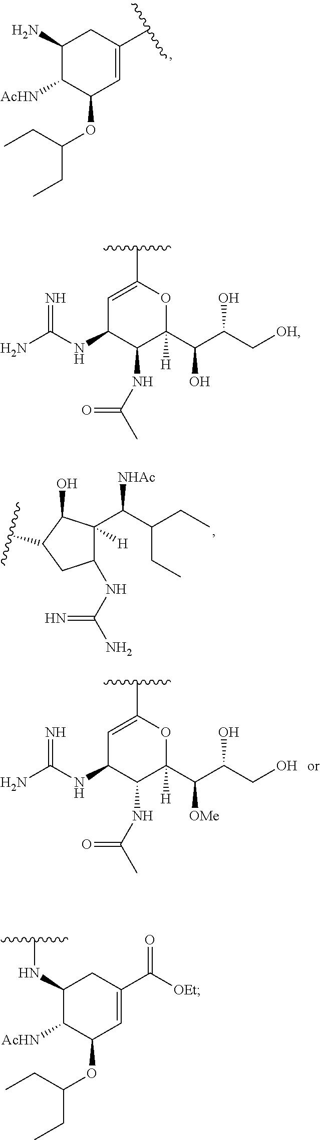

W₁ and W₂ are each independently null, O, S, NH, NR, or W₁ and W₂ can be taken together can form an imidazolidine or piperazine group, with the proviso that W₁ and W₂ can not be O simultaneously;

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

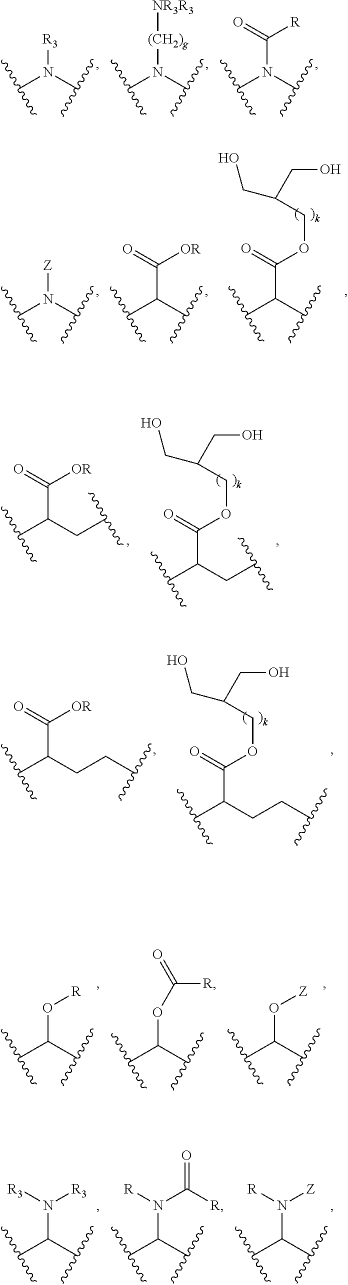

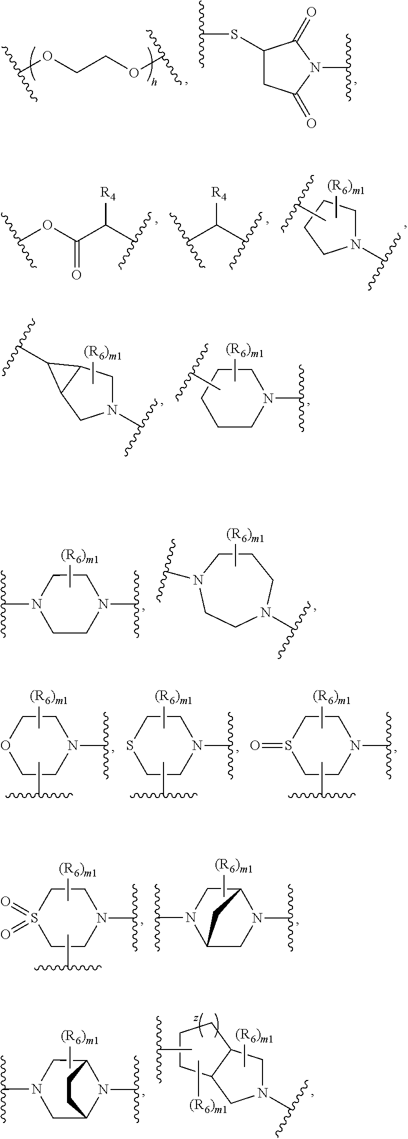

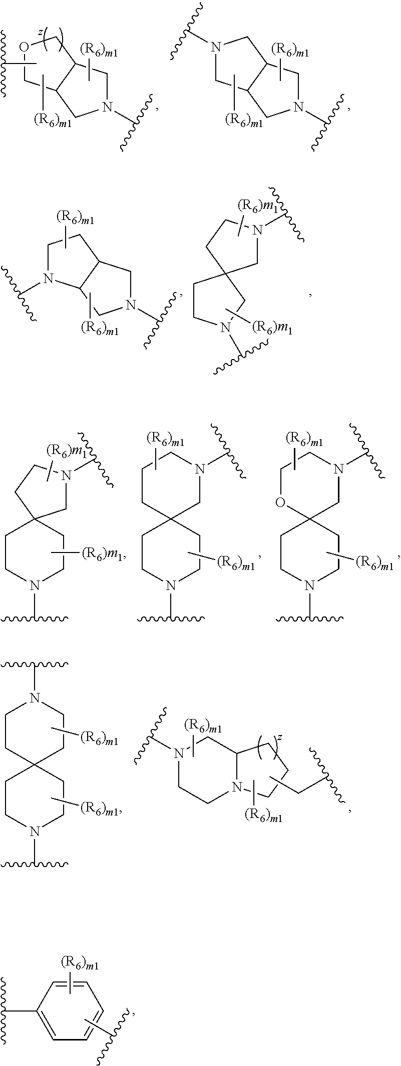

wherein the representation of L is not limited directionally left to right as is depicted, rather either the left side or the right side of L can be bound to the W₁ side of the compound of Formula IV;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

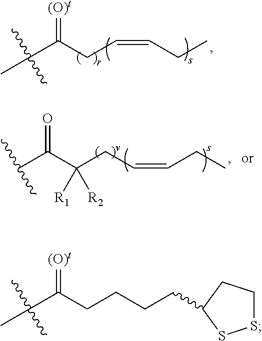

with the proviso that there is at least one

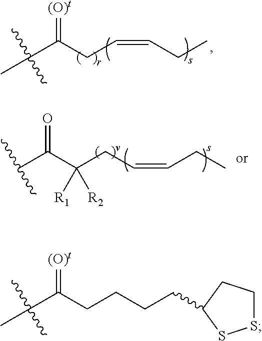

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

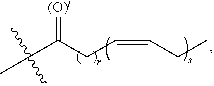

then t must be 0; and
when m, n, o, p, and q are each 0, and W₁ and W₂ are each null, then Z must not be

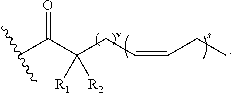

In another aspect, fatty acid antiviral conjugates of the Formula V are described:

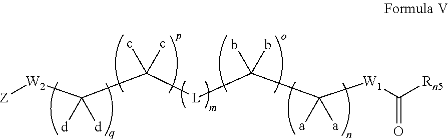

and pharmaceutically acceptable salts, hydrates, solvates, prodrugs, enantiomers, and stereoisomers thereof;

wherein

Rₙ₅ is

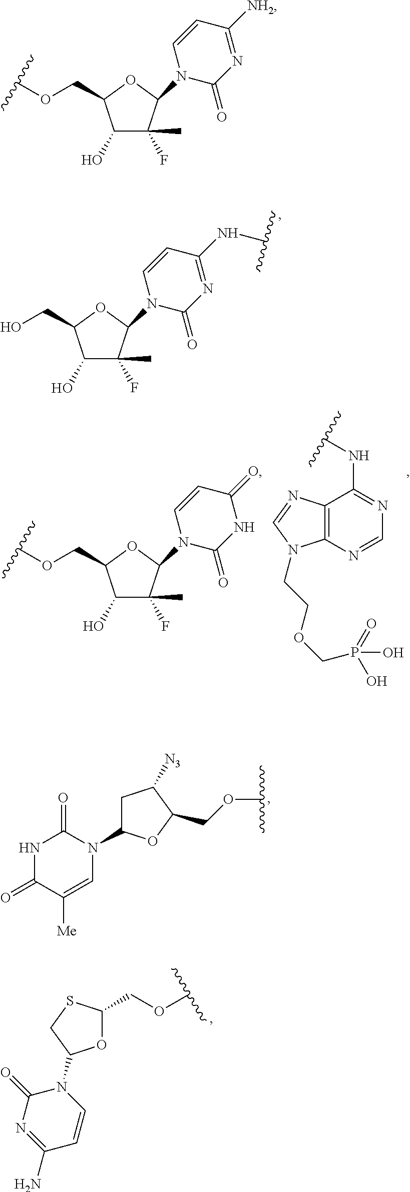

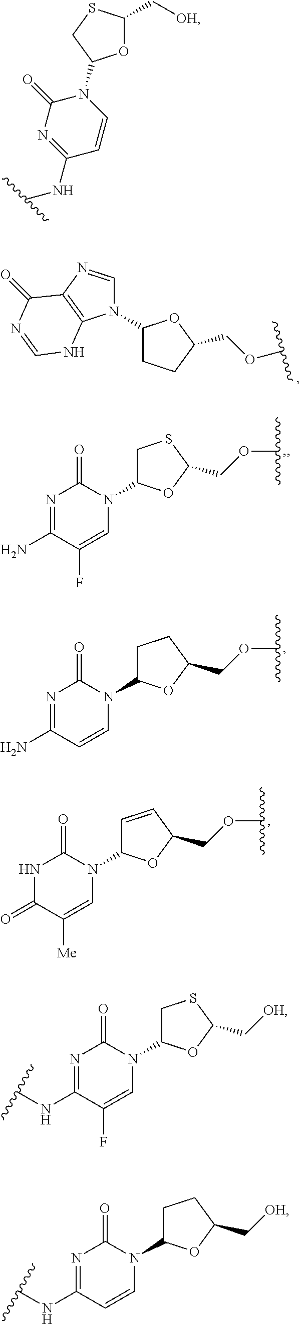

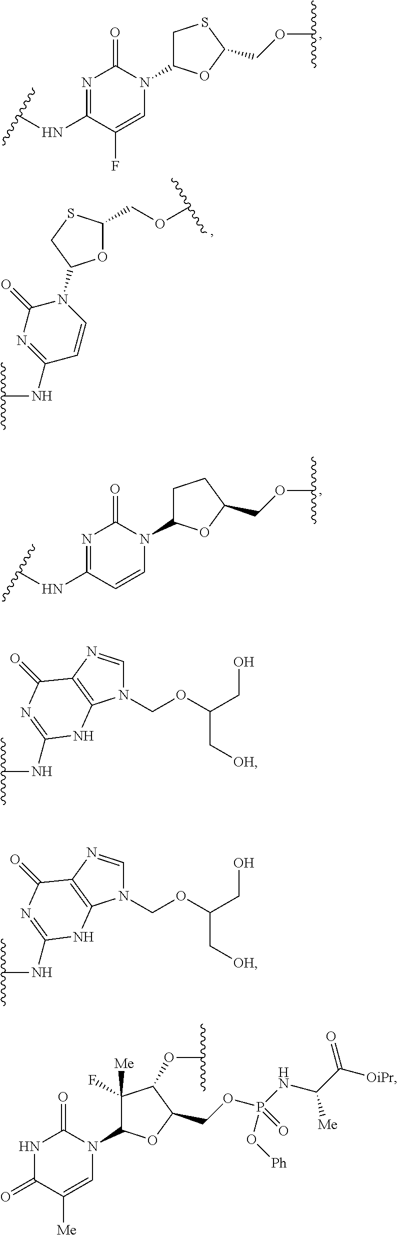

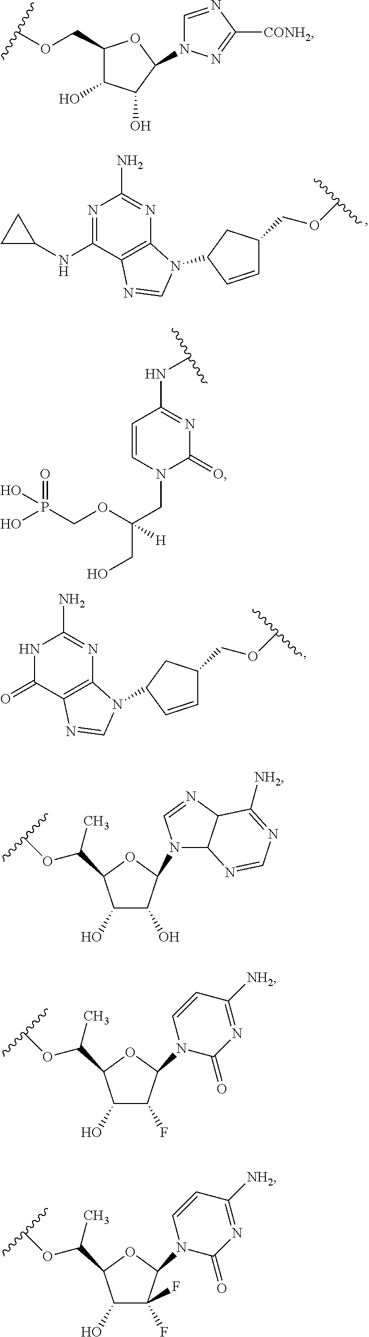

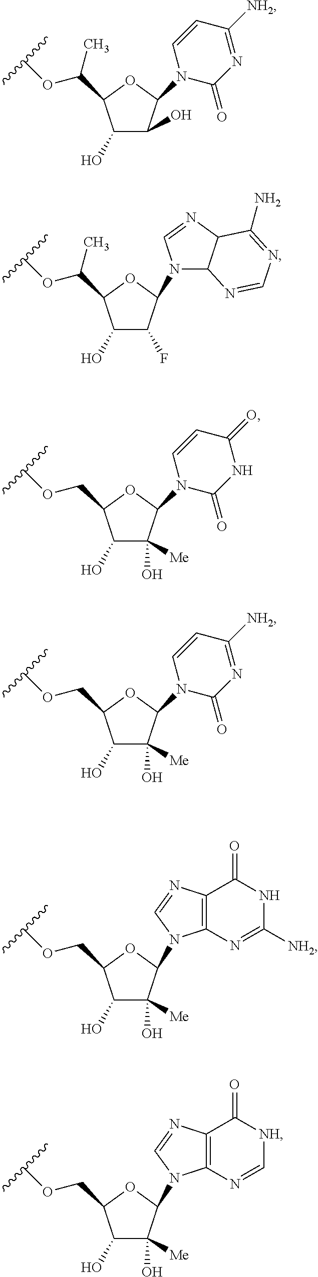 [SMILES: *OC(C)[C@H]1O[C@@H](n2ccc(N)nc2=O)[C@@H](O)[C@@H]1O ; *OC(C)[C@H]1O[C@@H](N2C=NC3C(N)=NC=NC32)[C@H](F)[C@@H]1O ; *OC[C@H]1O[C@@H](n2ccc(=O)[nH]c2=O)[C@](C)(O)[C@@H]1O ; *OC[C@H]1O[C@@H](n2ccc(N)nc2=O)[C@](C)(O)[C@@H]1O ; *OC[C@H]1O[C@@H](n2cnc3c(=O)[nH]c(N)nc32)[C@](C)(O)[C@@H]1O ; *OC[C@H]1O[C@@H](n2cnc3c(=O)[nH]cnc32)[C@](C)(O)[C@@H]1O]

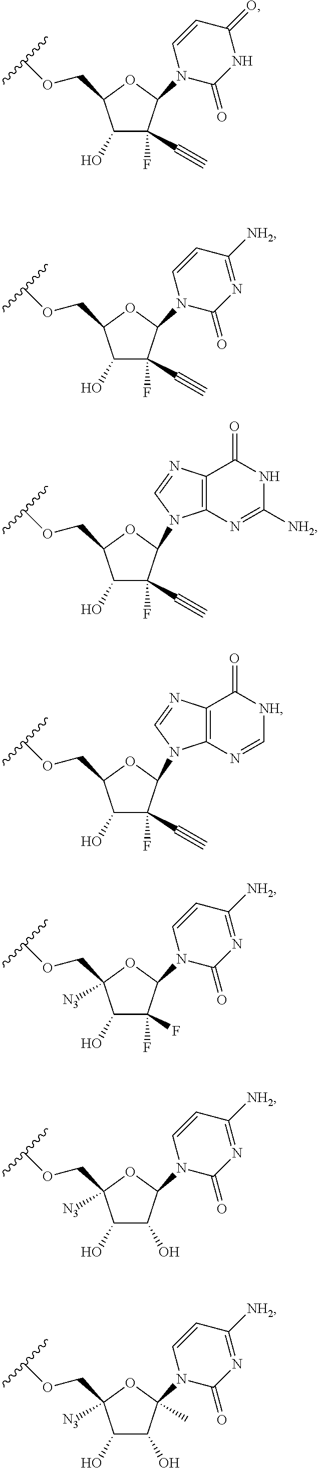

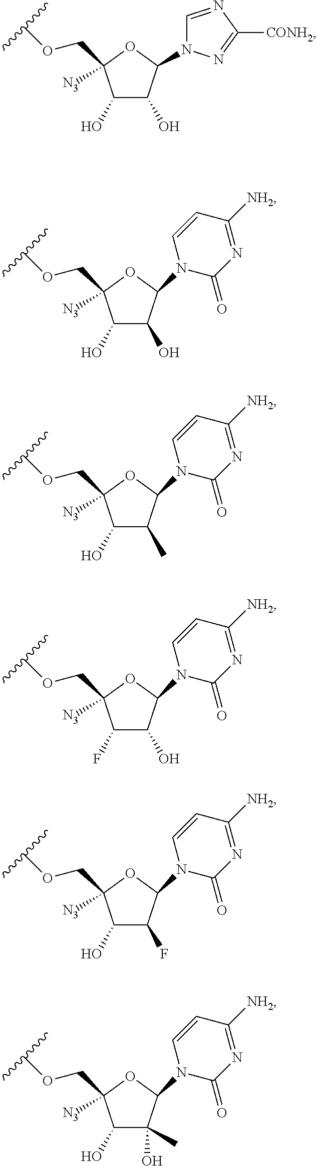 [SMILES: *OC[C@@]1(N=[N+]=[N-])O[C@@H](n2cnc(C(N)=O)n2)[C@H](O)[C@@H]1O ; *OC[C@@]1(N=[N+]=[N-])O[C@@H](n2ccc(N)nc2=O)[C@@H](O)[C@@H]1O ; *OC[C@@]1(N=[N+]=[N-])O[C@@H](n2ccc(N)nc2=O)[C@@H](C)[C@@H]1O ; *OC[C@@]1(N=[N+]=[N-])O[C@@H](n2ccc(N)nc2=O)[C@H](O)[C@@H]1F ; *OC[C@@]1(N=[N+]=[N-])O[C@@H](n2ccc(N)nc2=O)[C@@H](F)[C@@H]1O ; *OC[C@@]1(N=[N+]=[N-])O[C@@H](n2ccc(N)nc2=O)[C@](C)(O)[C@@H]1O]

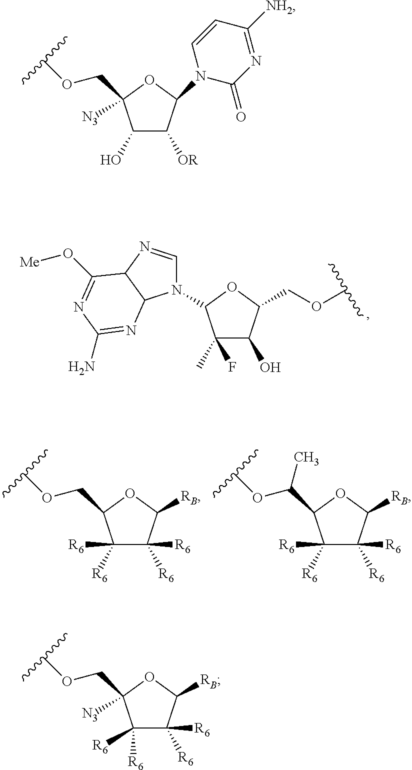

W₁ and W₂ are each independently null, O, S, NH, NR, or W₁ and W₂ can be taken together can form an imidazolidine or piperazine group, with the proviso that W₁ and W₂ can not be O simultaneously;

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

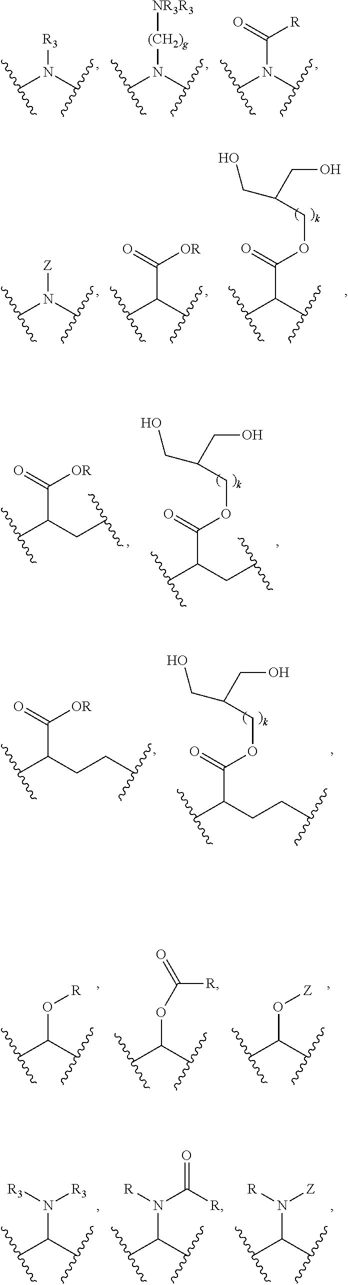

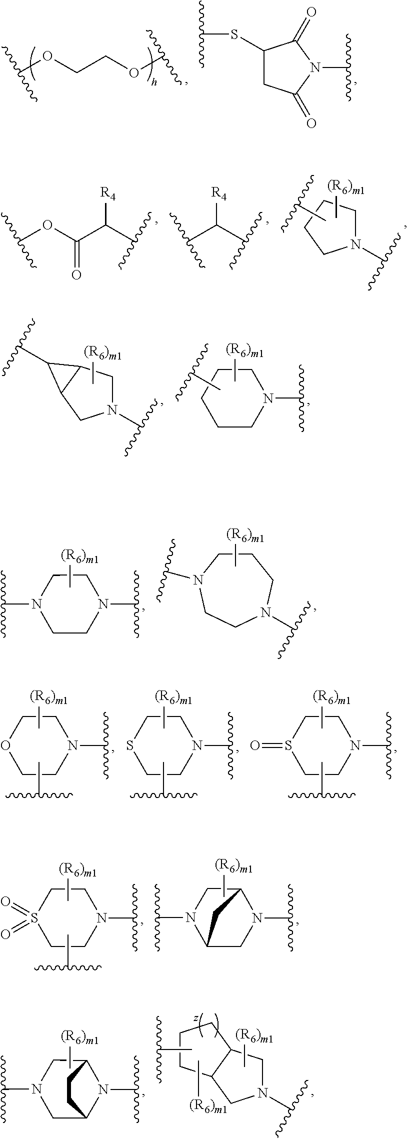

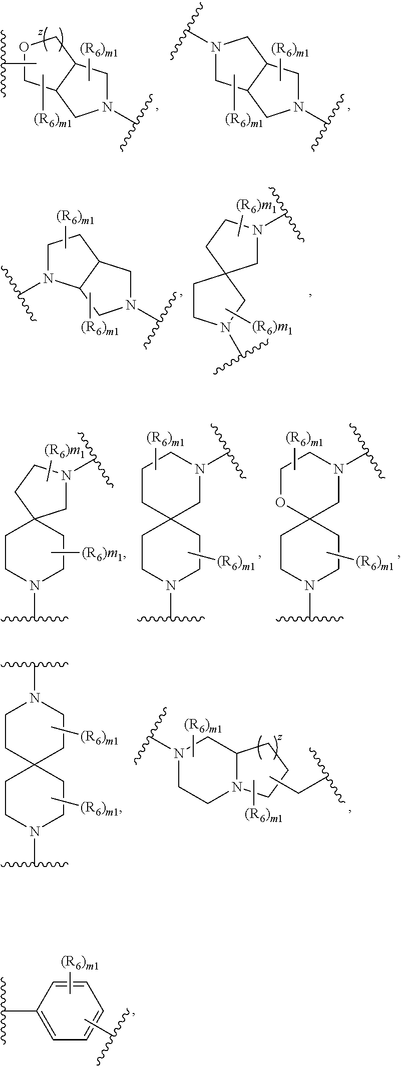

wherein the representation of L is not limited directionally left to right as is depicted, rather either the left side or the right side of L can be bound to the W₁ side of the compound of Formula V;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

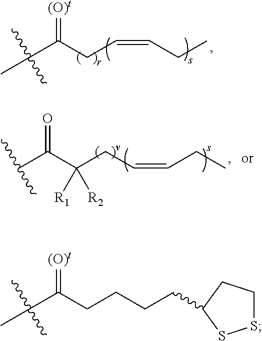

with the proviso that there is at least one

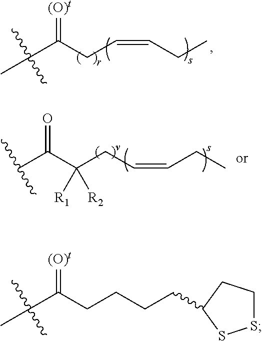

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

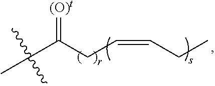

then t must be 0; and
when m, n, o, p, and q are each 0, and W₁ and W₂ are each null, then Z must not be

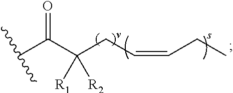

with the proviso that the compound is not

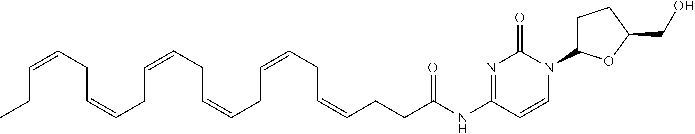

(4Z,7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-4,7,10,13,16,19-hexaenamide,

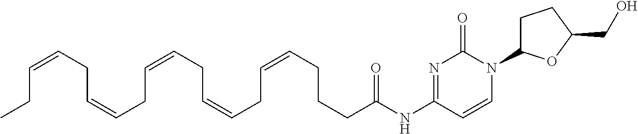

(5Z,8Z,11Z,14Z,17Z)—N-(1-((2S,5S)-5-(hydroxymethyl)tetrahydrofuran-2-yl)-2-oxo-1,2-dihydropyrimidin-4-yl)icosa-5,8,11,14,17-pentaenamide,

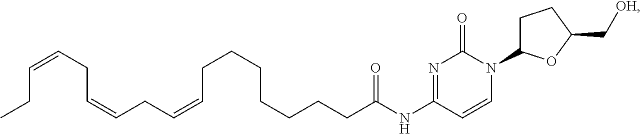

(9Z,12Z,15Z)—N-(1-((2S,5S)-5-(hydroxymethyl)tetrahydrofuran-2-yl)-2-oxo-1,2-dihydropyrimidin-4-yl)octadeca-9,12,15-trienamide,

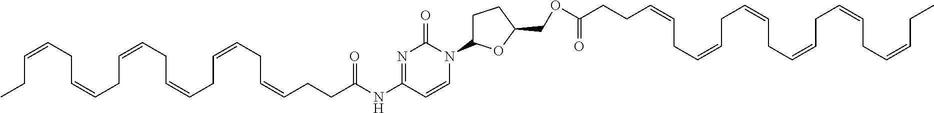

(4Z,7Z,10Z,13Z,16Z,19Z)-((2S,5S)-5-(4-((4Z,7Z,10Z,13Z,16Z,19Z)-docosa-4,7,10,13,16,19-hexaenamido)-2-oxopyrimidin-1(2H)-yl)tetrahydrofuran-2-yl)methyl docosa-4,7,10,13,16,19-hexaenoate,

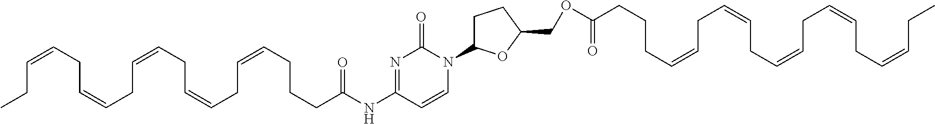

(5Z,8Z,11Z,14Z,17Z)-((2S,5S)-5-(4-((5Z,8Z,11Z,14Z,17Z)-icosa-5,8,11,14,17-pentaenamido)-2-oxopyrimidin-1(2H)-yl)tetrahydrofuran-2-yl)methyl icosa-5,8,11,14,17-pentaenoate,

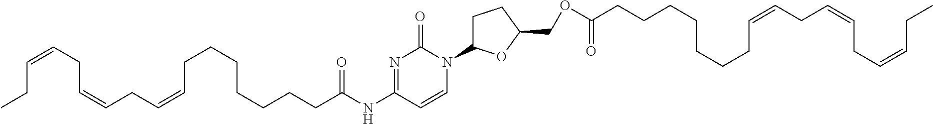

(9Z,12Z,15Z)-((2S,5S)-5-(4-((9Z,12Z,15Z)-octadeca-9,12,15-trienamido)-2-oxopyrimidin-1(2H)-yl)tetrahydrofuran-2-yl)methyl octadeca-9,12,15-trienoate,

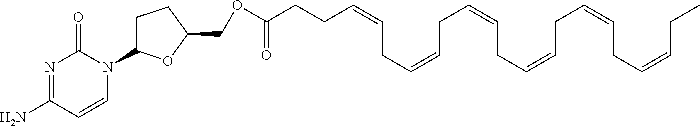

(4Z,7Z,10Z,13Z,16Z,19Z)-((2S,5S)-5-(4-amino-2-oxopyrimidin-1(2H)-yl)tetrahydrofuran-2-yl)methyl docosa-4,7,10,13,16,19-hexaenoate,

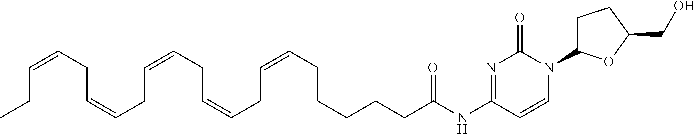

(7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-7,10,13,16,19-pentaenamide,

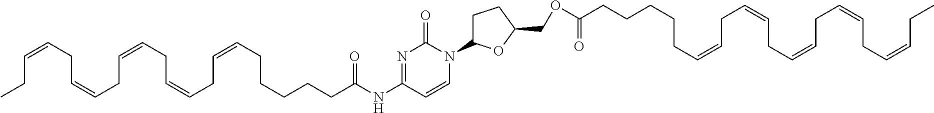

(7Z,10Z,13Z,16Z,19Z)-((2S,5S)-5-(4-((7Z,10Z,13Z,16Z,19Z)-docosa-7,10,13,16,19-pentaenamido)-2-oxopyrimidin-1(2H)-yl)tetrahydrofuran-2-yl)methyl docosa-7,10,13,16,19-pentaenoate,

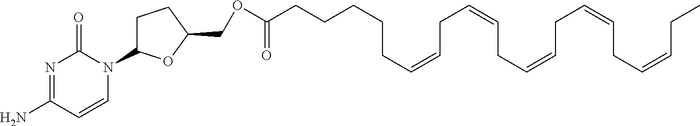

(7Z,10Z,13Z,16Z,19Z)-((2S,5S)-5-(4-amino-2-oxopyrimidin-1(2H)-yl)tetrahydrofuran-2-yl)methyl docosa-7,10,13,16,19-pentaenoate,

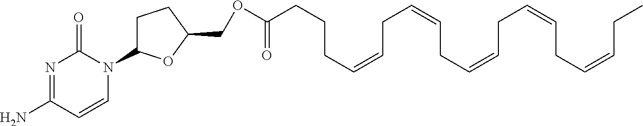

(5Z,8Z,11Z,14Z,17Z)-((2S,5S)-5-(4-amino-2-oxopyrimidin-1(2H)-yl)tetrahydrofuran-2-yl)methyl icosa-5,8,11,14,17-pentaenoate,

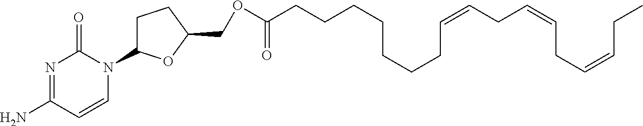

(9Z,12Z,15Z)-((2S,5S)-5-(4-amino-2-oxopyrimidin-1(2H)-yl)tetrahydrofuran-2-yl)methyl octadeca-9,12,15-trienoate, or

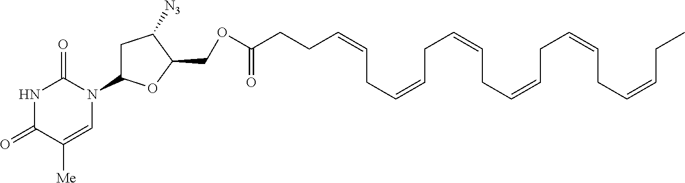

(4Z,7Z,10Z,13Z,16Z,19Z)-((2S,3S,5S)-3-azido-5-(5-methyl-2,4-dioxo-3,4-dihydropyrimidin-1(2H)-yl)tetrahydrofuran-2-yl)methyl docosa-4,7,10,13,16,19-hexaenoate.

In another aspect, fatty acid antiviral conjugates of the Formula VI are described:

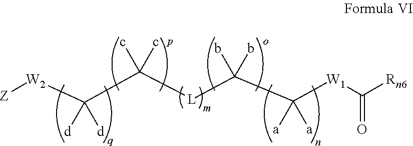

and pharmaceutically acceptable salts, hydrates, solvates, prodrugs, enantiomers, and stereoisomers thereof;

wherein

Rₙ₆ is

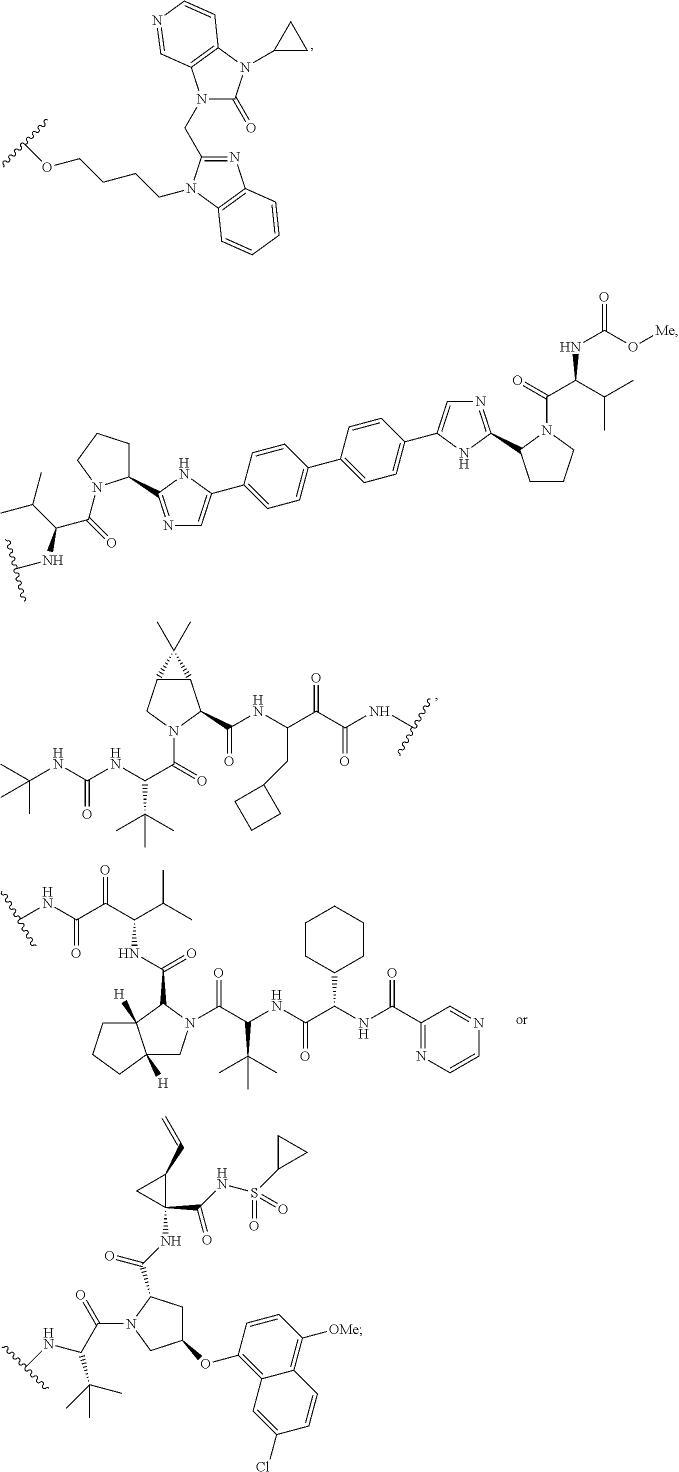

W₁ and W₂ are each independently null, O, S, NH, NR, or W₁ and W₂ can be taken together can form an imidazolidine or piperazine group, with the proviso that W₁ and W₂ can not be O simultaneously;

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

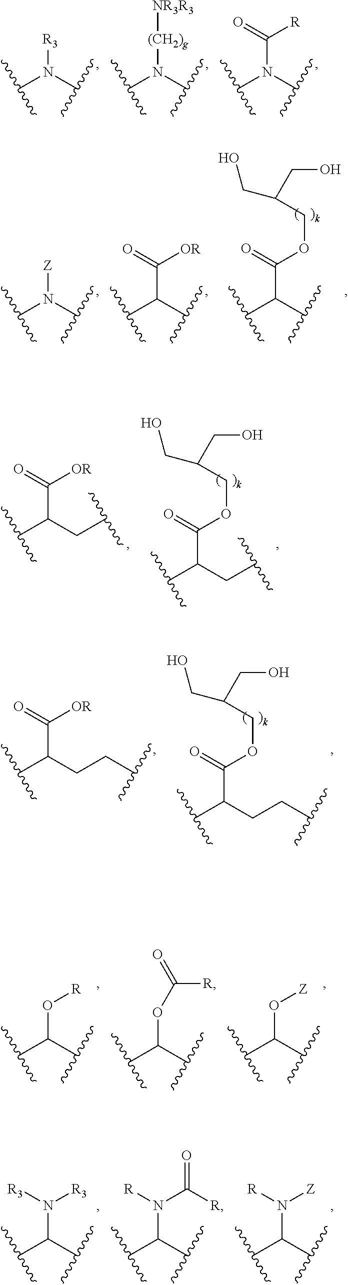

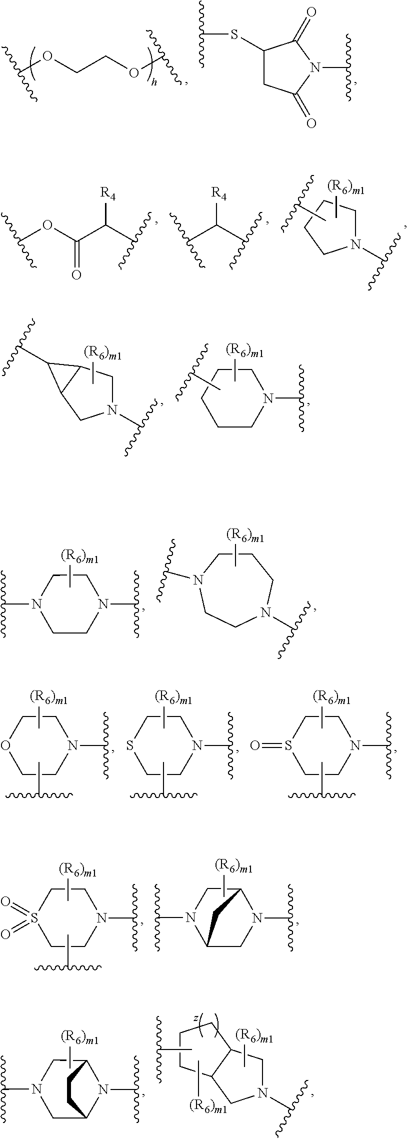

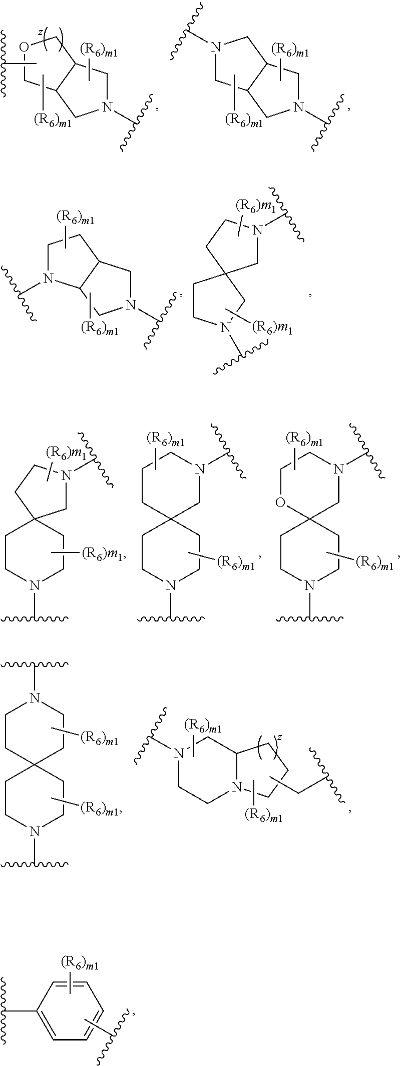

wherein the representation of L is not limited directionally left to right as is depicted, rather either the left side or the right side of L can be bound to the W₁ side of the compound of Formula VI;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

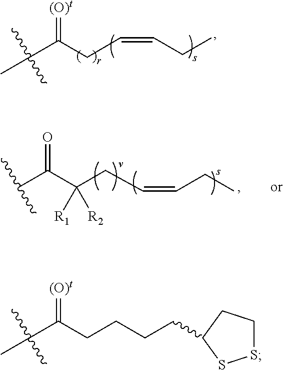

with the proviso that there is at least one

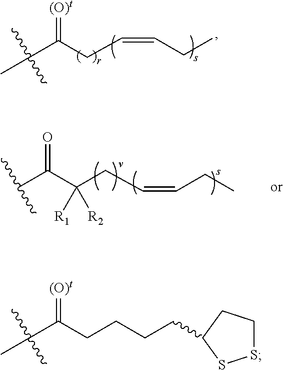

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

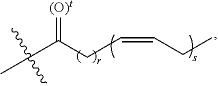

then t must be 0; and

when m, n, o, p, and q are each 0, and W₁ and W₂ are each null, then Z must not be

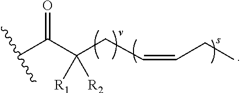

In another aspect, fatty acid antiviral conjugates of the Formula VII are described:

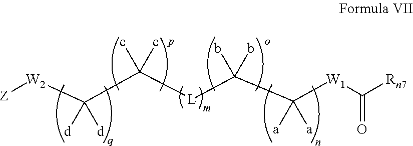

and pharmaceutically acceptable salts, hydrates, solvates, prodrugs, enantiomers, and stereoisomers thereof;

wherein

Rₙ₇ is

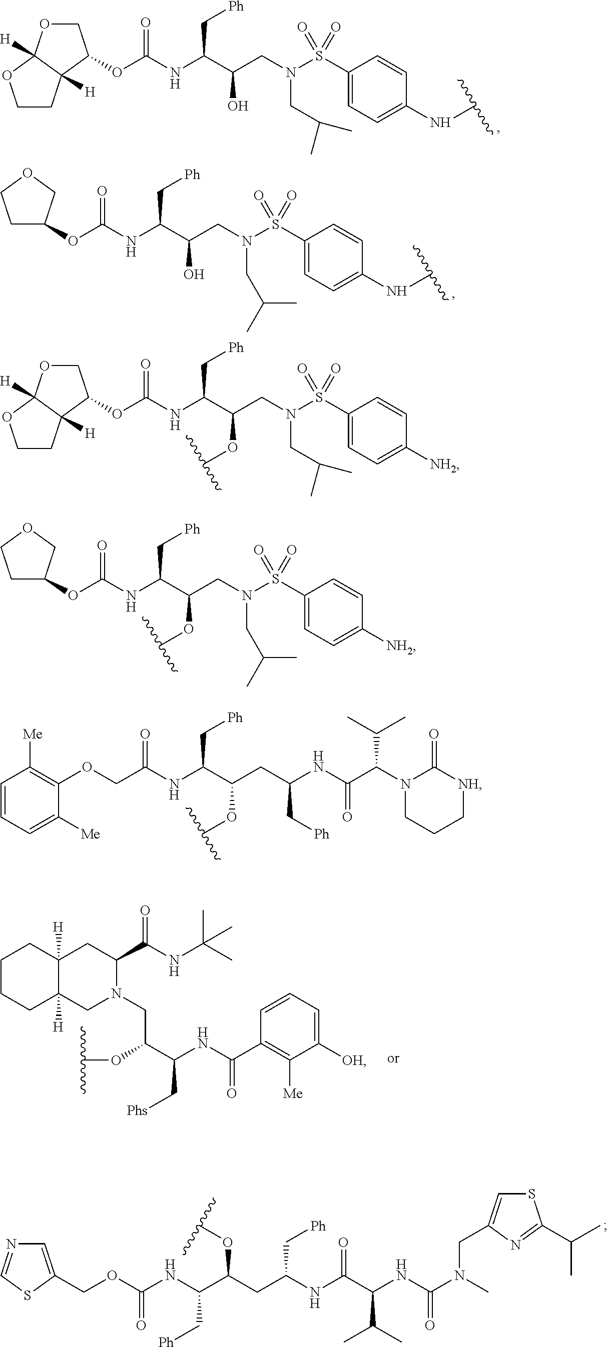

W₁ and W₂ are each independently null, O, S, NH, NR, or W₁ and W₂ can be taken together can form an imidazolidine or piperazine group, with the proviso that W₁ and W₂ can not be O simultaneously;

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

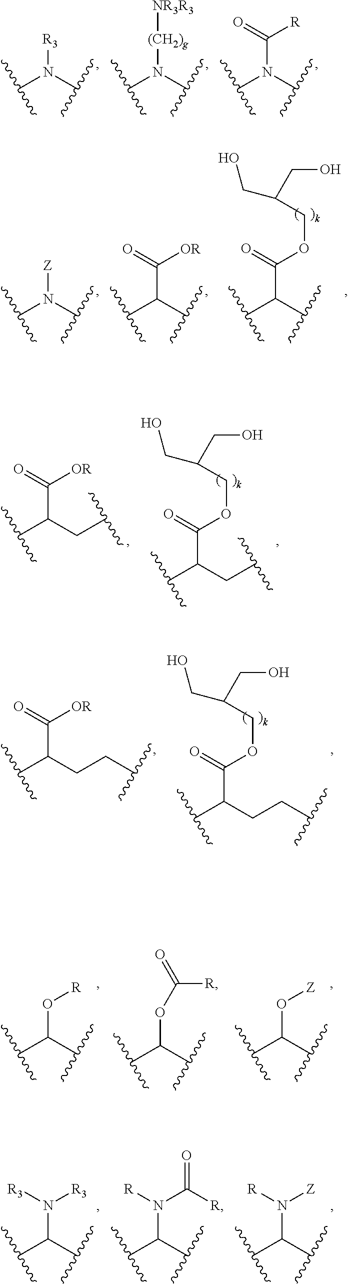

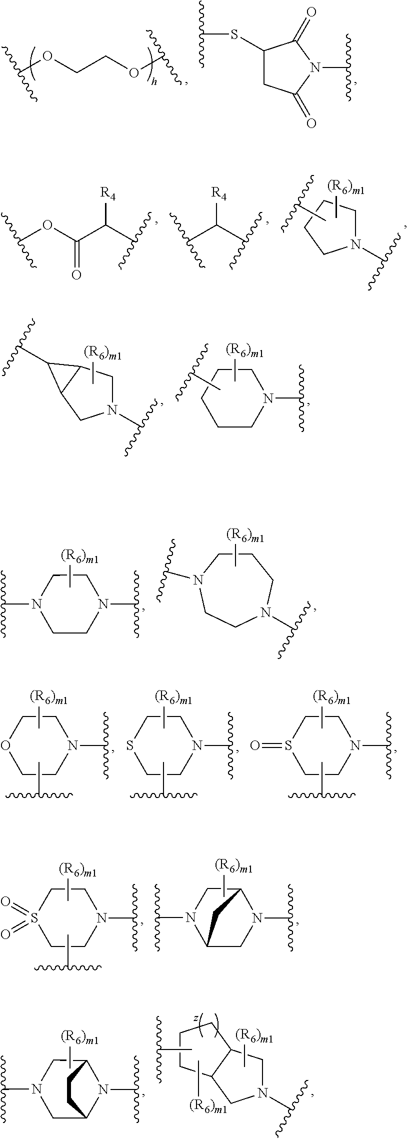

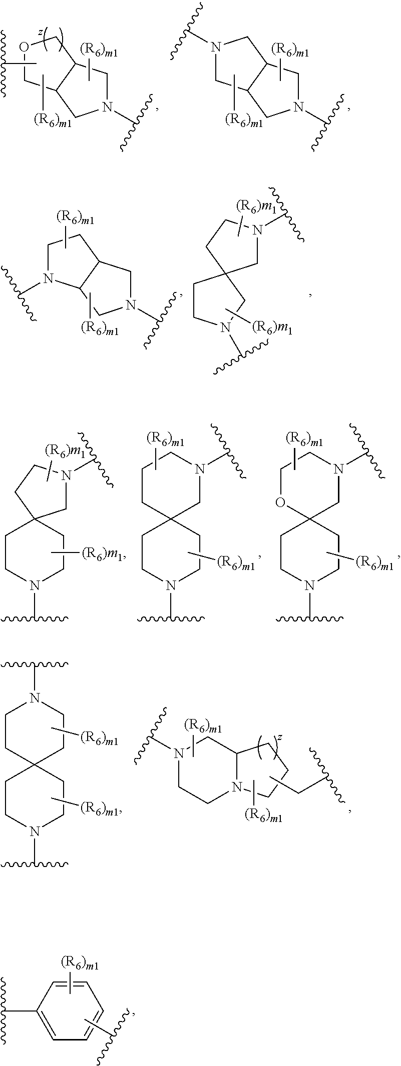

wherein the representation of L is not limited directionally left to right as is depicted, rather either the left side or the right side of L can be bound to the W₁ side of the compound of Formula VII;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

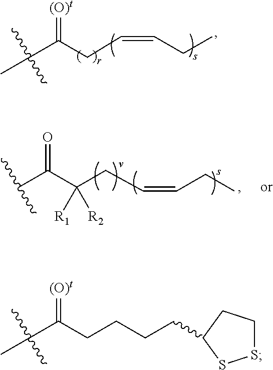

with the proviso that there is at least one

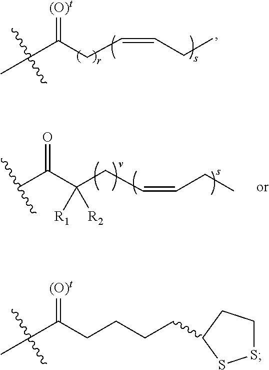

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

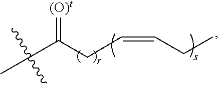

then t must be 0; and
when m, n, o, p, and q are each 0, and W₁ and W₂ are each null, then Z must not be

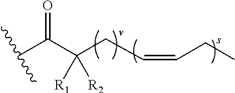

In another aspect, fatty acid antiviral conjugates of the Formula VIII are described:

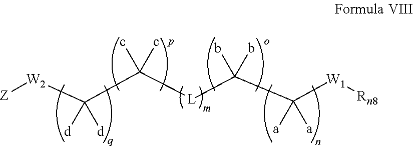

and pharmaceutically acceptable salts, hydrates, solvates, prodrugs, enantiomers, and stereoisomers thereof;

wherein Rₙ₈ is independently

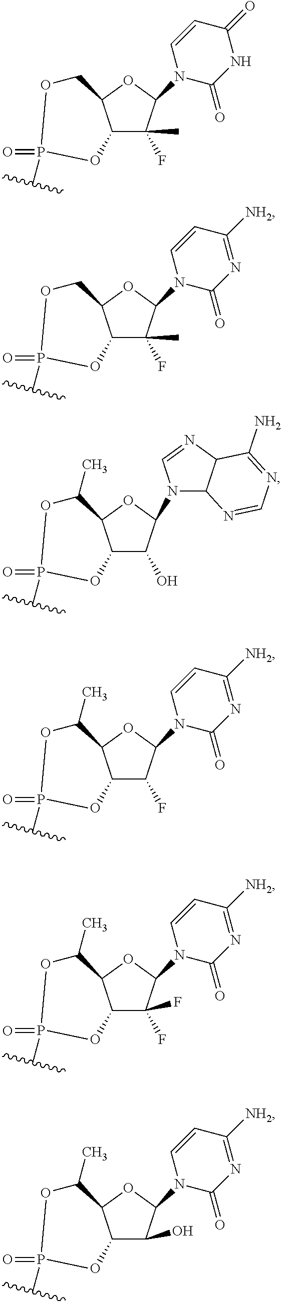

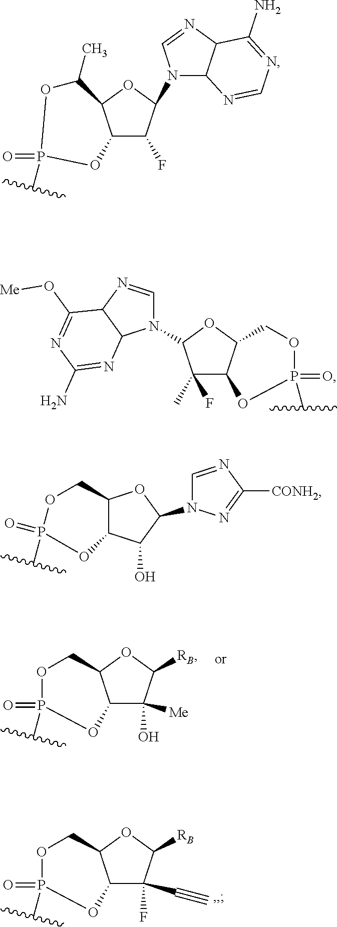

W₁ and W₂ are each independently null, O, S, NH, NR, or W₁ and W₂ can be taken together can form an imidazolidine or piperazine group, with the proviso that W₁ and W₂ can not be O simultaneously;

W₃ is each independently O or NR.

each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;

each n, o, p, and q is independently 0, 1 or 2;

each L is independently null, —O—, —S—, —S(O)—, —S(O)₂—, —S—S—, —(C₁-C₆alkyl)-, —(C₃-C₆cycloalkyl)-, a heterocycle, a heteroaryl,

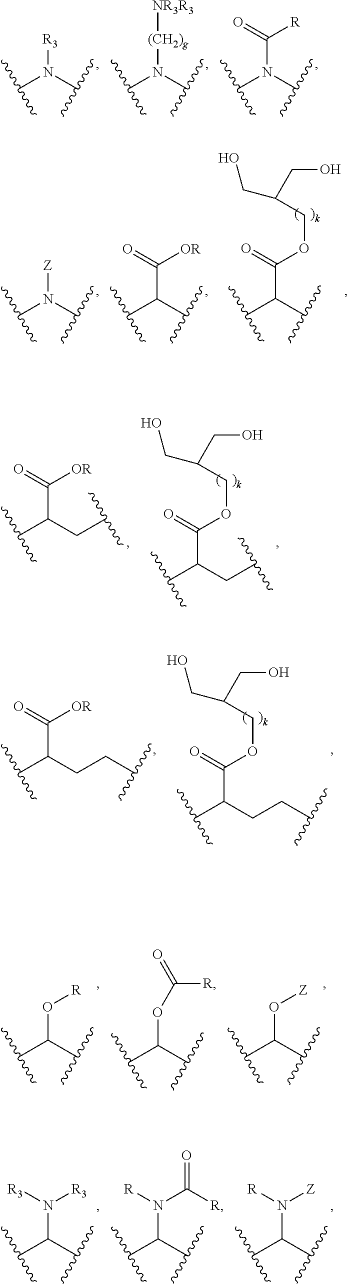

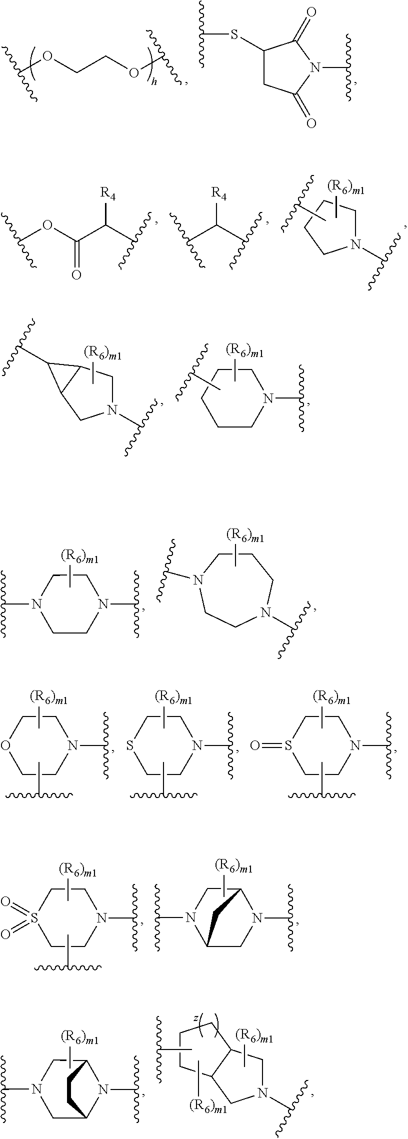

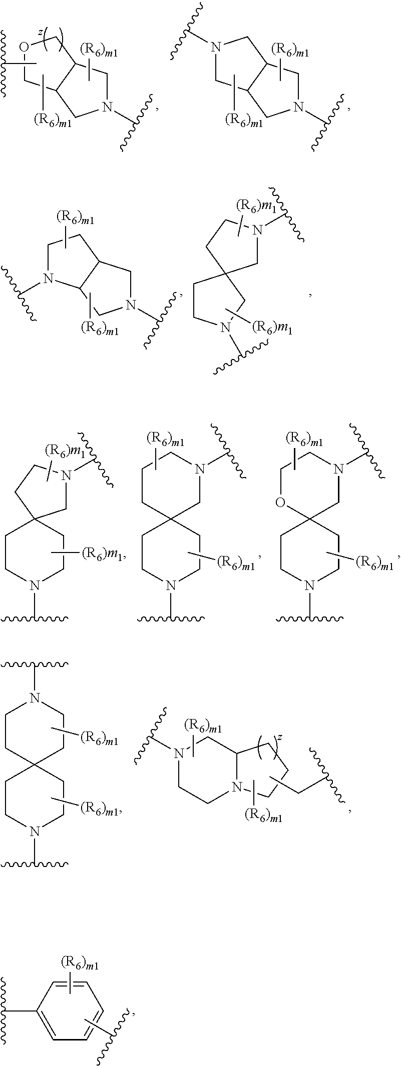

wherein the representation of L is not limited directionally left to right as is depicted, rather either the left side or the right side of L can be bound to the W₁ side of the compound of Formula VIII;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

R_{B} is independently

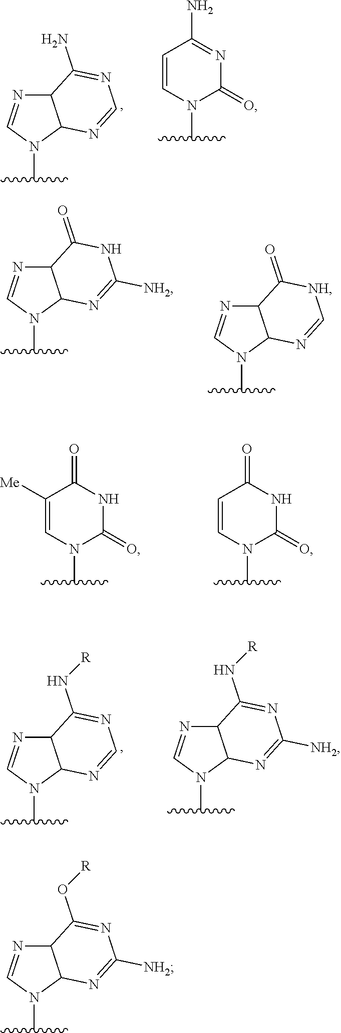

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each R₅ is independently H, aryl, heteroaryl, heterocyclic, straight or branched C₁-C₁₀ alkyl which can be optionally substituted with one or two groups selected from halogen, e, OH, NH₂, CO₂R, CONH₂, CONR₂, phenyl, C₆H₄OH, imidazole or arginine;

each Z is independently —H,

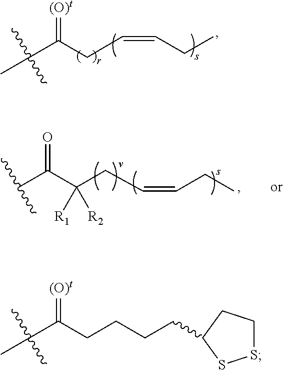

with the proviso that there is at least one

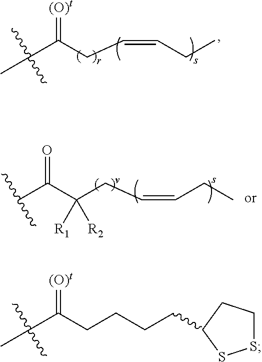

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

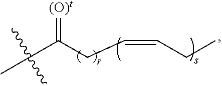

then t must be 0; and
when m, n, o, p, and q are each 0, and W₁ and W₂ are each null, then Z must not be

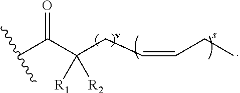

In Formula I, II, III, IV, V, VI, VII and VIII, any one or more of H may be substituted with a deuterium. It is also understood in Formula I, II, III, IV, V, VI, VII and VIII, that a methyl substituent can be substituted with a C₁-C₆ alkyl.

Also described are pharmaceutical formulations comprising at least one fatty acid antiviral conjugate.

Also described herein are methods of treating a disease susceptible to treatment with a fatty acid antiviral conjugate in a patient in need thereof by administering to the patient an effective amount of a fatty acid antiviral conjugate.

Also described herein are methods of treating or preventing a viral infection by administering to a patient in need thereof an effective amount of a fatty acid antiviral conjugate.

The invention also includes pharmaceutical compositions that comprise an effective amount of a fatty acid antiviral conjugate and a pharmaceutically acceptable carrier. The compositions are useful for treating or preventing a metabolic disease. The invention includes a fatty acid antiviral conjugate provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION OF THE INVENTION

The fatty acid antiviral conjugates have been designed to bring together at least one fatty acid and an antiviral agent into a single molecular conjugate. The activity of the fatty acid antiviral conjugates is greater than the sum of the individual components of the molecular conjugate, suggesting that the activity induced by the fatty acid conjugate is synergistic.

### DEFINITIONS

The following definitions are used in connection with the fatty acid antiviral conjugates:

The term “fatty acid antiviral conjugates” includes any and all possible isomers, stereoisomers, enantiomers, diastereomers, tautomers, pharmaceutically acceptable salts, hydrates, solvates, and prodrugs of the fatty acid antiviral conjugates described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In addition, the term “fatty acid” can also refer to medium chain fatty acids such as lipoic acid.

The term “antiviral agent” as used herein means any of the class of compounds known as either non-nucleotide antiviral agents or nucleotide antiviral agents, and any conjugates thereof. Examples of non-nucleoside antiviral agents include, but are not limited to, atazanavir, amprenavir, indinavir, imiquimod, lopinavir, nelfinavir, oseltamivir, ritonavir, saquinavir, rimantadine, darunavir, boceprevir, telaprevir, zanamivir, laninamivir, peramivir, VX-222, TMC 435, asunaprvir, danoprevir, daclatasvir, MK 5172, ABT-450, and GS 9190. Examples of nucleoside antiviral agents include, but are not limited to, abacavir, aciclovir, adefovir dipivoxil, carbovir, cidofovir, didanosine, emtricitabine, entecavir, lamivudine, famciclovir, ganciclovir, penciclovir, ribarivin, sorivudine, tenofovir, zalcitabine, stavudine, zidovudine (AZT), clevudine, telbivudine, INX-189, IDX-184, GS 6620, RG 7128, RG 7432 and PSI-7977.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharmaceutical compositions comprising an effective amount of a fatty acid antiviral conjugate and a pharmaceutically acceptable carrier. The invention includes a fatty acid antiviral conjugate provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate(1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a fatty acid antiviral conjugate is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug conjugate or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a fatty acid antiviral conjugate.

The following abbreviations are used herein and have the indicated definitions: Boc and BOC are tert-butoxycarbonyl, Boc₂O is di-tert-butyl dicarbonate, CDI is 1,1′-carbonyldiimidazole, DCC is N,N′-dicyclohexylcarbodiimide, DIEA is N,N-diisopropylethylamine, DMAP is 4-dimethylaminopyridine, DOSS is sodium dioctyl sulfosuccinate, EDC and EDCI are 1-ethyl-3-(3-dimethylaminopropyl)carbodiimide hydrochloride, EtOAc is ethyl acetate, h is hour, HATU is 2-(7-aza-1H-benzotriazole-1-yl)-1,1,3,3-tetramethyluronium hexafluorophosphate, HPMC is hydroxypropyl methylcellulose, min is minutes, Pd/C is palladium on carbon, TFA is trifluoroacetic acid, TGPS is tocopherol propylene glycol succinate, THF is tetrahydrofuran, and TNF is tumor necrosis factor.

### Compounds

In one aspect, a molecular conjugate is described which comprises an antiviral agent and a fatty acid directly or indirectly covalently linked, wherein the fatty acid is selected from the group consisting of omega-3 fatty acids, fatty acids that are metabolized in vivo to omega-3 fatty acids, and lipoic acid, and the conjugate is capable of hydrolysis to produce free antiviral agent and free fatty acid, with the proviso that the molecular conjugate is not

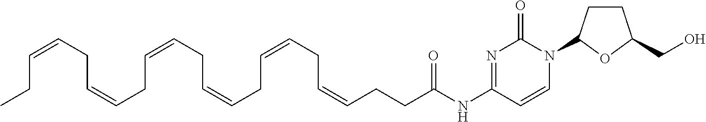

(4Z,7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-4,7,10,13,16,19-hexaenamide,

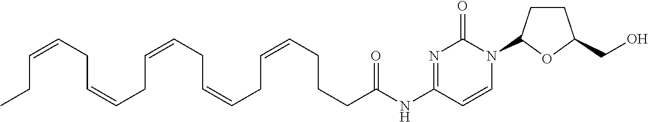

(5Z,8Z,11Z,14Z,17Z)—N-(1-((2S,5S)-5-(hydroxymethyl)tetrahydrofuran-2-yl)-2-oxo-1,2-dihydropyrimidin-4-yl)icosa-5,8,11,14,17-pentaenamide,

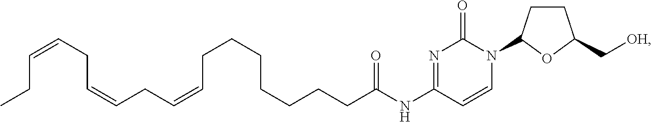

(9Z,12Z,15Z)—N-(1-((2S,5S)-5-(hydroxymethyl)tetrahydrofuran-2-yl)-2-oxo-1,2-dihydropyrimidin-4-yl)octadeca-9,12,15-trienamide,

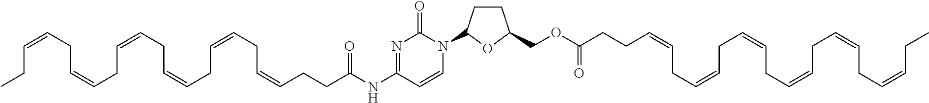

(4Z,7Z,10Z,13Z,16Z,19Z)-((2S,5S)-5-(4-((4Z,7Z,10Z,13Z,16Z,19Z)-docosa-4,7,10,13,16,19-hexaenamido)-2-oxopyrimidin-1(2H)-yl)tetrahydrofuran-2-yl)methyl docosa-4,7,10,13,16,19-hexaenoate,

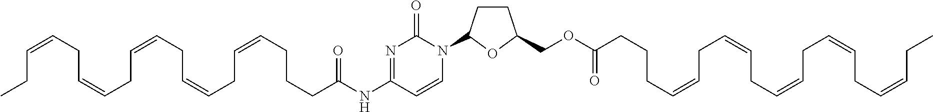

(5Z,8Z,11Z,14Z,17Z)-((2S,5S)-5-(4-((5Z,8Z,11Z,14Z,17Z)-icosa-5,8,11,14,17-pentaenamido)-2-oxopyrimidin-1(2H)-yl)tetrahydrofuran-2-yl)methyl icosa-5,8,11,14,17-pentaenoate,

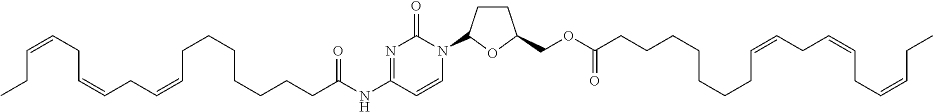

(9Z,12Z,15Z)-((2S,5S)-5-(4-((9Z,12Z,15Z)-octadeca-9,12,15-trienamido)-2-oxopyrimidin-1(2H)-yl)tetrahydrofuran-2-yl)methyl octadeca-9,12,15-trienoate,

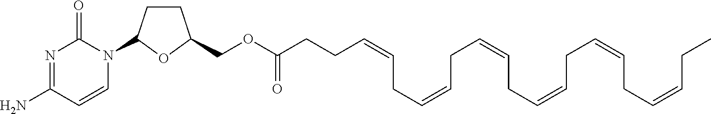

(4Z,7Z,10Z,13Z,16Z,19Z)-((2S,5S)-5-(4-amino-2-oxopyrimidin-1(2H)-yl)tetrahydrofuran-2-yl)methyl docosa-4,7,10,13,16,19-hexaenoate,

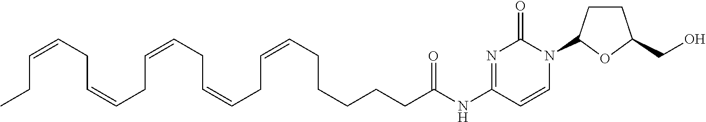

(7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-7,10,13,16,19-pentaenamide,

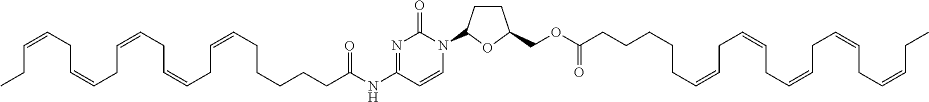

(7Z,10Z,13Z,16Z,19Z)-((2S,5S)-5-(4-((7Z,10Z,13Z,16Z,19Z)-docosa-7,10,13,16,19-pentaenamido)-2-oxopyrimidin-1(2H)-yl)tetrahydrofuran-2-yl)methyl docosa-7,10,13,16,19-pentaenoate,

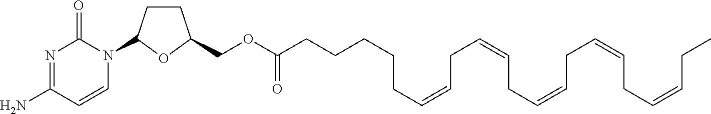

(7Z,10Z,13Z,16Z,19Z)-((2S,5S)-5-(4-amino-2-oxopyrimidin-1(2H)-yl)tetrahydrofuran-2-yl)methyl docosa-7,10,13,16,19-pentaenoate,

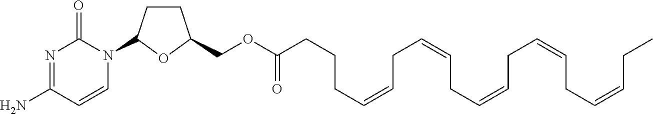

(5Z,8Z,11Z,14Z,17Z)-((2S,5S)-5-(4-amino-2-oxopyrimidin-1(2H)-yl)tetrahydrofuran-2-yl)methyl icosa-5,8,11,14,17-pentaenoate,

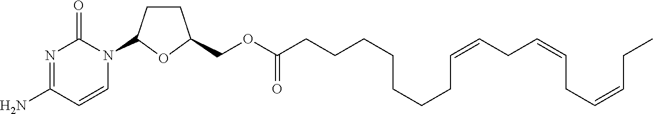

(9Z,12Z,15Z)-((2S,5S)-5-(4-amino-2-oxopyrimidin-1(2H)-yl)tetrahydrofuran-2-yl)methyl octadeca-9,12,15-trienoate, or

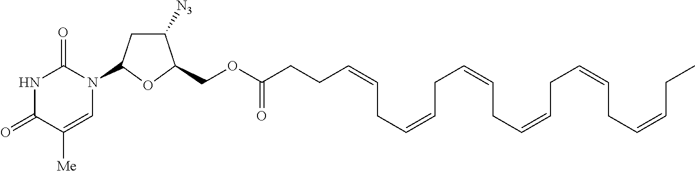

(4Z,7Z,10Z,13Z,16Z,19Z)-((2S,3S,5S)-3-azido-5-(5-methyl-2,4-dioxo-3,4-dihydropyrimidin-1(2H)-yl)tetrahydrofuran-2-yl)methyl docosa-4,7,10,13,16,19-hexaenoate.

In another aspect, a molecular conjugate is described which comprises a nucleoside antiviral agent and a fatty acid covalently linked via a phosphoramidate moiety, wherein the fatty acid is selected from the group consisting of omega-3 fatty acids, fatty acids that are metabolized in vivo to omega-3 fatty acids, and lipoic acid, and the conjugate is stable in the plasma and capable of hydrolysis to produce free antiviral and free fatty acid.

In some embodiments, the antiviral agent is selected from atazanavir, amprenavir, indinavir, imiquimod, lopinavir, nelfinavir, oseltamivir, ritonavir, saquinavir, rimantadine, darunavir, boceprevir, telaprevir, zanamivir, laninamivir, peramivir, VX-222, TMC 435, asunaprvir, danoprevir, MK 5172, ABT-450, and GS 9190. In some embodiments, the antiviral agent is selected from the group consisting of nucleoside antiviral agents that include, but are not limited to, abacavir, aciclovir, adefovir dipivoxil, carbovir, cidofovir, didanosine, emtricitabine, entecavir, lamivudine, famciclovir, ganciclovir, penciclovir, ribarivin, sorivudine, tenofovir, zalcitabine, stavudine, zidovudine (AZT), clevudine, telbivudine, INX-189, IDX-184, GS 6620, RG 7128, RG 7432 and PSI-7977.

In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, and lipoic acid. In other embodiments, the fatty acid is selected from eicosapentaenoic acid and docosahexaenoic acid. In some embodiments, the hydrolysis is enzymatic.

In some embodiments, the present invention provides fatty acid antiviral conjugates according to Formula I, II, III, IV, V, VI, VII and VIII:

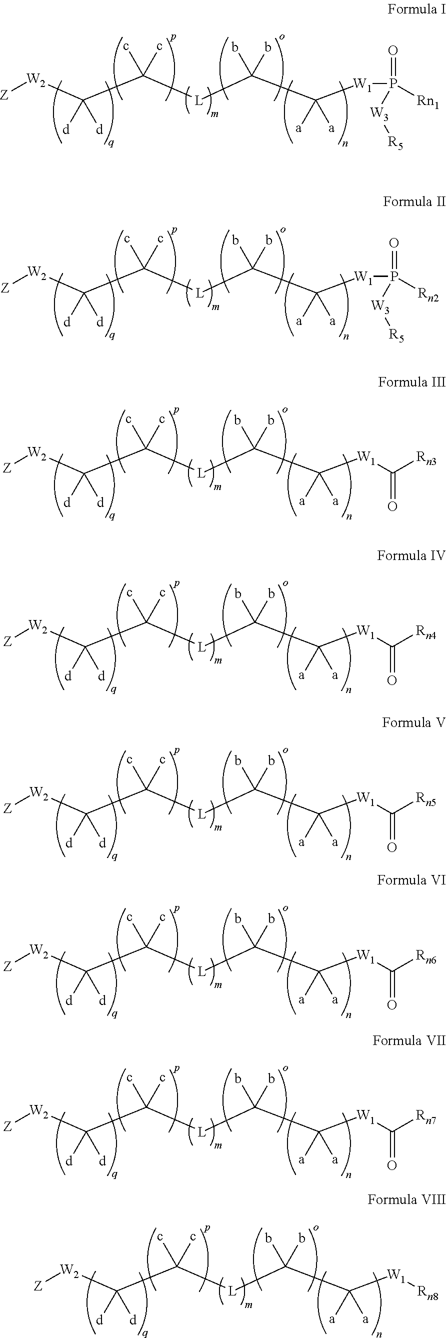

and pharmaceutically acceptable salts, hydrates, solvates, prodrugs, enantiomers and stereoisomers thereof;

wherein

W₁, W₂, a, c, b, d, e, k, m, m1, n, o, p, q, L, Z, Z′, r, s, t, v, z, Rₙ, Rₙ₁, Rₙ₂, Rₙ₃, Rₙ₄, Rₙ₅, Rₙ₆, Rₙ₇, Rₙ₈, R₁, R₂, R₃, R₄, R and R₆ are as defined above for Formula I-VIII,

with the proviso that there is at least one of

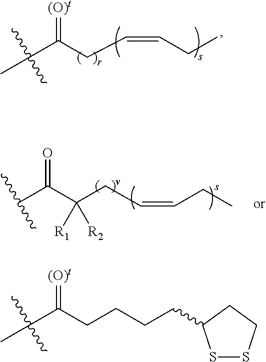

in the compound.

In some embodiments, one Z is

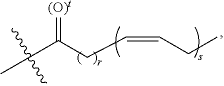

and r is 2.

In some embodiments, one Z is

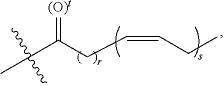

and r is 3.

In some embodiments, one Z is

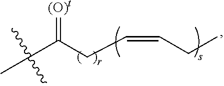

and r is 7.

In other embodiments, one Z is

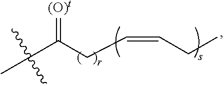

and s is 3.

In some embodiments, one Z is

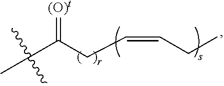

and s is 5.

In some embodiments, one Z is

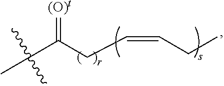

and s is 6.

In some embodiments, one Z is

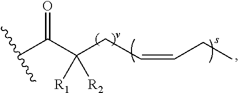

and v is 1.

In other embodiments, one Z is

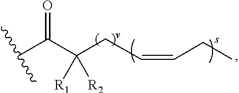

and v is 2.

In some embodiments, one Z is

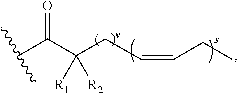

and v is 6.

In some embodiments, one Z is

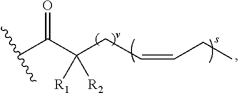

and s is 3.

In some embodiments, one Z is

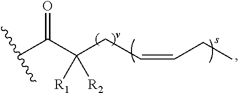

and s is 5.

In other embodiments, one Z is

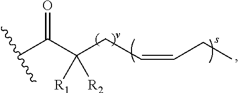

and s is 6.

In other embodiments, Z is

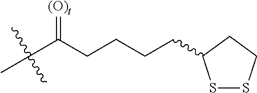

and t is 1.

In some embodiments, Z is

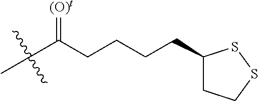

and t is 1.

In some embodiments, W₁ is NH.

In some embodiments, W₂ is NH.

In some embodiments, W₁ is O.

In some embodiments, W₂ is O.

In some embodiments, W₁ is null.

In some embodiments, W2 is null.

In some embodiments, W1 and W2 are each NH.

In some embodiments, W1 and W2 are each null.

In some embodiments, W1 is O and W2 is NH.

In some embodiments, W1 and W2 are each NR, and R is CH3.

In some embodiments, m is 0.

In other embodiments, m is 1.

In other embodiments, m is 2.

In some embodiments, L is —S— or —S—S—.

In some embodiments, L is —O—.

In some embodiments, L is —C(O)—.

In some embodiments, L is heteroaryl.

In some embodiments, L is heterocycle.

In some embodiments, L is

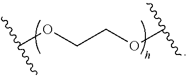

In some embodiments, L is

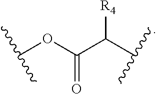

In some embodiments, L is

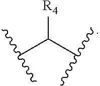

In some embodiments, L is

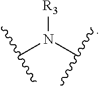

In some embodiments, L is

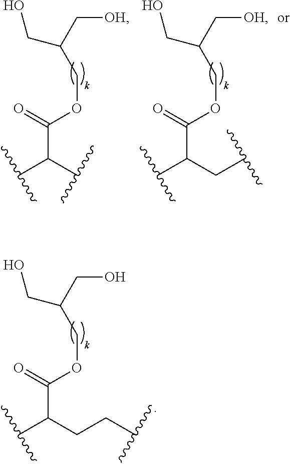

In some embodiments, L is

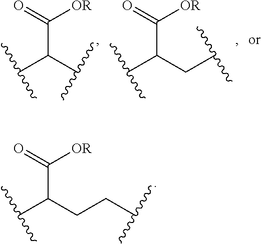

In some embodiments, L is

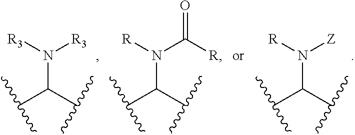

In some embodiments, L is

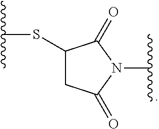 [SMILES: *SC1CC(=O)N(*)C1=O]

In some embodiments, L is

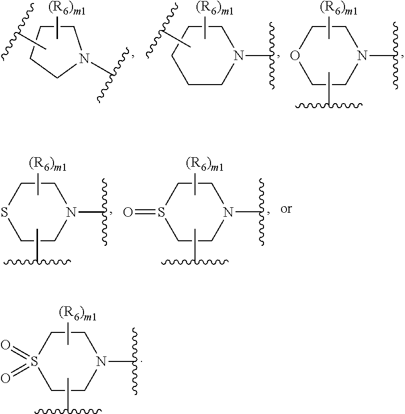

In some embodiments, L is

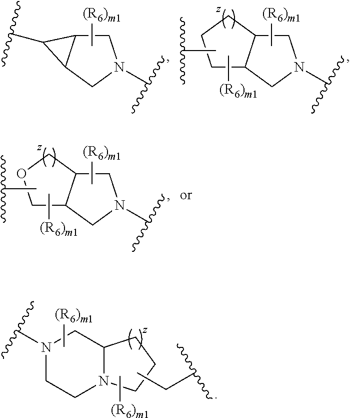

In some embodiments, L is

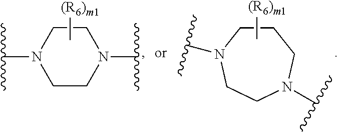

In some embodiments, L is

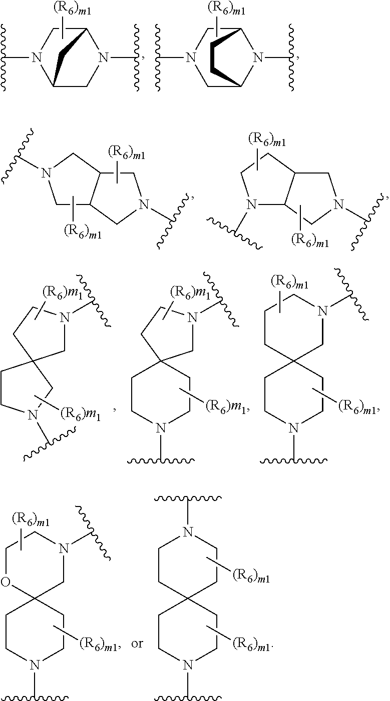

In other embodiments, one of n, o, p, and q is 1.

In some embodiments, two of n, o, p, and q are each 1.

In other embodiments, three of n, o, p, and q are each 1.

In some embodiments n, o, p, and q are each 1.

In some embodiments, one d is C(O)OR.

In some embodiments, r is 2 and s is 6.

In some embodiments, r is 3 and s is 5.

In some embodiments, t is 1.

In some of the foregoing embodiments, r is 2, s is 6 and t is 1.

In some of the foregoing embodiments, r is 3, s is 5 and t is 1.

In some of the foregoing embodiments, Z is

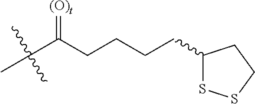

and

t is 1.

In some embodiments, Rₙ₂ is

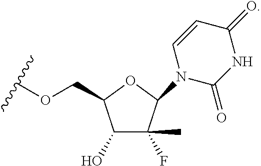 [SMILES: *OC[C@H]1O[C@@H](n2ccc(=O)[nH]c2=O)[C@](C)(F)[C@@H]1O]

In some embodiments, Rₙ₂ is

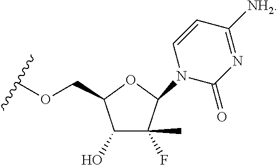 [SMILES: *OC[C@H]1O[C@@H](n2ccc(N)nc2=O)[C@](C)(F)[C@@H]1O]

In some embodiments, Rₙ₂ is

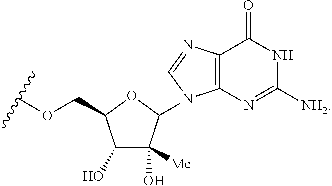 [SMILES: *OC[C@H]1OC(n2cnc3c(=O)[nH]c(N)nc32)[C@](C)(O)[C@@H]1O]

In some embodiments, Rₙ₂ is

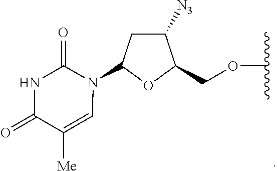

In some embodiments, Rₙ₂ is

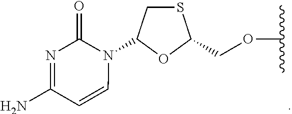

In some embodiments, Rₙ₂ is

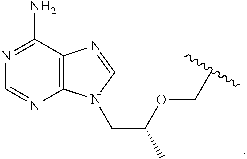 [SMILES: *CO[C@H](C)Cn1cnc2c(N)ncnc21]

In some embodiments, Rₙ₂ is

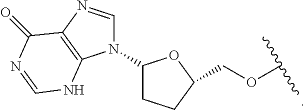

In some embodiments, Rₙ₂ is

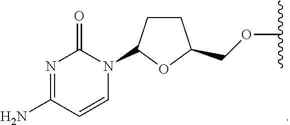

In some embodiments, Rₙ₂ is

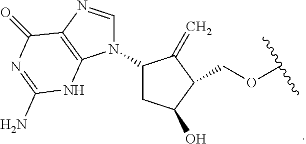

In some embodiments, Rₙ₂ is

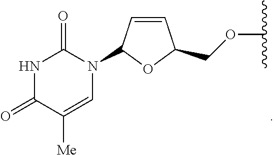

In some embodiments, Rₙ₂ is

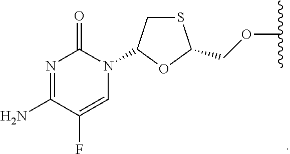

In some embodiments, Rₙ₂ is

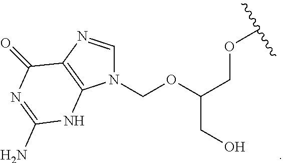 [SMILES: *OCC(CO)OCn1cnc2c(=O)nc(N)[nH]c21]

In some embodiments, Rₙ₂ is

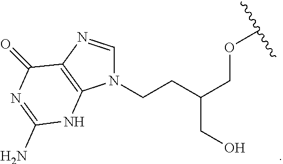 [SMILES: *OCC(CO)CCn1cnc2c(=O)nc(N)[nH]c21]

In some embodiments, Rₙ₂ is

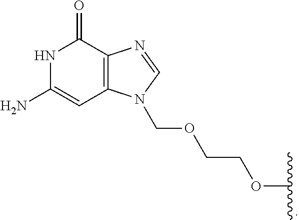 [SMILES: *OCCOCn1cnc2c(=O)[nH]c(N)cc21]

In some embodiments, Rₙ₂ is

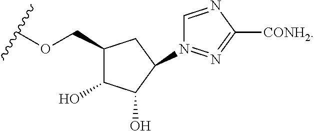

In some embodiments, Rₙ₂ is

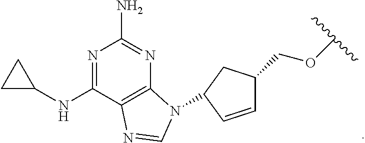

In some embodiments, Rₙ₂ is

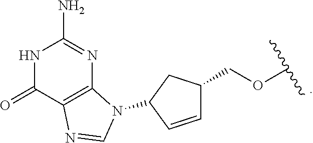

In some embodiments, Rₙ₂ is

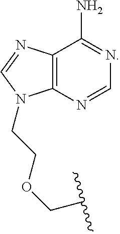 [SMILES: *COCCn1cnc2c(N)ncnc21]

In some embodiments, Rₙ₂ is

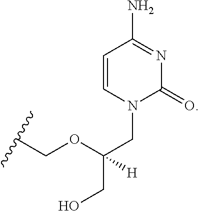 [SMILES: *CO[C@H](CO)Cn1ccc(N)nc1=O]

In some embodiments, Rₙ₂ is

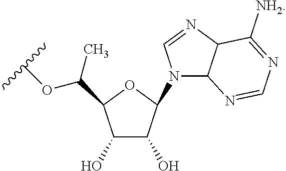 [SMILES: *OC(C)[C@H]1O[C@@H](N2C=NC3C(N)=NC=NC32)[C@H](O)[C@@H]1O]

In some embodiments, Rₙ₂ is

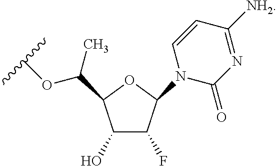 [SMILES: *OC(C)[C@H]1O[C@@H](n2ccc(N)nc2=O)[C@H](F)[C@@H]1O]

In some embodiments, Rₙ₂ is

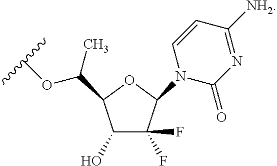

In some embodiments, Rₙ₂ is

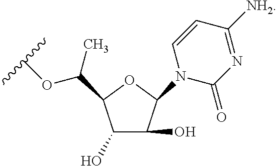 [SMILES: *OC(C)[C@H]1O[C@@H](n2ccc(N)nc2=O)[C@@H](O)[C@@H]1O]

In some embodiments, Rₙ₂ is

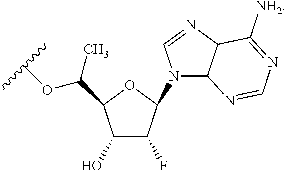 [SMILES: *OC(C)[C@H]1O[C@@H](N2C=NC3C(N)=NC=NC32)[C@H](F)[C@@H]1O]

In some embodiments, Rₙ₂ is

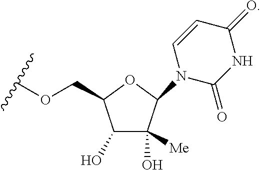 [SMILES: *OC[C@H]1O[C@@H](n2ccc(=O)[nH]c2=O)[C@](C)(O)[C@@H]1O]

In some embodiments, Rₙ₂ is

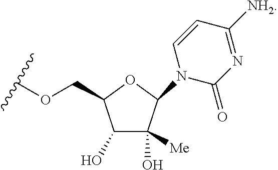 [SMILES: *OC[C@H]1O[C@@H](n2ccc(N)nc2=O)[C@](C)(O)[C@@H]1O]

In some embodiments, Rₙ₂ is

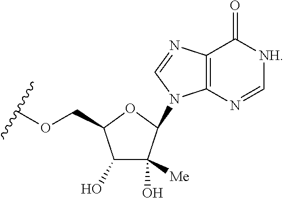 [SMILES: *OC[C@H]1O[C@@H](n2cnc3c(=O)[nH]cnc32)[C@](C)(O)[C@@H]1O]

In some embodiments, Rₙ₂ is

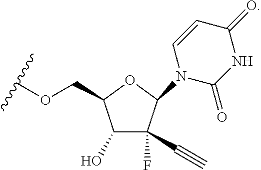 [SMILES: *OC[C@H]1O[C@@H](n2ccc(=O)[nH]c2=O)[C@@](F)(C#C)[C@@H]1O]

In some embodiments, Rₙ₂ is

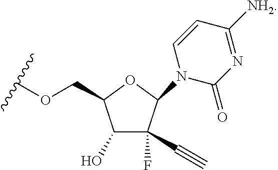 [SMILES: *OC[C@H]1O[C@@H](n2ccc(N)nc2=O)[C@@](F)(C#C)[C@@H]1O]

In some embodiments, Rₙ₂ is

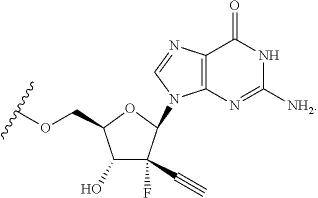 [SMILES: *OC[C@H]1O[C@@H](n2cnc3c(=O)[nH]c(N)nc32)[C@@](F)(C#C)[C@@H]1O]

In some embodiments, Rₙ₂ is

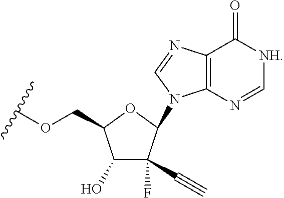 [SMILES: *OC[C@H]1O[C@@H](n2cnc3c(=O)[nH]cnc32)[C@@](F)(C#C)[C@@H]1O]

In some embodiments, Rₙ₂ is

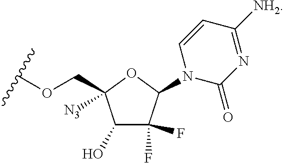

In some embodiments, Rₙ₂ is

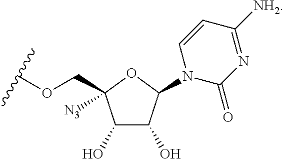 [SMILES: *OC[C@@]1(N=[N+]=[N-])O[C@@H](n2ccc(N)nc2=O)[C@H](O)[C@@H]1O]

In some embodiments, Rₙ₂ is

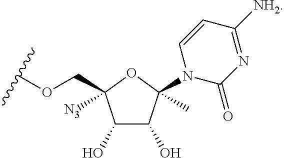 [SMILES: *OC[C@@]1(N=[N+]=[N-])O[C@@](C)(n2ccc(N)nc2=O)[C@H](O)[C@@H]1O]

In some embodiments, Rₙ₂ is

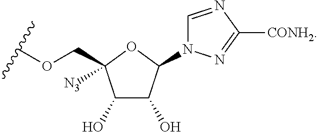 [SMILES: *OC[C@@]1(N=[N+]=[N-])O[C@@H](n2cnc(C(N)=O)n2)[C@H](O)[C@@H]1O]

In some embodiments, Rₙ₂ is

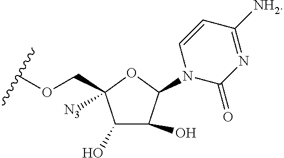 [SMILES: *OC[C@@]1(N=[N+]=[N-])O[C@@H](n2ccc(N)nc2=O)[C@@H](O)[C@@H]1O]

In some embodiments, Rₙ₂ is

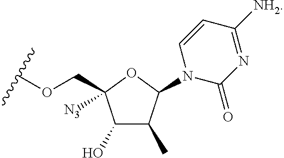 [SMILES: *OC[C@@]1(N=[N+]=[N-])O[C@@H](n2ccc(N)nc2=O)[C@@H](C)[C@@H]1O]

In some embodiments, Rₙ₂ is

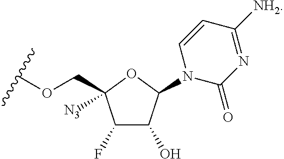 [SMILES: *OC[C@@]1(N=[N+]=[N-])O[C@@H](n2ccc(N)nc2=O)[C@H](O)[C@@H]1F]

In some embodiments, Rₙ₂ is

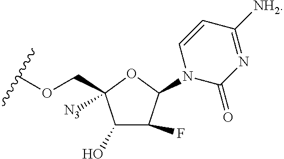 [SMILES: *OC[C@@]1(N=[N+]=[N-])O[C@@H](n2ccc(N)nc2=O)[C@@H](F)[C@@H]1O]

In some embodiments, Rₙ₂ is

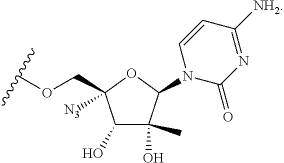 [SMILES: *OC[C@@]1(N=[N+]=[N-])O[C@@H](n2ccc(N)nc2=O)[C@](C)(O)[C@@H]1O]

In some embodiments, Rₙ₂ is

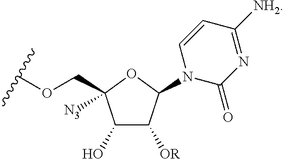

In some embodiments, Rₙ₂ is

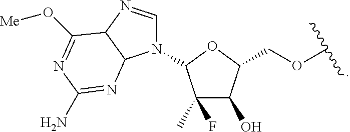

In some embodiments, Rₙ₄ is

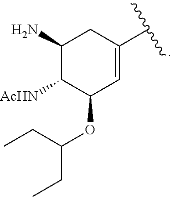 [SMILES: *C1=C[C@@H](OC(CC)CC)[C@H](NC(C)=O)[C@@H](N)C1]

In some embodiments, Rₙ₄ is

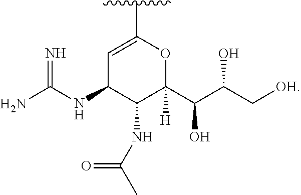 [SMILES: *C1=C[C@H](NC(=N)N)[C@@H](NC(C)=O)[C@H]([C@H](O)[C@H](O)CO)O1]

In some embodiments, Rₙ₄ is

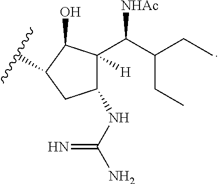 [SMILES: *[C@H]1C[C@@H](NC(=N)N)[C@H]([C@@H](NC(C)=O)C(CC)CC)[C@@H]1O]

In some embodiments, Rₙ₄ is

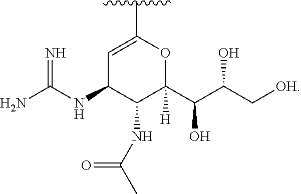 [SMILES: *C1=C[C@H](NC(=N)N)[C@@H](NC(C)=O)[C@H]([C@H](O)[C@H](O)CO)O1]

In some embodiments, Rₙ₄ is

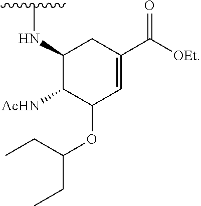 [SMILES: *N[C@H]1CC(C(=O)OCC)=CC(OC(CC)CC)[C@@H]1NC(C)=O]

In some embodiments, Rₙ₅ is

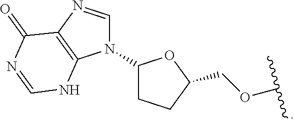

In some embodiments, Rₙ₅ is

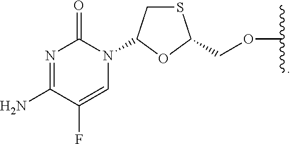

In some embodiments, Rₙ₅ is

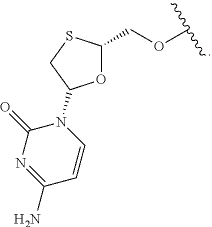

In some embodiments, Rₙ₅ is

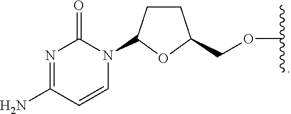

In some embodiments, Rₙ₅ is

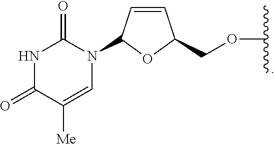

In some embodiments, Rₙ₅ is

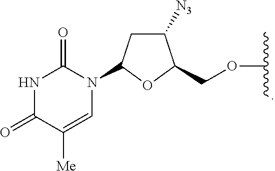

In some embodiments, Rₙ₅ is

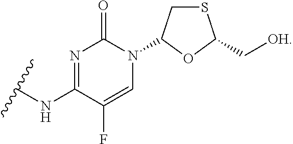

In some embodiments, Rₙ₅ is

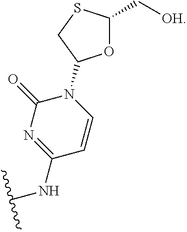

In some embodiments, Rₙ₅ is

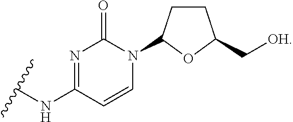

In some embodiments, Rₙ₅ is

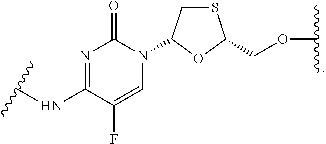

In some embodiments, Rₙ₅ is

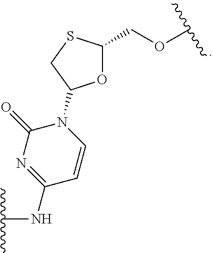

In some embodiments, Rₙ₅ is

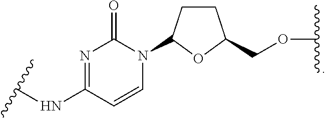

In some embodiments, Rₙ₅ is

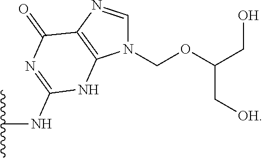 [SMILES: *Nc1nc(=O)c2ncn(COC(CO)CO)c2[nH]1]

In some embodiments, Rₙ₅ is

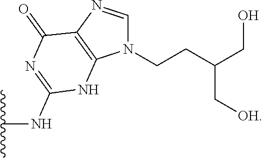 [SMILES: *Nc1nc(=O)c2ncn(CCC(CO)CO)c2[nH]1]

In some embodiments, Rₙ₅ is

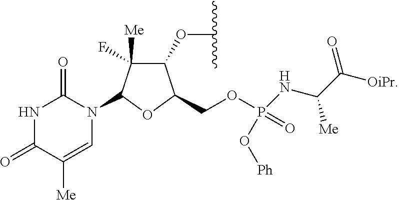

In some embodiments, Rₙ₅ is

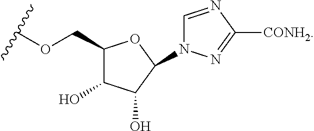 [SMILES: *OC[C@H]1O[C@@H](n2cnc(C(N)=O)n2)[C@H](O)[C@@H]1O]

In some embodiments, Rₙ₅ is

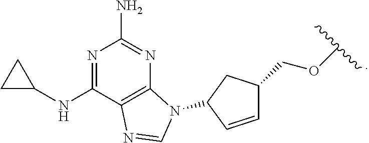

In some embodiments, Rₙ₅ is

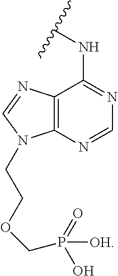 [SMILES: *Nc1ncnc2c1ncn2CCOCP(=O)(O)O]

In some embodiments, Rₙ₅ is

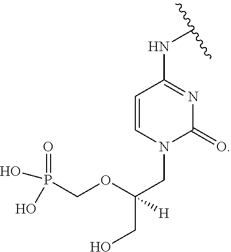 [SMILES: *Nc1ccn(C[C@@H](CO)OCP(=O)(O)O)c(=O)n1]

In some embodiments, Rₙ₅ is

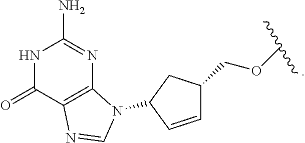

In some embodiments, Rₙ₅ is

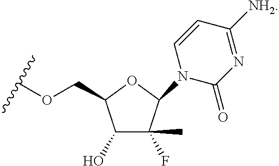 [SMILES: *OC[C@H]1O[C@@H](n2ccc(N)nc2=O)[C@](C)(F)[C@@H]1O]

In some embodiments, Rₙ₅ is

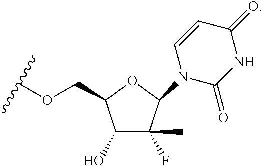 [SMILES: *OC[C@H]1O[C@@H](n2ccc(=O)[nH]c2=O)[C@](C)(F)[C@@H]1O]

In some embodiments, Rₙ₅ is

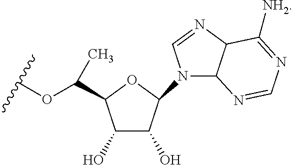 [SMILES: *OC(C)[C@H]1O[C@@H](N2C=NC3C(N)=NC=NC32)[C@H](O)[C@@H]1O]

In some embodiments, Rₙ₅ is

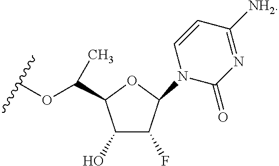 [SMILES: *OC(C)[C@H]1O[C@@H](n2ccc(N)nc2=O)[C@H](F)[C@@H]1O]

In some embodiments, Rₙ₅ is

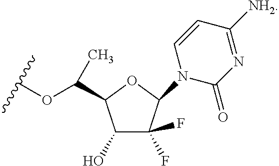

In some embodiments, Rₙ₅ is

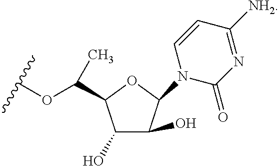 [SMILES: *OC(C)[C@H]1O[C@@H](n2ccc(N)nc2=O)[C@@H](O)[C@@H]1O]

In some embodiments, Rₙ₅ is

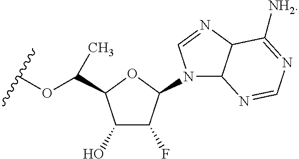 [SMILES: *OC(C)[C@H]1O[C@@H](N2C=NC3C(N)=NC=NC32)[C@H](F)[C@@H]1O]

In some embodiments, Rₙ₅ is

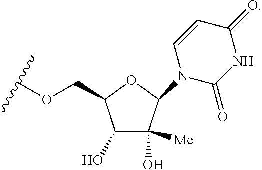 [SMILES: *OC[C@H]1O[C@@H](n2ccc(=O)[nH]c2=O)[C@](C)(O)[C@@H]1O]

In some embodiments, Rₙ₅ is

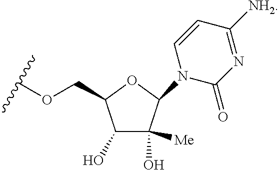 [SMILES: *OC[C@H]1O[C@@H](n2ccc(N)nc2=O)[C@](C)(O)[C@@H]1O]

In some embodiments, Rₙ₅ is

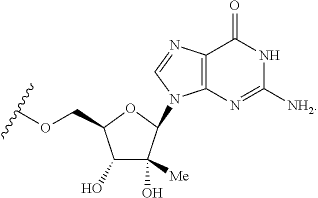 [SMILES: *OC[C@H]1O[C@@H](n2cnc3c(=O)[nH]c(N)nc32)[C@](C)(O)[C@@H]1O]

In some embodiments, Rₙ₅ is

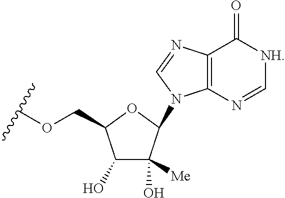 [SMILES: *OC[C@H]1O[C@@H](n2cnc3c(=O)[nH]cnc32)[C@](C)(O)[C@@H]1O]

In some embodiments, Rₙ₅ is

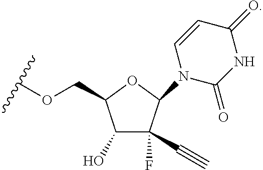 [SMILES: *OC[C@H]1O[C@@H](n2ccc(=O)[nH]c2=O)[C@@](F)(C#C)[C@@H]1O]

In some embodiments, Rₙ₅ is

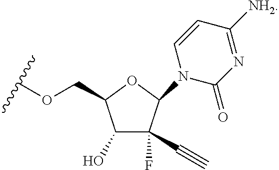 [SMILES: *OC[C@H]1O[C@@H](n2ccc(N)nc2=O)[C@@](F)(C#C)[C@@H]1O]

In some embodiments, Rₙ₅ is

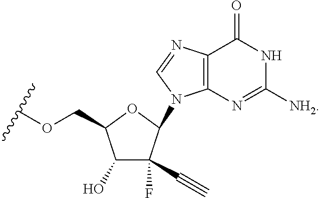 [SMILES: *OC[C@H]1O[C@@H](n2cnc3c(=O)[nH]c(N)nc32)[C@@](F)(C#C)[C@@H]1O]

In some embodiments, Rₙ₅ is

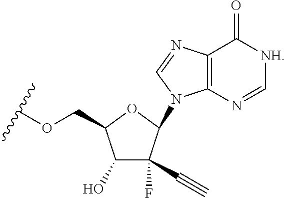 [SMILES: *OC[C@H]1O[C@@H](n2cnc3c(=O)[nH]cnc32)[C@@](F)(C#C)[C@@H]1O]

In some embodiments, Rₙ₅ is

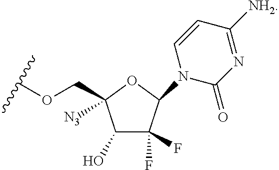

In some embodiments, Rₙ₅ is

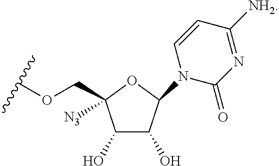 [SMILES: *OC[C@@]1(N=[N+]=[N-])O[C@@H](n2ccc(N)nc2=O)[C@H](O)[C@@H]1O]

In some embodiments, Rₙ₅ is

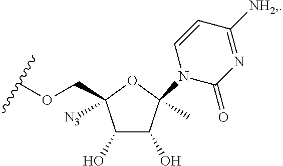 [SMILES: *OC[C@@]1(N=[N+]=[N-])O[C@@](C)(n2ccc(N)nc2=O)[C@H](O)[C@@H]1O]

In some embodiments, Rₙ₅ is

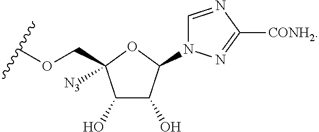 [SMILES: *OC[C@@]1(N=[N+]=[N-])O[C@@H](n2cnc(C(N)=O)n2)[C@H](O)[C@@H]1O]

In some embodiments, Rₙ₅ is

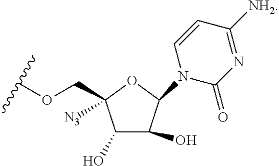 [SMILES: *OC[C@@]1(N=[N+]=[N-])O[C@@H](n2ccc(N)nc2=O)[C@@H](O)[C@@H]1O]

In some embodiments, Rₙ₅ is

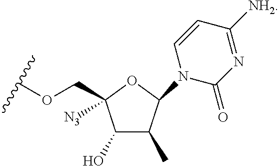 [SMILES: *OC[C@@]1(N=[N+]=[N-])O[C@@H](n2ccc(N)nc2=O)[C@@H](C)[C@@H]1O]

In some embodiments, Rₙ₅ is

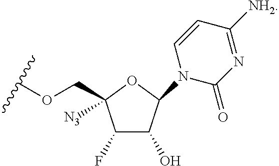 [SMILES: *OC[C@@]1(N=[N+]=[N-])O[C@@H](n2ccc(N)nc2=O)[C@H](O)[C@@H]1F]

In some embodiments, Rₙ₅ is

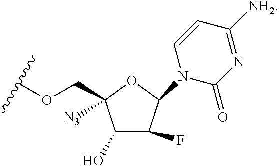 [SMILES: *OC[C@@]1(N=[N+]=[N-])O[C@@H](n2ccc(N)nc2=O)[C@@H](F)[C@@H]1O]

In some embodiments, Rₙ₅ is

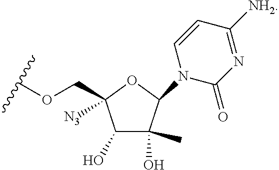 [SMILES: *OC[C@@]1(N=[N+]=[N-])O[C@@H](n2ccc(N)nc2=O)[C@](C)(O)[C@@H]1O]

In some embodiments, Rₙ₅ is

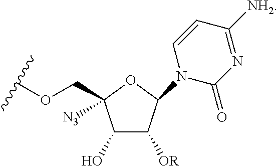

In some embodiments, Rₙ₅ is

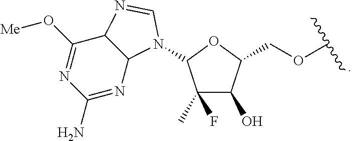

In some embodiments, Rₙ₆ is

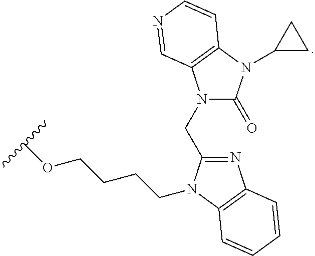 [SMILES: *OCCCCn1c(Cn2c(=O)n(C3CC3)c3ccncc32)nc2ccccc21]

In some embodiments, Rₙ₆ is

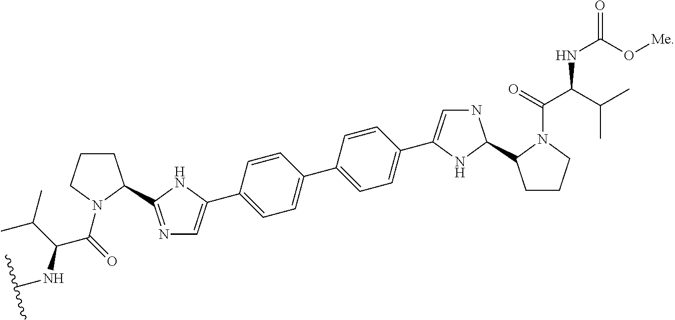 [SMILES: *N[C@H](C(=O)N1CCC[C@H]1c1ncc(-c2ccc(-c3ccc(C4=CN[C@@H](C5CCCN5C(=O)[C@@H](NC(=O)OC)C(C)C)N4)cc3)cc2)[nH]1)C(C)C]

In some embodiments, Rₙ₆ is

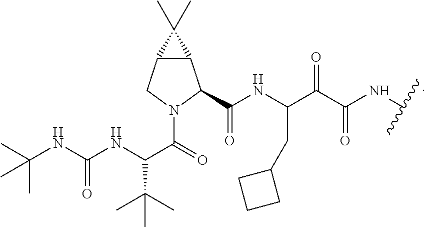

In some embodiments, Rₙ₆ is

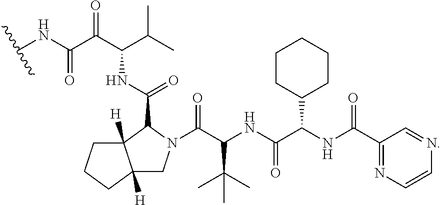

In some embodiments, Rₙ₆ is

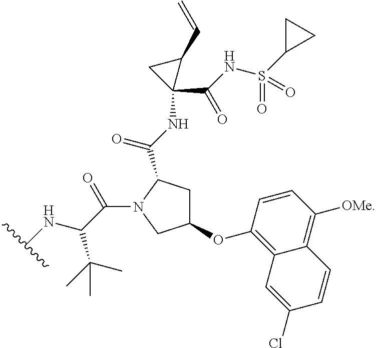

In some embodiments, Rₙ₇ is

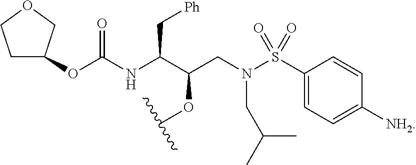 [SMILES: *O[C@H](CN(CC(C)C)S(=O)(=O)c1ccc(N)cc1)[C@H](Cc1ccccc1)NC(=O)O[C@H]1CCOC1]

In some embodiments, Rₙ₇ is

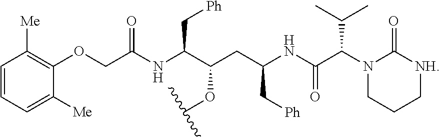 [SMILES: *O[C@@H](C[C@H](Cc1ccccc1)NC(=O)[C@H](C(C)C)N1CCCNC1=O)[C@H](Cc1ccccc1)NC(=O)COc1c(C)cccc1C]

In some embodiments, Rₙ₇ is

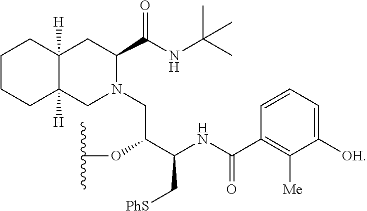

In some embodiments, Rₙ₇ is

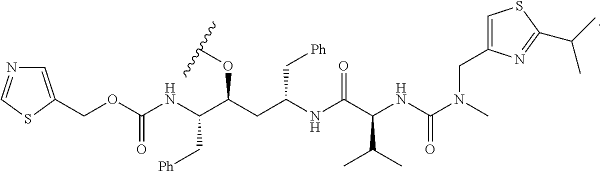 [SMILES: *O[C@@H](C[C@H](Cc1ccccc1)NC(=O)[C@@H](NC(=O)N(C)Cc1csc(C(C)C)n1)C(C)C)[C@H](Cc1ccccc1)NC(=O)OCc1cncs1]

In some embodiments, Rₙ₈ is

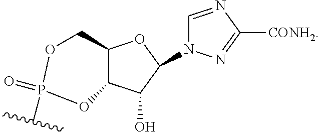

In some embodiments, Rₙ₈ is

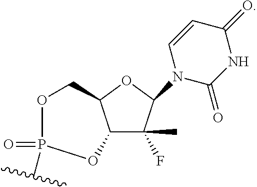

In some embodiments, Rₙ₈ is

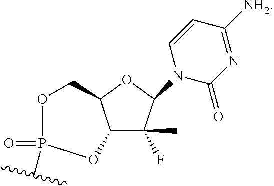

In some embodiments, Rₙ₈ is

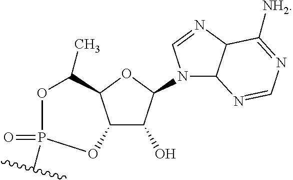

In some embodiments, Rₙ₈ is

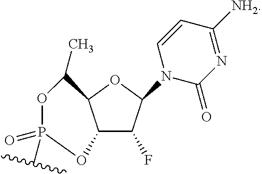

In some embodiments, Rₙ₈ is

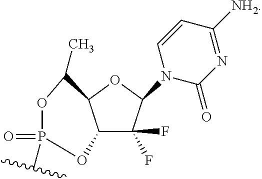

In some embodiments, Rₙ₈ is

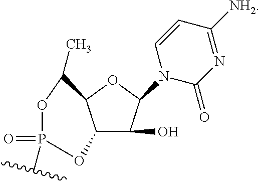

In some embodiments, Rₙ₈ is

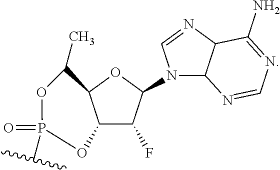

In some embodiments, Rₙ₈ is

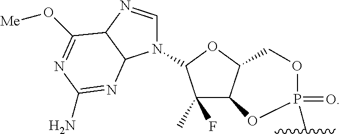

In some embodiments, Rₙ₈ is

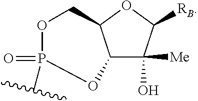

In some embodiments, Rₙ₈ is

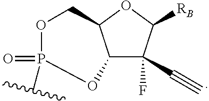

In Formula I, II, III, IV, V, VI, VII and VIII, any one or more of H may be substituted with a deuterium. It is also understood in Formula I, II, III, IV, V, VI, VII and VIII, that a methyl substituent can be substituted with a C₁-C₆ alkyl.

In other illustrative embodiments, compounds of Formula I, II, III, IV, V, VI, VII and VIII are as set forth below:

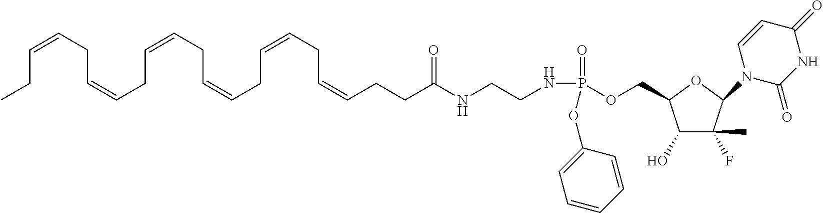 [SMILES: CC/C=C\C/C=C\C/C=C\C/C=C\C/C=C\C/C=C\CCC(=O)NCCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-1)

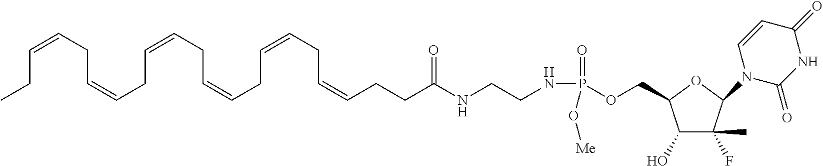 [SMILES: CC/C=C\C/C=C\C/C=C\C/C=C\C/C=C\C/C=C\CCC(=O)NCCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-2)

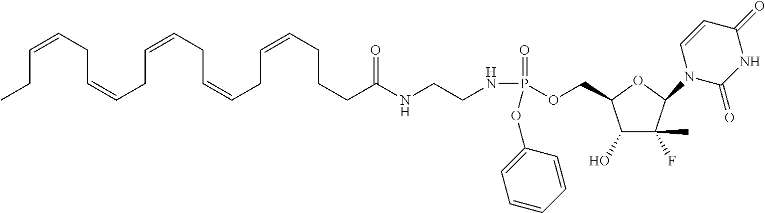 [SMILES: CC/C=C\C/C=C\C/C=C\C/C=C\C/C=C\CCCC(=O)NCCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((5Z,8Z,11Z,14Z,17Z)-icosa-5,8,11,14,17-pentaenamido)ethyl)phosphoramidate (II-3)

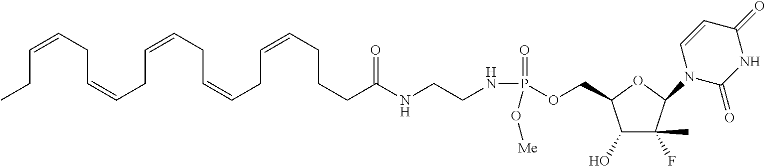 [SMILES: CC/C=C\C/C=C\C/C=C\C/C=C\C/C=C\CCCC(=O)NCCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((5Z,8Z,11Z,14Z,17Z)-icosa-5,8,11,14,17-pentaenamido)ethyl)phosphoramidate (II-4)

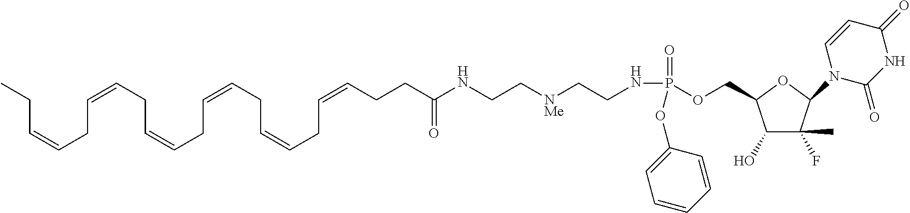 [SMILES: CC/C=C\C/C=C\C/C=C\C/C=C\C/C=C\C/C=C\CCC(=O)NCCN(C)CCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-5)

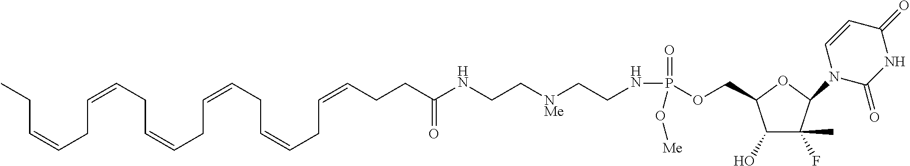 [SMILES: CC/C=C\C/C=C\C/C=C\C/C=C\C/C=C\C/C=C\CCC(=O)NCCN(C)CCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-6)

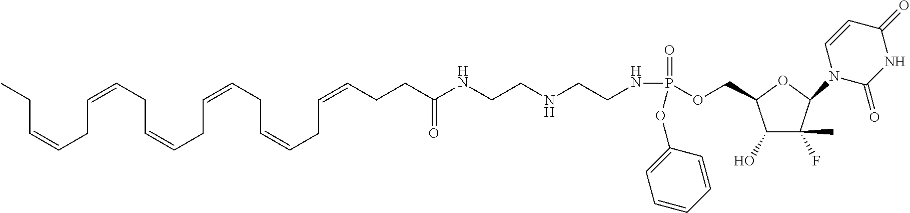 [SMILES: CC/C=C\C/C=C\C/C=C\C/C=C\C/C=C\C/C=C\CCC(=O)NCCNCCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((2-((4Z,7Z,10Z,13Z,16Z,19Z)-docosa-4,7,10,13,16,19-hexaenamido)ethyl)amino)ethyl)phosphoramidate (II-7)

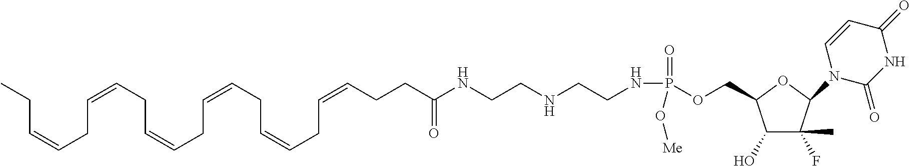 [SMILES: CC/C=C\C/C=C\C/C=C\C/C=C\C/C=C\C/C=C\CCC(=O)NCCNCCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((2-((4Z,7Z,10Z,13Z,16Z,19Z)-docosa-4,7,10,13,16,19-hexaenamido)ethyl)amino)ethyl)phosphoramidate (II-8)

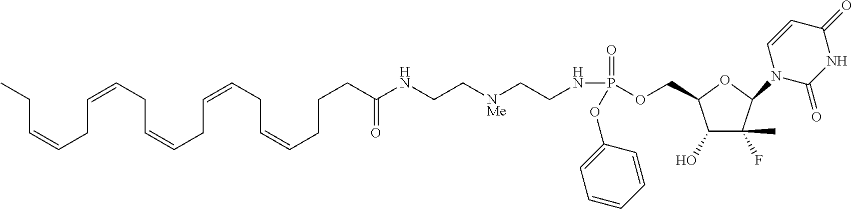 [SMILES: CC/C=C\C/C=C\C/C=C\C/C=C\C/C=C\CCCC(=O)NCCN(C)CCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1 (2H)-yl)-4-fluoro-3-hydroxy-4-methyltetrahydrofuran-2-yl)methyl phenyl (2-((2-((5Z,8Z,11Z,14Z,17Z)-icosa-5,8,11,14,17-pentaenamido)ethyl)(methyl)amino)ethyl)phosphoramidate (II-9)

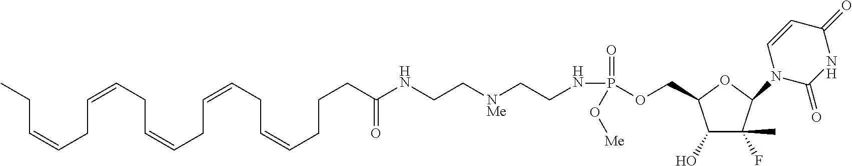 [SMILES: CC/C=C\C/C=C\C/C=C\C/C=C\C/C=C\CCCC(=O)NCCN(C)CCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1 (2H)-yl)-4-fluoro-3-hydroxy-4-methyltetrahydrofuran-2-yl)methyl methyl (2-((2-((5Z,8Z,11Z,14Z,17Z)-icosa-5,8,11,14,17-pentaenamido)ethyl)(methyl)amino)ethyl)phosphoramidate (II-10)

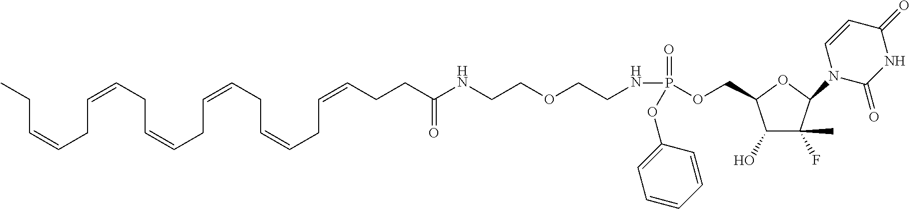 [SMILES: CC/C=C\C/C=C\C/C=C\C/C=C\C/C=C\C/C=C\CCC(=O)NCCOCCNP(=O)(OC[C@H]1O[C@@H](n2ccc(=O)[nH]c2=O)[C@](C)(F)[C@@H]1O)Oc1ccccc1]

((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-(2-((4Z,7Z,10Z,13Z,16Z,19Z)-docosa-4,7,10,13,16,19-hexaenamido)ethoxy)ethyl)phosphoramidate (II-11)

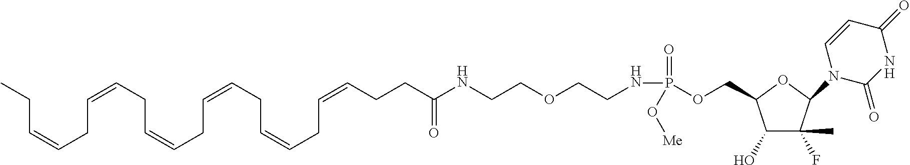 [SMILES: CC/C=C\C/C=C\C/C=C\C/C=C\C/C=C\C/C=C\CCC(=O)NCCOCCNP(=O)(OC)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-(2-((4Z,7Z,10Z,13Z,16Z,19Z)-docosa-4,7,10,13,16,19-hexaenamido)ethoxy)ethyl)phosphoramidate (II-12)

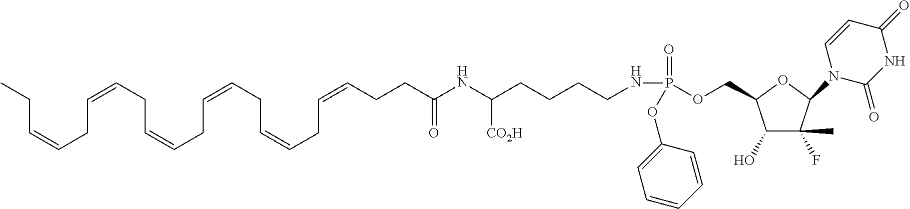 [SMILES: CC/C=C\C/C=C\C/C=C\C/C=C\C/C=C\C/C=C\CCC(=O)NC(CCCCNP(=O)(OC[C@H]1O[C@@H](n2ccc(=O)[nH]c2=O)[C@](C)(F)[C@@H]1O)Oc1ccccc1)C(=O)O]

6-(((((2R,3R,4R,5R)-5-(2,4-dioxo-3,4-dihydropyrimidin-1 (2H)-yl)-4-fluoro-3-hydroxy-4-methyltetrahydrofuran-2-yl)methoxy)(phenoxy)phosphoryl)amino)-2-((4Z,7Z,10Z,13Z,16Z,19Z)-docosa-4,7,10,13,16,19-hexaenamido)hexanoic acid (II-13)

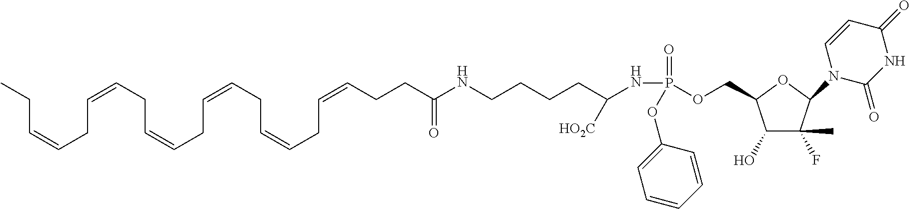 [SMILES: CC/C=C\C/C=C\C/C=C\C/C=C\C/C=C\C/C=C\CCC(=O)NCCCCC(NP(=O)(OC[C@H]1O[C@@H](n2ccc(=O)[nH]c2=O)[C@](C)(F)[C@@H]1O)Oc1ccccc1)C(=O)O]

2-(((((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)-6-((4Z,7Z,10Z,13Z,16Z,19Z)-docosa-4,7,10,13,16,19-hexaenamido)hexanoic acid (II-14)

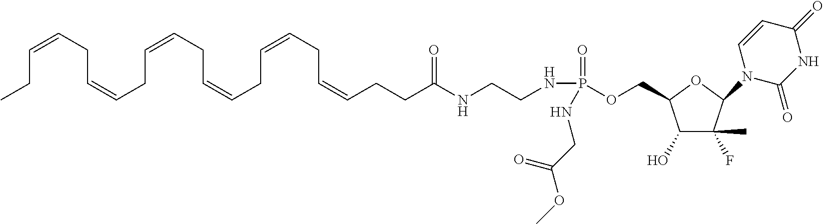 [SMILES: CC/C=C\C/C=C\C/C=C\C/C=C\C/C=C\C/C=C\CCC(=O)NCCNP(=O)(NCC(=O)OC)OC[C@H]1O[C@@H](n2ccc(=O)[nH]c2=O)[C@](C)(F)[C@@H]1O]

(12Z,15Z,18Z,21Z,24Z,27Z)-methyl 4-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)-4,9-dioxo-3,5,8-triaza-4-phosphatriaconta-12,15,18,21,24,27-hexaen-1-oate (II-15)

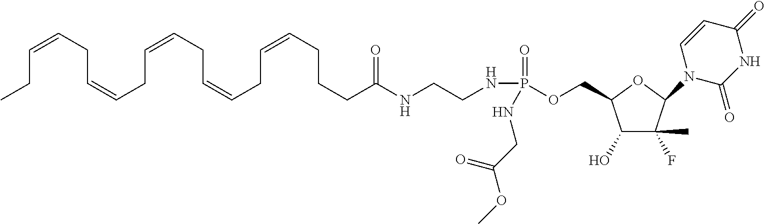 [SMILES: CC/C=C\C/C=C\C/C=C\C/C=C\C/C=C\CCCC(=O)NCCNP(=O)(NCC(=O)OC)OC[C@H]1O[C@@H](n2ccc(=O)[nH]c2=O)[C@](C)(F)[C@@H]1O]

(13Z,16Z,19Z,22Z,25Z)-methyl 4-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)-4,9-dioxo-3,5,8-triaza-4-phosphaoctacosa-13,16,19,22,25-pentaen-1-oate (II-16)

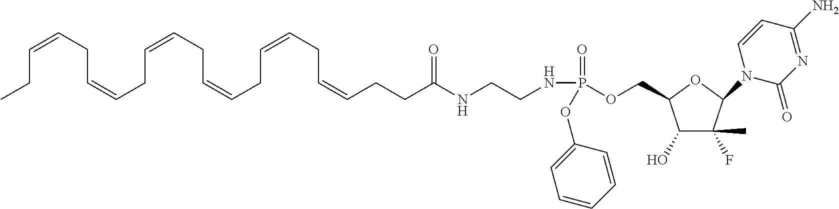 [SMILES: CC/C=C\C/C=C\C/C=C\C/C=C\C/C=C\C/C=C\CCC(=O)NCCNP(=O)(OC[C@H]1O[C@@H](n2ccc(N)nc2=O)[C@](C)(F)[C@@H]1O)Oc1ccccc1]

((2R,3R,4R,5R)-5-(4-amino-2-oxopyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-17)

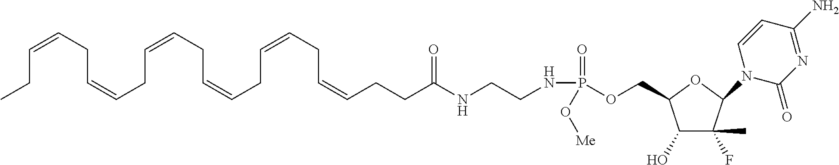 [SMILES: CC/C=C\C/C=C\C/C=C\C/C=C\C/C=C\C/C=C\CCC(=O)NCCNP(=O)(OC)OC[C@H]1O[C@@H](n2ccc(N)nc2=O)[C@](C)(F)[C@@H]1O]

((2R,3R,4R,5R)-5-(4-amino-2-oxopyrimidin-1(2H)-yl)-4-fluoro-3-hydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-18)

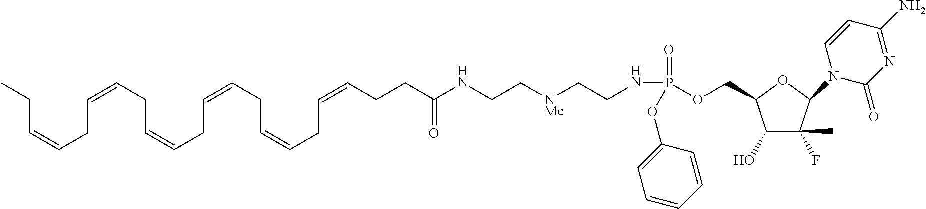 [SMILES: CC/C=C\C/C=C\C/C=C\C/C=C\C/C=C\C/C=C\CCC(=O)NCCN(C)CCNP(=O)(OC[C@H]1O[C@@H](n2ccc(N)nc2=O)[C@](C)(F)[C@@H]1O)Oc1ccccc1]

((2R,3R,4R,5R)-5-(4-amino-2-oxopyrimidin-1(2H)-yl)-4-fluoro-3-hydroxy-4-methyltetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-19)

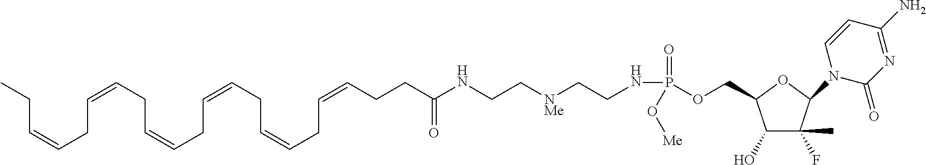 [SMILES: CC/C=C\C/C=C\C/C=C\C/C=C\C/C=C\C/C=C\CCC(=O)NCCN(C)CCNP(=O)(OC)OC[C@H]1O[C@@H](n2ccc(N)nc2=O)[C@](C)(F)[C@@H]1O]

((2R,3R,4R,5R)-5-(4-amino-2-oxopyrimidin-1(2H)-yl)-4-fluoro-3-hydroxy-4-methyltetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-20)

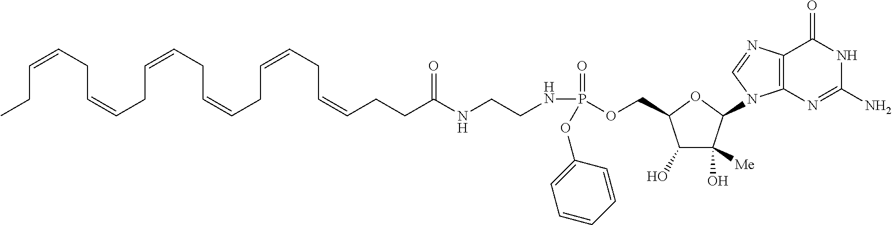 [SMILES: CC/C=C\C/C=C\C/C=C\C/C=C\C/C=C\C/C=C\CCC(=O)NCCNP(=O)(OC[C@H]1O[C@@H](n2cnc3c(=O)[nH]c(N)nc32)[C@](C)(O)[C@@H]1O)Oc1ccccc1]

((2R,3R,4R,5R)-5-(2-amino-6-oxo-1H-purin-9(6H)-yl)-3,4-dihydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-21)

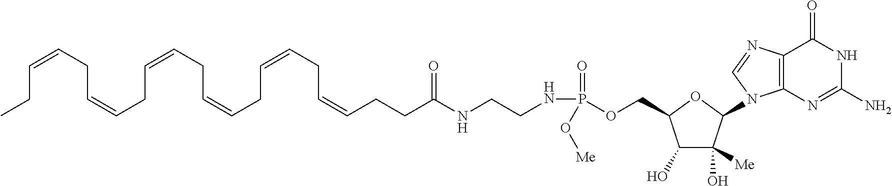 [SMILES: CC/C=C\C/C=C\C/C=C\C/C=C\C/C=C\C/C=C\CCC(=O)NCCNP(=O)(OC)OC[C@H]1O[C@@H](n2cnc3c(=O)[nH]c(N)nc32)[C@](C)(O)[C@@H]1O]

((2R,3R,4R,5R)-5-(2-amino-6-oxo-1H-purin-9(6H)-yl)-3,4-dihydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-22)

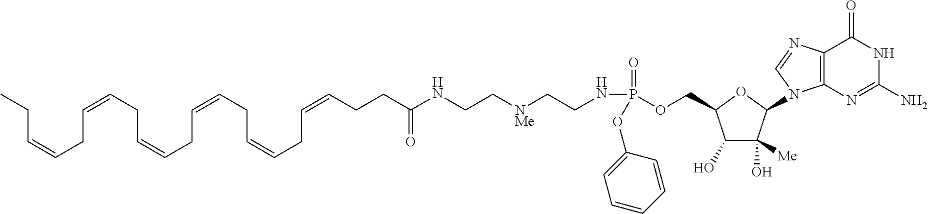 [SMILES: CC/C=C\C/C=C\C/C=C\C/C=C\C/C=C\C/C=C\CCC(=O)NCCN(C)CCNP(=O)(OC[C@H]1O[C@@H](n2cnc3c(=O)[nH]c(N)nc32)[C@](C)(O)[C@@H]1O)Oc1ccccc1]

((2R,3R,4R,5R)-5-(2-amino-6-oxo-1H-purin-9(6H)-yl)-3,4-dihydroxy-4-methyltetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-23)

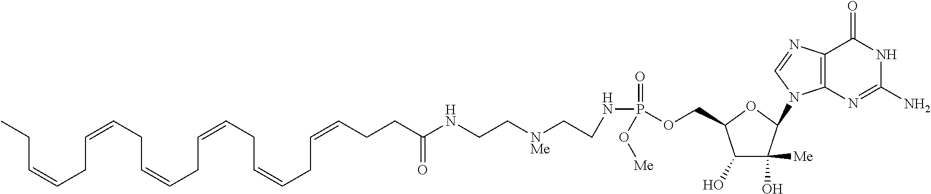 [SMILES: CC/C=C\C/C=C\C/C=C\C/C=C\C/C=C\C/C=C\CCC(=O)NCCN(C)CCNP(=O)(OC)OC[C@H]1O[C@@H](n2cnc3c(=O)[nH]c(N)nc32)[C@](C)(O)[C@@H]1O]

((2R,3R,4R,5R)-5-(2-amino-6-oxo-1H-purin-9(6H)-yl)-3,4-dihydroxy-4-methyltetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-24)

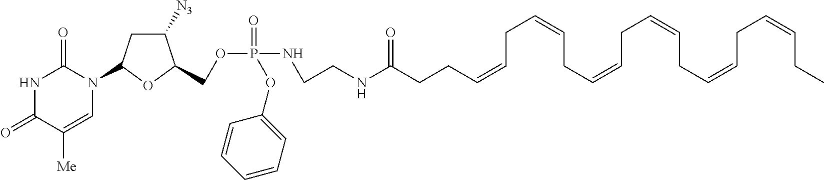

((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (II-25)

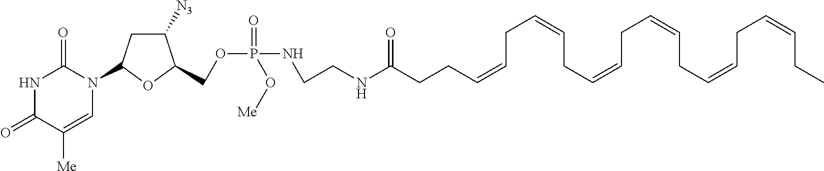

((2S,3S,5S)-3-azido-5-(5-methyl-2,4-dioxo-3,4-dihydropyrimidin-1(2H)-yl)tetrahydrofuran-2-yl)methyl methyl (2-((4Z,7Z,10Z,13Z,16Z,19Z)-docosa-4,7,10,13,16,19-hexaenamido)ethyl)phosphoramidate (II-26)

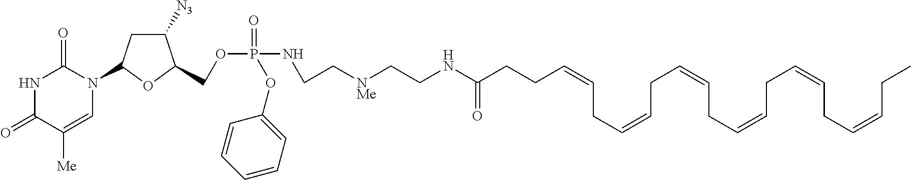

((2S,3S,5S)-3-azido-5-(5-methyl-2,4-dioxo-3,4-dihydropyrimidin-1(2H)-yl)tetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-27)

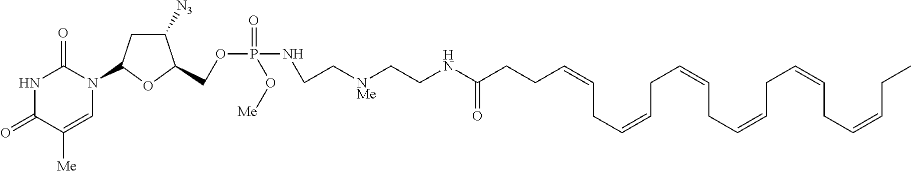

((2S,3S,5S)-3-azido-5-(5-methyl-2,4-dioxo-3,4-dihydropyrimidin-1(2H)-yl)tetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-28)

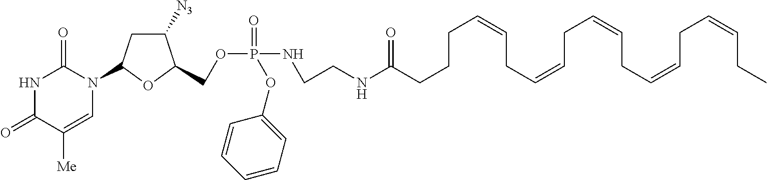

((2S,3S,5S)-3-azido-5-(5-methyl-2,4-dioxo-3,4-dihydropyrimidin-1(2H)-yl)tetrahydrofuran-2-yl)methyl phenyl (2-((5Z,8Z,11Z,14Z,17Z)-icosa-5,8,11,14,17-pentaenamido)ethyl)phosphoramidate (II-29)

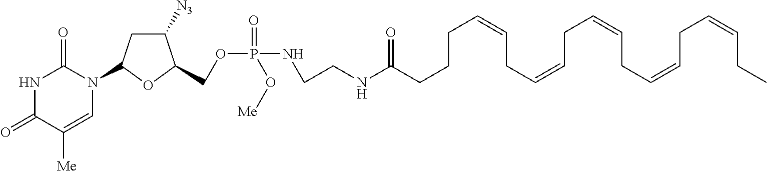

((2S,3S,5S)-3-azido-5-(5-methyl-2,4-dioxo-3,4-dihydropyrimidin-1(2H)-yl)tetrahydrofuran-2-yl)methyl methyl (2-((5Z,8Z,11Z,14Z,17Z)-icosa-5,8,11,14,17-pentaenamido)ethyl)phosphoramidate (II-30)

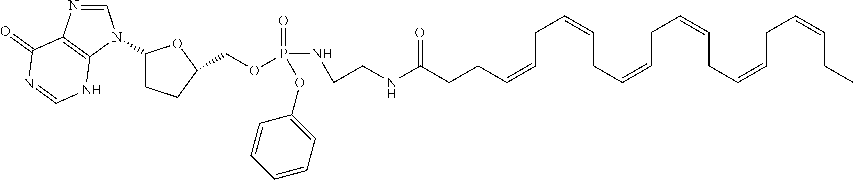

((2S,5R)-5-(6-oxo-3H-purin-9(6H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (II-31)

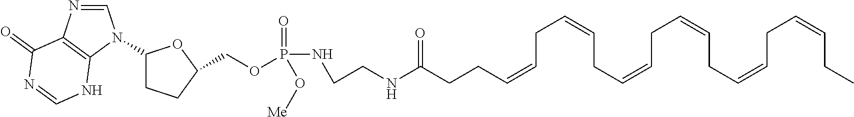

methyl (((2S,5R)-5-(6-oxo-3H-purin-9(6H)-yl)tetrahydrofuran-2-yl)methyl)(2-((4Z,7Z,10Z,13Z,16Z,19Z)-docosa-4,7,10,13,16,19-hexaenamido)ethyl)phosphoramidate (II-32)

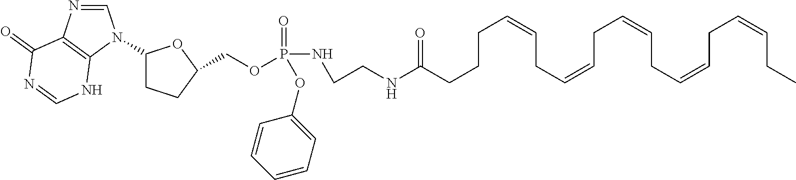

((2S,5R)-5-(6-oxo-3H-purin-9(6H)-yl)tetrahydrofuran-2-yl)methyl phenyl (2-((5Z,8Z,11Z,14Z,17Z)-icosa-5,8,11,14,17-pentaenamido)ethyl)phosphoramidate (II-33)

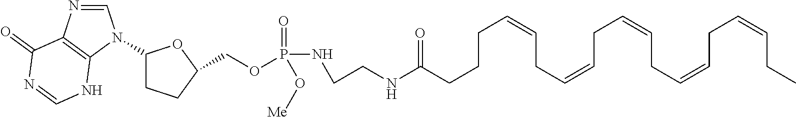

methyl (((2S,5R)-5-(6-oxo-3H-purin-9(6H)-yl)tetrahydrofuran-2-yl)methyl)(2-((5Z,8Z,11Z,14Z,17Z)-icosa-5,8,11,14,17-pentaenamido)ethyl)phosphoramidate (II-34)

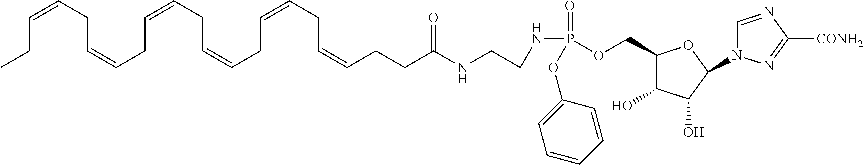 [SMILES: CC/C=C\C/C=C\C/C=C\C/C=C\C/C=C\C/C=C\CCC(=O)NCCNP(=O)(OC[C@H]1O[C@@H](n2cnc(C(N)=O)n2)[C@H](O)[C@@H]1O)Oc1ccccc1]

((2R,3S,4R,5R)-5-(3-carbamoyl-1H-1,2,4-triazol-1-yl)-3,4-dihydroxytetrahydrofuran-2-yl)methyl phenyl (2-((4Z,7Z,10Z,13Z,16Z,19Z)-docosa-4,7,10,13,16,19-hexaenamido)ethyl)phosphoramidate (II-35)

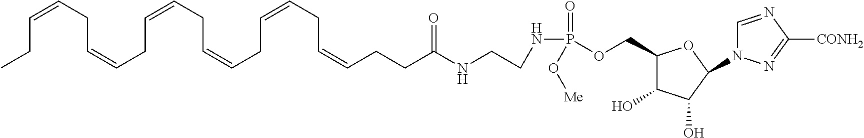 [SMILES: CC/C=C\C/C=C\C/C=C\C/C=C\C/C=C\C/C=C\CCC(=O)NCCNP(=O)(OC)OC[C@H]1O[C@@H](n2cnc(C(N)=O)n2)[C@H](O)[C@@H]1O]

((2R,3S,4R,5R)-5-(3-carbamoyl-1H-1,2,4-triazol-1-yl)-3,4-dihydroxytetrahydrofuran-2-yl)methyl methyl (2-((4Z,7Z,10Z,13Z,16Z,19Z)-docosa-4,7,10,13,16,19-hexaenamido)ethyl)phosphoramidate (II-36)

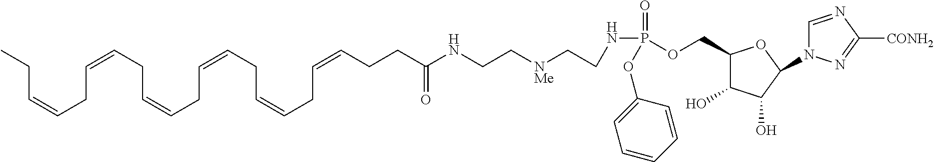 [SMILES: CC/C=C\C/C=C\C/C=C\C/C=C\C/C=C\C/C=C\CCC(=O)NCCN(C)CCNP(=O)(OC[C@H]1O[C@@H](n2cnc(C(N)=O)n2)[C@H](O)[C@@H]1O)Oc1ccccc1]

((2R,3S,4R,5R)-5-(3-carbamoyl-1H-1,2,4-triazol-1-yl)-3,4-dihydroxytetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-37)

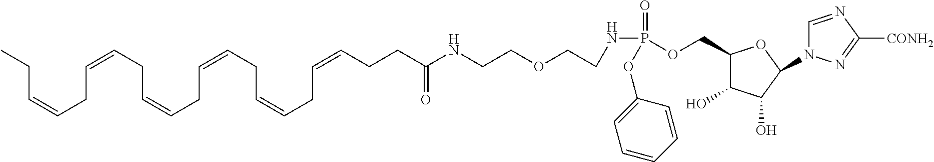 [SMILES: CC/C=C\C/C=C\C/C=C\C/C=C\C/C=C\C/C=C\CCC(=O)NCCOCCNP(=O)(OC[C@H]1O[C@@H](n2cnc(C(N)=O)n2)[C@H](O)[C@@H]1O)Oc1ccccc1]

((2R,3S,4R,5R)-5-(3-carbamoyl-1H-1,2,4-triazol-1-yl)-3,4-dihydroxytetrahydrofuran-2-yl)methyl phenyl (2-(2-((4Z,7Z,10Z,13Z,16Z,19Z)-docosa-4,7,10,13,16,19-hexaenamido)ethoxy)ethyl)phosphoramidate (II-38)

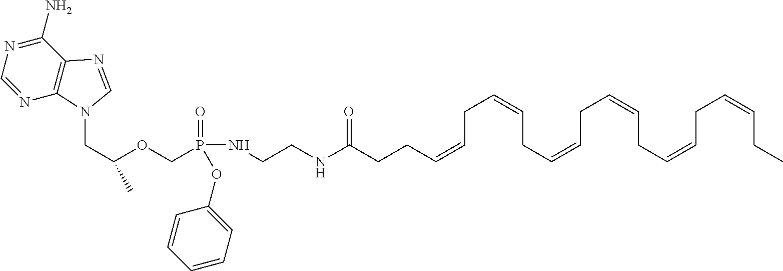 [SMILES: CC/C=C\C/C=C\C/C=C\C/C=C\C/C=C\C/C=C\CCC(=O)NCCNP(=O)(CO[C@H](C)Cn1cnc2c(N)ncnc21)Oc1ccccc1]

phenyl P—((((R)-1-(6-amino-9H-purin-9-yl)propan-2-yl)oxy)methyl)-N-(2-((4Z,7Z,10Z,13Z,16Z,19Z)-docosa-4,7,10,13,16,19-hexaenamido)ethyl)phosphonamidate (II-39)

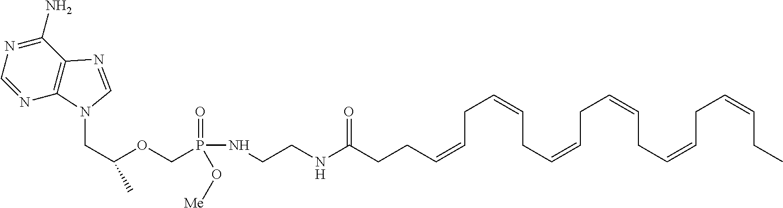 [SMILES: CC/C=C\C/C=C\C/C=C\C/C=C\C/C=C\C/C=C\CCC(=O)NCCNP(=O)(CO[C@H](C)Cn1cnc2c(N)ncnc21)OC]

methyl P-((((R)-1-(6-amino-9H-purin-9-yl)propan-2-yl)oxy)methyl)-N-(2-((4Z,7Z,10Z,13Z,16Z,19Z)-docosa-4,7,10,13,16,19-hexaenamido)ethyl)phosphonamidate (II-40)

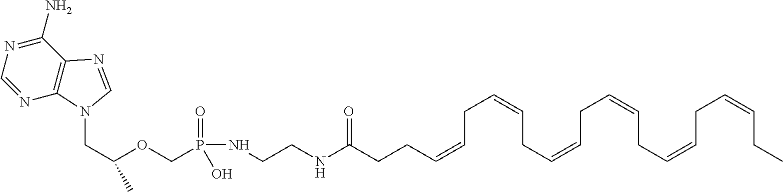 [SMILES: CC/C=C\C/C=C\C/C=C\C/C=C\C/C=C\C/C=C\CCC(=O)NCCNP(=O)(O)CO[C@H](C)Cn1cnc2c(N)ncnc21]

P-((((R)-1-(6-amino-9H-purin-9-yl)propan-2-yl)oxy)methyl)-N-(2-((4Z,7Z,10Z,13Z,16Z,19Z)-docosa-4,7,10,13,16,19-hexaenamido)ethyl)phosphonamidic acid (II-41)

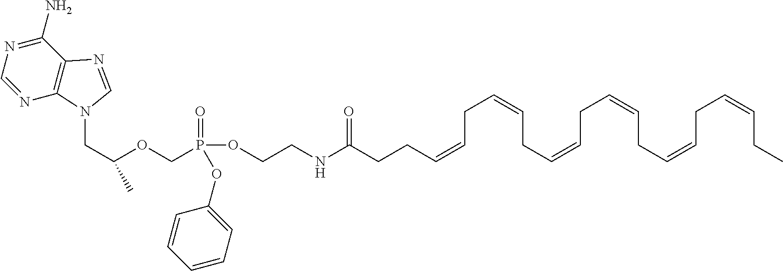 [SMILES: CC/C=C\C/C=C\C/C=C\C/C=C\C/C=C\C/C=C\CCC(=O)NCCOP(=O)(CO[C@H](C)Cn1cnc2c(N)ncnc21)Oc1ccccc1]

2-((4Z,7Z,10Z,13Z,16Z,19Z)-docosa-4,7,10,13,16,19-hexaenamido)ethyl phenyl ((((R)-1-(6-amino-9H-purin-9-yl)propan-2-yl)oxy)methyl)phosphonate (II-42)

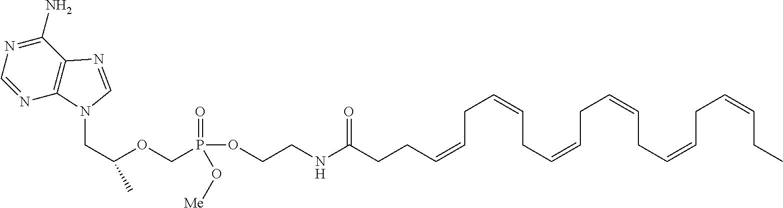 [SMILES: CC/C=C\C/C=C\C/C=C\C/C=C\C/C=C\C/C=C\CCC(=O)NCCOP(=O)(CO[C@H](C)Cn1cnc2c(N)ncnc21)OC]

2-((4Z,7Z,10Z,13Z,16Z,19Z)-docosa-4,7,10,13,16,19-hexaenamido)ethyl methyl ((((R)-1-(6-amino-9H-purin-9-yl)propan-2-yl)oxy)methyl)phosphonate (II-43)

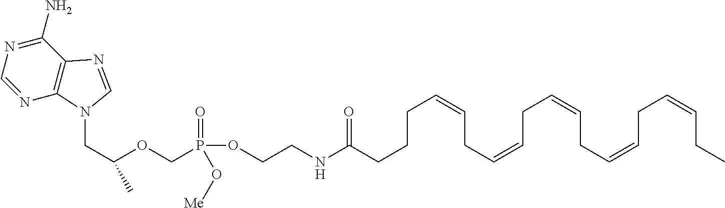 [SMILES: CC/C=C\C/C=C\C/C=C\C/C=C\C/C=C\CCCC(=O)NCCOP(=O)(CO[C@H](C)Cn1cnc2c(N)ncnc21)OC]

2-((5Z,8Z,11Z,14Z,17Z)-icosa-5,8,11,14,17-pentaenamido)ethyl methyl ((((R)-1-(6-amino-9H-purin-9-yl)propan-2-yl)oxy)methyl)phosphonate (II-44)

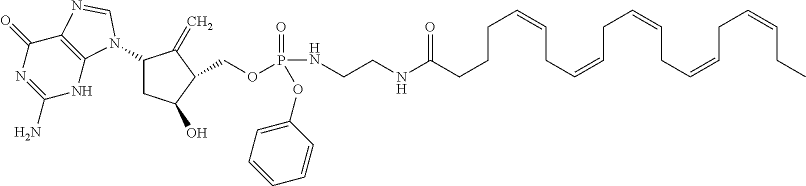

((1R,3S,5S)-3-(2-amino-6-oxo-3H-purin-9(6H)-yl)-5-hydroxy-2-methylenecyclopentyl)methyl phenyl (2-((4Z,7Z,10Z,13Z,16Z,19Z)-docosa-4,7,10,13,16,19-hexaenamido)ethyl)phosphoramidate (II-45)

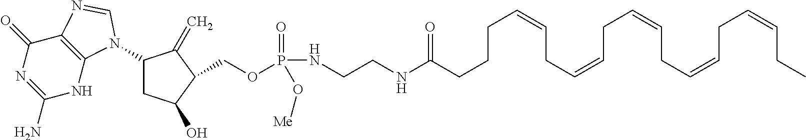

((1R,3S,5S)-3-(2-amino-6-oxo-3H-purin-9(6H)-yl)-5-hydroxy-2-methylenecyclopentyl)methyl methyl (2-((4Z,7Z,10Z,13Z,16Z,19Z)-docosa-4,7,10,13,16,19-hexaenamido)ethyl)phosphoramidate (II-46)

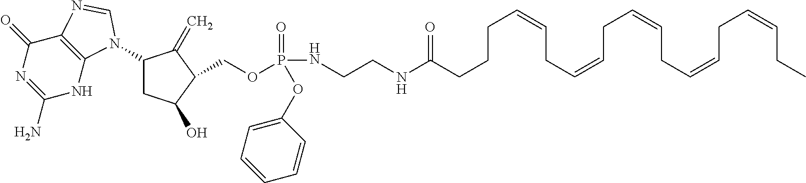

((1R,3S,5S)-3-(2-amino-6-oxo-3H-purin-9(6H)-yl)-5-hydroxy-2-methylenecyclopentyl)methyl phenyl (2-((5Z,8Z,11Z,14Z,17Z)-icosa-5,8,11,14,17-pentaenamido)ethyl)phosphoramidate (II-47)

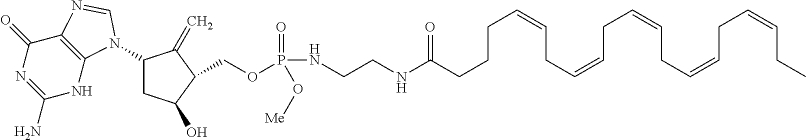

((1R,3S,5S)-3-(2-amino-6-oxo-3H-purin-9(6H)-yl)-5-hydroxy-2-methylenecyclopentyl)methyl methyl (2-((5Z,8Z,11Z,14Z,17Z)-icosa-5,8,11,14,17-pentaenamido)ethyl)phosphoramidate (II-48)

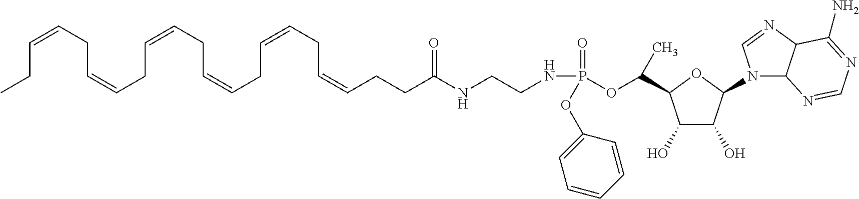 [SMILES: CC/C=C\C/C=C\C/C=C\C/C=C\C/C=C\C/C=C\CCC(=O)NCCNP(=O)(Oc1ccccc1)OC(C)[C@H]1O[C@@H](N2C=NC3C(N)=NC=NC32)[C@H](O)[C@@H]1O]

(1R)-1-((2S,3S,4R,5R)-5-(6-amino-4H-purin-9(5H)-yl)-3,4-dihydroxytetrahydrofuran-2-yl)ethyl phenyl (2-((4Z,7Z,10Z,13Z,16Z,19Z)-docosa-4,7,10,13,16,19-hexaenamido)ethyl)phosphoramidate (II-49)

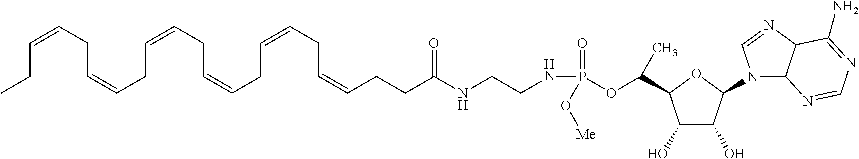 [SMILES: CC/C=C\C/C=C\C/C=C\C/C=C\C/C=C\C/C=C\CCC(=O)NCCNP(=O)(OC)OC(C)[C@H]1O[C@@H](N2C=NC3C(N)=NC=NC32)[C@H](O)[C@@H]1O]

(1R)-1-((2S,3S,4R,5R)-5-(6-amino-4H-purin-9(5H)-yl)-3,4-dihydroxytetrahydrofuran-2-yl)ethyl methyl (2-((4Z,7Z,10Z,13Z,16Z,19Z)-docosa-4,7,10,13,16,19-hexaenamido)ethyl)phosphoramidate (II-50)

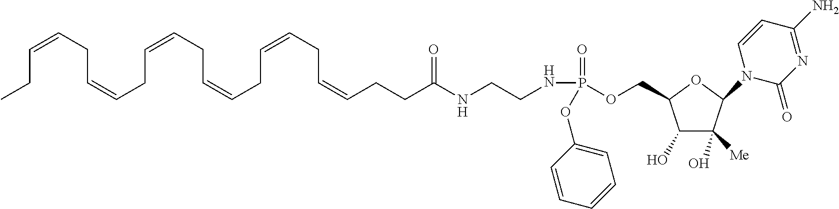 [SMILES: CC/C=C\C/C=C\C/C=C\C/C=C\C/C=C\C/C=C\CCC(=O)NCCNP(=O)(OC[C@H]1O[C@@H](n2ccc(N)nc2=O)[C@](C)(O)[C@@H]1O)Oc1ccccc1]

((2R,3R,4R,5R)-5-(4-amino-2-oxopyrimidin-1(2H)-yl)-3,4-dihydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-51)

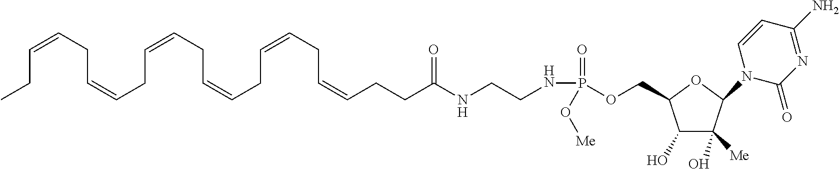 [SMILES: CC/C=C\C/C=C\C/C=C\C/C=C\C/C=C\C/C=C\CCC(=O)NCCNP(=O)(OC)OC[C@H]1O[C@@H](n2ccc(N)nc2=O)[C@](C)(O)[C@@H]1O]

((2R,3R,4R,5R)-5-(4-amino-2-oxopyrimidin-1(2H)-yl)-3,4-dihydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-52)

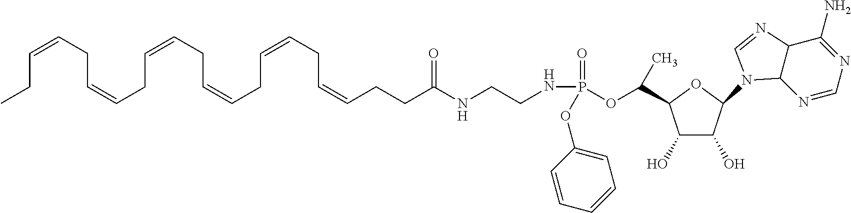 [SMILES: CC/C=C\C/C=C\C/C=C\C/C=C\C/C=C\C/C=C\CCC(=O)NCCNP(=O)(Oc1ccccc1)OC(C)[C@H]1O[C@@H](N2C=NC3C(N)=NC=NC32)[C@H](O)[C@@H]1O]

(1R)-1-((2S,3S,4R,5R)-5-(6-amino-4H-purin-9(5H)-yl)-3,4-dihydroxytetrahydrofuran-2-yl)ethyl phenyl (2-((4Z,7Z,10Z,13Z,16Z,19Z)-docosa-4,7,10,13,16,19-hexaenamido)ethyl)phosphoramidate (II-53)

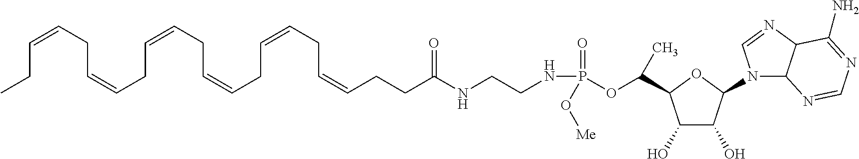 [SMILES: CC/C=C\C/C=C\C/C=C\C/C=C\C/C=C\C/C=C\CCC(=O)NCCNP(=O)(OC)OC(C)[C@H]1O[C@@H](N2C=NC3C(N)=NC=NC32)[C@H](O)[C@@H]1O]

(1R)-1-((2S,3S,4R,5R)-5-(6-amino-4H-purin-9(5H)-yl)-3,4-dihydroxytetrahydrofuran-2-yl)ethyl methyl (2-((4Z,7Z,10Z,13Z,16Z,19Z)-docosa-4,7,10,13,16,19-hexaenamido)ethyl)phosphoramidate (II-54)

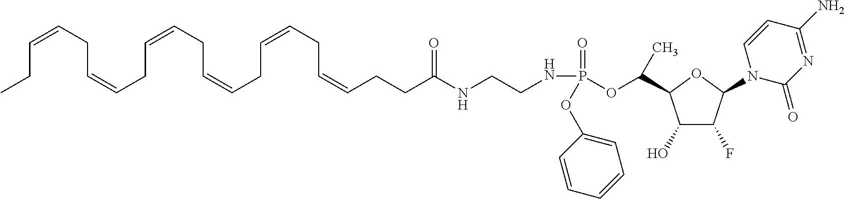 [SMILES: CC/C=C\C/C=C\C/C=C\C/C=C\C/C=C\C/C=C\CCC(=O)NCCNP(=O)(Oc1ccccc1)OC(C)[C@H]1O[C@@H](n2ccc(N)nc2=O)[C@H](F)[C@@H]1O]

(R)-1-((2S,3R,4R,5R)-5-(4-amino-2-oxopyrimidin-1(2H)-yl)-4-fluoro-3-hydroxytetrahydrofuran-2-yl)ethyl phenyl (2-((4Z,7Z,10Z,13Z,16Z,19Z)-docosa-4,7,10,13,16,19-hexaenamido)ethyl)phosphoramidate (II-55)

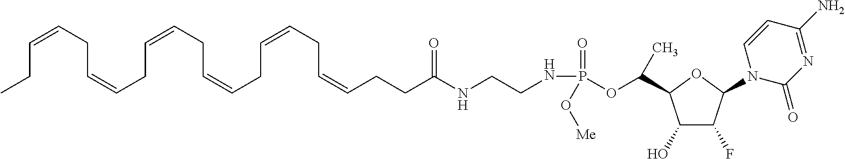 [SMILES: CC/C=C\C/C=C\C/C=C\C/C=C\C/C=C\C/C=C\CCC(=O)NCCNP(=O)(OC)OC(C)[C@H]1O[C@@H](n2ccc(N)nc2=O)[C@H](F)[C@@H]1O]

(R)-1-((2S,3R,4R,5R)-5-(4-amino-2-oxopyrimidin-1(2H)-yl)-4-fluoro-3-hydroxytetrahydrofuran-2-yl)ethyl methyl (2-((4Z,7Z,10Z,13Z,16Z,19Z)-docosa-4,7,10,13,16,19-hexaenamido)ethyl)phosphoramidate (II-56)

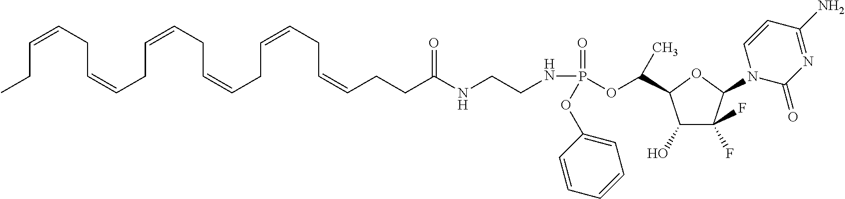

(R)-1-((2S,3R,5R)-5-(4-amino-2-oxopyrimidin-1(2H)-yl)-4,4-difluoro-3-hydroxytetrahydrofuran-2-yl)ethyl phenyl (2-((4Z,7Z,10Z,13Z,16Z,19Z)-docosa-4,7,10,13,16,19-hexaenamido)ethyl)phosphoramidate (II-57)

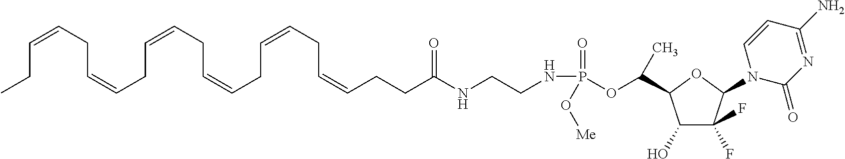

(R)-1-((2S,3R,5R)-5-(4-amino-2-oxopyrimidin-1(2H)-yl)-4,4-difluoro-3-hydroxytetrahydrofuran-2-yl)ethyl methyl (2-((4Z,7Z,10Z,13Z,16Z,19Z)-docosa-4,7,10,13,16,19-hexaenamido)ethyl)phosphoramidate (II-58)

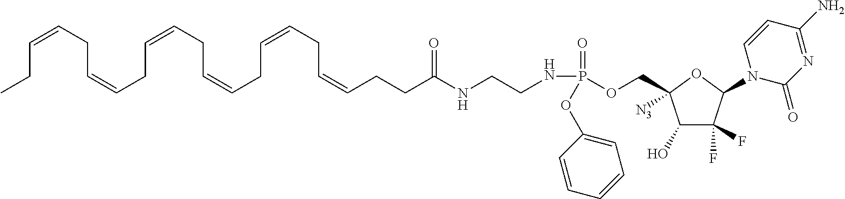

((2R,3R,5R)-5-(4-amino-2-oxopyrimidin-1(2H)-yl)-2-azido-4,4-difluoro-3-hydroxytetrahydrofuran-2-yl)methyl phenyl (2-((4Z,7Z,10Z,13Z,16Z,19Z)-docosa-4,7,10,13,16,19-hexaenamido)ethyl)phosphoramidate (II-59)

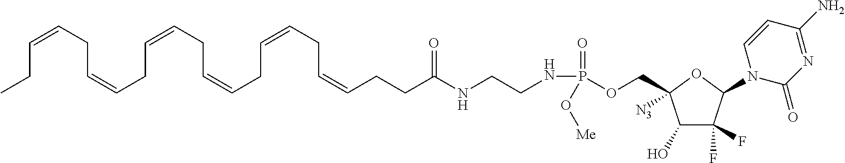

((2R,3R,5R)-5-(4-amino-2-oxopyrimidin-1(2H)-yl)-2-azido-4,4-difluoro-3-hydroxytetrahydrofuran-2-yl)methyl methyl (2-((4Z,7Z,10Z,13Z,16Z,19Z)-docosa-4,7,10,13,16,19-hexaenamido)ethyl)phosphoramidate (II-60)

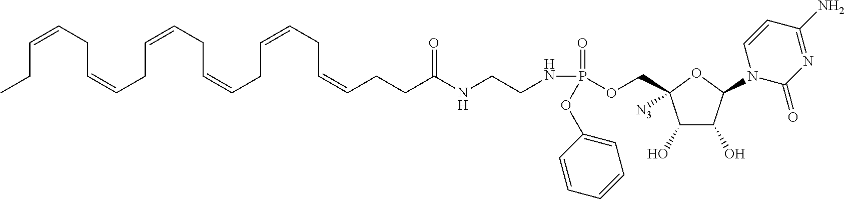 [SMILES: CC/C=C\C/C=C\C/C=C\C/C=C\C/C=C\C/C=C\CCC(=O)NCCNP(=O)(OC[C@@]1(N=[N+]=[N-])O[C@@H](n2ccc(N)nc2=O)[C@H](O)[C@@H]1O)Oc1ccccc1]

((2R,3S,4R,5R)-5-(4-amino-2-oxopyrimidin-1(2H)-yl)-2-azido-3,4-dihydroxytetrahydrofuran-2-yl)methyl phenyl (2-((4Z,7Z,10Z,13Z,16Z,19Z)-docosa-4,7,10,13,16,19-hexaenamido)ethyl)phosphoramidate (II-61)

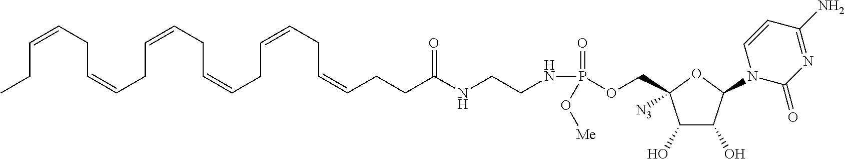 [SMILES: CC/C=C\C/C=C\C/C=C\C/C=C\C/C=C\C/C=C\CCC(=O)NCCNP(=O)(OC)OC[C@@]1(N=[N+]=[N-])O[C@@H](n2ccc(N)nc2=O)[C@H](O)[C@@H]1O]

((2R,3S,4R,5R)-5-(4-amino-2-oxopyrimidin-1(2H)-yl)-2-azido-3,4-dihydroxytetrahydrofuran-2-yl)methyl methyl (2-((4Z,7Z,10Z,13Z,16Z,19Z)-docosa-4,7,10,13,16,19-hexaenamido)ethyl)phosphoramidate (II-62)

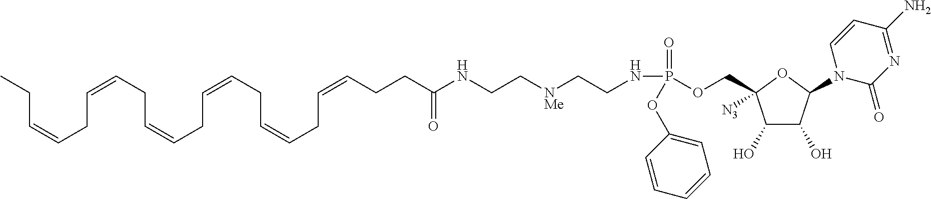 [SMILES: CC/C=C\C/C=C\C/C=C\C/C=C\C/C=C\C/C=C\CCC(=O)NCCN(C)CCNP(=O)(OC[C@@]1(N=[N+]=[N-])O[C@@H](n2ccc(N)nc2=O)[C@H](O)[C@@H]1O)Oc1ccccc1]

((2R,3S,4R,5R)-5-(4-amino-2-oxopyrimidin-1(2H)-yl)-2-azido-3,4-dihydroxytetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-63)

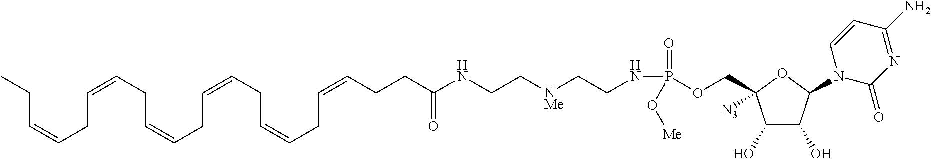 [SMILES: CC/C=C\C/C=C\C/C=C\C/C=C\C/C=C\C/C=C\CCC(=O)NCCN(C)CCNP(=O)(OC)OC[C@@]1(N=[N+]=[N-])O[C@@H](n2ccc(N)nc2=O)[C@H](O)[C@@H]1O]

((2R,3S,4R,5R)-5-(4-amino-2-oxopyrimidin-1(2H)-yl)-2-azido-3,4-dihydroxytetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-64)

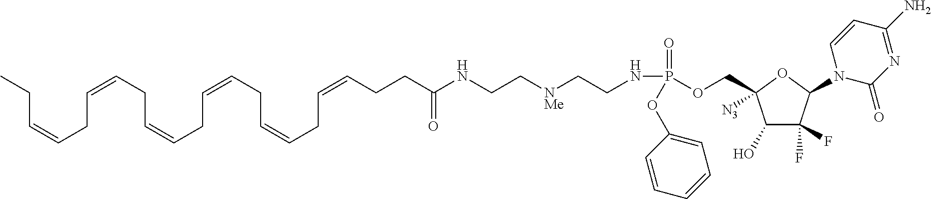

((2R,3R,5R)-5-(4-amino-2-oxopyrimidin-1(2H)-yl)-2-azido-4,4-difluoro-3-hydroxytetrahydrofuran-2-yl)methyl phenyl (2-((2-((4Z,7Z,10Z,13Z,16Z,19Z)-docosa-4,7,10,13,16,19-hexaenamido)ethyl)(methyl)amino)ethyl)phosphoramidate (II-65)

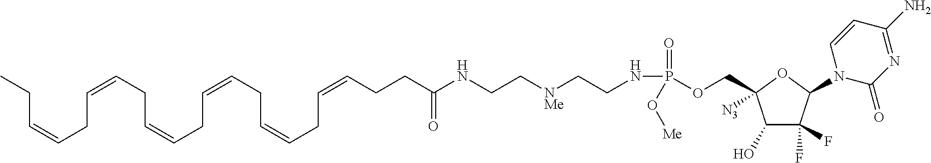

((2R,3R,5R)-5-(4-amino-2-oxopyrimidin-1(2H)-yl)-2-azido-4,4-difluoro-3-hydroxytetrahydrofuran-2-yl)methyl methyl (2-((2-((4Z,7Z,10Z,13Z,16Z,19Z)-docosa-4,7,10,13,16,19-hexaenamido)ethyl)(methyl)amino)ethyl)phosphoramidate (II-66)

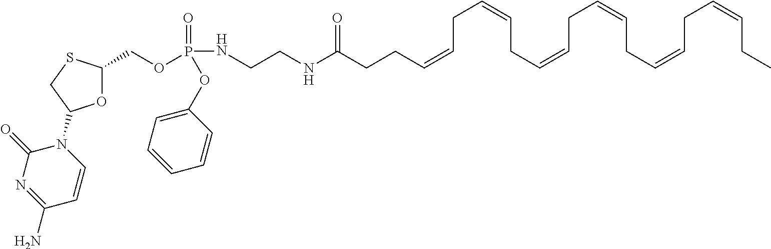

((2R,5S)-5-(4-amino-2-oxopyrimidin-1(2H)-yl)-1,3-oxathiolan-2-yl)methyl phenyl (2-((4Z,7Z,10Z,13Z,16Z,19Z)-docosa-4,7,10,13,16,19-hexaenamido)ethyl)phosphoramidate (II-67)

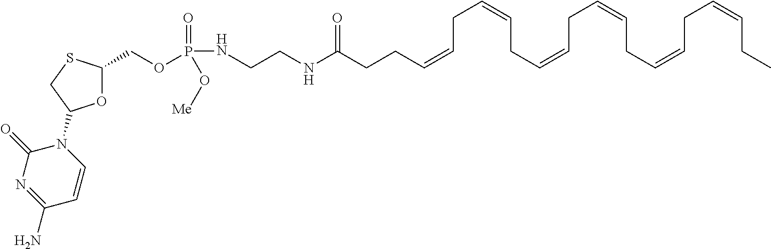

((2R,5S)-5-(4-amino-2-oxopyrimidin-1(2H)-yl)-1,3-oxathiolan-2-yl)methyl methyl (2-((4Z,7Z,10Z,13Z,16Z,19Z)-docosa-4,7,10,13,16,19-hexaenamido)ethyl)phosphoramidate (II-68)

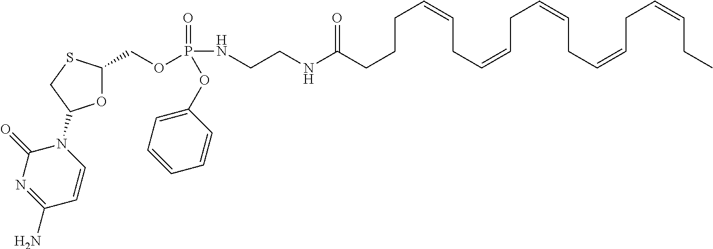

((2R,5S)-5-(4-amino-2-oxopyrimidin-1(2H)-yl)-1,3-oxathiolan-2-yl)methyl phenyl (2-((5Z,8Z,11Z,14Z,17Z)-icosa-5,8,11,14,17-pentaenamido)ethyl)phosphoramidate (II-69)

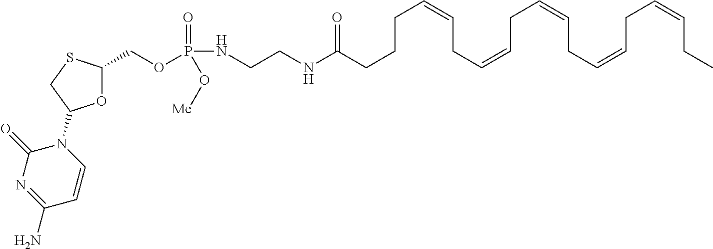

((2R,5S)-5-(4-amino-2-oxopyrimidin-1(2H)-yl)-1,3-oxathiolan-2-yl)methyl methyl (2-((5Z,8Z,11Z,14Z,17Z)-icosa-5,8,11,14,17-pentaenamido)ethyl)phosphoramidate (II-70)

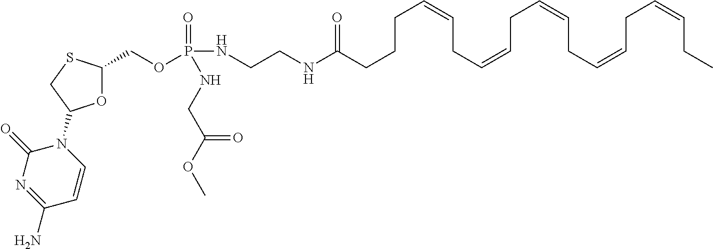

(13Z,16Z,19Z,22Z,25Z)-methyl 4-(((2R,5S)-5-(4-amino-2-oxopyrimidin-1(2H)-yl)-1,3-oxathiolan-2-yl)methoxy)-4,9-dioxo-3,5,8-triaza-4-phosphaoctacosa-13,16,19,22,25-pentaen-1-oate (II-71)

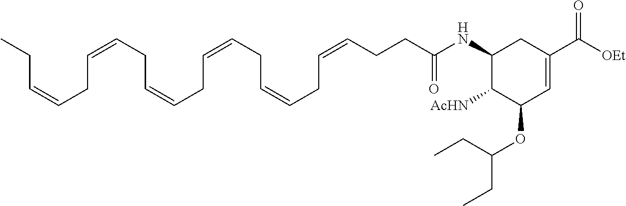 [SMILES: CC/C=C\C/C=C\C/C=C\C/C=C\C/C=C\C/C=C\CCC(=O)N[C@H]1CC(C(=O)OCC)=C[C@@H](OC(CC)CC)[C@@H]1NC(C)=O]

(3R,4R,5S)-ethyl 4-acetamido-5-((4Z,7Z,10Z,13Z,16Z,19Z)-docosa-4,7,10,13,16,19-hexaenamido)-3-(pentan-3-yloxy)cyclohex-1-enecarboxylate (IV-1)

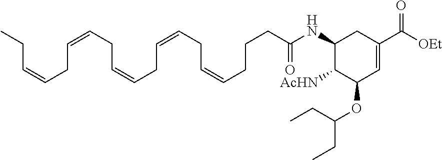 [SMILES: CC/C=C\C/C=C\C/C=C\C/C=C\C/C=C\CCCC(=O)N[C@H]1CC(C(=O)OCC)=C[C@@H](OC(CC)CC)[C@@H]1NC(C)=O]

(3R,4R,5S)-ethyl 4-acetamido-5-((5Z,8Z,11Z,14Z,17Z)-icosa-5,8,11,14,17-pentaenamido)-3-(pentan-3-yloxy)cyclohex-1-enecarboxylate (IV-2)

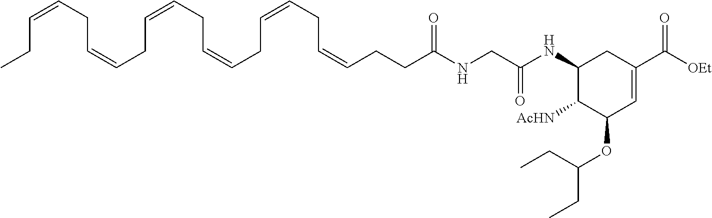 [SMILES: CC/C=C\C/C=C\C/C=C\C/C=C\C/C=C\C/C=C\CCC(=O)NCC(=O)N[C@H]1CC(C(=O)OCC)=C[C@@H](OC(CC)CC)[C@@H]1NC(C)=O]

(3R,4R,5S)-ethyl 4-acetamido-5-(2-((4Z,7Z,10Z,13Z,16Z,19Z)-docosa-4,7,10,13,16,19-hexaenamido)acetamido)-3-(pentan-3-yloxy)cyclohex-1-enecarboxylate (IV-3)

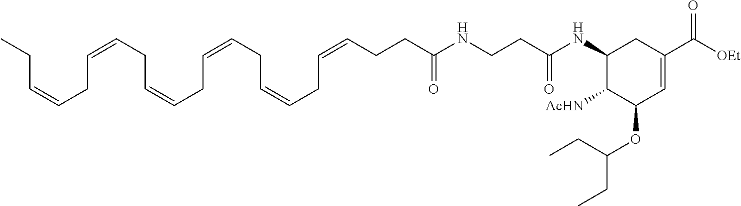 [SMILES: CC/C=C\C/C=C\C/C=C\C/C=C\C/C=C\C/C=C\CCC(=O)NCCC(=O)N[C@H]1CC(C(=O)OCC)=C[C@@H](OC(CC)CC)[C@@H]1NC(C)=O]

(3R,4R,5S)-ethyl 4-acetamido-5-(3-((4Z,7Z,10Z,13Z,16Z,19Z)-docosa-4,7,10,13,16,19-hexaenamido)propanamido)-3-(pentan-3-yloxy)cyclohex-1-enecarboxylate (IV-4)

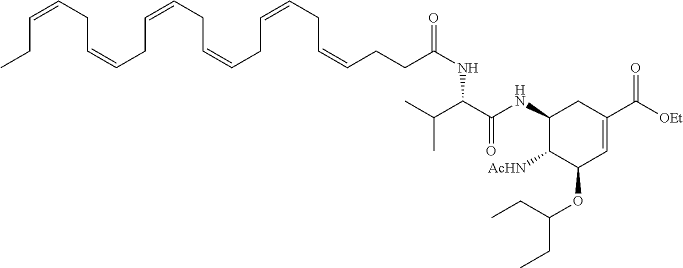 [SMILES: CC/C=C\C/C=C\C/C=C\C/C=C\C/C=C\C/C=C\CCC(=O)N[C@H](C(=O)N[C@H]1CC(C(=O)OCC)=C[C@@H](OC(CC)CC)[C@@H]1NC(C)=O)C(C)C]

(3R,4R,5S)-ethyl 4-acetamido-5-((S)-2-((4Z,7Z,10Z,13Z,16Z,19Z)-docosa-4,7,10,13,16,19-hexaenamido)-3-methylbutanamido)-3-(pentan-3-yloxy)cyclohex-1-enecarboxylate (IV-5)

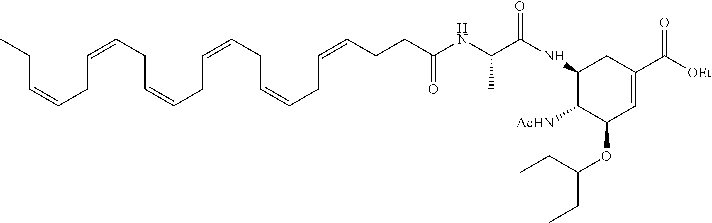 [SMILES: CC/C=C\C/C=C\C/C=C\C/C=C\C/C=C\C/C=C\CCC(=O)N[C@@H](C)C(=O)N[C@H]1CC(C(=O)OCC)=C[C@@H](OC(CC)CC)[C@@H]1NC(C)=O]

(3R,4R,5S)-ethyl 4-acetamido-5-((S)-2-((4Z,7Z,10Z,13Z,16Z,19Z)-docosa-4,7,10,13,16,19-hexaenamido)propanamido)-3-(pentan-3-yloxy)cyclohex-1-enecarboxylate (IV-6)

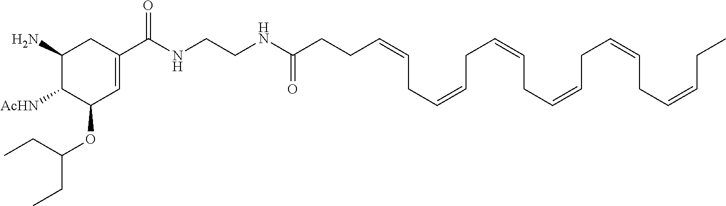 [SMILES: CC/C=C\C/C=C\C/C=C\C/C=C\C/C=C\C/C=C\CCC(=O)NCCNC(=O)C1=C[C@@H](OC(CC)CC)[C@H](NC(C)=O)[C@@H](N)C1]

(3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (IV-7)

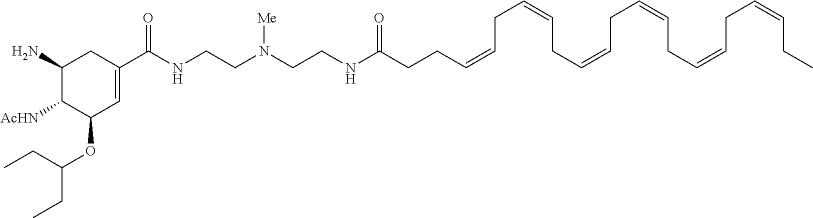 [SMILES: CC/C=C\C/C=C\C/C=C\C/C=C\C/C=C\C/C=C\CCC(=O)NCCN(C)CCNC(=O)C1=C[C@@H](OC(CC)CC)[C@H](NC(C)=O)[C@@H](N)C1]

(3R,4R,5S)-4-acetamido-5-amino-N-(2-((2-((4Z,7Z,10Z,13Z,16Z,19Z)-docosa-4,7,10,13,16,19-hexaenamido)ethyl)(methyl)amino)ethyl)-3-(pentan-3-yloxy)cyclohex-1-enecarboxamide (IV-8)

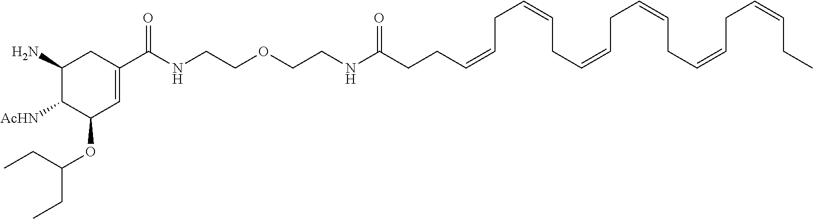 [SMILES: CC/C=C\C/C=C\C/C=C\C/C=C\C/C=C\C/C=C\CCC(=O)NCCOCCNC(=O)C1=C[C@@H](OC(CC)CC)[C@H](NC(C)=O)[C@@H](N)C1]

(3R,4R,5S)-4-acetamido-5-amino-N-(2-(2-((4Z,7Z,10Z,13Z,16Z,19Z)-docosa-4,7,10,13,16,19-hexaenamido)ethoxy)ethyl)-3-(pentan-3-yloxy)cyclohex-1-enecarboxamide (IV-9)

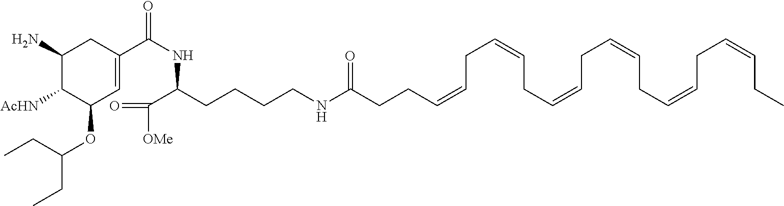 [SMILES: CC/C=C\C/C=C\C/C=C\C/C=C\C/C=C\C/C=C\CCC(=O)NCCCC[C@H](NC(=O)C1=C[C@@H](OC(CC)CC)[C@H](NC(C)=O)[C@@H](N)C1)C(=O)OC]

(S)-methyl 2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoate (IV-10)

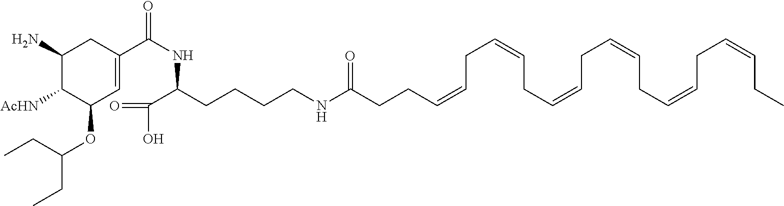 [SMILES: CC/C=C\C/C=C\C/C=C\C/C=C\C/C=C\C/C=C\CCC(=O)NCCCC[C@H](NC(=O)C1=C[C@@H](OC(CC)CC)[C@H](NC(C)=O)[C@@H](N)C1)C(=O)O]

(S)-2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoic acid (IV-11)

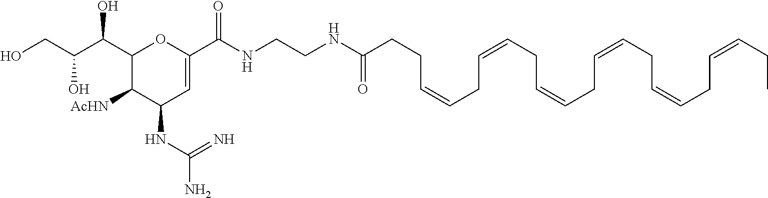 [SMILES: CC/C=C\C/C=C\C/C=C\C/C=C\C/C=C\C/C=C\CCC(=O)NCCNC(=O)C1=C[C@@H](NC(=N)N)[C@@H](NC(C)=O)C([C@H](O)[C@H](O)CO)O1]

(3R,4R)-3-acetamido-N-(2-((4Z,7Z,10Z,13Z,16Z,19Z)-docosa-4,7,10,13,16,19-hexaenamido)ethyl)-4-guanidino-2-((1R,2R)-1,2,3-trihydroxypropyl)-3,4-dihydro-2H-pyran-6-carboxamide (IV-12)

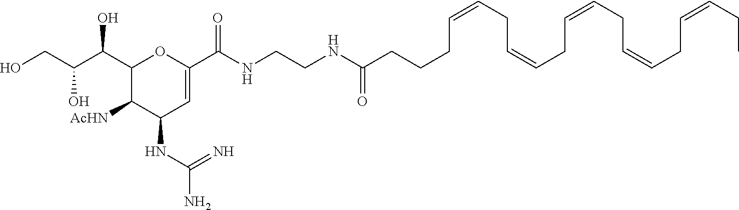 [SMILES: CC/C=C\C/C=C\C/C=C\C/C=C\C/C=C\CCCC(=O)NCCNC(=O)C1=C[C@@H](NC(=N)N)[C@@H](NC(C)=O)C([C@H](O)[C@H](O)CO)O1]

(3R,4R)-3-acetamido-4-guanidino-N-(2-((5Z,8Z,11Z,14Z,17Z)-icosa-5,8,11,14,17-pentaenamido)ethyl)-2-((1R,2R)-1,2,3-trihydroxypropyl)-3,4-dihydro-2H-pyran-6-carboxamide (IV-13)

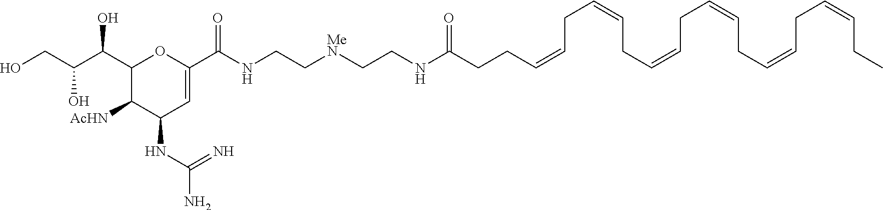 [SMILES: CC/C=C\C/C=C\C/C=C\C/C=C\C/C=C\C/C=C\CCC(=O)NCCN(C)CCNC(=O)C1=C[C@@H](NC(=N)N)[C@@H](NC(C)=O)C([C@H](O)[C@H](O)CO)O1]

(3R,4R)-3-acetamido-N-(2-((2-((4Z,7Z,10Z,13Z,16Z,19Z)-docosa-4,7,10,13,16,19-hexaenamido)ethyl)(methyl)amino)ethyl)-4-guanidino-2-((1R,2R)-1,2,3-trihydroxypropyl)-3,4-dihydro-2H-pyran-6-carboxamide (IV-14)

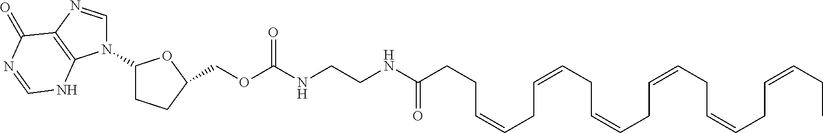

((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (V-1)

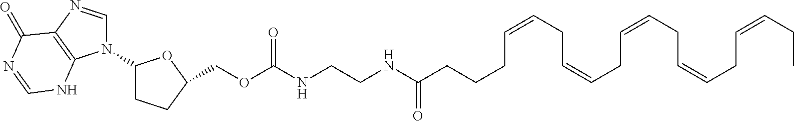

((2S,5R)-5-(6-oxo-3H-purin-9(6H)-yl)tetrahydrofuran-2-yl)methyl (2-((5Z,8Z,11Z,14Z,17Z)-icosa-5,8,11,14,17-pentaenamido)ethyl)carbamate (V-2)

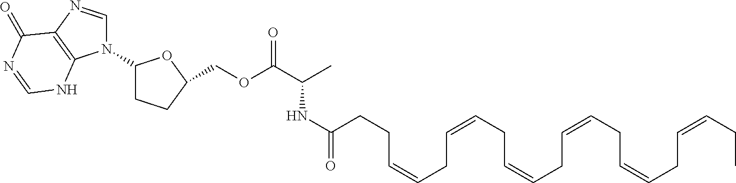

(S)-((2S,5R)-5-(6-oxo-3H-purin-9(6H)-yl)tetrahydrofuran-2-yl)methyl 2-((4Z,7Z,10Z,13Z,16Z,19Z)-docosa-4,7,10,13,16,19-hexaenamido)propanoate (V-3)

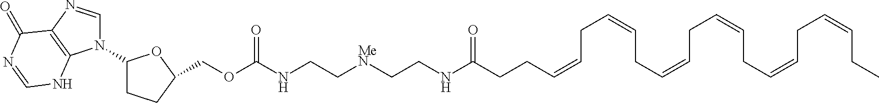

((2S,5R)-5-(6-oxo-3H-purin-9(6H)-yl)tetrahydrofuran-2-yl)methyl (2-((2-((4Z,7Z,10Z,13Z,16Z,19Z)-docosa-4,7,10,13,16,19-hexaenamido)ethyl)(methyl)amino)ethyl)carbamate (V-4)

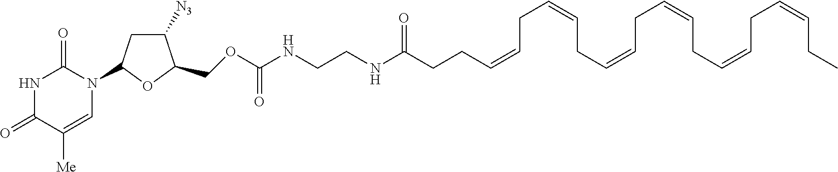

((2S,3S,5S)-3-azido-5-(5-methyl-2,4-dioxo-3,4-dihydropyrimidin-1(2H)-yl)tetrahydrofuran-2-yl)methyl (2-((4Z,7Z,10Z,13Z,16Z,19Z)-docosa-4,7,10,13,16,19-hexaenamido)ethyl)carbamate (V-5)

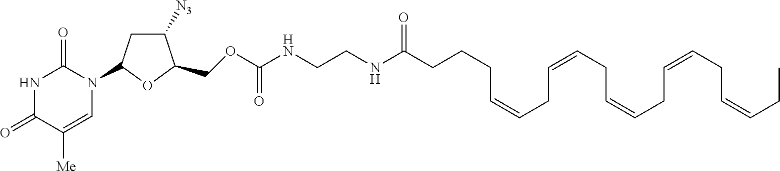

((2S,3S,5S)-3-azido-5-(5-methyl-2,4-dioxo-3,4-dihydropyrimidin-1 (2H)— yl)tetrahydrofuran-2-yl)methyl (2-((5Z,8Z,11Z,14Z,17Z)-icosa-5,8,11,14,17-pentaenamido)ethyl)carbamate (V-6)

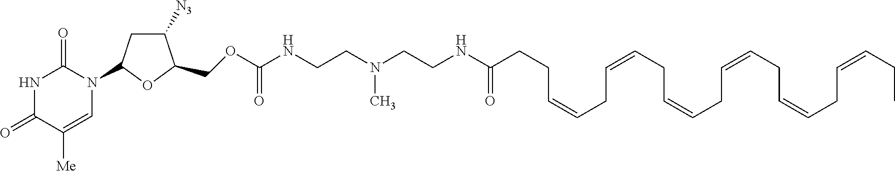

((2S,3S,5S)-3-azido-5-(5-methyl-2,4-dioxo-3,4-dihydropyrimidin-1(2H)-yl)tetrahydrofuran-2-yl)methyl (2-((2-((4Z,7Z,10Z,13Z,16Z,19Z)-docosa-4,7,10,13,16,19-hexaenamido)ethyl)(methyl)amino)ethyl)carbamate (V-7)

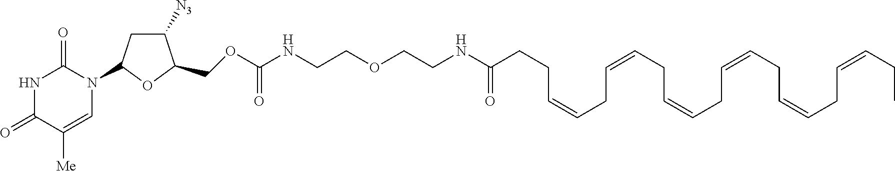

((2S,3S,5S)-3-azido-5-(5-methyl-2,4-dioxo-3,4-dihydropyrimidin-1 (2H)-yl)tetrahydrofuran-2-yl)methyl (2-(2-((4Z,7Z,10Z,13Z,16Z,19Z)-docosa-4,7,10,13,16,19-hexaenamido)ethoxy)ethyl)carbamate (V-8)

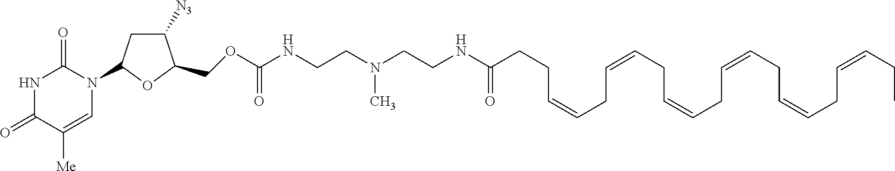

((2S,3S,5S)-3-azido-5-(5-methyl-2,4-dioxo-3,4-dihydropyrimidin-1(2H)-yl)tetrahydrofuran-2-yl)methyl (2-((2-((4Z,7Z,10Z,13Z,16Z,19Z)-docosa-4,7,10,13,16,19-hexaenamido)ethyl)(methyl)amino)ethyl)carbamate (V-7)

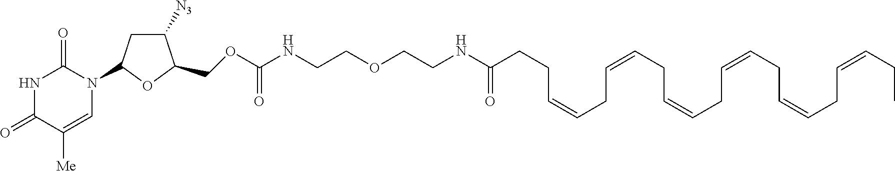

((2S,3S,5S)-3-azido-5-(5-methyl-2,4-dioxo-3,4-dihydropyrimidin-1(2H)-yl)tetrahydrofuran-2-yl)methyl (2-(2-((4Z,7Z,10Z,13Z,16Z,19Z)-docosa-4,7,10,13,16,19-hexaenamido)ethoxy)ethyl)carbamate (V-8)

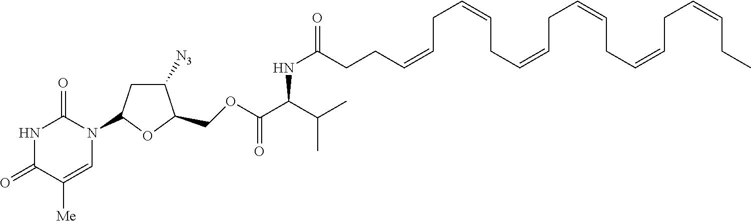

(S)-((2S,3S,5S)-3-azido-5-(5-methyl-2,4-dioxo-3,4-dihydropyrimidin-1(2H)-yl)tetrahydrofuran-2-yl)methyl 2-((4Z,7Z,10Z,13Z,16Z,19Z)-docosa-4,7,10,13,16,19-hexaenamido)-3-methylbutanoate (V-9)

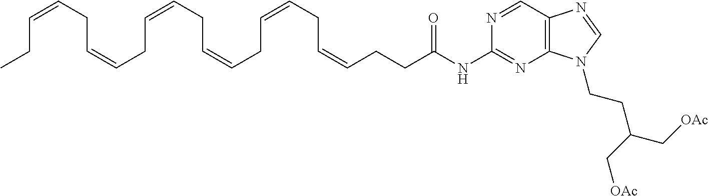 [SMILES: CC/C=C\C/C=C\C/C=C\C/C=C\C/C=C\C/C=C\CCC(=O)Nc1ncc2ncn(CCC(COC(C)=O)COC(C)=O)c2n1]

2-(2-(2-((4Z,7Z,10Z,13Z,16Z,19Z)-docosa-4,7,10,13,16,19-hexaenamido)-9H-purin-9-yl)ethyl)propane-1,3-diyl diacetate (V-10)

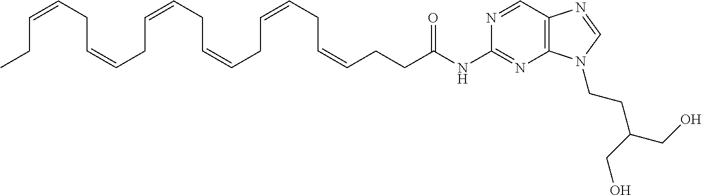 [SMILES: CC/C=C\C/C=C\C/C=C\C/C=C\C/C=C\C/C=C\CCC(=O)Nc1ncc2ncn(CCC(CO)CO)c2n1]

(4Z,7Z,10Z,13Z,16Z,19Z)—N-(9-(4-hydroxy-3-(hydroxymethyl)butyl)-9H-purin-2-yl)docosa-4,7,10,13,16,19-hexaenamide (V-11)

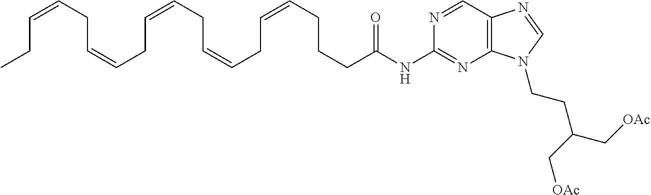 [SMILES: CC/C=C\C/C=C\C/C=C\C/C=C\C/C=C\CCCC(=O)Nc1ncc2ncn(CCC(COC(C)=O)COC(C)=O)c2n1]

2-(2-(2-((5Z,8Z,11Z,14Z,17Z)-icosa-5,8,11,14,17-pentaenamido)-9H-purin-9-yl)ethyl)propane-1,3-diyl diacetate (V-12)

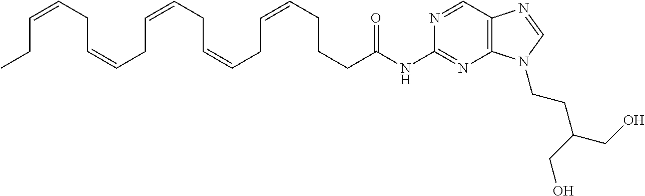 [SMILES: CC/C=C\C/C=C\C/C=C\C/C=C\C/C=C\CCCC(=O)Nc1ncc2ncn(CCC(CO)CO)c2n1]

(5Z,8Z,11Z,14Z,17Z)—N-(9-(4-hydroxy-3-(hydroxymethyl)butyl)-9H-purin-2-yl)icosa-5,8,11,14,17-pentaenamide (V-13)

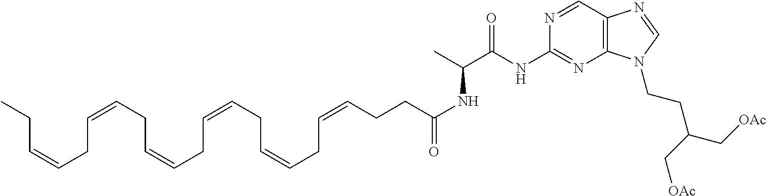 [SMILES: CC/C=C\C/C=C\C/C=C\C/C=C\C/C=C\C/C=C\CCC(=O)N[C@@H](C)C(=O)Nc1ncc2ncn(CCC(COC(C)=O)COC(C)=O)c2n1]

2-(2-(2-((S)-2-((4Z,7Z,10Z,13Z,16Z,19Z)-docosa-4,7,10,13,16,19-hexaenamido)propanamido)-9H-purin-9-yl)ethyl)propane-1,3-diyl diacetate (V-14)

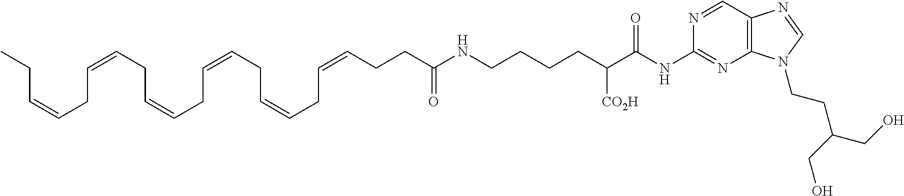 [SMILES: CC/C=C\C/C=C\C/C=C\C/C=C\C/C=C\C/C=C\CCC(=O)NCCCCC(C(=O)O)C(=O)Nc1ncc2ncn(CCC(CO)CO)c2n1]

6-((4Z,7Z,10Z,13Z,16Z,19Z)-docosa-4,7,10,13,16,19-hexaenamido)-2-((9-(4-hydroxy-3-(hydroxymethyl)butyl)-9H-purin-2-yl)carbamoyl)hexanoic acid (V-15)

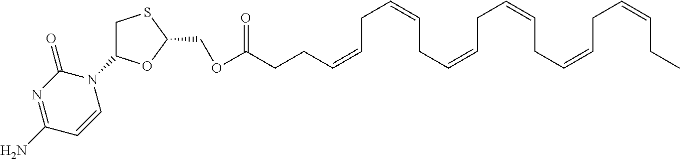

(4Z,7Z,10Z,13Z,16Z,19Z)-((2R,5S)-5-(4-amino-2-oxopyrimidin-1(2H)-yl)-1,3-oxathiolan-2-yl)methyl docosa-4,7,10,13,16,19-hexaenoate (V-16)

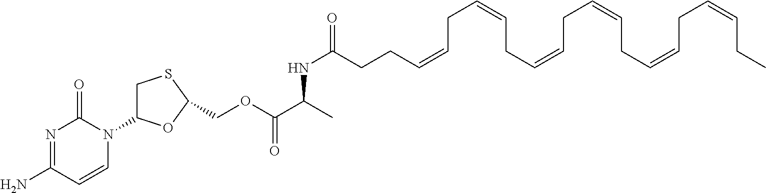

(S)-((2R,5S)-5-(4-amino-2-oxopyrimidin-1(2H)-yl)-1,3-oxathiolan-2-yl)methyl 2-((4Z,7Z,10Z,13Z,16Z,19Z)-docosa-4,7,10,13,16,19-hexaenamido)propanoate (V-17)

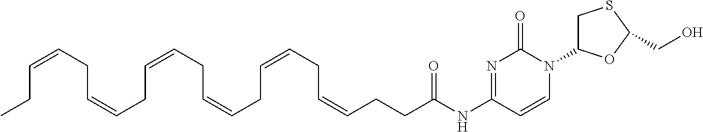

(4Z,7Z,10Z,13Z,16Z,19Z)—N-(1-((2R,5S)-2-(hydroxymethyl)-1,3-oxathiolan-5-yl)-2-oxo-1,2-dihydropyrimidin-4-yl)docosa-4,7,10,13,16,19-hexaenamide (V-18)

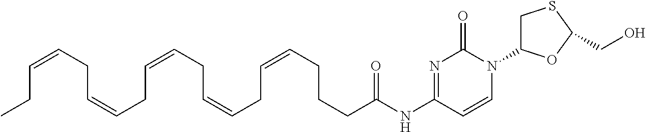

(5Z,8Z,11Z,14Z,17Z)—N-(1-((2R,5S)-2-(hydroxymethyl)-1,3-oxathiolan-5-yl)-2-oxo-1,2-dihydropyrimidin-4-yl)icosa-5,8,11,14,17-pentaenamide (V-19)

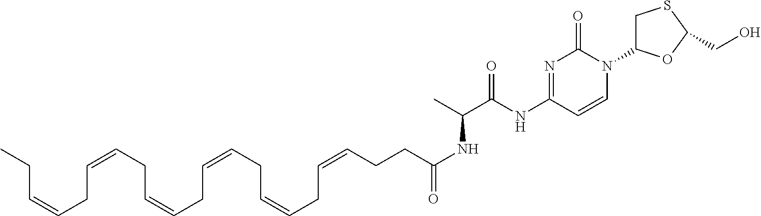

(4Z,7Z,10Z,13Z,16Z,19Z)—N—((S)-1-((1-((2R,5S)-2-(hydroxymethyl)-1,3-oxathiolan-5-yl)-2-oxo-1,2-dihydropyrimidin-4-yl)amino)-1-oxopropan-2-yl)docosa-4,7,10,13,16,19-hexaenamide (V-20)

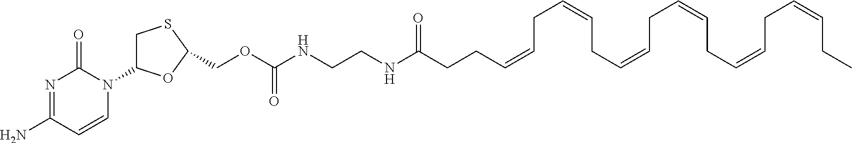

((2R,5S)-5-(4-amino-2-oxopyrimidin-1 (2H)-yl)-1,3-oxathiolan-2-yl)methyl (2-((4Z,7Z,10Z,13Z,16Z,19Z)-docosa-4,7,10,13,16,19-hexaenamido)ethyl)carbamate (V-21)

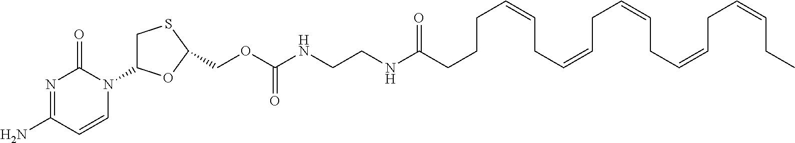

((2R,5S)-5-(4-amino-2-oxopyrimidin-1(2H)-yl)-1,3-oxathiolan-2-yl)methyl (2-((5Z,8Z,11Z,14Z,17Z)-icosa-5,8,11,14,17-pentaenamido)ethyl)carbamate (V-22)

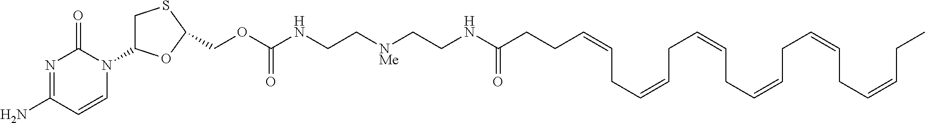

((2R,5S)-5-(4-amino-2-oxopyrimidin-1(2H)-yl)-1,3-oxathiolan-2-yl)methyl (2-((2-((4Z,7Z,10Z,13Z,16Z,19Z)-docosa-4,7,10,13,16,19-hexaenamido)ethyl)(methyl)amino)ethyl)carbamate (V-23)

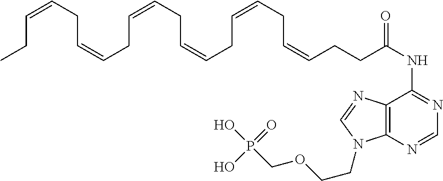 [SMILES: CC/C=C\C/C=C\C/C=C\C/C=C\C/C=C\C/C=C\CCC(=O)Nc1ncnc2c1ncn2CCOCP(=O)(O)O]

((2-(6-((4Z,7Z,10Z,13Z,16Z,19Z)-docosa-4,7,10,13,16,19-hexaenamido)-9H-purin-9-yl)ethoxy)methyl)phosphonic acid (V-24)

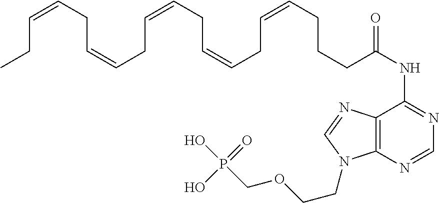 [SMILES: CC/C=C\C/C=C\C/C=C\C/C=C\C/C=C\CCCC(=O)Nc1ncnc2c1ncn2CCOCP(=O)(O)O]

((2-(6-((5Z,8Z,11Z,14Z,17Z)-icosa-5,8,11,14,17-pentaenamido)-9H-purin-9-yl)ethoxy)methyl)phosphonic acid (V-25)

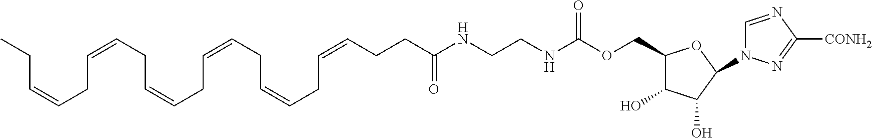 [SMILES: CC/C=C\C/C=C\C/C=C\C/C=C\C/C=C\C/C=C\CCC(=O)NCCNC(=O)OC[C@H]1O[C@@H](n2cnc(C(N)=O)n2)[C@H](O)[C@@H]1O]

((2R,3S,4R,5R)-5-(3-carbamoyl-1H-1,2,4-triazol-1-yl)-3,4-dihydroxytetrahydrofuran-2-yl)methyl (2-((4Z,7Z,10Z,13Z,16Z,19Z)-docosa-4,7,10,13,16,19-hexaenamido)ethyl)carbamate (V-26)

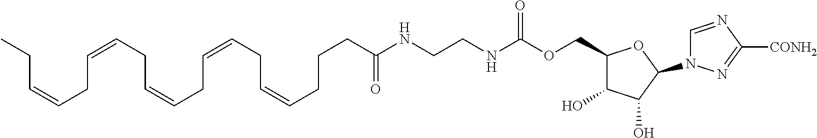 [SMILES: CC/C=C\C/C=C\C/C=C\C/C=C\C/C=C\CCCC(=O)NCCNC(=O)OC[C@H]1O[C@@H](n2cnc(C(N)=O)n2)[C@H](O)[C@@H]1O]

((2R,3S,4R,5R)-5-(3-carbamoyl-1H-1,2,4-triazol-1-yl)-3,4-dihydroxytetrahydrofuran-2-yl)methyl (2-((5Z,8Z,11Z,14Z,17Z)-icosa-5,8,11,14,17-pentaenamido)ethyl)carbamate (V-27)

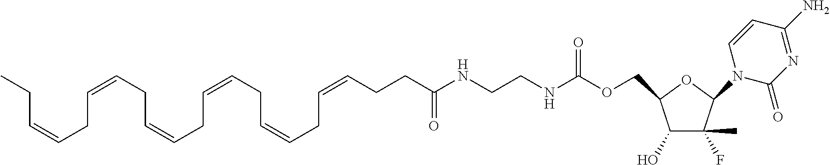 [SMILES: CC/C=C\C/C=C\C/C=C\C/C=C\C/C=C\C/C=C\CCC(=O)NCCNC(=O)OC[C@H]1O[C@@H](n2ccc(N)nc2=O)[C@](C)(F)[C@@H]1O]

((2R,3R,4R,5R)-5-(4-amino-2-oxopyrimidin-1(2H)-yl)-4-fluoro-3-hydroxy-4-methyltetrahydrofuran-2-yl)methyl (2-((4Z,7Z,10Z,13Z,16Z,19Z)-docosa-4,7,10,13,16,19-hexaenamido)ethyl)carbamate (V-28)

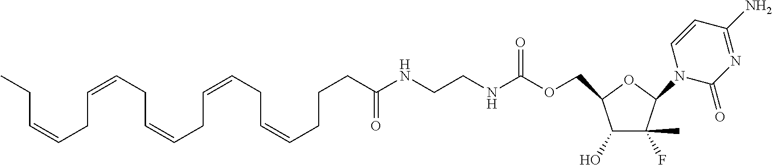 [SMILES: CC/C=C\C/C=C\C/C=C\C/C=C\C/C=C\CCCC(=O)NCCNC(=O)OC[C@H]1O[C@@H](n2ccc(N)nc2=O)[C@](C)(F)[C@@H]1O]

((2R,3R,4R,5R)-5-(4-amino-2-oxopyrimidin-1(2H)-yl)-4-fluoro-3-hydroxy-4-methyltetrahydrofuran-2-yl)methyl (2-((5Z,8Z,11Z,14Z,17Z)-icosa-5,8,11,14,17-pentaenamido)ethyl)carbamate (V-29)

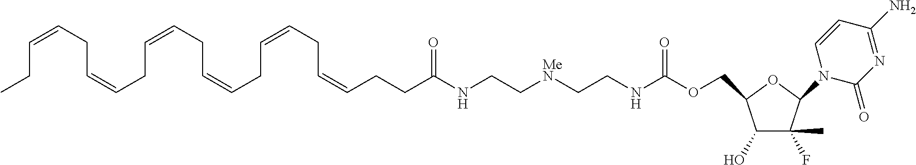 [SMILES: CC/C=C\C/C=C\C/C=C\C/C=C\C/C=C\C/C=C\CCC(=O)NCCN(C)CCNC(=O)OC[C@H]1O[C@@H](n2ccc(N)nc2=O)[C@](C)(F)[C@@H]1O]

((2R,3R,4R,5R)-5-(4-amino-2-oxopyrimidin-1(2H)-yl)-4-fluoro-3-hydroxy-4-methyltetrahydrofuran-2-yl)methyl (2-((2-((4Z,7Z,10Z,13Z,16Z,19Z)-docosa-4,7,10,13,16,19-hexaenamido)ethyl)(methyl)amino)ethyl)carbamate (V-30)

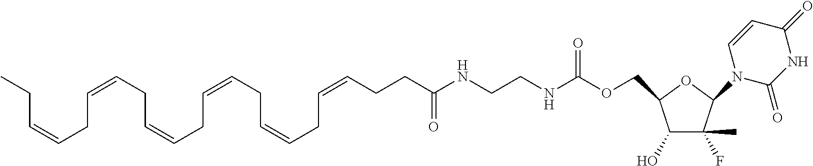 [SMILES: CC/C=C\C/C=C\C/C=C\C/C=C\C/C=C\C/C=C\CCC(=O)NCCNC(=O)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl (2-((4Z,7Z,10Z,13Z,16Z,19Z)-docosa-4,7,10,13,16,19-hexaenamido)ethyl)carbamate (V-31)

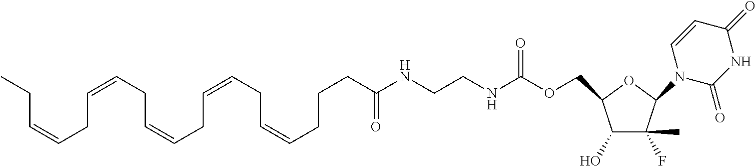 [SMILES: CC/C=C\C/C=C\C/C=C\C/C=C\C/C=C\CCCC(=O)NCCNC(=O)OC[C@H]1O[C@@H](n2ccc(=O)[nH]c2=O)[C@](C)(F)[C@@H]1O]

((2R,3R,4R,5R)-5-(2,4-dioxo-3,4-dihydropyrimidin-1(2H)-yl)-4-fluoro-3-hydroxy-4-methyltetrahydrofuran-2-yl)methyl (2-((5Z,8Z,11Z,14Z,17Z)-icosa-5,8,11,14,17-pentaenamido)ethyl)carbamate (V-32)

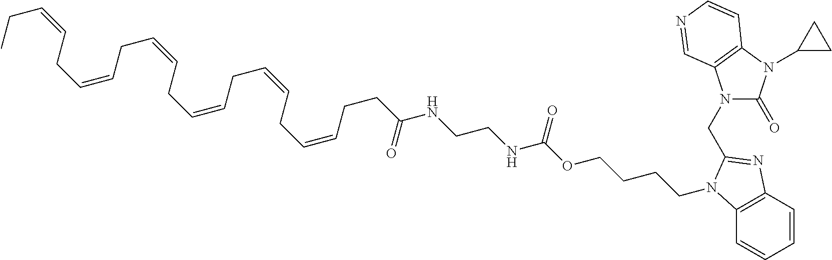 [SMILES: CC/C=C\C/C=C\C/C=C\C/C=C\C/C=C\C/C=C\CCC(=O)NCCNC(=O)OCCCCn1c(Cn2c(=O)n(C3CC3)c3ccncc32)nc2ccccc21]

4-(2-((1-cyclopropyl-2-oxo-1H-imidazo[4,5-c]pyridin-3 (2H)-yl)methyl)-1H-benzo [d]imidazol-1-yl)butyl (2-((4Z,7Z,10Z,13Z,16Z,19Z)-docosa-4,7,10,13,16,19-hexaenamido)ethyl)carbamate (VI-1)

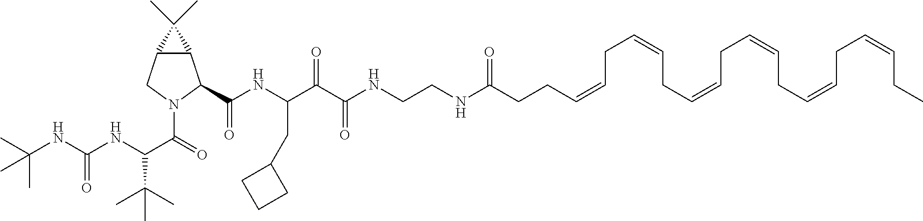

(1S,2S,5R)-3-((R)-2-(3-(tert-butyl)ureido)-3,3-dimethylbutanoyl)-N-(1-cyclobutyl-4-((2-((4Z,7Z,10Z,13Z,16Z,19Z)-docosa-4,7,10,13,16,19-hexaenamido)ethyl)amino)-3,4-dioxobutan-2-yl)-6,6-dimethyl-3-azabicyclo[3.1.0]hexane-2-carboxamide (VI-2)

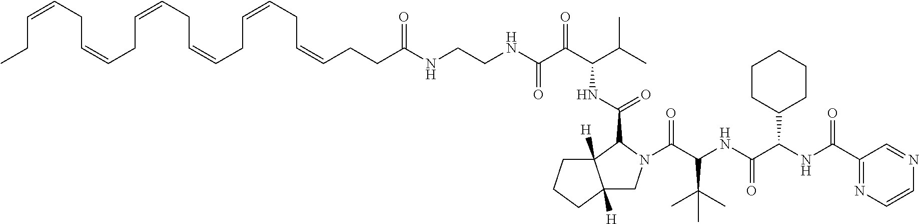

(1R,3aR,6aS)-2-(R)-2-((S)-2-cyclohexyl-2-(pyrazine-2-carboxamido)acetamido)-3,3-dimethylbutanoyl)-N-((S)-1-((2-((4Z,7Z,10Z,13Z,16Z,19Z)-docosa-4,7,10,13,16,19-hexaenamido)ethyl)amino)-4-methyl-1,2-dioxopentan-3-yl)octahydrocyclopenta[c]pyrrole-1-carboxamide (VI-3)

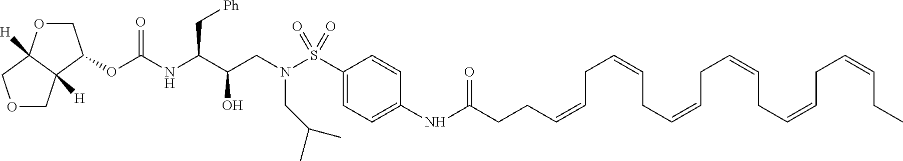 [SMILES: CC/C=C\C/C=C\C/C=C\C/C=C\C/C=C\C/C=C\CCC(=O)Nc1ccc(S(=O)(=O)N(CC(C)C)C[C@@H](O)[C@H](Cc2ccccc2)NC(=O)O[C@H]2CO[C@H]3COC[C@H]32)cc1]

(3R,3aS,6aR)-hexahydrofuro[2,3-b]furan-3-yl((2S,3R)-4-(4-((4Z,7Z,10Z,13Z,16Z,19Z)-docosa-4,7,10,13,16,19-hexaenamido)-N-isobutylphenylsulfonamido)-3-hydroxy-1-phenylbutan-2-yl)carbamate (VII-1)

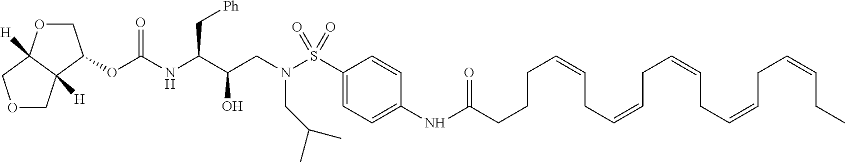 [SMILES: CC/C=C\C/C=C\C/C=C\C/C=C\C/C=C\CCCC(=O)Nc1ccc(S(=O)(=O)N(CC(C)C)C[C@@H](O)[C@H](Cc2ccccc2)NC(=O)O[C@H]2CO[C@H]3COC[C@H]32)cc1]

(3R,3aS,6aR)-hexahydrofuro[2,3-b]furan-3-yl((2S,3R)-3-hydroxy-4-(4-((5Z,8Z,11Z,14Z,17Z)-icosa-5,8,11,14,17-pentaenamido)-N-isobutylphenylsulfonamido)-1-phenylbutan-2-yl)carbamate (VII-2)

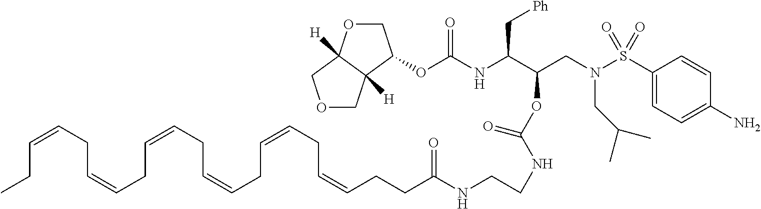 [SMILES: CC/C=C\C/C=C\C/C=C\C/C=C\C/C=C\C/C=C\CCC(=O)NCCNC(=O)O[C@H](CN(CC(C)C)S(=O)(=O)c1ccc(N)cc1)[C@H](Cc1ccccc1)NC(=O)O[C@H]1CO[C@H]2COC[C@H]21]

(3R,3aS,6aR)-hexahydrofuro[2,3-b]furan-3-yl((2S,3R)-4-(4-amino-N-isobutylphenylsulfonamido)-3-(((2-((4Z,7Z,10Z,13Z,16Z,19Z)-docosa-4,7,10,13,16,19-hexaenamido)ethyl)carbamoyl)oxy)-1-phenylbutan-2-yl)carbamate (VII-3)

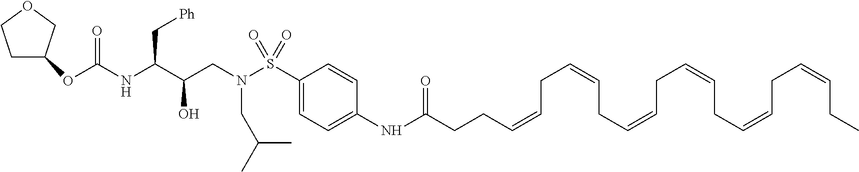 [SMILES: CC/C=C\C/C=C\C/C=C\C/C=C\C/C=C\C/C=C\CCC(=O)Nc1ccc(S(=O)(=O)N(CC(C)C)C[C@@H](O)[C@H](Cc2ccccc2)NC(=O)O[C@H]2CCOC2)cc1]

(S)-tetrahydrofuran-3-yl((2S,3R)-4-(4-((4Z,7Z,10Z,13Z,16Z,19Z)-docosa-4,7,10,13,16,19-hexaenamido)-N-isobutylphenylsulfonamido)-3-hydroxy-1-phenylbutan-2-yl)carbamate (VII-4)

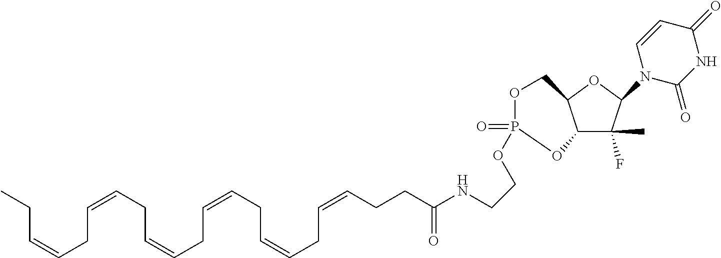

(4Z,7Z,10Z,13Z,16Z,19Z)—N-(2-(((4 aR,6R,7R,7aR)-6-(2,4-dioxo-3,4-dihydropyrimidin-1(2H)-yl)-7-fluoro-7-methyl-2-oxidotetrahydro-4H-furo[3,2-d][1,3,2]dioxaphosphinin-2-yl)oxy)ethyl)docosa-4,7,10,13,16,19-hexaenamide (VIII-1)

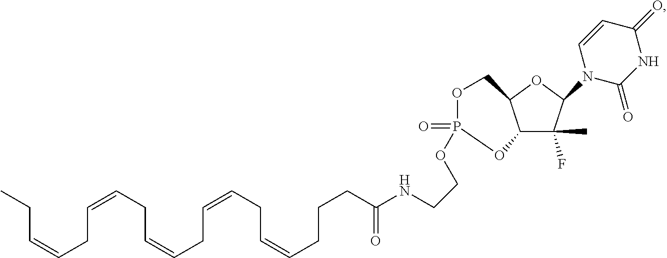

(5Z,8Z,11Z,14Z,17Z)—N-(2-(((4 aR,6R,7R,7aR)-6-(2,4-dioxo-3,4-dihydropyrimidin-1(2H)-yl)-7-fluoro-7-methyl-2-oxidotetrahydro-4H-furo[3,2-d][1,3,2]dioxaphosphinin-2-yl)oxy)ethyl)icosa-5,8,11,14,17-pentaenamide (VIII-2)

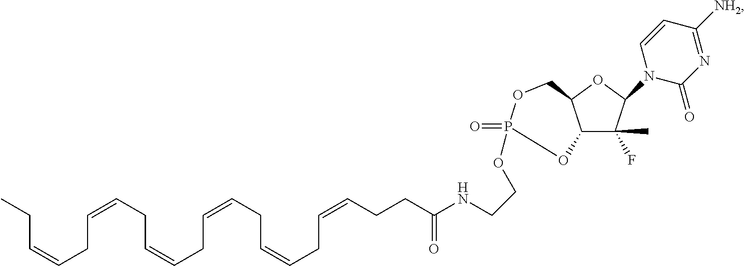

(4Z,7Z,10Z,13Z,16Z,19Z)—N-(2-(((4aR,6R,7R,7aR)-6-(4-amino-2-oxopyrimidin-1 (2H)-yl)-7-fluoro-7-methyl-2-oxidotetrahydro-4H-furo[3,2-d][1,3,2]dioxaphosphinin-2-yl)oxy)ethyl)docosa-4,7,10,13,16,19-hexaenamide (VIII-3)

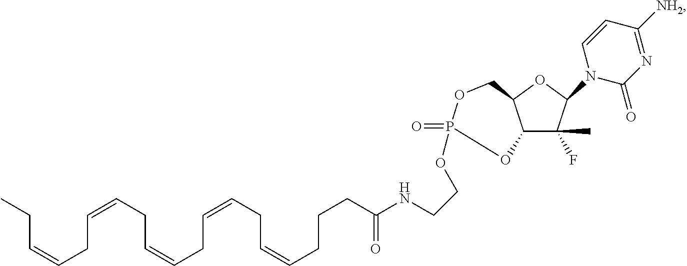

(5Z,8Z,11Z,14Z,17Z)—N-(2-(((4 aR,6R,7R,7 aR)-6-(4-amino-2-oxopyrimidin-1 (2H)-yl)-7-fluoro-7-methyl-2-oxidotetrahydro-4H-furo[3,2-d][1,3,2]dioxaphosphinin-2-yl)oxy)ethyl)icosa-5,8,11,14,17-pentaenamide (VIII-4)

### Methods for Using Fatty Acid Antiviral Conjugates

Provided herein are methods for inhibiting, preventing, or treating a viral infection. Non-limiting examples of which include influenza, swine flu, human immunodeficient virus (HIV), Hepatitis B (HBV), Hepatitis C(HCV), Herpes Simplex virus I and II (HSV-1, HSV-2), cytomegalovirus (CMV), varicella-zoster virus (VZV), Epstein Barr virus (EBV), human parainfluenza virus, human papillomavirus (HPV), Dengue virus, notovirus, rotavirus, ebola virus, influenza virus A, B and C. Additional subtypes of influenza virus A include H1N1, H2N2, H3N2, H5N1, H7N7, H1N2, H9N2, H7N2, H7N3 and H10N7.

The invention also includes pharmaceutical compositions useful for treating or preventing a viral infection. The compositions are suitable for internal use and comprise an effective amount of a fatty acid antiviral conjugate and a pharmaceutically acceptable carrier. The fatty acid antiviral conjugates are especially useful in that they demonstrate very low peripheral toxicity or no peripheral toxicity.

In some embodiments, the subject is administered an effective amount of a fatty acid antiviral conjugate.

The fatty acid antiviral conjugates can each be administered in amounts that are sufficient to treat a viral infection or prevent the development thereof in subjects.

Administration of the fatty acid antiviral conjugates can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid antiviral conjugate and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or conjugates thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid antiviral conjugate is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid antiviral conjugates.

The fatty acid antiviral conjugates can be also formulated as a suppository that can be prepared from fatty emulsions or suspensions; using polyalkylene glycols such as propylene glycol, as the carrier.

The fatty acid antiviral conjugates can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are herein incorporated by reference in their entirety.

Fatty acid antiviral conjugates can also be delivered by the use of monoclonal antibodies as individual carriers to which the fatty acid antiviral conjugates are coupled. The fatty acid antiviral conjugates can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the fatty acid antiviral conjugates can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid antiviral conjugates are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90% of the fatty acid antiviral conjugate by weight or volume.

The dosage regimen utilizing the fatty acid antiviral conjugate is selected in accordance with a variety of factors including type, species, age, weight, sex and medical condition of the patient; the severity of the condition to be treated; the route of administration; the renal or hepatic function of the patient; and the particular fatty acid antiviral conjugate employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the fatty acid antiviral conjugate per day. Compositions for in vivo or in vitro use can contain about 20, 50, 75, 100, 150, 250, 500, 750, 1,000, 1,250, 2,500, 3,500, or 5,000 mg of the fatty acid antiviral conjugate. In one embodiment, the compositions are in the form of a tablet that can be scored. Effective plasma levels of the fatty acid antiviral conjugate can range from about 5 ng/mL to about 5,000 ng/mL. Appropriate dosages of the fatty acid antiviral conjugates can be determined as set forth in Goodman, L. S.; Gilman, A. The Pharmacological Basis of Therapeutics, 5th ed.; MacMillan: New York, 1975, pp. 201-226.

Fatty acid antiviral conjugates can be administered in a single daily dose, or the total daily dosage can be administered in divided doses of two, three or four times daily. Furthermore, fatty acid antiviral conjugates can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid antiviral conjugate ranges from about 0.1% to about 15%, w/w or w/v.

Combination Therapy:

In certain antiviral therapy, it is a common practice to sometimes use a combination of two or more antiviral agents in order to achieve the most effective treatment. In the treatment of HIV, a combination of three or 4 different agents are sometimes used in the HAART approach (highly active antiretroviral therapy). Agents that can be used in HAART come from a number of different classes and include: 1) entry inhibitors (non-limiting examples include maraviroc and enfuvirtide); 2) CCR5 receptor antagonists (non-limiting examples include aplaviroc and vicriviroc); 3) non-nucleoside reverse transcriptase inhibitors (non-limiting examples include efavirenz, nevirapine, delavirdine, etravirine and rilpivirine); 4) nucleoside reverse transcriptase inhibitors (non-limiting examples include zidovudine, didanosine, zalcitabine, stavudine, lamivudine, abacavir, emtricitabine, entecavir and apricitabine); 5) protease inhibitors (non-limiting examples include saquinavir, ritonavir, indinavir, nelfinavir, amprenavir, lopinavir, atazanavir, fosamprenavir, tipranavir and darunavir); 6) integrase inhibitors (non-limiting examples include raltegravir, elvitegravir and MK-2048); 7) maturation inhibitors (non-limiting examples include bevirimat and MPC-9055). In some embodiments, the compounds of the invention, namely fatty acid antiviral conjugates, can be used in combination with one or more agents listed above in order to achieve the most effective anti-HIV treatment.

The most effective treatment for hepatitis C (HepC or HCV) sometimes involves the combination of one or more agents. These agents also can come from different classes and include: 1) nucleoside polymerase inhibitors (non-limiting examples include ribavirin, INX-189, GS-7977, IDX-184, GS 6620, RG7432 and mericitabine); 2) non-nucleoside polymerase inhibitors (non-limiting examples include GS 9190, GS 9669, VX-222, ABT-333, and ABT-072); 3) NS3 protease inhibitors (non-limiting examples include GS 9256, GS 9451, ACH-1625, ACH-2684 and BI 201335); 4) NS5a protease inhibitors (non-limiting examples include GS5885, IDX-719, ACH-2928 and daclatasvir); 5) NS5b protease inhibitors (non-limiting example includes BI 207127); 6) TLR-7 agonists (non-limiting example includes GS 9620); 7) cyclophilin inhibitors (non-limiting example includes DEB025); 8) protease inhibitors (non-limiting examples include TMC435, ABT-450, MK 5172, danoprevir, telaprevir, boceprevir and asunaprevir); 9) interferon (non-limiting examples include peginterferon-lambda-1a, recombinant interferon alpha-2b). In some embodiments, the compounds of the invention, namely fatty acid antiviral conjugates, can be used in combination with one or more agents listed above in order to achieve the most effective anti-HCV treatment.

### Methods for Making the Fatty Acid Antiviral Conjugates

Examples of synthetic pathways useful for making fatty acid antiviral conjugates of Formula I-VIII are set forth in the Examples below and generalized in Schemes 1-6.

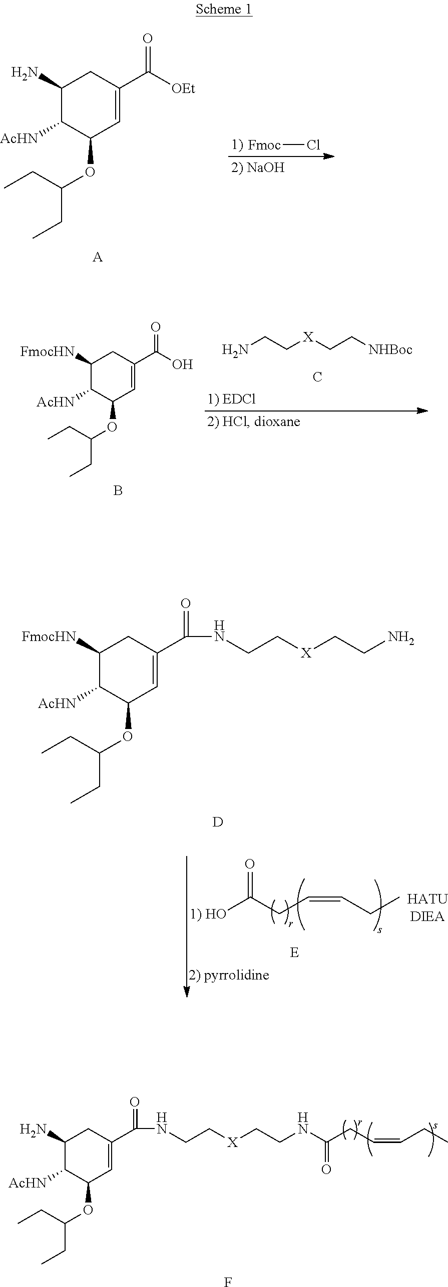

wherein R₄, r, and s are as defined above.

In scheme 1, compound A represents oseltamivir. To those familiar in the art, other antiviral agents with a carboxylic acid group can also be subjected to the same chemistry in order to prepare the appropriate fatty acid antiviral agents. Examples of antiviral agents that have a carboxylic acid group include, but are not limited to, zanamivir, peramivir and laninamivir. In Scheme 1, the mono-BOC protected amine of the formula C can be obtained from commercial sources or prepared according to known procedures, depending on the group X (wherein X can be —NR⁴—, —NC(O)R—, —O—, —S—, —CH(OH)—, —OCH₂CH₂O—). The mono-BOC protected amine C (wherein X=—NR⁴—) can be prepared according to the procedures outlined in Krapcho et al. Synthetic Commun. 1990, 20, 2559-2564. The mono-BOC protected amine C (wherein X═NC(O)R,) can be prepared according to the procedures outlined in Andruszkiewicz et al. Synthetic Commun. 2008, 38, 905-913. The mono-BOC protected amine C (wherein X═O or CH(OH)) can be prepared according to the procedures outlined in Dahan et al. J. Org. Chem. 2007, 72, 2289-2296. The mono-BOC protected amine C (wherein X═S or OCH₂CH₂O) can be obtained from commercial sources.

The basic amino group in compound A can be protected first by converting to the Fmoc derivativeaccording to known procedures outlined in Greene's Protecting Groups in Organic Synthesis (Wiley, 3^{rd} edition). The ester group is then hydrolyzed to the corresponding acid group by treatment with NaOH or LiOH. The resulting acid derivative B is then coupled with the amine C using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound D. Compound D can be coupled with a fatty acid of formula E using HATU in the presence of a tertiary amine such as DIEA. To those familiar in the art, the fatty acid D can also be substituted with lipoic acid in this scheme and in the subsequent schemes. The Fmoc protecting group can then be removed by treatment with a base such as pyrrolidine or diethylamine in THF to afford compounds of the formula F.

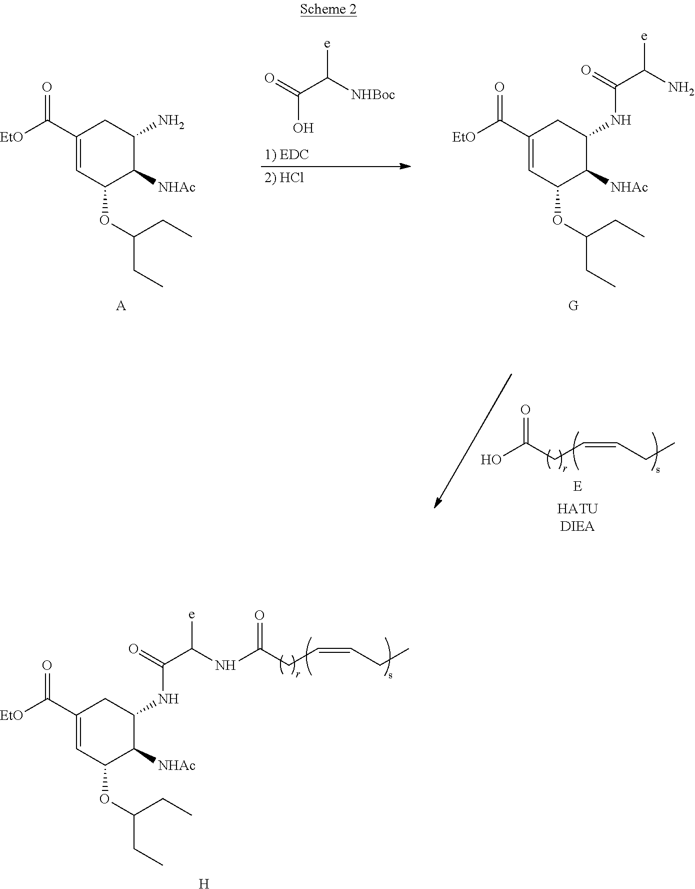

wherein e, r and s are as defined above.

Compound A can be coupled with a BOC-protected amino acid in the presence of EDC, followed by treatment with HCl to remove the BOC group, in order to form compounds of the formula G. Compound G can then be coupled with a fatty acid of the formula E in order to prepare compounds of the formula H. To those familiar in the art, compound A represents oseltamivir. Other antiviral agents with an amino group can be subjected to the same chemistry depicted in Scheme 2. Examples of antiviral agents that have an amino group include, but are not limited to, abacavir, adefonir, cidofovir, emtricitabine, entecavir, lamivudine, ganciclovir, penciclovir, and zalcitabine.

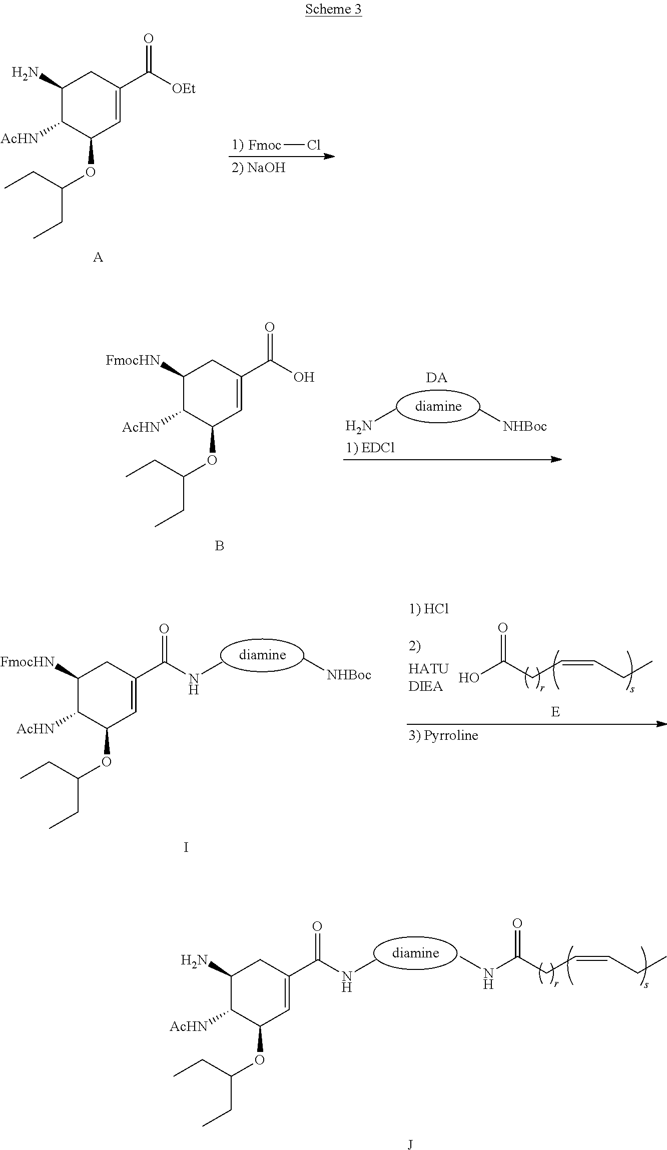

wherein r and s are as defined above.

In scheme 3, compound A represents oseltamivir. To those familiar in the art, other antiviral agents with a carboxylic acid group can also be subjected to the same chemistry in order to prepare the appropriate fatty acid antiviral agents. Examples of antiviral agents that have a carboxylic acid group include, but are not limited to, zanamivir, peramivir and laninamivir. The basic amino group in compound A can be protected first by converting to the Fmoc derivative according to known procedures outlined in Greene's Protecting Groups in Organic Synthesis (Wiley, 3^{rd} edition). The ester group is then hydrolyzed to the corresponding acid group by treatment with NaOH or LiOH. The resulting acid derivative B is then coupled with a BOC-protected diamine of the general formula DA to obtain the BOC-protected amide cderivative of the general formula I. After treatment with HCl in dioxane, the resulting amine can be coupled with a fatty acid of the formula E. The resulting compound can be treated with a base such as pyrrolidine or diethylamine in THF to remove the Fmoc protecting group. A variety of BOC-protected diamines are commercially available. Examples of which include, but are not limited to, tert-butyl (2-aminoethyl)carbamate and tert-butyl piperazine-1-carboxylate. The following diamines can be prepared according to the procedures outlined in the corresponding references:

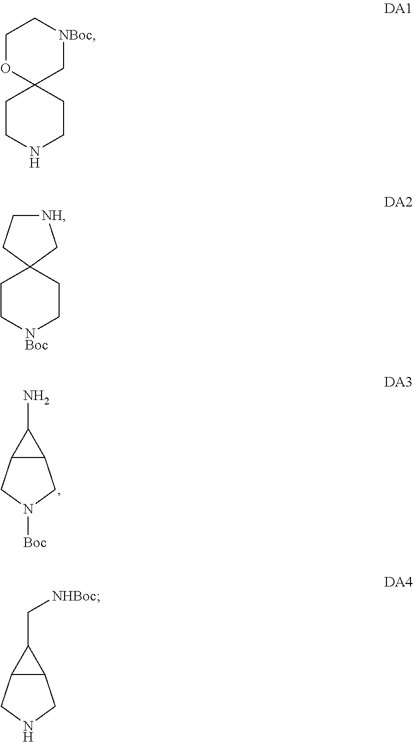 [SMILES: CC(C)(C)OC(=O)N1CCOC2(CCNCC2)C1 ; CC(C)(C)OC(=O)N1CCC2(CCNC2)CC1 ; CC(C)(C)OC(=O)N1CC2C(N)C2C1 ; CC(C)(C)OC(=O)NCC1C2CNCC21]

diamine DA1, Stocks et al, Bioorganic and Medicinal Chemistry Letters 2010, p. 7458; diamine DA2, Fritch et al, Bioorganic and Medicinal Chemistry Letters 2010, p. 6375; diamine DA3 and DA4, Moffat et al, J. Med. Chem. 2010, 53, p. 8663-86′78). To those familiar in the art, detailed procedures to prepare a variety of mono-protected diamines can also be found in the following references: WO 2004092172, WO 2004092171, and WO 2004092173.

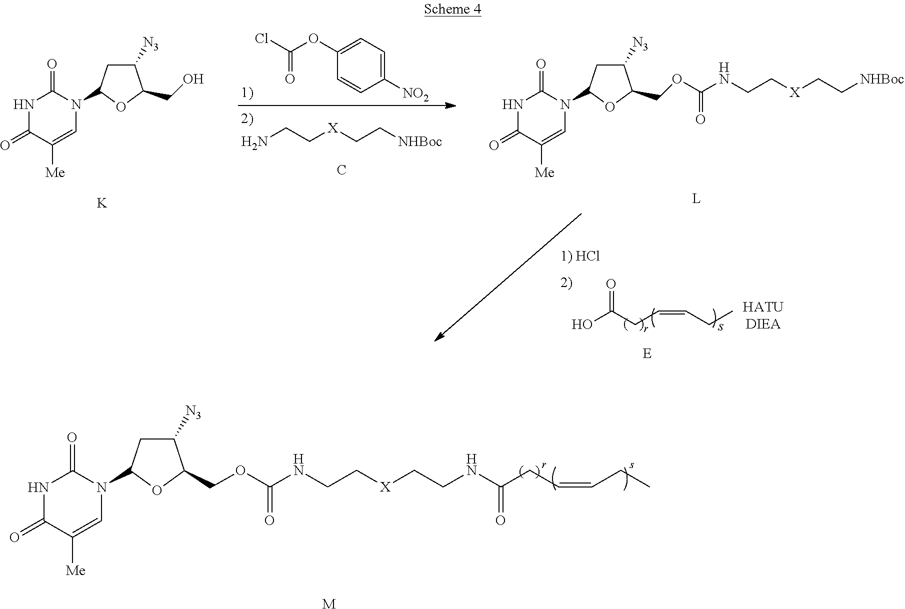

wherein R₄, r and s are as defined above.

In scheme 4, compound K represents zidovudine (AZT). To those familiar in the art, other antiviral agents with a free hydroxyl group can also be subjected to the same chemistry in order to prepare the appropriate fatty acid antiviral agents. Examples of antiviral agents that have a free hydroxyl group include, but are not limited to, didanosine, emtricitabine, lamivudine, zalcitabine, stavudine, PSI 7977, amprenavir, atazanavir, indinavir, lopinavir, nelfinavir, ritonavir, daruvavir, and saquinavir. In Scheme 4, the mono-BOC protected amine of the formula C can be obtained from commercial sources or prepared according to known procedures, depending on the group X (wherein X can be —NR⁴—, —NC(O)R—, —O—, —S—, —CH(OH)—, —OCH₂CH₂O—). The mono-BOC protected amine C (wherein X═—NR⁴—) can be prepared according to the procedures outlined in Krapcho et al. Synthetic Commun. 1990, 20, 2559-2564. The mono-BOC protected amine C (wherein X═NC(O)R,) can be prepared according to the procedures outlined in Andruszkiewicz et al. Synthetic Commun. 2008, 38, 905-913. The mono-BOC protected amine C (wherein X═O or CH(OH)) can be prepared according to the procedures outlined in Dahan et al. J. Org. Chem. 2007, 72, 2289-2296. The mono-BOC protected amine C (wherein X═S or OCH₂CH₂O) can be obtained from commercial sources. Compound K can be reacted first with 4-nitrochloroformate, in the presence of a tertiary amine such as triethylamine, followed by the reaction with a mono-Boc protected amine of the formula C in order to obtain compounds of the formula L. The Boc protecting group can be removed by treatment with HCl, and the resulting amine can be coupled with a fatty acid of the formula E using HATU in the presence of DIEA to obtain compounds of the general formula M.

To those familiar in the art, the nucleoside K can be replaced with any other nucleosides of the general formula:

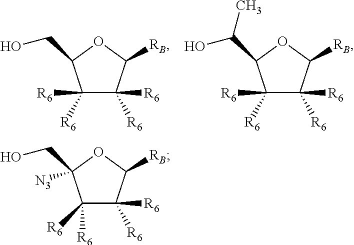

R₆ is as defined above and R_{B} can independently be anyone of the following bases:

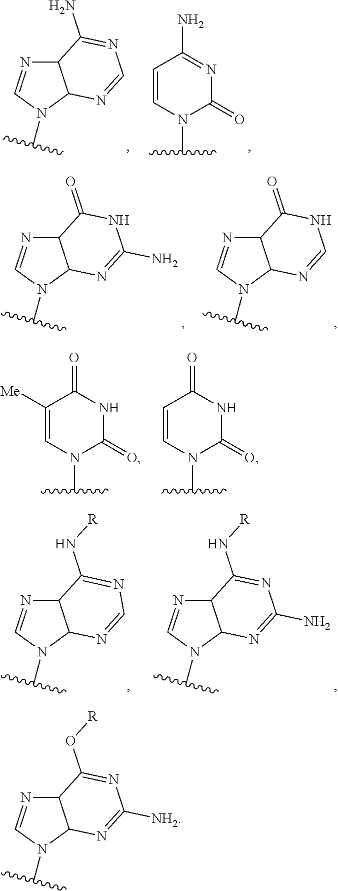

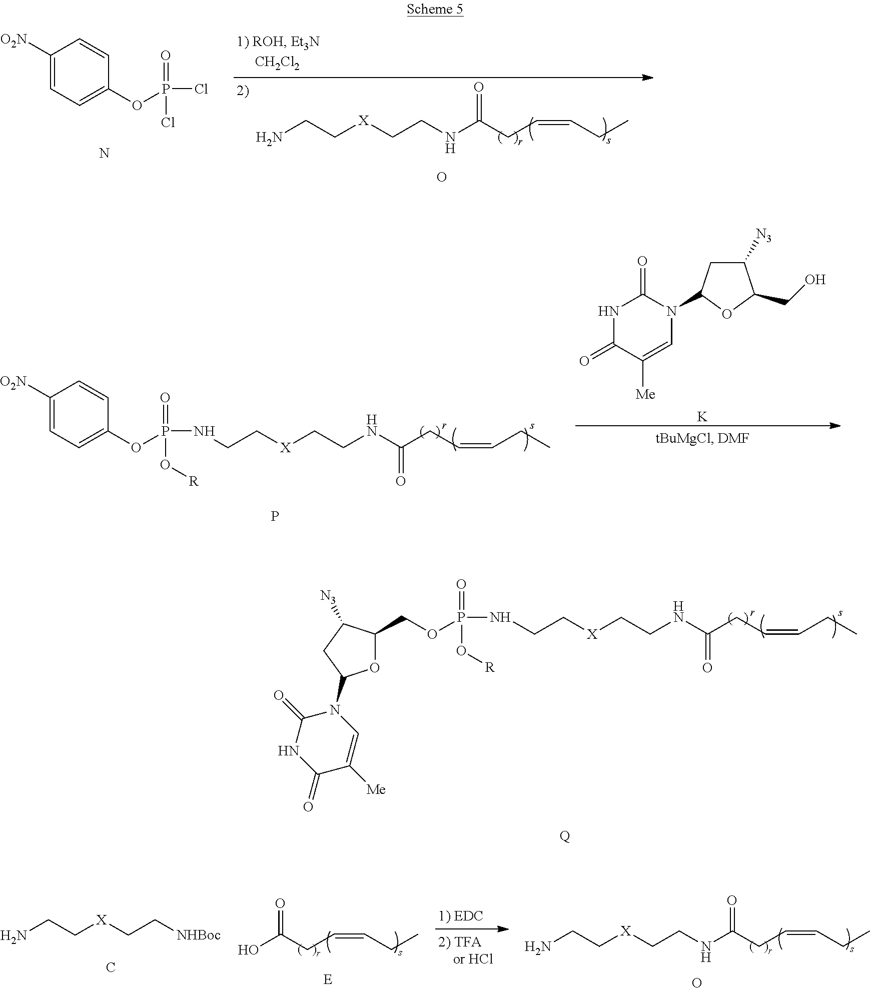

Wherein R, X, r and s are as defined above.

The commercially available 4-nitrophenyl phosphorodichloridate N can be coupled first with an alcohol of the general formula ROH, in the presence of a base such as triethylamine, in a solvent such as CH₂Cl₂, to displace the first Cl group. The second Cl group can be displaced with an fatty acid amine of the general formula O in order to prepare a 4-nitrophenyl phosphate conjugate of the general formula P. Fatty acid amine of the general formula O, in turn, can be prepared by coupling a BOC-protected diamine of the general formula C with a fatty acid of the general formula E in the presence of EDC or HATU, followed by treatment with an acid such as TFA or HCl in EtOAc or dioxane. BOC-protected diamine of the general formula C can be prepared according to the procedures described in Scheme 4. Compound P can be coupled with a nucleoside K, in the presence of tert-butylmagnesium chloride, in a solvent such as DMF, to afford the phosphoramidate of the general formula Q. To those familiar in the art, the nucleoside K can also be replaced with any other nucleosides of the general formula shown in Scheme 4.

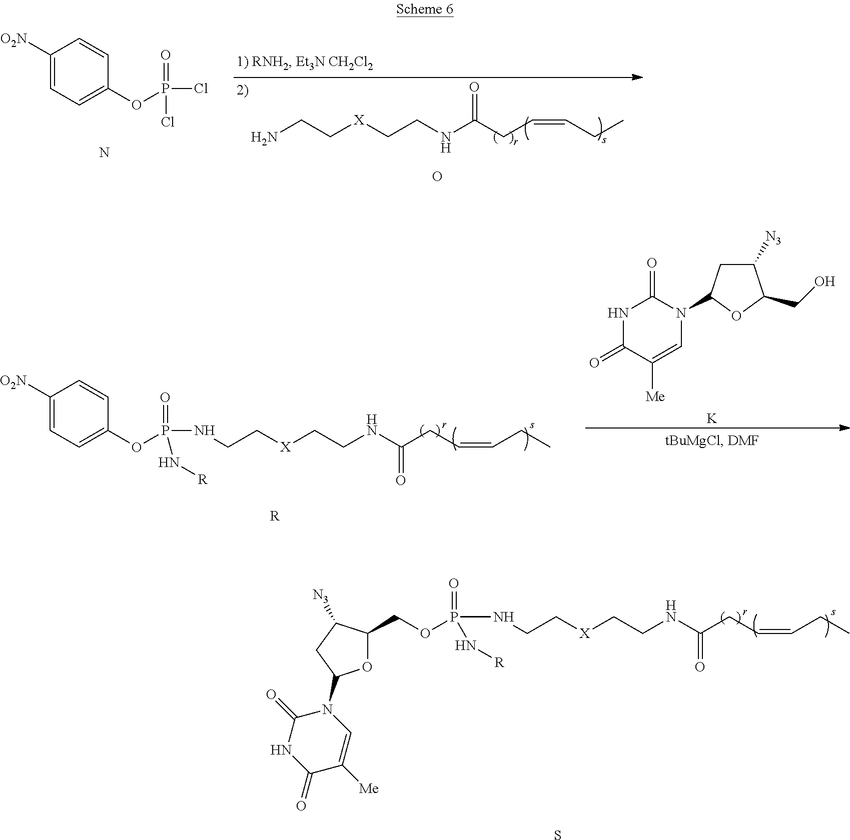

Wherein R, X, r and s are as defined above.

The commercially available 4-nitrophenyl phosphorodichloridate N can be coupled first with an amine of the general formula RNH₂, in the presence of a base such as triethylamine, in a solvent such as CH₂Cl₂, to displace the first Cl group. The second Cl group can be displaced with an fatty acid amine of the general formula O in order to prepare a 4-nitrophenyl phosphate conjugate of the general formula R. To those familiar in the art, the amine RNH₂ can also be a naturally occurring amino acid ester such as glycine methyl ester, alanine methyl ester, valine ethyl ester etc. . . . . The phosphate intermediate R can be coupled with the nucleoside K in the presence of tert-butylmagnesium chloride, in a solvent such as DMF, to afford the phosphoramidate of the general formula S. To those familiar in the art, the nucleoside K can also be replaced with any other nucleosides of the general formula shown in Scheme 4.

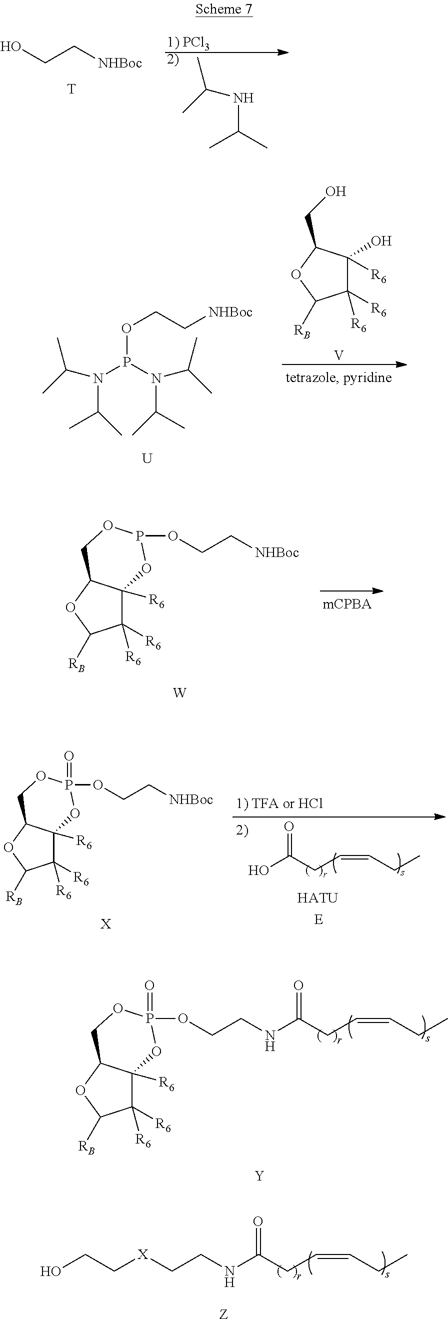

Wherein R_{B}, R₆, r and s are as defined above.

The amino alcohol T can be coupled with PCl₃, followed by reaction with an excess of diisopropylamine to afford the intermediate U. This is then coupled with 3′,5′ nucleoside of the formula V in the presence of tetrazole and pyridine to afford compounds of the general formula W. This compound can be treated with mCPBA to afford the cyclic phosphate derivative X. The BOC group in compound X can be removed with treatment with an acid such as TFA or HCl. The resulting amine can then be coupled with a fatty acid of the formula E to afford compounds of the general formula Y. To those familiar in the art, amine T can be replaced with an amino derivative of the general formula Z. Additional details to prepare amino derivative of the formula Z are shown in 4.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effect of the Compounds of the Invention on the Pro-Inflammatory Cytokine TNF-α

An influenza A viral infection, such as H5N1, often induces pro-inflammatory cytokine dysregulation. As described in WO 2007/101111, an increased level of TNF-α and other cytokines from macrophages are believed to be relevant to the severity of illness in patients with influenza A infection, particularly the unusual clinical presentation and severity of illness in patients with H5N1 “avian flu”. An assay that measures the effect of the fatty acid antiviral conjugates on the production of TNF-α can be particularly useful.

The purpose of this assay is to measure the ability of small molecules to inhibit the secretion of TNFα in cultured macrophages stimulated with lipopolysaccharide (LPS). Treatment of macrophages with LPS activates inflammatory cytokine pathways primarily through the TLR4-NFκB signaling axis. Compounds of the invention inhibit the transcriptional activation of NFκB and thus decrease the production and release of TNFα. Dexamethasone, a potent agonist of the glucocorticoid receptor is used a positive control for inhibition of TNFα release.

Day 1: Seed RAW 264.7 macrophages into 96 well culture plates. Remove culture media from RAW 264.7 cell growing in a 75 mm² tissue culture flask (cells should be at ˜70% confluence) and add 10 mL of warmed complete growth media (DMEM+10% FBS+1× pen/step). The cells are scraped into suspension using a sterile plate scraper and homogenized by pipetting up and down with a 10 mL serological pipette. The cell concentration is determined using a clinical hematoctyometer. Cells are then diluted to 150,000 cells per mL into growth media. The diluted cells are then transferred to a sterile reagent reservoir and 100 μl of cell suspension is pipetted into each well of a 96 well culture plate using a multichannel pipette (15,000 cells/well). Plates are then incubated at 37° C. under normal tissue culture growth conditions (37° C., humidified CO₂ chamber).

Day 2: The test compound sample plate is prepared. Test compounds are prepared in growth media. Compounds are delivered to media from 1000× stocks in 100% DMSO (e.g. for a 10 μM final concentration of test compound, deliver 2 μl of 10 mM test compound to 2 mL of media). At least 150 μl of 1× compound in media is added to 96 well sample plate. The perimeter wells of the 96 well plate are not used to avoid edge effects. Twelve sample wells are prepared with media plus 0.1% DMSO (these samples will serve as the vehicle controls; LPS-stimulated and non-stimulated; 10 μM dexamethasone is used as a positive control). Culture plates are then returned to the growth incubator for 2 hours. Cells are stimulated afterwards by adding 25 μl of 50 ng/mL LPS is added to every well (except the 6 unstimulated vehicle control wells: final concentration of 10 ng/mL LPS. Plates are returned to growth incubator for 3 hours. Afterwards, 100 μl of media supernatant is removed and transferred to a 96 well v-bottom sample plate. The media supernatant plate is centrifuged for 5 minutes at 1,000 rpm in a swing-bucket centrifuge, pelleting any cellular debris that may remain in supernatant. 80 μl of supernatant is removed from sample plate and transferred to a fresh v-bottom 96 well plate. Cell viability is measured using Celltiter-glo kit. By measuring cell viability, a given compound's effects on TNFα secretion can determine whether effects are due to cytotoxicity or to true inhibition of inflammatory signaling. Add 100 μl of Celltiter-glo reagent to each well of the cell culture plate and afterwards measure the luminescence signal (CPS) of the plate using the Victor 5 plate reader (0.3 second read; 60 second plate shaking prior to read). Cell viability of a given compound at a given concentration is computed as follows:

Cell viability=CPS Sample/(Average CPS unstimulated controls)*100

Use 20 μl of media supernatant per well for TNFα ELISA. Follow Invitrogen/Biosource manufacture's protocol for the mouse TNFα ELISA. Chromogen development is typically conducted for 20-30 minutes as described in the manufacturer's protocol. After addition of stop solution, measure OD 450 nm using the Victor 5 plate reader (0.1 second/well scan). Determine the TNFα secretion percent of control. The following formula is used to determine the TNFα secretion percent of control:

100×(OD 450 nm Sample X)−(Average OD 450 nm unstimulated vehicle controls)(Average OD 450 nm LPS stimulated vehicle controls)−(Average OD 450 nm unstimulated vehicle controls)

For each test compound, TNFα secretion percent of control can be plotted as a function of compound concentration using a four parameter dose-response curve fit equation (XLFIT Model #205):

fit=(A+((B−A)/(1+((C/x)̂D))))

inv=(C/((((B−A)/(y−A))−1)̂(1/D)))

res=(y−fit)

### Example 2

### Effect of the Compounds of the Invention on the Inhibition of the Growth of the Influenza Strain A/WS/33 from MDCK Cells

The influenza strain A/WS/33 is commercially available from American Type Culture Collection (ATCC) (Manassas, Va.). This strain was isolated from a patient with influenza. Recommended hosts for the influenza strain A/WS/33 include chicken, embryo, ferrets and mouse. MDCK cells are epithelial-like cells derived from a kidney of a normal adult femal cocker spaniel. These cells have been shown to support the growth of various types of virus, including influenza A virus. MDCK cells can be used to produce high titer stocks of A/WS/33 according to the procedures outlined in WO 2007/101111. An MDCK-based immunofocus assay can be used to quantitate infectious virus in the supernatant. MDCK cells (5×10⁵/well) are plated in 24 well plates and cultured overnight in virus growth medium containing DME media base (#10-0,3-CV, MediaTech, Herndon Va.) with 10% fetal bovine serum, 25 mM HEPES buffer (#25-060-CL, Mediatech), 1:100 antibiotic/antimycotic solution (#A5955-Sigma-Aldrich), 1.8 μg/mL bovine serum albumin (#A7906Sigma-Aldrich), and 2 mg/mL trypsin (#3740, Worthington, Lakewood, N.J.). Cells are then washed twice in the same medium without fetal bovine serum. Serial dilutions of virus-containing supernatants are then added for 30 min, followed by an overlay of virus growth medium with 0.6% tragacanth gum (#104792, MP Biomedicals Inc, Solon Ohio). After 24 h and 48 h of incubation the overlay is aspirated, the cells are rinsed with PBS and fixed with 50:50 acetone/methanol. The cells are then stained with anti-HA antibody for focus detection.

### Example 3

### Effect of the Compounds of the Invention in an HW-1 Cytoprotection Assay

Cytoprotection assays are commonly used for evaluating the antiviral efficacy of test compounds against a variety of viruses in different cell lines. The HIV Cytoprotection assay uses CEM-SS cells and the IIIB strain of HIV-1. Briefly, virus and cells are mixed in the presence of test compound and incubated for 6 days. The virus is pre-titered such that control wells exhibit 70 to 95% loss of cell viability due to virus replication. Therefore, antiviral effect or cytoprotection is observed when compounds prevent virus replication. Each assay plate contains cell control wells (cells only), virus control wells (cells plus virus), compound toxicity control wells (cells plus compound only), compound colorimetric control wells (compound only), as well as experimental wells (compound plus cells plus virus). Cytoprotection and compound cytotoxicity are assessed by MTS (CellTiter®96 Reagent, Promega, Madison Wis.) dye reduction. The % reduction in viral cytopathic effects (CPE) is determined and reported; IC₅₀ (concentration inhibiting virus replication by 50%), TC₅₀ (concentration resulting in 50% cell death) and a calculated TI (therapeutic index TC₅₀/IC₅₀) are provided along with a graphical representation of the antiviral activity and compound cytotoxicity when compounds are tested in dose-response. Each assay includes the HIV reverse transcriptase inhibitor AZT as a positive control.

### Cell Preparation:

CEM-SS cells were obtained from the NIH AIDS Research and Reference Reagent Program and are routinely passaged in T-75 flasks using standard tissue culture techniques based on the specifications provided by the supplier. On the day preceding the assay, the cells are split 1:2 to assure they are in an exponential growth phase at the time of infection. Total cell number and percent viability determinations are performed using a hemacytometer and trypan blue exclusion. Cell viability must be greater than 95% for the cells to be utilized in the assay. The cells are re-suspended at 5×10⁴ cells/mL in tissue culture medium and added to the drug-containing 96-well microtiter plates in a volume of 50 μl.

### Virus Preparation:

The viruses used for this assay are CXCR4-tropic laboratory virus strains. The most commonly used strains are HIV-1_{RF} and HIV-1_{IIB} (each obtained from the NIH AIDS Research and Reference Reagent Program). For each assay, a pre-titered aliquot of virus is removed from the freezer (−80° C.) and allowed to thaw slowly to room temperature in a biological safety cabinet. The virus is re-suspended and diluted into tissue culture medium such that the amount of virus added to each well in a volume of 50 μl is the amount determined to give between 85 to 95% cell killing at 6 days post-infection. TCID₅₀ calculations by endpoint titration in the assay indicates that the multiplicity of infection of these assays is approximately 0.01.

### Plate Format:

Each plate contains cell control wells (cells only), virus control wells (cells plus virus), drug cytotoxicity wells (cells plus drug only), drug colorimetric control wells (drug only), background control wells (media only), as well as experimental wells (drug plus cells plus virus). Samples are evaluated for antiviral efficacy with triplicate measurements using 6 concentrations at half-log dilutions (12 concentrations can also be performed) in order to determine IC₅₀ values and with duplicate measurements to determine cellular cytotoxicity, if detectable.

### Solubilization Protocol:

100% EtOH is added to the compounds of the invention such that the EtOH stock concentration is 50 mM. The 10× solutions in FBS can be prepared as follows: a) FBS (490 μL) (Gibco #10437, lot #1009392) is added to a 1.5 mL eppendorf tube for every compound to be tested; b) EtOH stock solutions (10 μL) is added to each tube for a 1 mM Compound, 2% EtOH 10× stock (Add such that the entire content is ejected out of the tip, vortex the eppendorf tube, and then pump mix to remove any residual EtOH stock solution that remains in the tip); c) EtOH is added to FBS in the same ratio (2% EtOH) such that there is sufficient amount to provide vehicle controls for the assay and any compound dilutions that are going to be tested; d) The 10×FBS stock and 2% EtOH solutions are sonicated for 1 hour in a sonicating water bath. (VWR Ultrasonics Cleaner, Model #B8500A-DTH with “Sonics Power” set to HI); e) The 1:2 serial dilutions of the 1 mM 10× stock solution with FBS/2% EtOH are prepared such that the concentrations of the 10× stock solutions are 1 mM, 0.5 mM, 0.25 mM, etc., in FBS with 2% EtOH. Medium is aspirated off cells and replaced with serum free medium. The 10× stock solutions are diluted 1:10 into medium such that the final concentration of the dilution series are 100 μM, 50 μM, 25 μM, etc., all with 0.2% final EtOH.

Table 1 summarizes the IC₅₀ for selected fatty acid antiviral conjugates in this HIV-1 assay against the IIIB strain. In this table, a +++ signifies an IC₅₀ value of <100 nM and a + signifies an IC₅₀ of ≧100 nM.

TABLE 1
Summary of IC₅₀ against HIV-1_{IIIB} strain.

| | IC₅₀ HIV-1_{IIIB} |
|---|---|
| Compound | strain (nM) |
| II-25 | +++ |
| II-30 | + |
| II-67 | +++ |
| V-5 | + |
| V-18 | + ++ |
| V-21 | + ++ |
| VII-1 | + ++ |
| VII-2 | + ++ |

### Example 4

### Effect of the Compounds of the Invention in an HCV Replicon Assay

The HCV replicon assay can be carried out in the same manner described in WO 2010/018140, WO 2011/123586, Okuse et al Antivir. Res. 2005, 65, p. 23, Blight et al Science 2000, 290, p. 1972, Korba and Gerin Antivir. Res. 1992, 19, p. 55). Huh-7 cells containing HCV Con 1 subgenomic replicon (GS4.1 cells) are grown in Dulbecco's Modified Eagle Medium (DMEM) supplemented with 10% fetal bovine serum (FBS), 2 mM L-glutamine, 110 mg/L sodium pyruvate, 1× non-essential amino acids, 100 U/mL penicillin-streptomycin and 0.5 mg/mL G418 (Invitrogen). For dose-response testing, the cells are seeded in 96-well plates at 7.5×10³ cells/well in a volume of 50 μM and incubated at 37° C./5% CO₂. Three hours after plating, 50 μL of ten 2-fold serial dilutions of compounds are added and cell cultures are incubated at 37° C./5% CO₂ in the presence of 0.5% DMSO. The compounds of the invention can be solubilized in FBS media according to the procedure outlined in example 3, substituting DMSO for EtOH. In this assay, Huh-7 cells lacking the HCV replicon serve as negative control. The cells are incubated in the presence of compounds for 72 hours after which they are monitored for expression of the NS4A protein by enzyme-linked immunosorbent assay (ELISA). For this, the plates are fixed for 1 min with 1:1 acetone:methanol, washed twice with phosphate-buffered saline (PBS), 0.1% Tween 20, blocked for 1 hour at room temperature with TNE buffer containing 10% FBS and then incubated for 2 h at 37° C. with the anti-NS4A mouse clonal antibody A-236 (Virogen) diluted in the same buffer. After washing three times with PBS, 0.1% Tween 20, the cells are incubated 1 hour at 37° C. with anti-mouse immunoglobulin G-peroxide conjugate in TNE, 10% FBS. After washing as described above, the reaction is developed with O-phenylalanine (Zymed). The reaction is stopped after 30 minutes with 2 N H₂SO₄ and the absorbance is read at 492 nm using a Sunrise Tecan spectrophotometer. EC₅₀ values are determined from the % inhibition vs concentration data using a sigmoidal non-linear regression analysis based on four parameters with Tecan Magellan software. For cytotoxicity evaluation, GS4.1 cells are treated with compounds as described and cellular viability can be monitored using a Cell Titer 96 AQᵤₑₒᵤₛ One Solution Cell Proliferation Assay (Promega). CC₅₀ values can be determined from the % cytotoxicity vs concentration data with Tecan Magellan software as described above.

An alternative HCV replicon assay used to assess inhibitory activity against HCV NS5B polymerase is described in Clark et al, J. Med. Chem. 2005, 48, p. 5504.

### Example 5

### Effect of the Compounds of the Invention in RSV Antiviral Assays

The RSV antiviral assay can be carried out using the procedures detailed in WO 2012/040124 and Sidwell et al Appl. Microbiol. 1971, 22, p. 797-801. With the CPE reduction assay, HEp-2 cells (ATCC) at a concentration of 6000 cells/well are infected with RSV Long strain (ATCC) at a multiplicity of infection (m.o.i.) of 0.01, and each of the test compounds are provided to duplicate wells at final concentrations starting from 30 μM using ⅓ stepwise dilutions. For each compound, two wells are set aside as uninfected, untreated cell controls (CC), and two wells per test compounds received virus only as a control for virus replication (VC). The assay is stopped after 6 days, before all of the cells in the virus-infected untreated control wells exhibited signs of cytopathology (giant cell formation, syncytia). At the end of the incubation, 20 μL of cell counting kit-8 reagent (CCK-8, Dojindo Molecular Technologies, Inc.) is added to each well. After 4 hr incubation, the absorbance is measured in each well according to manufacturer's instruction, and the 50% effective concentration (EC₅₀) is calculated by using regression analysis, based on the mean O.D. at each concentration of compound.

RT-PCR based assays are performed in HEp-2 cells (ATCC: CCL-23) at a concentration of 20,000 cells/well are plated in 96 well plates and incubated overnight. Each of the test compounds are ⅓ serially diluted and dosed to HEp-2 cells in duplicates. The highest final concentration for each compound is 30 μM. After 24 hour compound pre-incubation, RSV A2 (ATCC: VR-1540) at MOI of 0.1 is added. Two wells per compound are set aside as uninfected, untreated cell controls (CC), and four wells per test compound receive virus only as a control for virus replication (VC). The assay is stopped 4 days after virus infection and conditioned media is removed for viral RNA isolation. The quantities of the RSV virus are measured by real time PCR using a set of RSV specific primers and probes. The data are analyzed with Prism software with EC50 defined as drug concentration that reduce the viral load 50% from the viral control (VC).

### Example 6

### Determination of Triphosphate Levels in Primary Hepatocytes Upon Treatment with Compounds of the Invention

Primary hepatocytes (human, rat, dog or monkey) are seeded (5,000,000 cells) into T75 flasks in DMEM containing 10% FBS and primary cell plating medium (CellzDirect, Inc.) respectively. After overnight incubation to allow the cells to attach, the cells are incubated for up to 24 h at 37° C. in a 5% CO₂ atmosphere in the fresh medium containing 100 μM of the compound of the invention of the general formula II or general formula V. At selected times, extracellular medium is removed and the cell layer is washed with cold PBS. After trypsinization, cells are counted and centrifuged at 1200 rpm for 5 min. The cell pellets are resuspended in 1 mL of cold 60% methanol and incubated overnight at −20° C. The samples are centrifuged at 14,000 rpm for 5 min, and the supernatants are collected and dried using a SpeedVac concentrator, then stored at −20° C. For the determination of the active triphosphate metabolite level, residues are suspended in 100 mL of water and 50 mL aliquots are injected into the LC/MS/MS.

### Example 7

### Rat In Vivo Pharmacokinetic and Tissue Distribution Determination of Compounds of the Invention

Compounds of the invention can be dosed orally by gavage in the appropriate vehicle at a dose of 300 mg/kg of the compounds of the invention. Sprague Dawley rats will be dosed twice daily (BID) by oral gavage for 4.5 days. Body weight will be measured and recorded daily for each rat. Clinical observations will be monitored daily for each rat. Tissue and plasma will be collected at 2 hours post last dose. The following tissues will be collected from each rat at termination, snap frozen and stored at ⁻80° C.: liver and spleen. The maximum volume of blood will be collected upon termination for processing to plasma. After 4 days of dosing, serial blood samples are collected at 0.5, 1, 2, 4, 6 and 8 hour in order to determine the PK parameters. Blood samples were obtained by venipuncture into polypropylene tubes containing K₂EDTA (10 mL, 0.5 M) and kept on ice for processing by centrifugation. Plasma samples are quick-frozen over dry ice and kept at −70° C. until LC/MS/MS analysis. Tissue samples, once harvested, are weighed and snap-frozen in liquid nitrogen. Frozen liver samples are homogenized in three volumes of ice cold 70% MeOH containing 20 mM EDTA/EGTA. The amount of the active metabolite triphosphate can be quantitated by LC/MS/MS. Plasma and liver concentrations versus time data can be analyzed by noncompartmental approaches using the appropriate WinNonlin software program.

### Example 8

### Effects of Compounds of the Invention on NF-κB Levels in RAW 264.7 Macrophages

RAW 264.7 cells stably expressing a 3×NFkB response element-drive luciferase reporter were seeded into 96 well plates in sera-free medium (Optimem) 18 hours prior to compound application. Compounds of the invention were prepared by first making 100 mM stock solutions in EtOH. Stock solutions were then diluted 1:100 in low LPS FBS (Gemini BenchMark 100-106), mixed vigorously and allowed to incubate at room temperature for 30 minutes. 1:2 serial dilutions were then made in FBS supplemented with 1% EtOH, mixed vigorously, and again allowed to incubate at room temperature for 30 minutes before adding to RAW 264.7 reporter cells (final concentrations: 10% FBS, 100 uM highest compound dilution, 0.1% EtOH) for a 2 hour pretreatment prior to stimulation with LPS. Cells were then stimulated with 200 ng/ml LPS or vehicle control for 3 hours in the presence of the compounds of the invention. A set of six vehicles was left unstimulated with LPS in order to measure the assay floor. AlamarBlue viability dye (Invitrogen) was added to cells simultaneously with the delivery of LPS (final AlamarBlue concentration of 10%). After the 3 h incubation period with LPS, cell viability was measured by reading fluorescence (excitation 550 nm, emission 595 nm) with a Perkin Elmer Victor V plate reader. Then cell media was aspirated from each well. Luciferase signal was then developed by addition of the Britelite Plus reagent (Perkin Elmer). Luciferase activity was measured with the Perkin Elmer Victor V plate reader. NF-κB activity was expressed as a percent of the vehicle control wells (stimulated with LPS). Compounds were tested at 6 dose point titrations in triplicate to determine IC₅₀ values.

Table 2 summarizes the IC₅₀ values for a number of fatty acid antiviral conjugates in this NF-κB luciferase reporter assay. A (−) indicates that the compound showed no inhibitory activity ≦200 μM. A (+) indicates that the compound showed inhibitory activity between >50 μM and ≦200 μM. A (++) indicates that the compound showed inhibitory activity at ≦50 μM.

TABLE 2
IC₅₀ values in the NF-kB luciferase reporter assay

| | NF-kB inhibitory |
|---|---|
| Compound | activity IC₅₀ μM |
| Lamivudine | − |
| Zidovudine | − |
| Darunavir | − |
| Entecavir | − |
| Famciclovir | − |
| II-25 | + + |
| II-30 | + + |
| II-48 | + |
| II-67 | + + |
| II-68 | + |
| II-69 | + |
| II-70 | + |
| V-5 | + + |
| V-8 | + |
| V-9 | + |
| V-10 | + |
| V-11 | + |
| V-12 | + |
| V-18 | − |
| V-21 | + |
| V-30 | ++ |
| VII-1 | + |
| VII-2 | ++ |

### Compounds

The following non-limiting compound examples serve to illustrate further embodiments of the fatty acid antiviral conjugates. It is to be understood that any embodiments listed in the Examples section are embodiments of the fatty acid antiviral conjugates and, as such, are suitable for use in the methods and compositions described above.

### Example 9

### Preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (II-25)

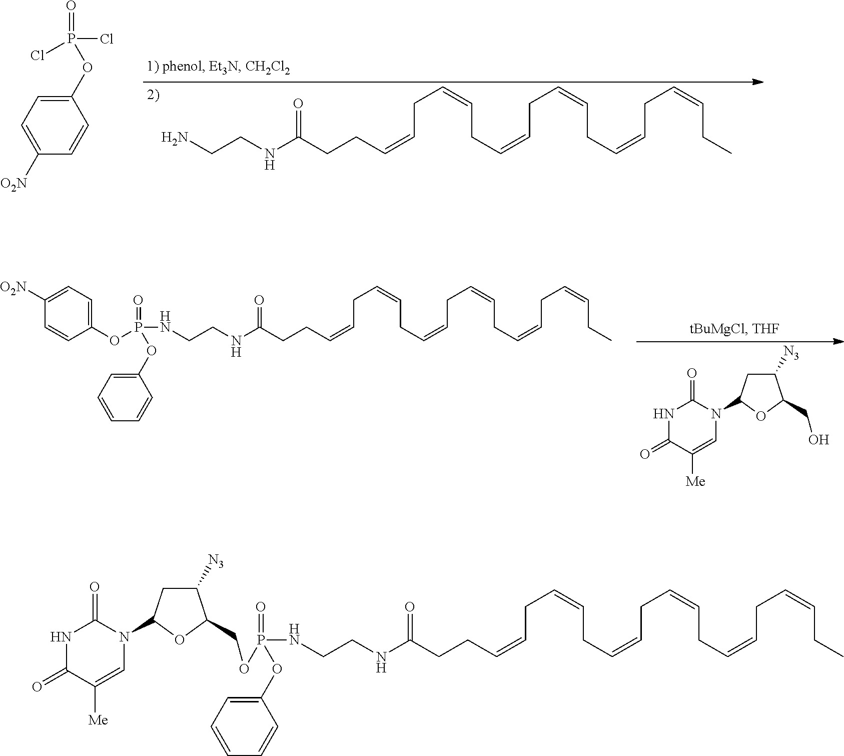

To a solution of 4-nitrophenyl phosphorodichloridate (0.8 g, 3.13 mmol) in CH₂Cl₂ (10 mL) under nitrogen was added a solution of phenol (293 mg, 3.13 mol) and triethylamine (0.48 mL, 3.44 mmol) in CH₂Cl₂ (10 mL) at −78° C. over a period of 20 min. The resulting reaction mixture was stirred at this temperature for 30 min and then slowly transferred to another round-bottom flask containing (4Z,7Z,10Z,13Z,16Z,19Z)—N-(2-aminoethyl)docosa-4,7,10,13,16,19-hexaenamide (1.16 g, 3.13 mmol) in CH₂Cl₂ (10 mL) at 0° C. (4Z,7Z,10Z,13Z,16Z,19Z)—N-(2-Aminoethyl)docosa-4,7,10,13,16,19-hexaenamide, in turn, was prepared according to the procedure outlined in WO 2012115695. To this mixture was added a second lot of triethylamine (0.96 mL, 6.56 mmol) over a period of 15 min. The resulting reaction mixture was stirred at 0° C. for 1 h and then concentrated under reduced pressure. The residue was triturated with ethyl acetate (20 mL), and the white solid was filtered off. The filtrate was concentrated under reduced pressure to give the crude product as a yellow oil. Purification by column chromatography (gradient elution, 0-60% ethyl acetate/hexanes) afforded 1.38 g of 4-nitrophenyl phenyl (2-((4Z,7Z,10Z,13Z,16Z,19Z)-docosa-4,7,10,13,16,19-hexaenamido)ethyl)phosphoramidate (68%).

¹H NMR (400 MHz, CDCl₃) δ 0.95-1.00 (t, J=7.6 Hz, 3H), 2.05-2.09 (m, 2H), 2.13-2.16 (m, 2H), 2.35-2.37 (m, 2H), 2.79-2.85 (m, 10H), 3.24-3.27 (m, 2H), 3.35-3.37 (m, 2H), 3.80-3.85 (m, 1H), 5.29-5.41 (m, 13H), 5.90 (s, 1H), 7.21-7.26 (m, 3H), 7.33-7.41 (m, 4H), 8.22-8.25 (d, J=8.8 Hz, 2H).

To a stirred solution of 4-nitrophenyl phenyl (2-((4Z,7Z,10Z,13Z,16Z,19Z)-docosa-4,7,10,13,16,19-hexaenamido)ethyl)phosphoramidate (200 mg, 0.75 mmol) in dry THF (10 mL) was added a 1.7 M solution of tert-butylmagnesium chloride in THF (0.53 mL, 0.90 mmol) over a period of 3 min at room temperature. The white suspension was stirred at this temperature for 30 min, and then a solution of zidovudine (582 mg, 0.90 mmol) in THF (6 mL) was added. The resulting reaction mixture was stirred at room temperature for 24 h. The reaction mixture was quenched with H₂O (1 mL), solvent was evaporated, and the residue was purified by preparative HPLC to afford 140 mg of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (24%).

MS calculated for C₄₀H₅₄N₇O₇P: 775.87. found: 776.3 [M+H]⁺.

¹H NMR (400 MHz, CDCl₃, 1:1 mixture of diasteromers) δ 0.95-0.99 (t, J=7.6 Hz, 3H), 1.88-1.90 (d, J=5.2 Hz, 3H), 2.05-2.09 (m, 2H), 2.14-2.18 (m, 2H), 2.31-2.39 (m, 4H), 2.79-2.84 (m, 10H), 3.12-3.17 (m, 2H), 3.30-3.34 (m, 2H), 3.76-3.78 (m, 1H), 4.03 (s, 1H), 4.27-4.37 (m, 3H), 5.29-5.41 (m, 12H), 5.98-6.01 (m, 1H), 6.06-6.12 (m, 1H), 7.16-7.23 (m, 3H), 7.31-7.35 (m, 3H), 7.80-8.81 (m, 1H).

### Example 10

### Preparation of ((2S,3S,5S)-3-azido-5-(5-methyl-2,4-dioxo-3,4-dihydropyrimidin-1(2H)-yl)tetrahydrofuran-2-yl)methyl methyl (2-((4Z,7Z,10Z,13Z,16Z,19Z)-docosa-4,7,10,13,16,19-hexaenamido)ethyl)phosphoramidate (II-26)

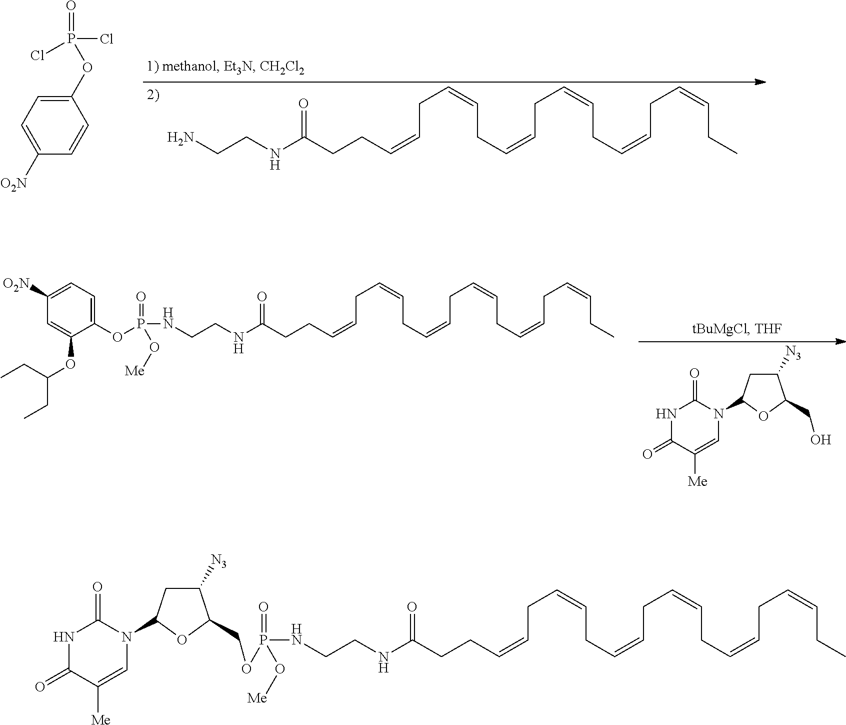

To a solution of 4-nitrophenyl phosphorodichloridate (1.0 g, 3.91 mmol) in CH₂Cl₂ (10 mL) under nitrogen was added a solution of CH₃OH (125 mg, 3.91 mol) and triethylamine (0.6 mL, 4.30 mmol) in CH₂Cl₂ (10 mL) at −78° C. over a period of 20 min. The resulting reaction mixture was stirred at this temperature for 30 min and then transferred to another round-bottom flask containing 4Z,7Z,10Z,13Z,16Z,19Z)—N-(2-aminoethyl)docosa-4,7,10,13,16,19-hexaenamide (1.18 g, 50 mmol) in CH₂Cl₂ (10 mL) at 0° C. To this mixture was added a second lot of triethylamine (1.2 mL, 8.2 mmol) over a period of 15 min. The resulting reaction mixture was stirred at 0° C. for 1 h, and then concentrated under reduced pressure. The residue was triturated with ethyl acetate (20 mL), and the white solid was filtered off. The filtrate was concentrated under reduced pressure to afford the crude product as a yellow oil. Purification by chromatography (gradient elution using 0-60% ethyl acetate/hexanes) afforded 0.9 g of methyl (4-nitrophenyl)(2-((4Z,7Z,10Z,13Z,16Z,19Z)-docosa-4,7,10,13,16,19-hexaenamido)ethyl)phosphoramidate (39%).

¹H NMR (300 MHz, CDCl₃) δ 0.95-1.01 (t, J=7.5 Hz, 3H), 2.05-2.11 (m, 2H), 2.20-2.26 (t, J=7.35 Hz, 2H), 2.37-2.42 (m, 2H), 2.80-2.86 (m, 10H), 3.14-3.21 (m, 2H), 3.37-3.41 (m, 2H), 3.83-3.87 (d, J=11.4 Hz, 3H), 5.31-5.47 (m, 12H), 5.92 (s, 1H), 7.38-7.42 (d, J=9.0 Hz, 2H), 7.38-7.42 (d, J=9.0 Hz, 2H).

To a stirred solution of methyl (4-nitrophenyl)(2-((4Z,7Z,10Z,13Z,16Z,19Z)-docosa-4,7,10,13,16,19-hexaenamido)ethyl)phosphoramidate (200 mg, 0.75 mmol) in dry THF (10 mL) was added a 1.7 M solution of tert-butylmagnesium chloride in THF (0.88 mL, 1.5 mmol). The white suspension was stirred at this temperature for 30 min, and then a solution of zidovudine (500 mg, 0.90 mmol) in THF (3 mL) was added. The resulting reaction mixture was stirred at this temperature for 18 h. The reaction mixture was quenched with H₂O (1 mL), solvent was concentrated under reduced pressure. The resulting residue was purified by preparative HPLC to afford 85 mg of ((2S,3S,5S)-3-azido-5-(5-methyl-2,4-dioxo-3,4-dihydropyrimidin-1(2H)-yl)tetrahydrofuran-2-yl)methyl methyl (2-((4Z,7Z,10Z,13Z,16Z,19Z)-docosa-4,7,10,13,16,19-hexaenamido)ethyl)phosphoramidate (16%).

MS calculated for C₃₅H₅₂N₇O₇P: 713.8. found: 714.4 [M+H]⁺.

¹H NMR (400 MHz, CDCl₃) δ 0.86-0.88 (t, J=3.6 Hz, 3H), 1.93 (s, 3H), 2.03-2.11 (m, 2H), 2.23-2.27 (m, 2H), 2.38-2.43 (m, 2H), 2.79-2.85 (m, 10H), 3.04-3.12 (m, 2H), 3.35-3.41 (m, 3H), 3.73-3.76 (d, J=11.6 Hz, 3H), 4.02-4.03 (m, 1H), 4.19-4.26 (m, 2H), 4.37-4.41 (m, 1H), 5.30-5.42 (m, 12H), 6.08-6.15 (m, 2H), 7.34-7.38 (m, 1H), 8.54-8.62 (m, 1H).

### Example 11

### Preparation of (2S,3S,5S)-3-azido-5-(5-methyl-2,4-dioxo-3,4-dihydropyrimidin-1(2H)-yl)tetrahydrofuran-2-yl)methyl phenyl (2-((5Z,8Z,11Z,14Z,17Z)-icosa-5,8,11,14,17-pentaenamido)ethyl)phosphoramidate (II-29)

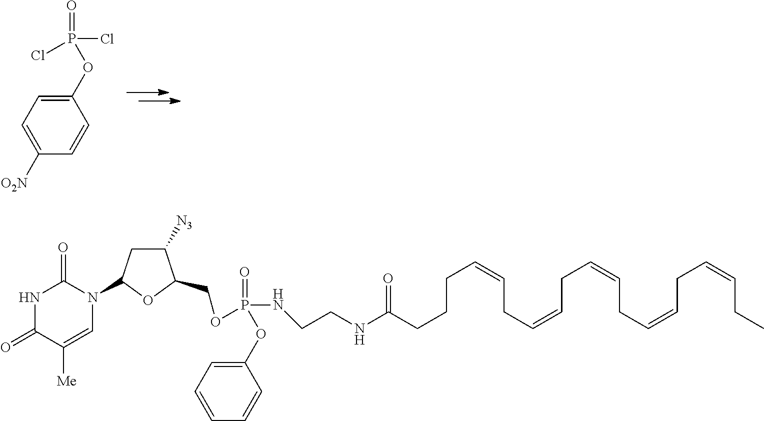

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (example 9) was used. (5Z,8Z,11Z,14Z,17Z)—N-(2-Aminoethyl)icosa-5,8,11,14,17-pentaenamide was used instead of 4Z,7Z,10Z,13Z,16Z,19Z)—N-(2-aminoethyl)docosa-4,7,10,13,16,19-hexaenamide. Purification by preparative HPLC afforded ((2S,3S,5S)-3-azido-5-(5-methyl-2,4-dioxo-3,4-dihydropyrimidin-1(2H)-yl)tetrahydrofuran-2-yl)methyl phenyl (2-((5Z,8Z,11Z,14Z,17Z)-icosa-5,8,11,14,17-pentaenamido)ethyl)phosphoramidate.

MS calculated for C₃₈H₅₂N₇O₇P: 749.83. found: 750.3 [M+H]+.

¹H NMR (400 MHz, CDCl₃) δ 0.94-0.99 (t, J=7.6 Hz, 3H), 1.62-1.70 (m, 3H), 1.88-1.90 (d, J=5.2 Hz, 3H), 2.06-2.13 (m, 6H), 2.31-2.39 (m, 2H), 2.76-2.84 (m, 8H), 3.13-3.17 (m, 2H), 3.30-3.34 (m, 2H), 3.81-3.84 (m, 1H), 4.03 (s, 1H), 4.27-4.37 (m, 3H), 5.29-5.41 (m, 10H), 5.98-6.01 (m, 1H), 6.06-6.12 (m, 1H), 7.16-7.23 (m, 3H), 7.31-7.35 (m, 3H), 7.80-8.81 (m, 1H).

### Example 12

### Preparation of (2S,3S,5S)-3-azido-5-(5-methyl-2,4-dioxo-3,4-dihydropyrimidin-1(2H)-yl)tetrahydrofuran-2-yl)methyl methyl (2-((5Z,8Z,11Z,14Z,17Z)-icosa-5,8,11,14,17-pentaenamido)ethyl)phosphoramidate (II-30)

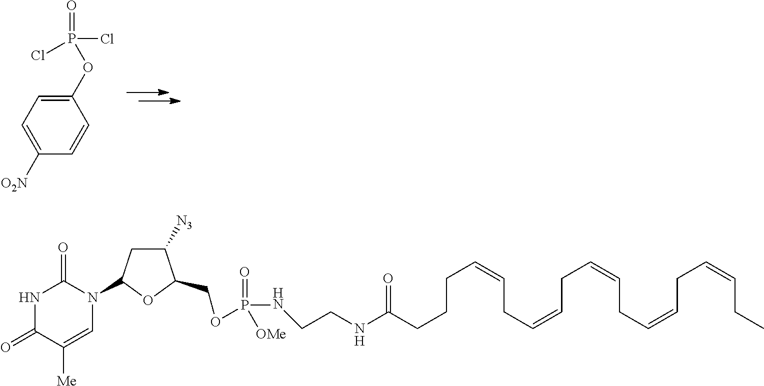

The same experimental procedure detailed in the preparation of methyl (4-nitrophenyl)(2-((4Z,7Z,10Z,13Z,16Z,19Z)-docosa-4,7,10,13,16,19-hexaenamido)ethyl)phosphoramidate (example 10) was used. (5Z,8Z,11Z,14Z,17Z)—N-(2-Aminoethyl)icosa-5,8,11,14,17-pentaenamide was used instead of 4Z,7Z,10Z,13Z,16Z,19Z)—N-(2-aminoethyl)docosa-4,7,10,13,16,19-hexaenamide. Purification by preparative HPLC afforded ((2S,3S,5S)-3-azido-5-(5-methyl-2,4-dioxo-3,4-dihydropyrimidin-1(2H)-yl)tetrahydrofuran-2-yl)methyl methyl (2-((5Z,8Z,11Z,14Z,17Z)-icosa-5,8,11,14,17-pentaenamido)ethyl)phosphoramidate.

MS calculated for C₃₃H₅₀N₇O₇P: 687.77. found: 688.4 [M+H]⁺.

¹H NMR (400 MHz, CDCl₃) δ 0.86-0.90 (t, J=6.6 Hz, 3H), 1.25-1.30 (m, 2H), 1.92 (s, 3H), 2.03-2.13 (m, 4H), 2.19-2.26 (m, 2H), 2.41-2.46 (m, 2H), 2.70-2.99 (m, 9H), 3.06-3.09 (m, 3H), 3.35-3.37 (m, 2H), 3.71-3.77 (d, J=10 Hz, 3H), 4.02 (s, 1H), 4.19-4.37 (m, 2H), 4.36-4.39 (m, 1H), 5.28-5.38 (m, 11H), 6.05-6.23 (m, 2H), 7.26-6.34 (m, 1H), 8.83-8.86 (m, 1H).

### Example 13

### Preparation of ((2R,5S)-5-(4-amino-2-oxopyrimidin-1(2H)-yl)-1,3-oxathiolan-2-yl)methyl phenyl (2-((4Z,7Z,10Z,13Z,16Z,19Z)-docosa-4,7,10,13,16,19-hexaenamido)ethyl)phosphoramidate (II-67)

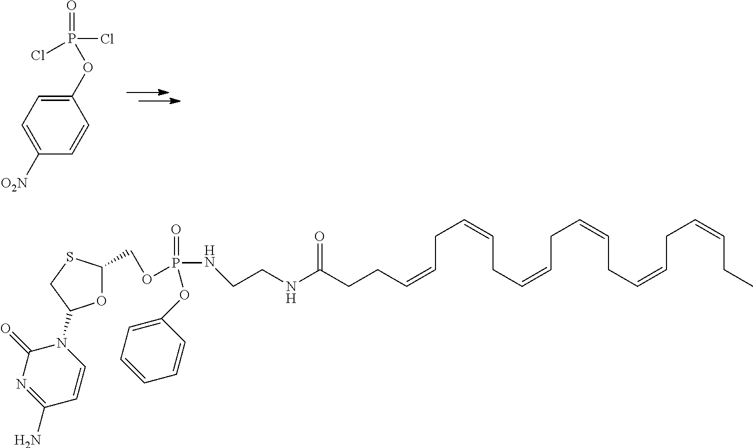

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (example 9) was used. Lamivudine was used instead of zidovudine. Purification by preparative HPLC afforded ((2R,5S)-5-(4-amino-2-oxopyrimidin-1(2H)-yl)-1,3-oxathiolan-2-yl)methyl phenyl (2-((4Z,7Z,10Z,13Z,16Z,19Z)-docosa-4,7,10,13,16,19-hexaenamido)ethyl)phosphoramidate.

MS calculated for C₃₈H₅₂N₅O₆PS: 737.89. found: 738.3 [M+H]⁺.

¹H NMR (400 MHz, CDCl₃) δ 0.95-0.99 (t, J=7.6 Hz, 3H), 2.03-2.11 (m, 2H), 2.15-2.21 (m, 2H), 2.33-2.39 (m, 2H), 2.79-2.84 (m, 10H), 3.00-3.07 (m, 1H), 3.13-3.21 (m, 2H), 3.34-3.36 (m, 2H), 3.46-3.52 (m, 1H), 4.30-4.41 (m, 2H), 4.42-4.45 (m, 0.5H), 4.90-4.94 (m, 0.5H), 5.27-5.37 (m, 13H), 5.68-5.73 (m, 1H), 6.31-6.36 (m, 1H), 6.68-6.70 (m, 0.5H), 6.99-7.01 (m, 0.5H), 7.13-7.22 (m, 3H), 7.26-7.35 (m, 2H), 7.72-7.79 (m, 1H).

### Example 14

### Preparation of ((2R,5S)-5-(4-amino-2-oxopyrimidin-1(2H)-yl)-1,3-oxathiolan-2-yl)methyl phenyl (2-((5Z,8Z,11Z,14Z,17Z)-icosa-5,8,11,14,17-pentaenamido)ethyl)phosphoramidate (II-68)

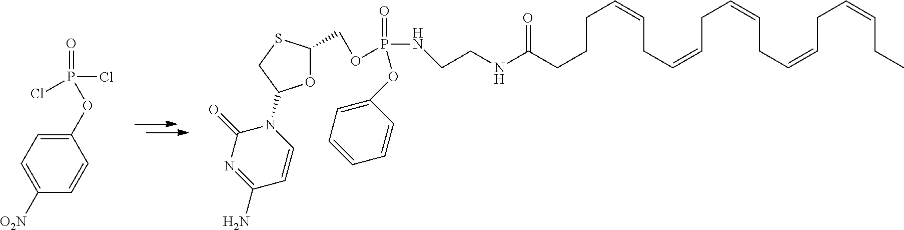

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (example 9) was used. Lamivudine and (5Z,8Z,11Z,14Z,17Z)—N-(2-aminoethyl)icosa-5,8,11,14,17-pentaenamide were the corresponding starting materials. Purification by preparative HPLC afforded ((2R,5S)-5-(4-amino-2-oxopyrimidin-1(2H)-yl)-1,3-oxathiolan-2-yl)methyl phenyl (2-((5Z,8Z,11Z,14Z,17Z)-icosa-5,8,11,14,17-pentaenamido)ethyl)phosphoramidate.

MS calculated for C36H50N5O6PS: 711.85. found: 712.3 [M+H]⁺.

¹H NMR (400 MHz, CDCl₃) δ 0.94-0.99 (t, J=7.6 Hz, 3H), 1.63-1.69 (m, 2H), 1.84 (s, 2H), 2.03-2.17 (m, 6H), 2.76-2.83 (m, 10H), 3.01-3.05 (m, 1H), 3.14-3.19 (m, 2H), 3.33-3.36 (m, 2H), 3.45-3.50 (m, 1H), 4.30-4.36 (m, 2H), 4.87-4.90 (m, 0.5H), 5.07-5.14 (m, 0.5H), 5.30-5.41 (m, 1H), 5.5.73-5.78 (m, 1H), 6.30-6.34 (m, 1H), 6.84-6.86 (m, 0.5H), 7.01-7.03 (m, 0.5H), 7.14-7.22 (m, 3H), 7.26-7.34 (m, 2H), 7.71-7.78 (m, 1H).

### Example 15

### Preparation of ((2R,5S)-5-(4-amino-2-oxopyrimidin-1(2H)-yl)-1,3-oxathiolan-2-yl)methyl methyl (2-((4Z,7Z,10Z,13Z,16Z,19Z)-docosa-4,7,10,13,16,19-hexaenamido)ethyl)phosphoramidate (II-69)

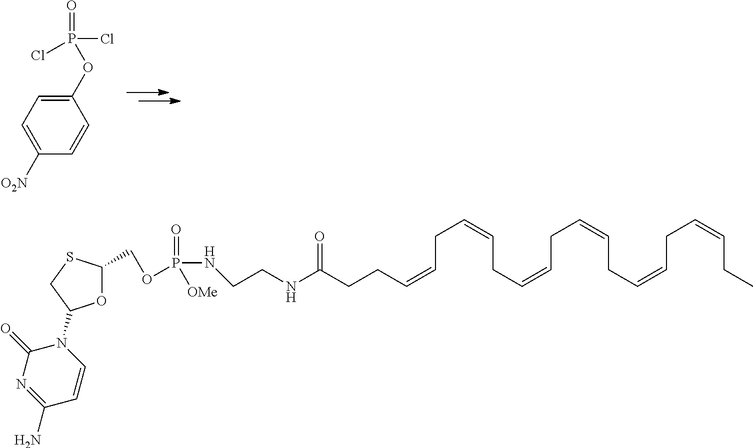

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl methyl (2-((4Z,7Z,10Z,13Z,16Z,19Z)-docosa-4,7,10,13,16,19-hexaenamido)ethyl)phosphoramidate (example 10) was used. Lamivudine was used instead of zidovudine. Purification by preparative HPLC afforded ((2R,5S)-5-(4-amino-2-oxopyrimidin-1(2H)-yl)-1,3-oxathiolan-2-yl)methyl methyl (2-((4Z,7Z,10Z,13Z,16Z,19Z)-docosa-4,7,10,13,16,19-hexaenamido)ethyl)phosphoramidate.

MS calculated for C₃₃H₅₀N₅O₆PS: 675.8. found: 676.3 [M+H]⁺.

¹H NMR (400 MHz, CDCl₃) δ 0.95-1.01 (t, J=7.5 Hz, 3H), 2.05-2.11 (m, 3H), 2.24-2.26 (m, 2H), 2.38-2.41 (m, 2H), 2.80-2.85 (m, 10H), 3.06-3.10 (m, 2H), 3.36-3.38 (m, 2H), 3.51-3.53 (m, 1H), 3.71-3.74 (d, J=11.2 Hz, 3H), 4.21-4.36 (m, 2H), 4.58-4.78 (m, 1H), 5.27-5.37 (m, 13H), 5.84-5.97 (m, 1H), 5.84-5.97 (dd, J=5.2 Hz, 1H), 6.32-6.36 (m, 1H), 7.11-7.22 (m, 1H), 7.78-7.89 (dd, J=7.4 Hz, 2H).

### Example 16

### Preparation of ((2R,5S)-5-(4-amino-2-oxopyrimidin-1(2H)-yl)-1,3-oxathiolan-2-yl)methyl methyl (2-((5Z,8Z,11Z,14Z,17Z)-icosa-5,8,11,14,17-pentaenamido)ethyl)phosphoramidate (II-70)

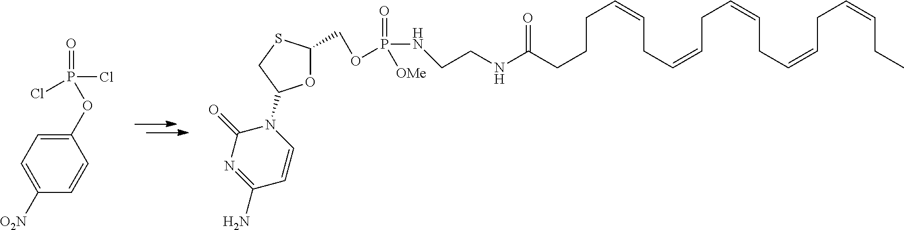

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (example 9) was used. Lamivudine and (5Z,8Z,11Z,14Z,17Z)—N-(2-aminoethyl)icosa-5,8,11,14,17-pentaenamide were the corresponding starting materials. Purification by preparative HPLC afforded ((2R,5S)-5-(4-amino-2-oxopyrimidin-1(2H)-yl)-1,3-oxathiolan-2-yl)methyl methyl (2-((5Z,8Z,11Z,14Z,17Z)-icosa-5,8,11,14,17-pentaenamido)ethyl)phosphoramidate.

MS calculated for C31H₄₈N₅O₆PS: 649.8. found: 650.3 [M+H]⁺.

¹H NMR (400 MHz, CDCl₃) δ 0.95-1.01 (t, J=7.5 Hz, 3H), 1.67-1.75 (m, 2H), 2.03-2.23 (m, 6H), 2.78-2.85 (m, 8H), 3.06-3.15 (m, 3H), 3.34-3.38 (m, 2H), 3.49-3.55 (m, 1H), 3.82-3.87 (d, J=11.4 Hz, 3H), 4.21-4.37 (m, 2H), 4.68-4.96 (m, 1H), 5.30-5.44 (m, 11H), 5.81-5.87 (m, 1H), 6.33-6.37 (s, 1H), 7.07-7.23 (m, 1H), 7.76-7.85 (d, J=7.5 Hz, 1H).

### Example 17

### Preparation of (3R,4R,5S)-ethyl 4-acetamido-5-((4Z,7Z,10Z,13Z,16Z,19Z)-docosa-4,7,10,13,16,19-hexaenamido)-3-(pentan-3-yloxy)cyclohex-1-enecarboxylate (IV-1)

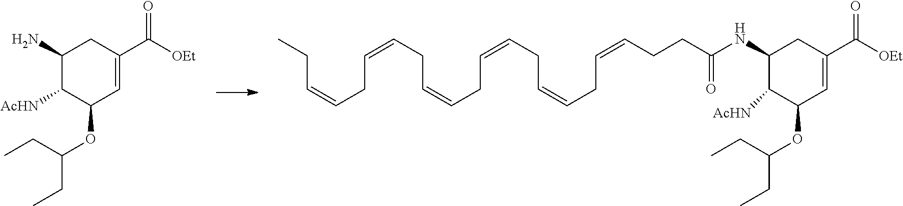 [SMILES: CCOC(=O)C1=C[C@@H](OC(CC)CC)[C@H](NC(C)=O)[C@@H](N)C1 ; CC/C=C\C/C=C\C/C=C\C/C=C\C/C=C\C/C=C\CCC(=O)N[C@H]1CC(C(=O)OCC)=C[C@@H](OC(CC)CC)[C@@H]1NC(C)=O]

(4Z,7Z,10Z,13Z,16Z,19Z)-Docosa-4,7,10,13,16,19-hexaenoic acid (197 mg, 0.64 mmol) was taken up in 10 mL of CH₂Cl₂ along with HOBt (95 mg, 0.70 mmol), EDCI (135 mg, 70 mmol), tamiflu (200 mg, 0.64 mmol) and TEA (194 mg, 1.92 mmol). The resulting reaction mixture was stirred at room temperature overnight. It was then diluted with CH₂Cl₂ (10 mL) and washed with saturated aq. NH₄Cl (3×10 mL) and brine (3×10 mL). The organic layer was dried over anhydrous Na₂SO₄ and concentrated under reduced pressure. The resulting residue was purified by HLPC to afford 160 mg of (3R,4R,5S)-ethyl 4-acetamido-5-((4Z,7Z,10Z,13Z,16Z,19Z)-docosa-4,7,10,13,16,19-hexaenamido)-3-(pentan-3-yloxy)cyclohex-1-enecarboxylate (Yield: 42.8%).

MS calculated for C₃₈H₅₈N₂O₅: 622.88. Found: 623.50 [M+H]⁺.

¹H NMR (400 MHz, CDCl₃) δ 0.87-0.99 (m, 9H), 1.25-1.31 (t, J=7.2 Hz, 3H), 1.49-1.56 (m, 4H), 1.98 (s, 3H), 2.06-2.10 (m, 2H), 2.17-2.22 (m, 2H), 2.33-2.38 (m, 2H), 2.73-2.86 (m, 11H), 3.35-3.39 (m, 1H), 3.99-4.13 (m, 3H), 4.12-4.23 (m, 2H),5.30-5.42 (m, 12H), 5.63-5.66 (d, J=8 Hz, 1H), 6.36-6.39 (d, J=8 Hz, 1H), 6.80 (s, 1H).

### Example 18

### Preparation of (3R,4R,5S)-ethyl 4-acetamido-5-((5Z,8Z,11Z,14Z,17Z)-icosa-5,8,11,14,17-pentaenamido)-3-(pentan-3-yloxy)cyclohex-1-enecarboxylate (IV-2)

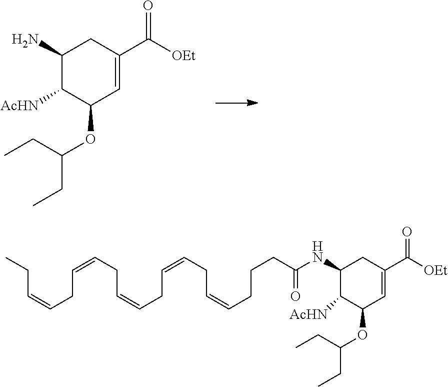 [SMILES: CCOC(=O)C1=C[C@@H](OC(CC)CC)[C@H](NC(C)=O)[C@@H](N)C1 ; CC/C=C\C/C=C\C/C=C\C/C=C\C/C=C\CCCC(=O)N[C@H]1CC(C(=O)OCC)=C[C@@H](OC(CC)CC)[C@@H]1NC(C)=O]

The same experimental procedure detailed in the preparation of (3R,4R,5S)-ethyl 4-acetamido-5-((4Z,7Z,10Z,13Z,16Z,19Z)-docosa-4,7,10,13,16,19-hexaenamido)-3-(pentan-3-yloxy)cyclohex-1-enecarboxylate was used. (5Z,8Z,11Z,14Z,17Z)-Eicosa-5,8,11,14,17-pentaenoic acid was used instead of (4Z,7Z,10Z,13Z,16Z,19Z)-docosa-4,7,10,13,16,19-hexaenoic acid.

MS calculated for C₃₆H₅₆N₂O₅: 596.84. Found: 597.50 [M+H]⁺.

1H NMR (400 MHz, CDCl₃) δ 0.87-0.93 (m, 6H), 0.95-0.99 (t, J=7.6 Hz, 3H), 1.25-1.31 (t, J=7.6 Hz, 3H), 1.49-1.56 (m, 4H), 1.98 (s, 3H), 2.06-2.17 (m, 6H), 2.33-2.38 (m, 1H), 2.73-2.86 (m, 9H), 3.35-3.39 (m, 1H), 3.99-4.13 (m, 3H), 4.12-4.23 (m, 2H), 5.30-5.42 (m, 10H), 5.63-5.66 (d, J=8 Hz, 1H), 6.36-6.39 (d, J=7.6 Hz, 1H), 6.80 (s, 1H).

### Example 19

### Preparation of (3R,4R,5S)-ethyl 4-acetamido-5-(2-((4Z,7Z,10Z,13Z,16Z,19Z)-docosa-4,7,10,13,16,19-hexaenamido)acetamido)-3-(pentan-3-yloxy)cyclohex-1-enecarboxylate (IV-3)

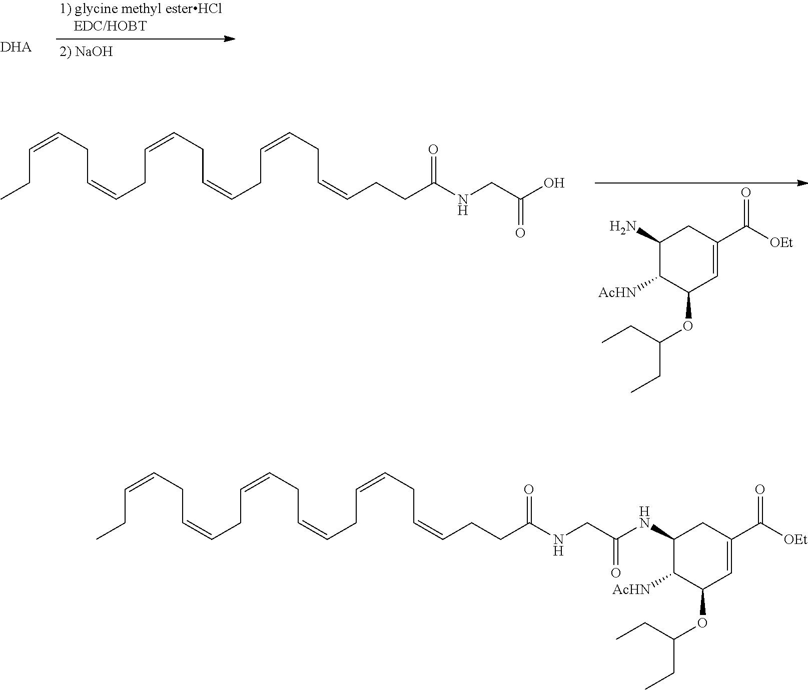 [SMILES: "DHA" ; CC/C=C\C/C=C\C/C=C\C/C=C\C/C=C\C/C=C\CCC(=O)NCC(=O)O ; CCOC(=O)C1=C[C@@H](OC(CC)CC)[C@H](NC(C)=O)[C@@H](N)C1 ; CC/C=C\C/C=C\C/C=C\C/C=C\C/C=C\C/C=C\CCC(=O)NCC(=O)N[C@H]1CC(C(=O)OCC)=C[C@@H](OC(CC)CC)[C@@H]1NC(C)=O]

To a suspension of glycine methyl ester hydrochloride (4 g, 44.9 mmol), EDC (9.47 g, 49.4 mmol), HOBt (6.67 g, 49.4 mmol) and Et₃N (13.6 g, 0.135 mol) in 100 mL of CH₂Cl₂ was added DHA (14 g, 42.7 mmol). The resulting reaction mixture was stirred at room temperature for 18 h. The reaction mixture was washed with saturated aq. NH₄Cl (3×200 mL) and brine (3×200 mL). The organic layer was dried over anhydrous Na₂SO₄ and concentrated under reduced pressure. The resulting residue was purified by silica gel chromatography to afford 9.8 g of methyl 2-((4Z,7Z,10Z,13Z,16Z,19Z)-docosa-4,7,10,13,16,19-hexaenamido)acetate (55%). To the solution of methyl 2-((4Z,7Z,10Z,13Z,16Z,19Z)-docosa-4,7,10,13,16,19-hexaenamido)acetate (3 g, 7.52 mmol) in 50 mL of THF was added 20 mL of aq. NaOH (5N). The resulting reaction mixture was stirred at room temperature for 2 h and then acidified to pH=2 with 6 N HCl. The aqueous phase was extracted with EtOAc. The combined organic layers were washed with brine, dried over Na₂SO₄, filtered and concentrated under reduced pressure. The resulting residue was purified by silica gel chromatography to afford 2.54 g of 2-((4Z,7Z,10Z,13Z,16Z,19Z)-docosa-4,7,10,13,16,19-hexaenamido)acetic acid (88%).

2-((4Z,7Z,10Z,13Z,16Z,19Z)-Docosa-4,7,10,13,16,19-hexaenamido)acetic acid (580 mg, 1.5 mmol) was taken up in 30 mL of CH₂Cl₂ along with HOBt (220 mg, 1.7 mmol), EDCI (330 mg, 1.7 mmol), tamiflu (500 mg, 1.6 mmol) and Et₃N (450 mg, 4.5 mmol). The resulting reaction mixture was stirred at room temperature overnight. It was then diluted with CH₂Cl₂ (30 mL) and washed with saturated aq.NH₄Cl (3×30 mL) and brine (3×30 mL). The organic layer was dried over anhydrous Na₂SO₄ and concentrated under reduced pressure. The resulting residue was purified by silica gel chromatography to afford 370 mg of (3R,4R,5S)-ethyl 4-acetamido-5-(2-((4Z,7Z,10Z,13Z,16Z,19Z)-docosa-4,7,10,13,16,19-hexaenamido)acetamido)-3-(pentan-3-yloxy)cyclohex-1-enecarboxylate (36%).

MS calculated for C₄₀H₆₁N₃O₆:679.9. Found: 680.6 [M+H]⁺.

1H NMR (400 MHz, CDCl₃) δ 0.87-1.01 (m, 9H), 1.27-1.32 (t, J=9.6 Hz, 3H), 1.49-1.55 (m, 4H),1.99 (s, 3H),2.08-2.11 (m, 2H), 2.32-2.45 (m, 5H), 2.78-2.85 (m, 11H), 3.36-3.40 (m, 1H), 3.86-3.92 (m, 2H), 4.04-4.08 (m, 3H), 4.17-4.24 (m, 2H), 5.31-5.43 (m, 12H), 5.92-5.94 (d, J=9.6 Hz, 1H), 6.29 (s, 1H), 6.80 (s, 1H), 7.11-7.13 (d, J=9.6, 1H).

### Example 20

### Preparation of (3R,4R,5S)-ethyl 4-acetamido-5-(3-((4Z,7Z,10Z,13Z,16Z,19Z)-docosa-4,7,10,13,16,19-hexaenamido)propanamido)-3-(pentan-3-yloxy)cyclohex-1-enecarboxylate (IV-4)

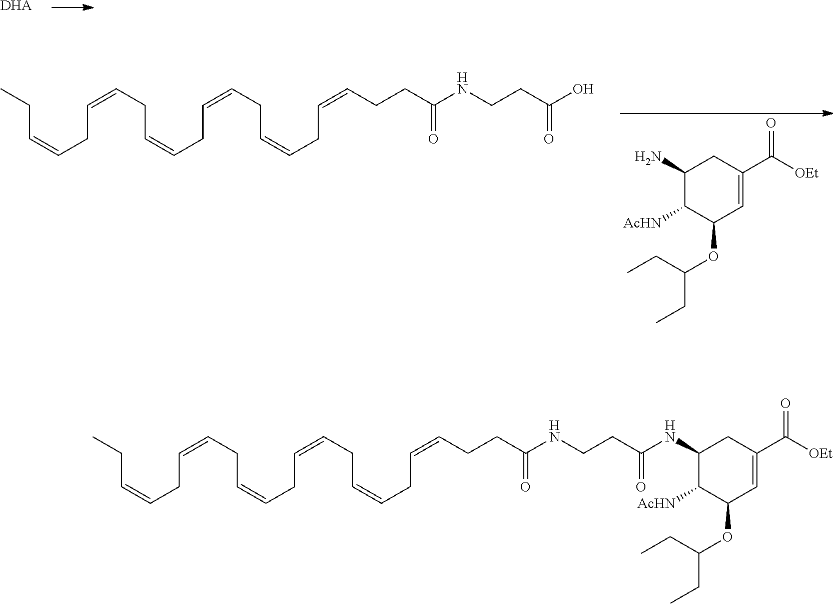 [SMILES: "DHA" ; CC/C=C\C/C=C\C/C=C\C/C=C\C/C=C\C/C=C\CCC(=O)NCCC(=O)O ; CCOC(=O)C1=C[C@@H](OC(CC)CC)[C@H](NC(C)=O)[C@@H](N)C1 ; CC/C=C\C/C=C\C/C=C\C/C=C\C/C=C\C/C=C\CCC(=O)NCCC(=O)N[C@H]1CC(C(=O)OCC)=C[C@@H](OC(CC)CC)[C@@H]1NC(C)=O]

The same experimental procedure detailed in the preparation of (3R,4R,5S)-ethyl 4-acetamido-5-(2-((4Z,7Z,10Z,13Z,16Z,19Z)-docosa-4,7,10,13,16,19-hexaenamido)acetamido)-3-(pentan-3-yloxy)cyclohex-1-enecarboxylate (example 19) was used. Beta-alanine methyl ester was used instead of glycine methyl ester. Purification by silica gel chromatography afforded (3R,4R,5S)-ethyl 4-acetamido-5-(3-((4Z,7Z,10Z,13Z,16Z,19Z)-docosa-4,7,10,13,16,19-hexaenamido)propanamido)-3-(pentan-3-yloxy)cyclohex-1-enecarboxylate.

MS calculated for C₄₁H₆₃N₃O₆: 694.0. Found: 694.50 [M+H]⁺.

1H NMR (400 MHz, CDCl₃) δ 0.84-0.99 (m, 9H), 1.28-1.31 (t, J=9.6 Hz, 3H), 1.50-1.56 (m, 4H), 1.99 (s, 3H), 2.03-2.13 (m, 2H), 2.20-2.31 (m, 2H), 2.36-2.42 (m, 5H), 2.71-2.86 (m, 11H), 3.37-3.53 (m, 3H), 4.10-4.25 (m, 2H), 5.28-5.38 (m, 12H), 5.78-5.80 (d, J=9.6 Hz, 1H), 6.46 (s, 1H), 6.76-6.81 (m, 2H).

### Example 21

### Preparation of (3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (IV-7)

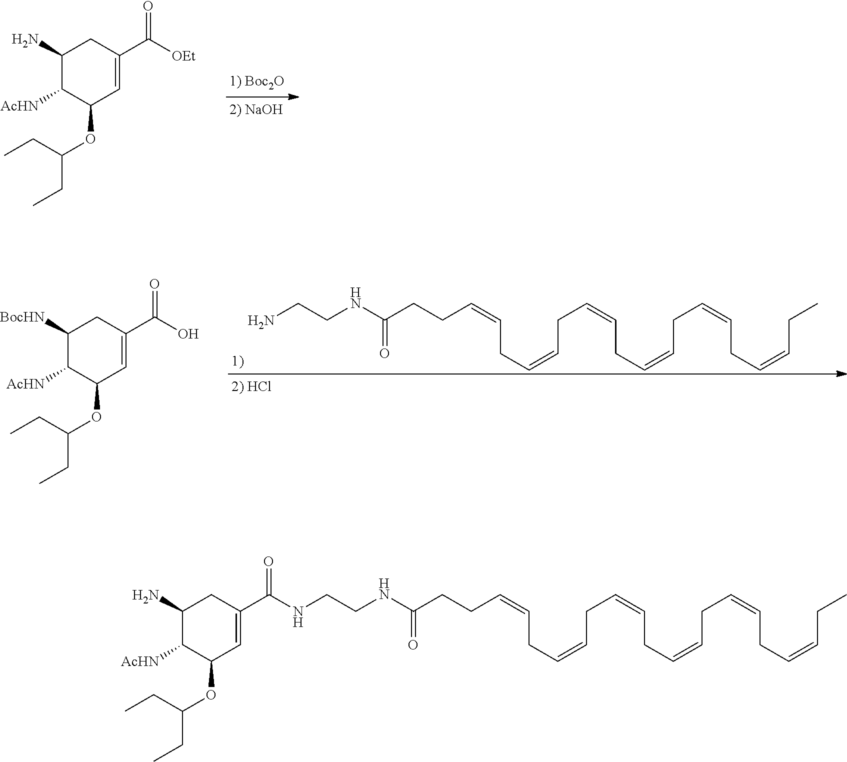

Tamiflu (3.12 g, 10 mmol) was taken up in 50 mL of CH₃OH and triethylamine (3.03 g, 30 mmol) was added slowly at 0° C. Di(tert-butyl) carbonate (2.40 g, 11 mmol) was then added. The resulting reaction mixture was stirred at room temperature for 18 h. The reaction mixture was concentrated under reduced pressure and the residue was diluted with CH₂Cl₂ (50 mL) and washed with saturated aq.NH₄Cl (3×50 mL) and brine (3×50 mL). The organic layer was dried over anhydrous Na₂SO₄ and concentrated under reduced pressure. The resulting residue was purified by silica gel chromatography to afford 3.56 g of (3R,4R,5S)-ethyl 4-acetamido-5-((tert-butoxycarbonyl)amino)-3-(pentan-3-yloxy)cyclohex-1-enecarboxylate (86%). A solution of (3R,4R,5S)-ethyl 4-acetamido-5-((tert-butoxycarbonyl)amino)-3-(pentan-3-yloxy)cyclohex-1-enecarboxylate (3.56 g, 8.64 mmol) in 50 mL of THF was treated with 25 mL of aq. NaOH (5N). The resulting reaction mixture was stirred at room temperature for 2 h. Then the solution was acidified to pH=2 with 6 N HCl. The aqueous phase was extracted with EtOAc. The combined organic layers were washed with brine, dried over Na₂SO₄, filtered and concentrated under reduced pressure. The resulting residue was purified by silica gel chromatography to afford 3.17 g of (3R,4R,5S)-4-acetamido-5-((tert-butoxycarbonyl)amino)-3-(pentan-3-yloxy)cyclohex-1-enecarboxylic acid (95%).

(4Z,7Z,10Z,13Z,16Z,19Z)—N-(2-Aminoethyl)docosa-4,7,10,13,16,19-hexaenamide (1.56 mmol) was taken up in 50 mL of CH₂Cl₂ along with HOBt (0.232 g, 1.72 mmol), EDC (0.33 g, 1.72 mmol), (3R,4R,5S)-4-acetamido-5-((tert-butoxycarbonyl)amino)-3-(pentan-3-yloxy)cyclohex-1-enecarboxylic acid (0.6 g, 1.56 mmol) and Et₃N (0.473 g, 4.68 mmol). The resulting reaction mixture was stirred at room temperature for 18 h. It was then diluted with CH₂Cl₂ (50 mL) and washed with saturated aq.NH₄Cl (3×50 mL) and brine (3×50 mL). The organic layer was dried over anhydrous Na₂SO₄ and concentrated under reduced pressure. The resulting residue was purified by HLPC to afford 0.32 g of tert-butyl ((1S,5R,6R)-6-acetamido-3-((2-((4Z,7Z,10Z,13Z,16Z,19Z)-docosa-4,7,10,13,16,19-hexaenamido)ethyl)carbamoyl)-5-(pentan-3-yloxy)cyclohex-3-en-1-yl)carbamate (28%).

tert-Butyl ((1S,5R,6R)-6-acetamido-3-((2-((4Z,7Z,10Z,13Z,16Z,19Z)-docosa-4,7,10,13,16,19-hexaenamido)ethyl)carbamoyl)-5-(pentan-3-yloxy)cyclohex-3-en-1-yl)carbamate (0.32 g, 0.5 mmol) was treated with a solution of EA/HCl (10 mL). Then the solution was stirred at room temperature for 2 h. Enough saturated aqueous NaHCO₃ was added to adjust the pH=8. The aqueous phase was extracted with EtOAc. The organic phase was washed with brine, then dried over Na₂SO₄, filtered and concentrated to afford 0.29 g of (3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (93%).

MS calculated for C₃₈H₅₈N₂O₅: 636.91. Found: 637.50 [M+H]⁺.

1H NMR (400 MHz, DMSO) δ 0.84-0.86 (m, 6H), 0.89-0.93 (t, J=7.6 Hz, 3H), 1.19-1.19 (t, J=2 Hz, 2H), 1.30-1.47 (m, 4H),1.87 (s, 3H), 1.96-2.11 (m, 4H), 2.33 (s, 1H), 2.66-2.82 (m, 11H), 3.13-3.22 (m, 5H), 3.64-3.42 (m, 1H), 4.07-4.09 (d, J=8 Hz, 1H), 5.24-5.41 (m, 12H), 6.33 (s, 1H), 7.21 (s, 1H), 6.93 (s, 1H), 7.98-8.00 (d, J=8 Hz, 1H), 8.13 (s, 1H).

### Example 22

### Preparation of (3R,4R,5S)-4-acetamido-5-amino-N-(2-((2-((4Z,7Z,10Z,13Z,16Z,19Z)-docosa-4,7,10,13,16,19-hexaenamido)ethyl)(methyl)amino)ethyl)-3-(pentan-3-yloxy)cyclohex-1-enecarboxamide (IV-8)

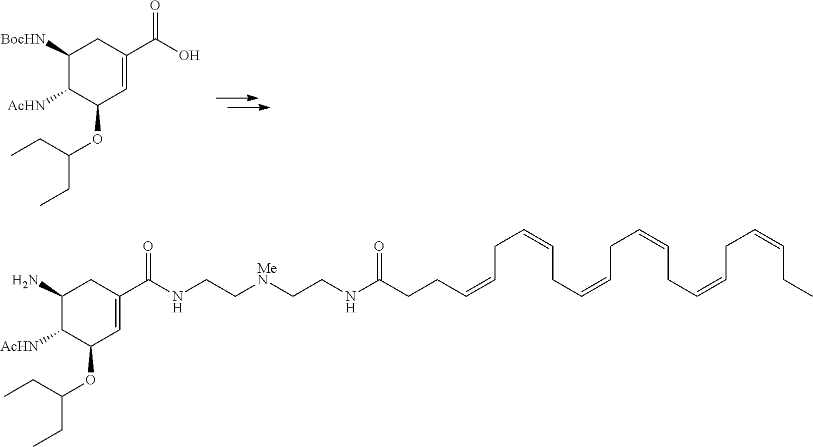 [SMILES: CCC(CC)O[C@@H]1C=C(C(=O)O)C[C@H](NC(=O)OC(C)(C)C)[C@H]1NC(C)=O ; CC/C=C\C/C=C\C/C=C\C/C=C\C/C=C\C/C=C\CCC(=O)NCCN(C)CCNC(=O)C1=C[C@@H](OC(CC)CC)[C@H](NC(C)=O)[C@@H](N)C1]

The same experimental procedure outlined in the preparation of (3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (example 21) was used. (4Z,7Z,10Z,13Z,16Z,19Z)—N-(2-((2-Amino ethyl)(methyl)amino)ethyl)docosa-4,7,10,13,16,19-hexaenamide was used as the appropriate starting material.

MS calculated for C₄₁H₆₇N₅O₄: 694.00. Found: 694.50 [M+H]⁺.

1H NMR (400 MHz, CDCl₃) δ 0.84-1.00 (m, 9H), 1.44-1.56 (m, 4H), 1.96 (s, 3H), 2.03-2.10 (m, 2H), 2.13-2.15 (m, 2H), 2.16-2.27 (m, 6H), 2.67-2.74 (m, 1H), 2.74-2.85 (m, 10H), 3.20-3.31 (m, 1H), 3.33-3.41 (m, 5H), 3.48 (s, 3H), 3.83-3.89 (m, 1H), 4.09-4.12 (m, 1H), 5.29-5.44 (m, 12H), 5.73-5.76 (d, J=7.2 Hz, 1H), 6.12 (s, 1H), 6.33 (s, 1H), 6.45 (s, 1H).

### Example 24

### Preparation of (3R,4R,5S)-4-acetamido-5-amino-N-(2-(2-((4Z,7Z,10Z,13Z,16Z,19Z)-docosa-4,7,10,13,16,19-hexaenamido)ethoxy)ethyl)-3-(pentan-3-yloxy)cyclohex-1-enecarboxamide (IV-9)

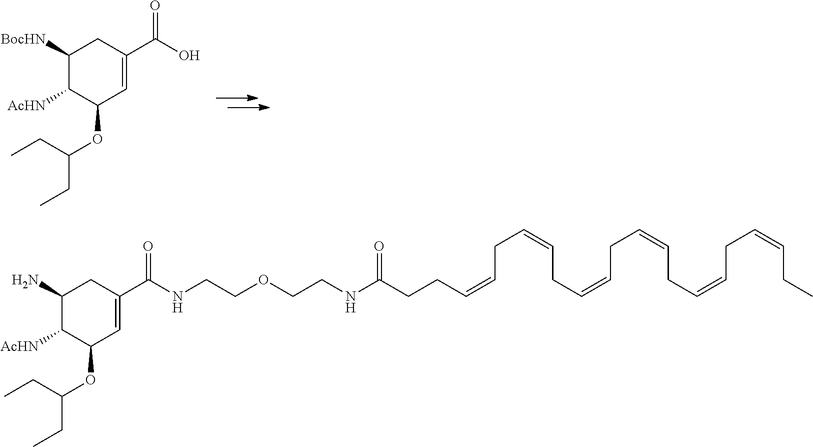 [SMILES: CCC(CC)O[C@@H]1C=C(C(=O)O)C[C@H](NC(=O)OC(C)(C)C)[C@H]1NC(C)=O ; CC/C=C\C/C=C\C/C=C\C/C=C\C/C=C\C/C=C\CCC(=O)NCCOCCNC(=O)C1=C[C@@H](OC(CC)CC)[C@H](NC(C)=O)[C@@H](N)C1]

The same experimental procedure outlined in the preparation of (3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (example 21) was used. (4Z,7Z,10Z,13Z,16Z,19Z)—N-(2-(2-Aminoethoxy)ethyl)docosa-4,7,10,13,16,19-hexaenamide was used as the appropriate starting material.

MS calculated for C₄₀H₆₄N₄O₅: 680.96. Found: 681.50 [M+H]⁺.

1H NMR (400 MHz, CDCl₃) δ 0.80-10.85 (m, 6H), 0.90-0.93 (t, J=7.2 Hz, 3H), 1.23 (s, 1H), 1.35-1.48 (m, 4H), 1.88 (s, 3H), 2.01-2.05 (m, 2H), 2.09-2.11 (m, 2H), 2.22-2.24 (m, 2H), 2.25-2.27 (m, 2H), 2.70-2.82 (m, 11H), 3.15-3.20 (m, 2H), 3.24-3.33 (m, 3H), 3.35-3.44 (m, 3H), 3.74-3.77 (m, 1H), 4.14-4.16 (m, 1H), 5.26-5.38 (m, 12H), 6.36 (s, 1H), 7.86-7.89 (m, 1H), 8.13-8.20 (m, 5H).

### Example 25

### Preparation of (S)-methyl 2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoate (IV-10)

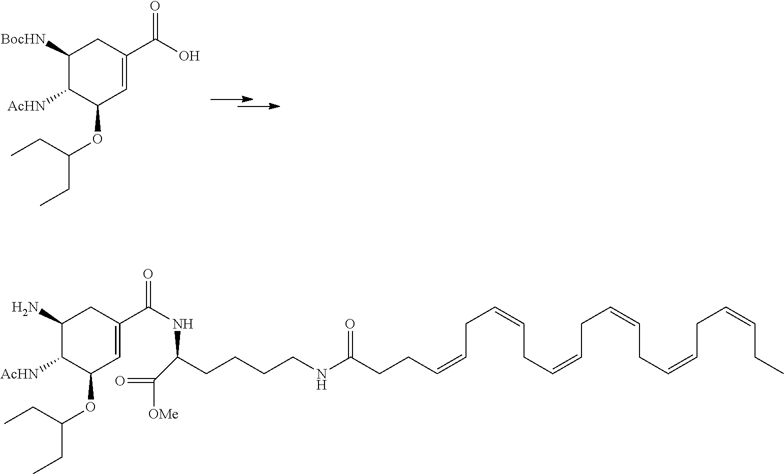 [SMILES: CCC(CC)O[C@@H]1C=C(C(=O)O)C[C@H](NC(=O)OC(C)(C)C)[C@H]1NC(C)=O ; CC/C=C\C/C=C\C/C=C\C/C=C\C/C=C\C/C=C\CCC(=O)NCCCC[C@H](NC(=O)C1=C[C@@H](OC(CC)CC)[C@H](NC(C)=O)[C@@H](N)C1)C(=O)OC]

The same experimental procedure outlined in the preparation of (3R,4R,5S)-4-acetamido-5-amino-N-(2-((4Z,7Z,10Z,13Z,16Z,19Z)-docosa-4,7,10,13,16,19-hexaenamido)ethyl)-3-(pentan-3-yloxy)cyclohex-1-enecarboxamide (example 21) was used. (S)-Methyl 2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoate was used as the appropriate starting material.

MS calculated for C₄₃H₆₈N₄O₆: 737.00. Found: 737.50 [M+H]⁺.

1H NMR (400 MHz, CDCl₃) δ 0.87-0.93 (m, 6H), 0.95-0.99 (t, J=7.6 Hz, 3H), 1.25-1.31 (m, 2H), 1.35-1.47 (m, 6H), 1.50-1.57 (m, 3H), 1.65-1.69-1.47 (m, 1H),1.86 (s, 3H), 1.88-1.90 (m, 2H), 2.13-2.17 (m, 3H), 2.37-2.42 (m, 2H), 2.70-2.75 (m, 1H), 2.79-2.84 (m, 10H), 3.19-3.25 (m, 3H), 3.33-3.37 (m, 1H), 3.57-3.60 (m, 1H), 3.74-3.76 (d, J=6.8 Hz, 3H), 4.14-4.16 (d, J=7.6 Hz, 1H), 4.62-4.65 (m, 3H), 5.29-5.43 (m, 12H), 5.55 (s, 1H), 5.62 (s, 1H),6.38 (d, 1H), 6.39 (s, 1H).

### Example 26

### Preparation of (S)-2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoic acid (IV-11)

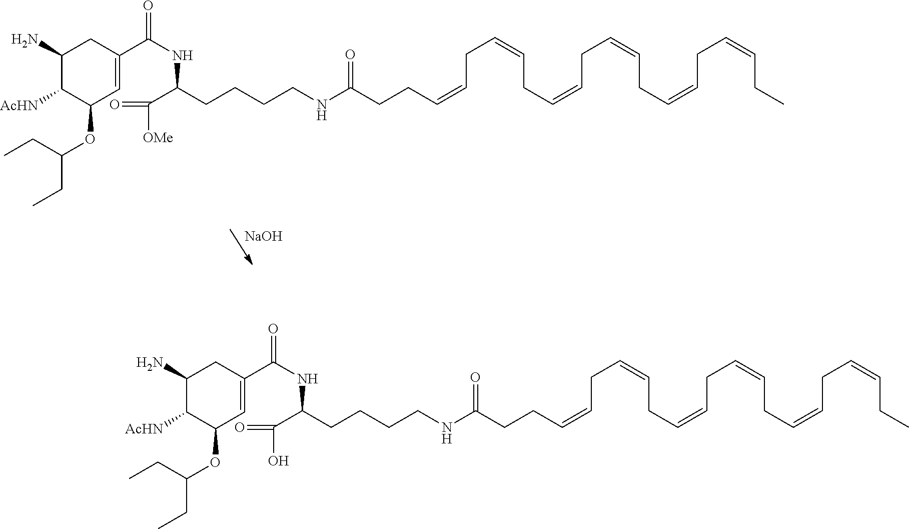

A solution containing (S)-methyl 2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoate (0.2 g, 0.272 mmol) in 10 mL of THF was treated with 5 mL of aq. NaOH (5N). The resulting reaction mixture was stirred at room temperature for 2 h and then acidified to pH=2 with 6 N HCl. The aqueous phase was extracted with EtOAc. The combined organic layers were washed with brine, dried over Na₂SO₄, filtered and concentrated under reduced pressure. Purification by silica gel chromatography afforded (S)-2-((3R,4R,5S)-4-acetamido-5-amino-3-(pentan-3-yloxy)cyclohex-1-enecarboxamido)-6-((4Z,7Z,10Z,13Z,16Z,19Z)-docosa-4,7,10,13,16,19-hexaenamido)hexanoic acid.

MS calculated for C₄₂H₆₆N₄O₆: 723.00. Found: 723.50 [M+H]⁺.

¹H NMR (400 MHz, MeOD) δ 0.73-0.89 (m, 15H), 1.30-1.50 (m, 6H), 1.69 (s, 2H), 1.83 (m, 2H), 1.94-2.00 (m, 2H), 2.09-2.13 (m, 3H), 2.25-2.2.28 (m, 3H), 2.40 (m, 3H), 3.04-3.07 (m, 11H), 3.20-3.32 (m, 3H), 3.44-3.50 (m, 3H), 3.84-3.89 (m, 2H), 4.29-4.32 (m, 2H), 5.21-5.31 (m, 12H), 6.45 (s, 1H).

### Example 27

### Preparation of (3R,4R)-3-acetamido-N-(2-((4Z,7Z,10Z,13Z,16Z,19Z)-docosa-4,7,10,13,16,19-hexaenamido)ethyl)-4-guanidino-2-((1R,2R)-1,2,3-trihydroxypropyl)-3,4-dihydro-2H-pyran-6-carboxamide (IV-12)

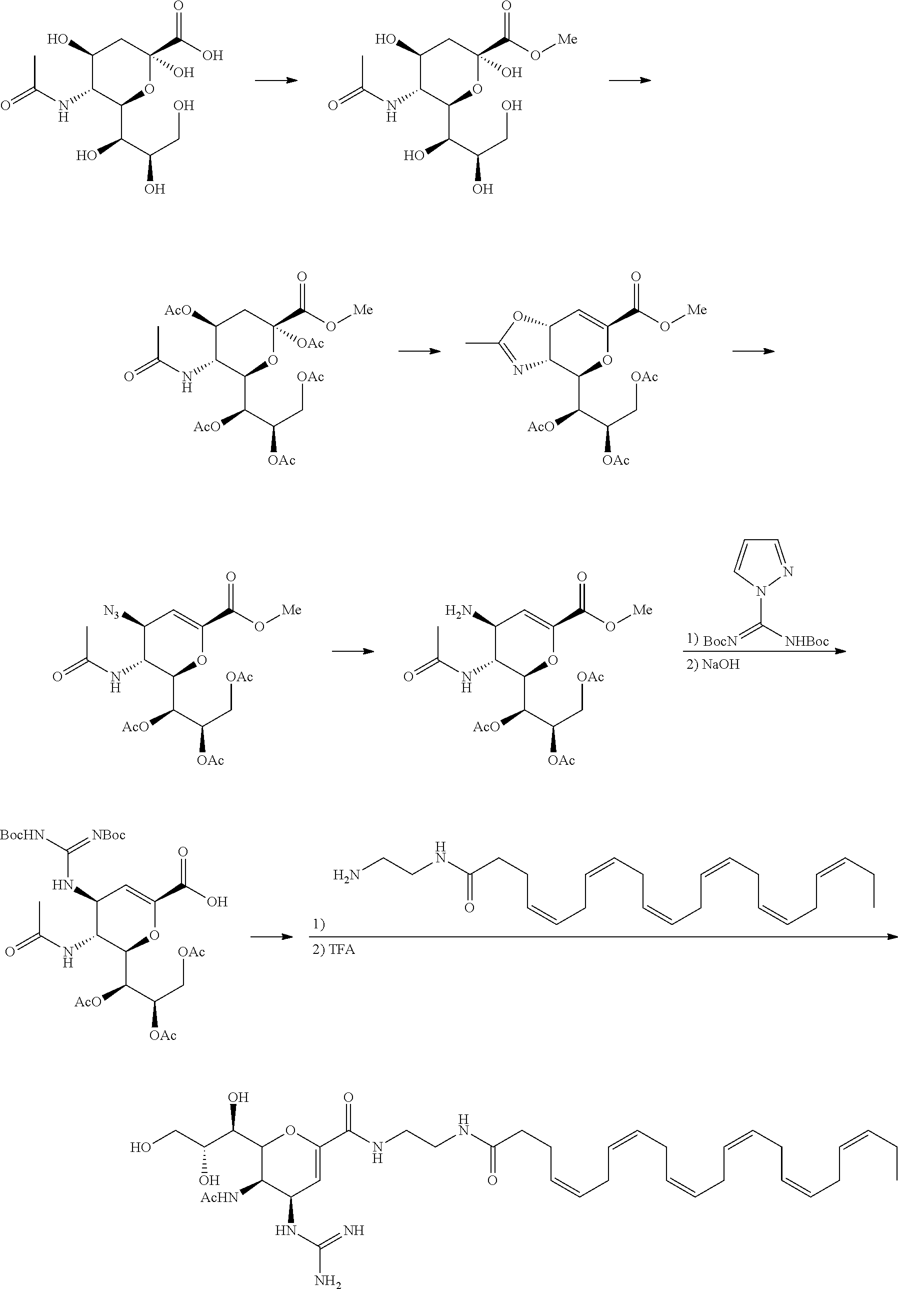 [0800] (2S,4S,5R,6R)-5-Acetamido-2,4-dihydroxy-6-((1R,2R)-1,2,3-trihydroxypropyl)tetrahydro-2H-pyran-2-carboxylic acid (25 g, 80.90 mmol) was added to a mixture of dry Dowex 50W-X4(H⁺, 8 g) and anhydrous CH₃OH (1500 mL) while stirring. The resulting mixture was then stirred at room temperature overnight. The resin was then filtered over Celite and the resulting solution was concentrated to afford 25.5 g of (2S,4S,5R,6R)-methyl 5-acetamido-2,4-dihydroxy-6-((1R,2R)-1,2,3-trihydroxypropyl)tetrahydro-2H-pyran-2-carboxylate (98%).

MS calculated for C₁₂H₂₁NO₉: 323.2. found: 324.1[M+H]⁺.

A solution containing (2S,4S,5R,6R)-methyl 5-acetamido-2,4-dihydroxy-6-((1R,2R)-1,2,3-trihydroxypropyl)tetrahydro-2H-pyran-2-carboxylate (25 g, 77.39 mmol) in pyridine was treated with DMAP (0.24 g, 0.43 mmol). The resulting mixture was cooled in an ice-water-bath while acetic anhydride (72.39 mL) was added dropwise over a period of 15 min. The resulting reaction mixture was warmed to room temperature and then concentrated under reduced pressure. The resulting residue was taken up in EtOAc (300 mL) and washed with 2 M HCl (2*100 mL), saturated aq. sodium hydrogen carbonate (3×100 mL), and brine (100 mL). The organic layer was then dried (Na₂SO₄) and concentrated under reduced pressure to afford ((1S,2R)-1-((2R,3R,4S,6R)-3-acetamido-4,6-diacetoxy-6-(methoxycarbonyl)tetrahydro-2H-pyran-2-yl)propane-1,2,3-triyl triacetate.

¹H NMR (400 MHz, CD₃OD) δ 1.76 (s, 3H), 1.82-2.09 (m, 20H), 2.40-2.44 (m, 1H), 3.66 (s, 3H), 3.90-4.09 (m, 4H), 4.33-4.37 (m, 1H), 4.95-4.99 (m, 1H), 5.05-5.10 (m, 1H), 5.28-5.31 (m, 1H)

(1S,2R)-1-((2R,3R,4S,6R)-3-Acetamido-4,6-diacetoxy-6-(methoxycarbonyl)tetrahydro-2H-pyran-2-yl)propane-1,2,3-triyl triacetate was taken up in warm EtOAc (300 mL) and then cooled to about 30° C. while TMSOTf (133.73 mL) was added dropwise during 10 min with stirring of the mixture under N₂ atmosphere. After the addition was complete the temperature was raised to 52° C. over a period of 20 min. After 3 h at this temperature the reaction mixture was allowed to cool to room temperature and poured into a vigorously stirred mixture of ice-cold saturated aq. sodium hydrogen carbonate (300 mL). The two layers were separated and the aqueous layer was further extracted with EtOAc. The combined organic layers were dried (Na₂SO₄) and concentrated under reduced pressure. The resulting residue was purified by column chromatography (mixture of EtAOc/pentanes) to give (1S,2R)-1-((3aR,4R,7aR)-6-(methoxycarbonyl)-2-methyl-4,7a-dihydro-3aH-pyrano[3,4-d]oxazol-4-yl)propane-1,2,3-triyl triacetate (yield: 16%).

MS calculated for C₂₈H₂₃NO₁₀: 413.3. Found: 414.2[M+H]⁺.

¹H NMR (400 MHz, CDCl₃) δ 1.94-2.08 (m, 12H), 3.35-3.38 (m, 1H), 3.75 (s, 3H), 3.86-3.89 (m, 1H), 4.02-4.18 (m, 1H), 4.51-4.52 (m, 1H), 4.74-4.77 (m, 1H), 5.37 (s, 1H), 5.56-5.58 (m, 1H), 6.31-6.32 (m, 1H).

A solution containing (1S,2R)-1-((3aR,4R,7aR)-6-(methoxycarbonyl)-2-methyl-4,7a-dihydro-3aH-pyrano[3,4-d]oxazol-4-yl)propane-1,2,3-triyl triacetate (5.0 g, 12.09 mmol) and azidotrimethylsilane (2.4 mL) in tert-butyl alcohol (50 mL) under nitrogen was stirred under reflux over a steam bath for 10 h. The reaction mixture was allowed to cool to room temperature and aq. sodium nitrite (12 g, 60 mL) was added, followed by dropwise addition of 6N HCl (5 mL) over a period of 30 min to give vigorous evolution of gases. EtOAc and water were then added and the organic layer was separated and washed with water. The combined aqueous layers were extracted with EA and the combined organic layers were washed with 6% aq. NaHCO₃ followed by brine. The combined organic layers were dried (Na₂SO₄) and concentrated under reduced pressure to afford 5.1 g of (1S,2R)-1-((2R,3R,4S)-3-acetamido-4-azido-6-(methoxycarbonyl)-3,4-dihydro-2H-pyran-2-yl)propane-1,2,3-triyl triacetate (96%). A solution of (1S,2R)-1-((2R,3R,4S)-3-acetamido-4-azido-6-(methoxycarbonyl)-3,4-dihydro-2H-pyran-2-yl)propane-1,2,3-triyl triacetate (5.1 g, 11.5 mmol) in ethanol (300 mL) was hydrogenated with Lindlar's catalyst for 8 h (1 atmospheric pressure). The reaction mixture was filtered through Celite and the filtrate was concentrated under reduced pressure. The resulting residue was dissolved in THF (50 mL). This solution was mixed with tert-butyl (((tert-butoxycarbonyl)amino)(1H-pyrazol-1-yl)methylene)carbamate (13.1 g, 42.21 mmol) and the resulting reaction mixture was stirred at room temperature for 18 h. It was then diluted with aq. NH₄Cl and extracted with EtOAc. The combined organic layers were washed with brine, dried over Na₂SO₄, and concentrated under reduced pressure. Purification by silica gel chromatography (gradient elution, 1:1 EtOAc/pentane to 100% EtOAc) to afford 4.0 g of (1S,2R)-1-((2R,3R,4S)-3-acetamido-4-(2,3-bis(tert-butoxycarbonyl)guanidino)-6-(methoxycarbonyl)-3,4-dihydro-2H-pyran-2-yl)propane-1,2,3-triyl triacetate (50%). (1S,2R)-1-((2R,3R,4S)-3-Acetamido-4-(2,3-bis(tert-butoxycarbonyl)guanidino)-6-(methoxycarbonyl)-3,4-dihydro-2H-pyran-2-yl)propane-1,2,3-triyl triacetate (4.0 g, 5.94 mmol) was dissolved in methanol (20 mL) and treated with 1N NaOH (6 mL) with cooling in an ice-bath. The reaction was stirred for 30 min and neutralized with 1N HCl. Then the reaction was concentrated under reduced pressure and the residue was re-dissolved in methanol and filtered. The filtrate was concentrated under reduced pressure to afford 3.0 g of (2R,3R,4S)-3-acetamido-4-(2,3-bis(tert-butoxycarbonyl)guanidino)-2-((1S,2R)-1,2,3-triacetoxypropyl)-3,4-dihydro-2H-pyran-6-carboxylic acid (95%).

A mixture containing (2R,3R,4S)-3-acetamido-4-(2,3-bis(tert-butoxycarbonyl)guanidino)-2-((1S,2R)-1,2,3-triacetoxypropyl)-3,4-dihydro-2H-pyran-6-carboxylic acid (0.5 g, 0.94 mmol), (4Z,7Z,10Z,13Z,16Z,19Z)—N-(2-aminoethyl)docosa-4,7,10,13,16,19-hexaenamide (0.38 g, 1.03 mmol), HATU (0.39 g, 1.03 mmol) and DIEA (0.36 g, 2.81 mmol) in DMF (10 mL) was stirred at room temperature for 18 h. The reaction mixture was diluted with EtOAc (250 mL) and washed with aq. NH₄Cl (3×20 mL) and brine (3×20 mL). The organic layer was dried over anhydrous Na₂SO₄ and concentrated under reduced pressure. The resulting residue was purified by silica gel chromatography (95% CH₂Cl₂/5% MeOH) to afford 300 mg of the BOC-protected intermediate (36%). This material was then treated with CH₂Cl₂ (100 mL) and TFA (10 mL) and the reaction mixture was stirred at room temperature for 8 h and then concentrated under reduced pressure. The resulting residue was purified by preparative HPLC to afford 25 mg of (3R,4R)-3-acetamido-N-(2-((4Z,7Z,10Z,13Z,16Z,19Z)-docosa-4,7,10,13,16,19-hexaenamido)ethyl)-4-guanidino-2-((1R,2R)-1,2,3-trihydroxypropyl)-3,4-dihydro-2H-pyran-6-carboxamide as the TFA salt (9%).

MS calculated for C₃₆H₅₆N₆O₇: 684.8. Found: 685.2 [M+H]⁺.

¹H NMR (400 MHz, CD₃OD) δ 0.81-0.89 (t, J=7.5 Hz, 3H), 1.91 (s, 3H), 1.96-2.00 (m, 2H), 2.11-2.15 (m, 10H), 2.25-2.28 (m, 2H), 2.70-2.77 (m, 10H), 3.20-3.29 (m, 3H), 3.59-3.64 (m, 2H), 3.71-3.74 (m, 2H), 4.11-4.14 (m, 1H), 4.29-4.33 (m, 2H), 5.21-5.31 (m, 12H), 5.61 (s, 1H).

### Example 28

### Preparation of (3R,4R)-3-acetamido-4-guanidino-N-(2-((5Z,8Z,11Z,14Z,17Z)-icosa-5,8,11,14,17-pentaenamido)ethyl)-2-((1R,2R)-1,2,3-trihydroxypropyl)-3,4-dihydro-2H-pyran-6-carboxamide (IV-13)

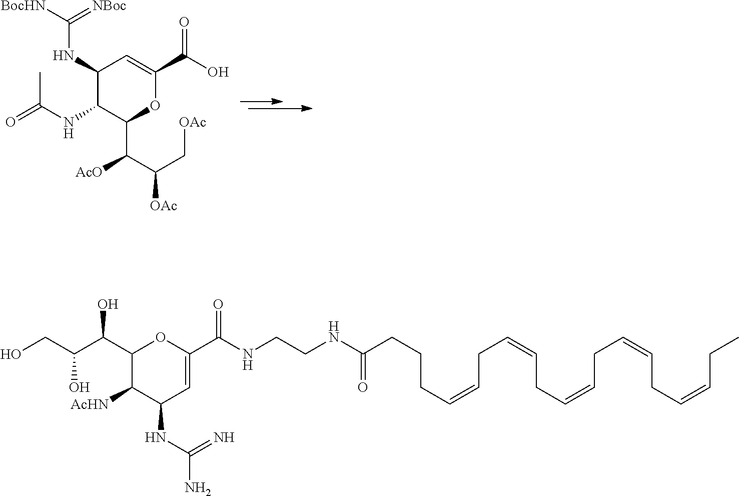

The same experimental procedure detailed in the preparation of (3R,4R)-3-acetamido-N-(2-((4Z,7Z,10Z,13Z,16Z,19Z)-docosa-4,7,10,13,16,19-hexaenamido)ethyl)-4-guanidino-2-((1R,2R)-1,2,3-trihydroxypropyl)-3,4-dihydro-2H-pyran-6-carboxamide was used, substituting (5Z,8Z,11Z,14Z,17Z)—N-(2-aminoethyl)icosa-5,8,11,14,17-pentaenamide as the appropriate starting material.

MS calculated for C₃₄H₅₄N₆O₇: 658.8. Found: 659.4 [M+H]⁺.

¹H NMR (400 MHz, CD₃OD) δ 0.82-0.89 (t, J=7.5 Hz, 3H), 1.54-1.58 (m, 2H), 1.91 (s, 3H), 1.96-2.00 (m, 4H), 2.08-2.12 (m, 2H), 2.11-2.15 (m, 8H), 3.20-3.31 (m, 4H), 3.59-3.64 (m, 2H), 3.71-3.78 (m, 2H), 4.09-4.14 (m, 1H), 4.30-4.33 (m, 2H), 5.21-5.29 (m, 10H), 5.61 (s, 1H).

### Example 29

### Preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (V-1)

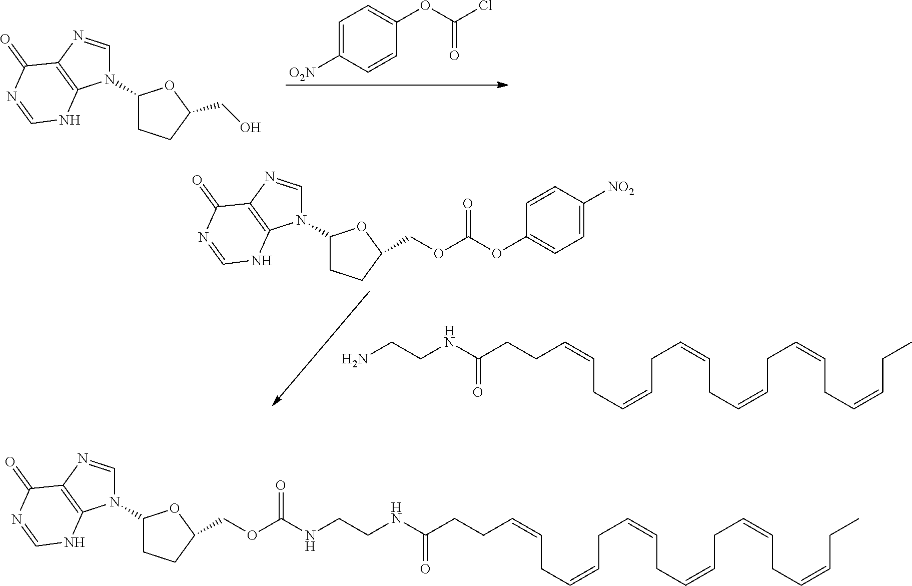

A mixture containing didanosine (0.15 g, 0.63 mmol), Et₃N (0.19 g, 1.9 mmol) and 2 (0.15 g, 0.76 mmol) in DMF (5 mL) was stirred at room temperature for 18 h. It was then diluted with EtOAc (50 mL) and then washed with saturated aq. NH₄Cl (3×10 mL) and brine (3×10 mL). The organic layer was dried over anhydrous Na₂SO₄ and concentrated under reduced pressure. The resulting residue was purified by silica gel chromatography to afford 0.16 g of 4-nitrophenyl (((2S,5R)-5-(6-oxo-3H-purin-9(6H)-yl)tetrahydrofuran-2-yl)methyl)carbonate (63%). A mixture containing 4-nitrophenyl (((2S,5R)-5-(6-oxo-3H-purin-9(6H)-yl)tetrahydrofuran-2-yl)methyl)carbonate (80 mg, 0.199 mmol), DIEA (38 mg, 0.29 mmol), DMAP (36 mg, 0.29 mmol) and (4Z,7Z,10Z,13Z,16Z,19Z)—N-(2-aminoethyl)docosa-4,7,10,13,16,19-hexaenamide (80 mg, 0.219 mmol) in 3 mL of DMF was stirred at room temperature for 18 h. The reaction mixture was diluted with EtOAc (50 mL) and washed with saturated aq. NH₄Cl (3×10 mL) and brine (3×10 mL). The organic layer was dried over anhydrous Na₂SO₄ and concentrated under reduced pressure. The resulting residue was purified by silica gel chromatography to afford 110 mg of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (87%).

MS calculated for C₃₅H₄₈N₆O₅: 632.79. Found: 633.1 [M+H]⁺.

¹H NMR (400 MHz, CD₃OD) δ 0.83-0.87 (t, J=7.6 Hz, 3H), 1.95-2.14 (m, 6H), 2.23-2.26 (m, 2H), 2.42-2.47 (m, 2H), 2.69-2.74 (m, 10H), 3.08-3.21 (m, 4H), 4.09-4.12 (m, 1H), 4.22-4.28 (m, 2H),5.17-0.28 (m, 12H), 6.18-6.20 (m, 1H), 7.93 (s, 1H), 8.14 (s, 1H).

### Example 30

### Preparation of ((2S,5R)-5-(6-oxo-3H-purin-9(6H)-yl)tetrahydrofuran-2-yl)methyl (2-((5Z,8Z,11Z,14Z,17Z)-icosa-5,8,11,14,17-pentaenamido)ethyl)carbamate (V-2)

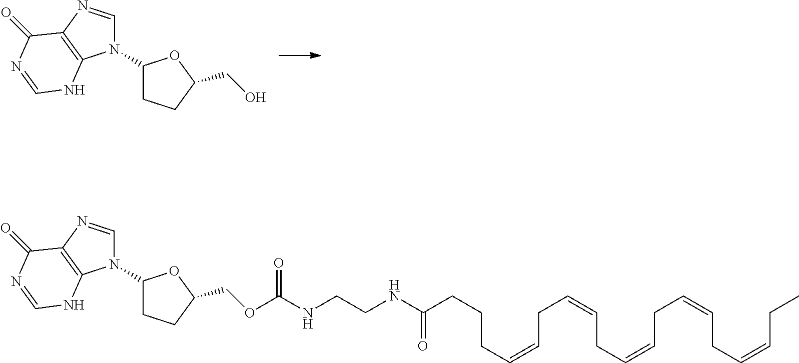

The same experimental procedure detailed in the preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (example 29) was used. (5Z,8Z,11Z,14Z,17Z)—N-(2-Aminoethyl)icosa-5,8,11,14,17-pentaenamide was used as the appropriate starting material.

MS calculated for C₃₃H₄₆N₆O₅: 606.3. Found: 607.1 [M+H]⁺.

¹H NMR (400 MHz, CD₃OD) δ 0.93-0.98 (t, J=10 Hz, 3H), 1.62-1.67 (m, 2H), 2.02-2.23 (m, 8H), 2.52-2.54 (m, 2H), 2.80-2.83 (m, 8H), 3.21-3.32 (m, 4H), 4.19-4.37 (m, 3H), 5.28-5.34 (m, 10H), 6.28 (s, 1H), 8.08 (s, 1H), 8.26 (s, 1H).

### Example 31

### Preparation of (S)-((2S,5R)-5-(6-oxo-3H-purin-9(6H)-yl)tetrahydrofuran-2-yl)methyl 2-((4Z,7Z,10Z,13Z,16Z,19Z)-docosa-4,7,10,13,16,19-hexaenamido)propanoate (V-3)

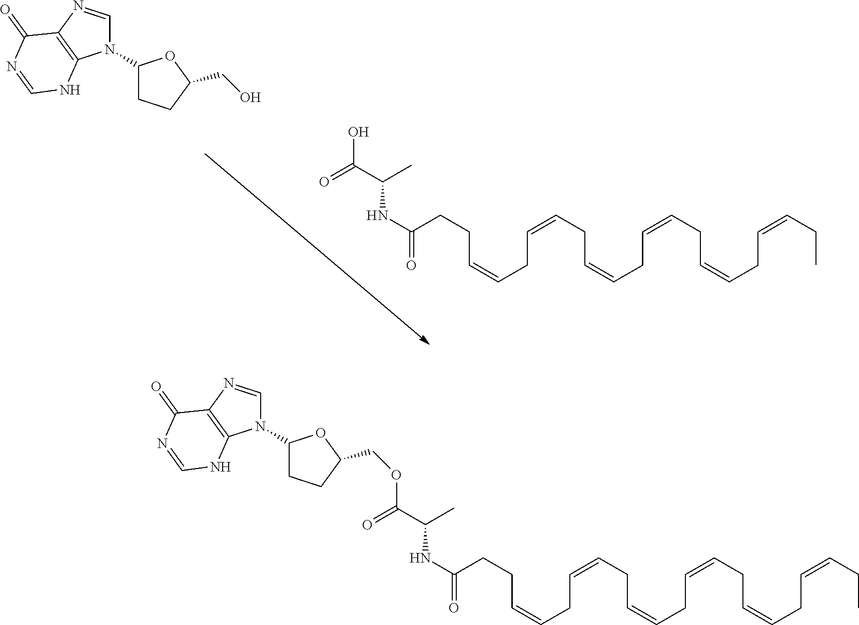

A mixture containing didanosine (100 mg, 0.42 mmol), DCC (348 mg, 1.69 mmol), DMAP (5.12 mg, 0.042 mmol) and (S)-2-((4Z,7Z,10Z,13Z,16Z,19Z)-docosa-4,7,10,13,16,19-hexaenamido)propanoic acid in 3 mL of DMF was stirred at room temperature for 18 h. S)-2-((4Z,7Z,10Z,13Z,16Z,19Z)-Docosa-4,7,10,13,16,19-hexaenamido)propanoic acid, in turn, was prepared according to the procedure outlined in US 20110212958. The reaction mixture was diluted with EtOAc (50 mL) and the organic layer was washed with saturated aq. NH₄Cl (3×10 mL) and brine (3×10 mL). The organic layer was dried over anhydrous Na₂SO₄ and concentrated under reduced pressure. The resulting residue was purified by silica gel chromatography to afford 100 mg of (S)-((2S,5R)-5-(6-oxo-3H-purin-9(6H)-yl)tetrahydrofuran-2-yl)methyl 2-((4Z,7Z,10Z,13Z,16Z,19Z)-docosa-4,7,10,13,16,19-hexaenamido)propanoate (87%).

MS calculated for C₃₅H₄₇N₅O₅: 617.7. Found: 618.1 [M+H]⁺.

¹H NMR (400 MHz, CDCl₃) δ 0.85-0.92 (t, J=7.8 Hz, 3H), 1.18-1.35 (m, 3H), 1.96-2.34 (m, 8H), 2.48-2.54 (m, 2H), 2.73-2.77 (m, 10H), 4.23-4.49 (m, 4H), 5.22-5.35 (m, 12H), 6.04-6.23 (m, 2H), 8.05-8.11 (m, 2H).

### Example 32

### Preparation of (2S,3S,5S)-3-azido-5-(5-methyl-2,4-dioxo-3,4-dihydropyrimidin-1(2H)-yl)tetrahydrofuran-2-yl)methyl (2-((4Z,7Z,10Z,13Z,16Z,19Z)-docosa-4,7,10,13,16,19-hexaenamido)ethyl)carbamate (V-5)

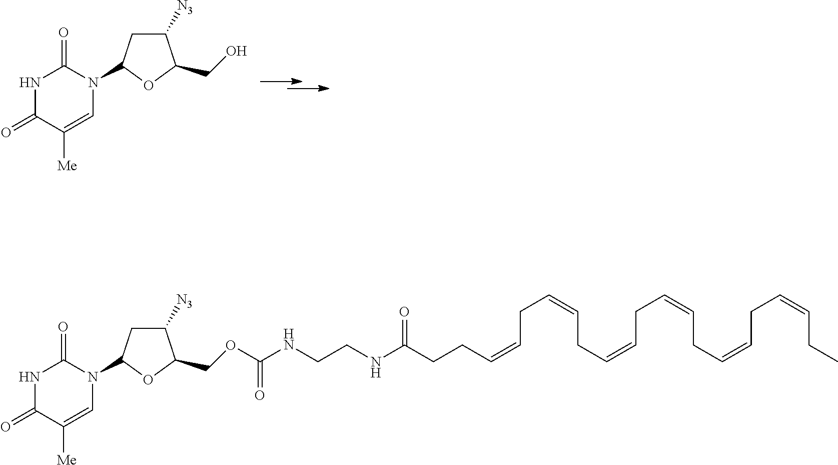

The same experimental procedure detailed in the preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (example 29) was used. Zidovudine was used as the appropriate starting material.

MS calculated for C₃₅H₄₉N₇O₆: 663.8. Found: 664.1 [M+H]⁺.

¹H NMR (400 MHz, CDCl₃) δ 0.95-1.00 (t, J=10.4 Hz, 3H), 1.94 (s, 3H), 2.06-2.10 (m, 2H), 2.22-2.27 (m, 2H), 2.37-2.45 (m, 4H), 2.80-2.85 (m, 10H), 3.32-3.42 (m, 4H), 4.04-4.07 (m, 1H), 4.26-4.34 (m, 3H), 5.30-5.44 (m, 12H), 5.72 (s, 1H), 6.04-6.08 (m, 2H), 7.21 (s, 1H), 8.90 (s, 1H).

### Example 33

### Preparation of (2S,3S,5S)-3-azido-5-(5-methyl-2,4-dioxo-3,4-dihydropyrimidin-1(2H)-yl)tetrahydrofuran-2-yl)methyl (2-((2-((4Z,7Z,10Z,13Z,16Z,19Z)-docosa-4,7,10,13,16,19-hexaenamido)ethyl)(methyl)amino)ethyl)carbamate (V-7)

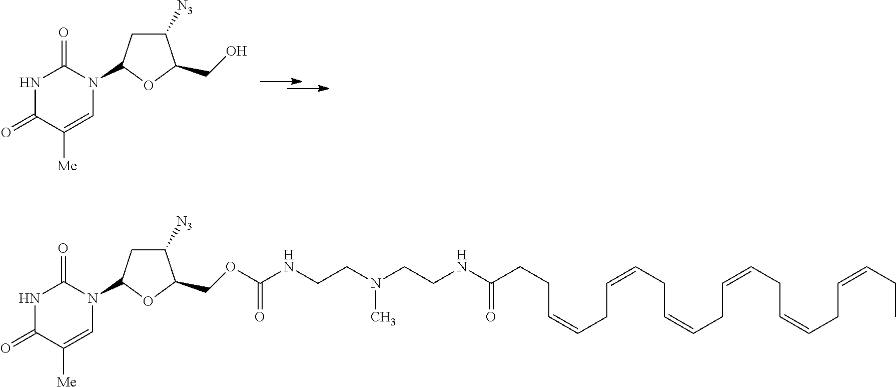

The same experimental procedure detailed in the preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (example 29) was used. Zidovudine and (4Z,7Z,10Z,13Z,16Z,19Z)—N-(2-((2-aminoethyl)(methyl)amino)ethyl)docosa-4,7,10,13,16,19-hexaenamide were used as the appropriate starting materials.

MS calculated for C₃₈H₅₆N₈O₆: 720.9. Found: 721.8 [M+H]⁺.

¹H NMR (400 MHz, CDCl₃) δ 0.95-1.00 (t, J=10 Hz, 3H), 1.92 (s, 3H), 2.03-2.13 (m, 3H), 2.19-2.27 (m, 4H), 2.36-2.57 (m, 8H), 2.79-2.85 (m, 10H),3.26-3.39 (m, 4H), 4.05-4.09 (m, 1H), 4.25-4.39 (m, 2H), 5.29-5.44 (m, 12H), 5.76-5.79 (m, 1H), 6.06-6.15 (m, 1H).

### Example 34

### Preparation of (2S,3S,5S)-3-azido-5-(5-methyl-2,4-dioxo-3,4-dihydropyrimidin-1(2H)-yl)tetrahydrofuran-2-yl)methyl (2-(2-((4Z,7Z,10Z,13Z,16Z,19Z)-docosa-4,7,10,13,16,19-hexaenamido)ethoxy)ethyl)carbamate (V-8)

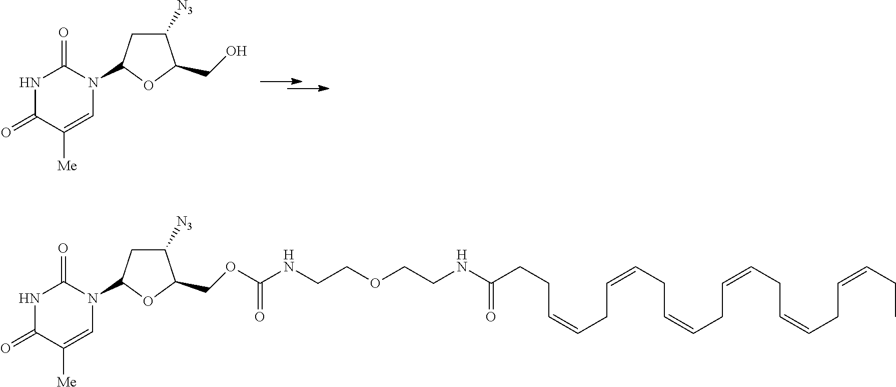

The same experimental procedure detailed in the preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (example 29) was used. Zidovudine and (4Z,7Z,10Z,13Z,16Z,19Z)—N-(2-(2-aminoethoxy)ethyl)docosa-4,7,10,13,16,19-hexaenamide were used as the starting materials.

MS calculated for C₃₇H₅₃N₇O₇: 707.8. Found: 708.5 [M+H]⁺.

¹H NMR (400 MHz, CDCl₃) δ 095-1.00 (t, J=10 Hz, 3H), 1.78 (s, 3H), 2.03-2.13 (m, 3H), 2.23-2.27 (m, 2H), 2.38-2.47 (m, 4H), 2.79-2.85 (m, 10H), 3.39-3.58 (m, 8H), 4.06-4.11 (m, 1H), 4.26-4.42 (m, 3H),5.29-5.45 (m, 12H), 5.57-5.60 (m, 1H), 6.04-6.15 (m, 2H), 7.25-7.27 (m, 1H), 9.02 (s, 1H).

### Example 35

### Preparation of (S)-((2S,3S,5S)-3-azido-5-(5-methyl-2,4-dioxo-3,4-dihydropyrimidin-1(2H)-yl)tetrahydrofuran-2-yl)methyl 2-((4Z,7Z,10Z,13Z,16Z,19Z)-docosa-4,7,10,13,16,19-hexaenamido)-3-methylbutanoate (V-9)

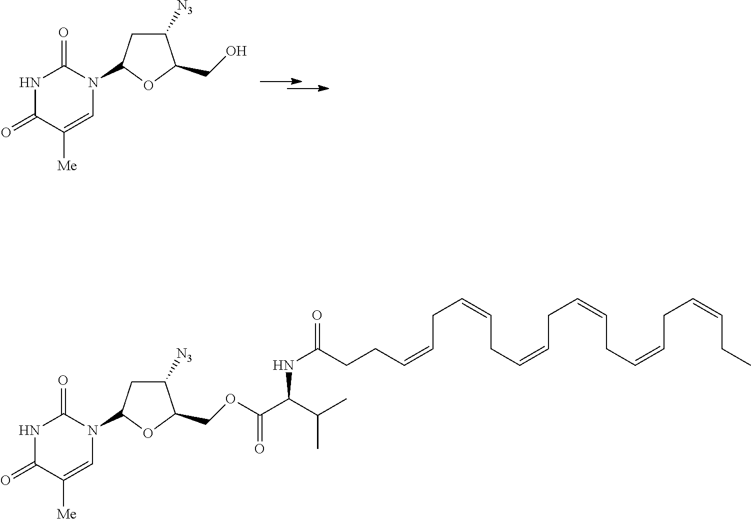

The same experimental procedure outlined in the preparation of (S)-((2S,5R)-5-(6-oxo-3H-purin-9(6H)-yl)tetrahydrofuran-2-yl)methyl 2-((4Z,7Z,10Z,13Z,16Z,19Z)-docosa-4,7,10,13,16,19-hexaenamido)propanoate was used. Zidovudine was used as the appropriate starting material. MS calculated for C₃₇H₅₂N₆O₆: 676.8. Found: 677.5 [M+H]⁺.

¹H NMR (400 MHz, CDCl₃) δ 0.85-0.92 (m, 10H), 1.86-2.11 (m, 8H), 2.23-2.40 (m, 6H), 2.72-3.87 (m, 10H), 3.87-4.00 (m, 1H), 4.13-4.38 (m, 3H), 4.51-4.67 (m, 1H), 5.21-5.38 (m, 12H), 5.87-6.13 (m, 2H), 7.09-7.29 (m, 2H), 8.63 (m, 1H).

### Example 36

### Preparation of (4Z,7Z,10Z,13Z,16Z,19Z)-((2R,5S)-5-(4-amino-2-oxopyrimidin-1(2H)-yl)-1,3-oxathiolan-2-yl)methyl docosa-4,7,10,13,16,19-hexaenoate (V-16)

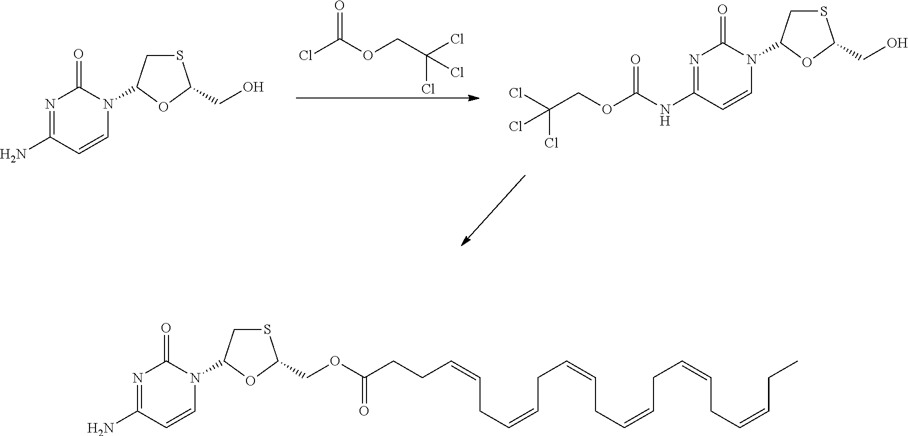

To a solution of lamivudine (6.0 g, 26.17 mmol) in DMF (50 mL) under nitrogen at room temperature was added DMAP (3.19 g, 26.17 mmol), pyridine (3.10 g, 39.26 mmol) and 2,2,2-trichloroethyl carbonochloridate (5.50 g, 26.17 mmol). The resulting reaction mixture was stirred at room temperature for 18 h and then concentrated under reduced pressure. The resulting residue was dissolved in EtOAc (250 mL) and washed with saturated aq. NH₄Cl (3×50 mL) and brine (3×50 mL), dried over anhydrous Na₂SO₄ and concentrated under reduced pressure. The resulting residue was purified by silica gel chromatography to afford 5.2 g of 2,2,2-trichloroethyl (1-((2R,5S)-2-(hydroxymethyl)-1,3-oxathiolan-5-yl)-2-oxo-1,2-dihydropyrimidin-4-yl)carbamate (49%).

A mixture containing 2,2,2-trichloroethyl (1-((2R,5S)-2-(hydroxymethyl)-1,3-oxathiolan-5-yl)-2-oxo-1,2-dihydropyrimidin-4-yl)carbamate (1 g, 2.47 mmol), DCC (1.0 g, 4.84 mmol), DMAP (0.30 g, 2.47 mmol), HOBT (0.35 g, 2.47 mmol), and DHA (0.80 g, 2.47 mmol) in 10 mL of CH₂Cl₂/DMF was stirred at room temperature for 18 h. The organic layer was washed with aq. HCl (5%, 30 mL) and brine (3×30 mL), dried over anhydrous Na₂SO₄ and concentrated under reduced pressure. The resulting residue was purified by silica gel chromatography to afford 1.2 g of the ester intermediate (64%).

This ester intermediate (1.2 g, 1.67 mmol) was taken up in THF (20 mL) and zinc (1.0 g, 16.7 mmol) was added under N₂ and stirred at room temperature for 2 h. The Zinc, in turn, was freshly washed in sequence twice each with 10% HCl, water, and THF), followed by 1M Na₂HPO₄ (8 mL). The solids were filtered and washed with THF, and the combined filtrates were concentrated under reduced pressure. The resulting residue was purified by silica gel chromatography to afford 370 mg of (4Z,7Z,10Z,13Z,16Z,19Z)-((2R,5S)-5-(4-amino-2-oxopyrimidin-1(2H)-yl)-1,3-oxathiolan-2-yl)methyl docosa-4,7,10,13,16,19-hexaenoate (40%).

MS calculated for C₃₀H₄ᵢN₃O₄S: 539.7. found: 540.2 [M+H]⁺.

¹H NMR (300 MHz, CDCl₃) δ 0.95-1.00 (t, J=5.6 Hz, 3H), 2.06-2.09 (m, 3H), 2.41-2.44 (m, 4H), 2.79-2.85 (m, 10H), 3.07-3.12 (dd, J=4.1 Hz, 1H), 3.52-3.57 (dd, J=4.3 Hz, 1H), 4.38-4.41 (m, 1H), 4.38-4.41 (m, 1H), 5.32-5.43 (m, 13H), 5.74-5.77 (d, J=5.4 Hz, 1H), 6.33-6.36 (t, J=3.5 Hz, 1H), 5.99-6.99 (d, J=5.7 Hz, 1H).

### Example 37

### Preparation of (S)-((2R,5S)-5-(4-amino-2-oxopyrimidin-1(2H)-yl)-1,3-oxathiolan-2-yl)methyl 2-((4Z,7Z,10Z,13Z,16Z,19Z)-docosa-4,7,10,13,16,19-hexaenamido)propanoate (V-17)

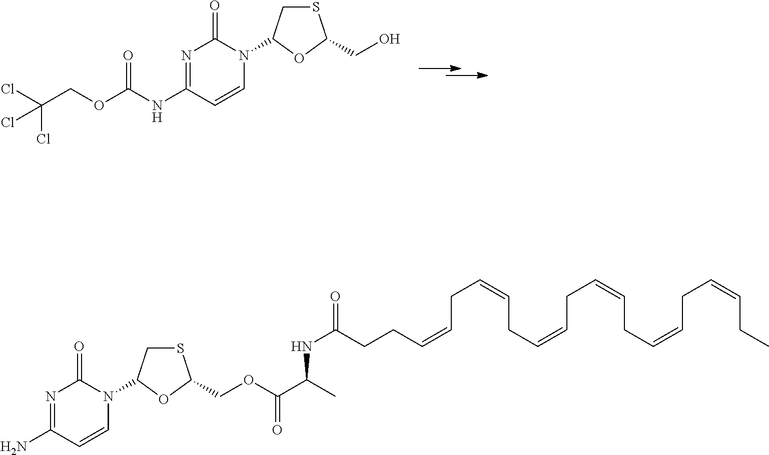

The same experimental procedure detailed in the preparation of (4Z,7Z,10Z,13Z,16Z,19Z)-((2R,5S)-5-(4-amino-2-oxopyrimidin-1(2H)-yl)-1,3-oxathiolan-2-yl)methyl docosa-4,7,10,13,16,19-hexaenoate (example 36) was used. (S)-2-((4Z,7Z,10Z,13Z,16Z,19Z)-Docosa-4,7,10,13,16,19-hexaenamido)propanoic acid was used as the appropriate starting material.

MS calculated for C₃₃H₄₆N₄O₅S: 610.8. found: 611.3 [M+H]⁺.

¹H NMR (400 MHz, CDCl₃) δ 0.95-1.00 (t, J=7.6 Hz, 3H), 1.42-1.45 (d, J=5.6 Hz, 1H), 2.05-2.09 (m, 2H), 2.28-2.30 (m, 2H), 2.39-2.43 (m, 2H), 2.79-2.85 (m, 10H), 3.09-3.10 (m, 1H), 3.55-3.599 (m, 1H), 4.38-4.41 (m, 1H), 4.38-4.41 (m, 1H), 5.32-5.43 (m, 13H), 5.74-5.77 (d, J=5.4 Hz, 1H), 6.33-6.36 (t, J=3.5 Hz, 1H), 5.99-6.99 (d, J=5.7 Hz, 1H).

### Example 38

### Preparation of ((2R,5S)-5-(4-amino-2-oxopyrimidin-1(2H)-yl)-1,3-oxathiolan-2-yl)methyl (2-((4Z,7Z,10Z,13Z,16Z,19Z)-docosa-4,7,10,13,16,19-hexaenamido)ethyl)carbamate (V-21)

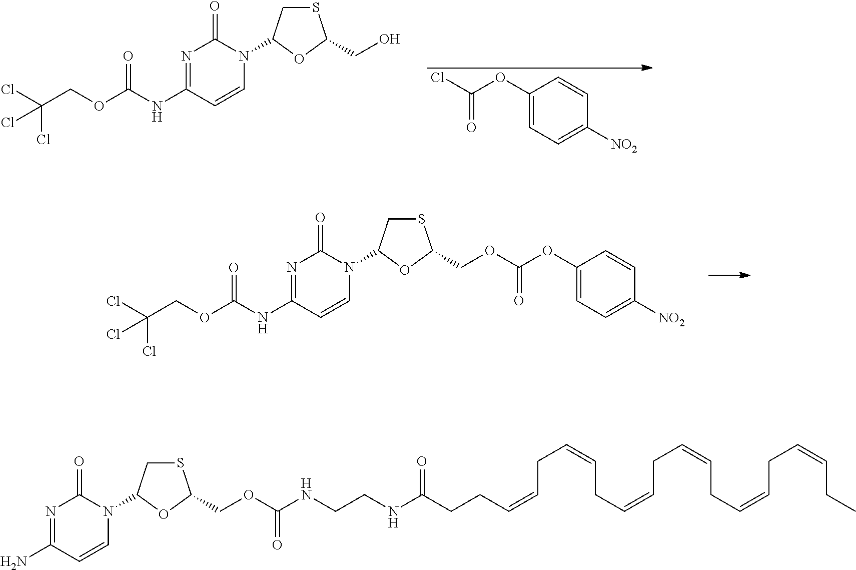

A mixture containing 2,2,2-trichloroethyl (1-((2R,5S)-2-(hydroxymethyl)-1,3-oxathiolan-5-yl)-2-oxo-1,2-dihydropyrimidin-4-yl)carbamate (2.0 g, 4.95 mmol), Et₃N (1.49 g, 4.85 mmol) and 4-nitrophenyl carbonochloridate (1.19 g, 5.91 mmol) in CH₂Cl₂ (20 mL) was stirred at room temperature for 18 h. The resulting reaction mixture was diluted with CH₂Cl₂ (60 mL), washed with saturated aq. NH₄Cl (3×20 mL) and brine (3×20 mL), dried over anhydrous Na₂SO₄ and concentrated under reduced pressure. The resulting residue was purified by silica gel chromatography to afford 1.0 g of 2,2,2-trichloroethyl (1-((2R,5S)-2-(4(4-nitrophenoxy)carbonyl)oxy)methyl)-1,3-oxathiolan-5-yl)-2-oxo-1,2-dihydropyrimidin-4-yl)carbamate (35%).

MS calculated for C₁₈H₁₄Cl₁₃N₄O₉S: 568.74. found: 569.1 [M+H]⁺.

A mixture containing 2,2,2-trichloroethyl (1-((2R,5S)-2-((((4-nitrophenoxy)carbonyl)oxy)methyl)-1,3-oxathiolan-5-yl)-2-oxo-1,2-dihydropyrimidin-4-yl)carbamate (0.5 g, 0.87 mmol), DIEA (0.33 g, 2.61 mmol), DMAP (0.16 g, 0.96 mmol) and (4Z,7Z,10Z,13Z,16Z,19Z)—N-(2-aminoethyl)docosa-4,7,10,13,16,19-hexaenamide (0.39 g, 1.05 mmol) in 20 mL of CH₂Cl₂ was stirred at room temperature for 18 h. The resulting reaction mixture was diluted with CH₂Cl₂ (60 mL) and the organic layer was washed with saturated aq. NH₄Cl (3×20 mL) and brine (3×20 mL), dried over anhydrous Na₂SO₄ and concentrated under reduced pressure. The resulting residue was purified by silica gel chromatography to afford 300 mg of 2,2,2-trichloroethyl (1-((2R,5S)-2-((((2-((4Z,7Z,10Z,13Z,16Z,19Z)-docosa-4,7,10,13,16,19-hexaenamido)ethyl)carbamoyl)oxy)methyl)-1,3-oxathiolan-5-yl)-2-oxo-1,2-dihydropyrimidin-4-yl)carbamate (43%).

MS calculated for C₃₆H₄₈Cl₃N₅O₇S: 801.21. found: 801.8 [M+H]⁺.

A mixture containing 2,2,2-trichloroethyl (1-((2R,5S)-2-((((2-((4Z,7Z,10Z,13Z,16Z,19Z)-docosa-4,7,10,13,16,19-hexaenamido)ethyl)carbamoyl)oxy)methyl)-1,3-oxathiolan-5-yl)-2-oxo-1,2-dihydropyrimidin-4-yl)carbamate (0.3 g, 0.37 mmol), Zn (0.24 g, 3.7 mmol), and Na₂HPO₄ (0.52 g, 3.7 mmol) in 10 mL of THF was stirred at room temperature for 3 hours. The reaction mixture was filtered and the filtrate was concentrated under reduced pressure. The resulting residue was purified by silica gel chromatography to afford 100 mg of ((2R,5S)-5-(4-amino-2-oxopyrimidin-1(2H)-yl)-1,3-oxathiolan-2-yl)methyl (2-((4Z,7Z,10Z,13Z,16Z,19Z)-docosa-4,7,10,13,16,19-hexaenamido)ethyl)carbamate (43%).

MS calculated for C₃₃H₄₇N₅O₅S: 625.8. found: 626.1 [M+H]⁺.

¹H NMR (400 MHz, CDCl₃) δ 0.95-1.00 (t, J=7.8 Hz, 3H), 2.03-2.10 (m, 2H), 2.23-2.27 (m, 2H), 2.37-2.41 (m, 2H), 2.83-2.85 (m, 1H), 3.36-3.52 (m, 5H), 4.43 (s, 2H), 5.33-5.39 (m, 13H), 5.91-5.93 (m, 1H), 6.32-6.35 (m, 2H), 6.53 (s, 1H), 7.75-7.77 (m, 1H).

### Example 39

### Preparation of ((2R,5S)-5-(4-amino-2-oxopyrimidin-1(2H)-yl)-1,3-oxathiolan-2-yl)methyl (2-((5Z,8Z,11Z,14Z,17Z)-icosa-5,8,11,14,17-pentaenamido)ethyl)carbamate (V-22)

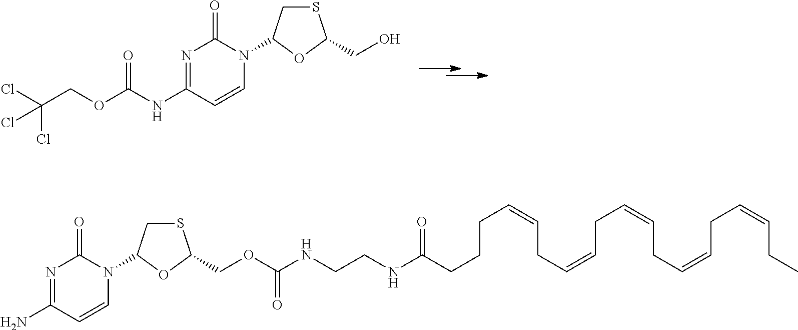

The same experimental procedure detailed in the preparation of ((2R,5S)-5-(4-amino-2-oxopyrimidin-1(2H)-yl)-1,3-oxathiolan-2-yl)methyl (2-((4Z,7Z,10Z,13Z,16Z,19Z)-docosa-4,7,10,13,16,19-hexaenamido)ethyl)carbamate was used. (5Z,8Z,11Z,14Z,17Z)—N-(2-Aminoethyl)icosa-5,8,11,14,17-pentaenamide was used as the appropriate starting material.

MS calculated for C₃₃H₄₇N₅O₅S: 625.8. found: 626.1 [M+H]⁺.

¹H NMR (400 MHz, CDCl₃) δ 0.90-1.01 (t, J=8.5 Hz, 3H), 1.67-1.75 (m, 2H), 1.88-2.22 (m, 6H), 2.80-2.84 (m, 8H), 3.10-3.16 (m, 1H), 3.36-3.53 (m, 5H), 4.44 (s, 1H), 5.32-5.42 (m, 11H), 5.90-5.92 (m, 1H), 6.32-6.38 (m, 3H), 7.76-7.79 (m, 1H).

### Example 40

### Preparation of (4Z,7Z,10Z,13Z,16Z,19Z)—N-(1-((2R,5S)-2-(hydroxymethyl)-1,3-oxathiolan-5-yl)-2-oxo-1,2-dihydropyrimidin-4-yl)docosa-4,7,10,13,16,19-hexaenamide (V-18)

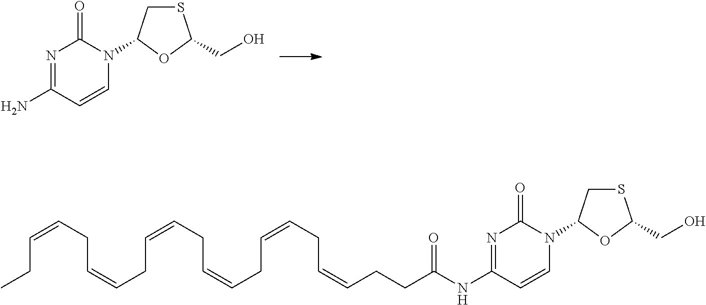 [SMILES: Nc1ccn([C@@H]2CS[C@H](CO)O2)c(=O)n1 ; CC/C=C\C/C=C\C/C=C\C/C=C\C/C=C\C/C=C\CCC(=O)Nc1ccn([C@@H]2CS[C@H](CO)O2)c(=O)n1]

Lamivudine (0.8 g, 3.49 mmol), DCC (1.0 g, 6.88 mmol), DMAP (0.30 g, 3.49 mmol), HOBT (0.35 g, 3.49 mmol), and DHA (0.80 g, 3.49 mmol) were suspended in 10 mL of 1:1 CH₂Cl₂/DMF and stirred at room temperature for 18 h. The organic layer was washed with aq. HCl (5%, 30 mL) and brine (3×30 mL), dried over anhydrous Na₂SO₄ and concentrated under reduced pressure. The resulting residue was purified by silica gel chromatography to afford 405 mg of (4Z,7Z,10Z,13Z,16Z,19Z)—N-(1-((2R,5S)-2-(hydroxymethyl)-1,3-oxathiolan-5-yl)-2-oxo-1,2-dihydropyrimidin-4-yl)docosa-4,7,10,13,16,19-hexaenamide (24%).

MS calculated for C₃₀H₄₁N₃O₄S: 539.7. found: 540.2 [M+H]⁺.

¹H NMR (400 MHz, CDCl₃) δ 0.95-1.00 (t, J=7.2 Hz, 3H), 2.05-2.09 (m, 2H), 2.42-2.50 (m, 4H), 2.79-2.85 (m, 10H), 3.20-3.25 (dd, J=5.3 Hz, 1H), 3.61-3.66 (dd, J=6.0 Hz, 1H), 3.94-3.99 (dd, J=5.6 Hz, 1H), 4.13-4.17 (dd, J=5.1 Hz, 1H), 5.32-5.43 (m, 13H), 6.33-6.35 (m, 1H), 7.42-7.45 (d, J=7.2 Hz, 1H).

### Example 41

### Preparation of (4Z,7Z,10Z,13Z,16Z,19Z)—N—((S)-1-((1-(2R,5S)-2-(hydroxymethyl)-1,3-oxathiolan-5-yl)-2-oxo-1,2-dihydropyrimidin-4-yl)amino)-1-oxopropan-2-yl)docosa-4,7,10,13,16,19-hexaenamide (V-20)

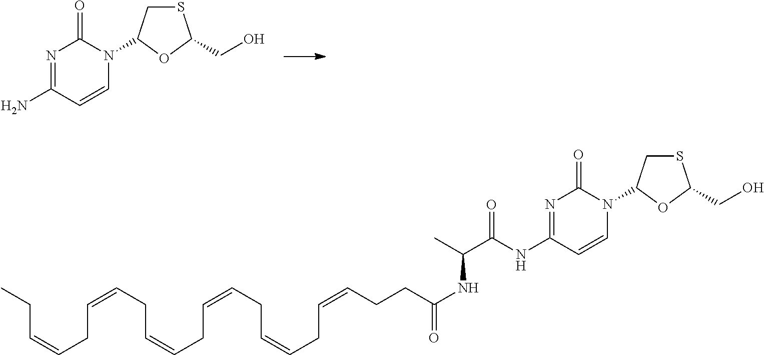

The same experimental procedure detailed in the preparation of (4Z,7Z,10Z,13Z,16Z,19Z)—N-(1-((2R,5S)-2-(hydroxymethyl)-1,3-oxathiolan-5-yl)-2-oxo-1,2-dihydropyrimidin-4-yl)docosa-4,7,10,13,16,19-hexaenamide (example 40) was used. (S)-2-((4Z,7Z,10Z,13Z,16Z,19Z)-Docosa-4,7,10,13,16,19-hexaenamido)propanoic acid was used as the appropriate starting material.

MS calculated for C₃₃H₄₆N₄O₅S: 610.8. found: 611.3 [M+H]⁺.

¹H NMR (400 MHz, CDCl₃) δ 0.95-0.99 (t, J=7.4 Hz, 3H), 1.44-1.45 (m, 4H), 2.03-2.11 (m, 2H), 2.29-2.33 (m, 2H), 2.41-2.46 (m, 2H), 2.79-2.85 (m, 10H), 3.24-3.28 (m, 1H), 3.62-3.67 (m, 1H), 3.93-3.98 (m, 1H), 4.14-4.18 (m, 1H), 5.29-5.43 (m, 13H), 6.32-6.34 (m, 2H), 7.39 (m, 1H), 8.39-8.41 (m, 1H).

### Example 42

### Preparation of 2-(2-(2-((4Z,7Z,10Z,13Z,16Z,19Z)-docosa-4,7,10,13,16,19-hexaenamido)-9H-purin-9-yl)ethyl)propane-1,3-diyl diacetate (V-10)

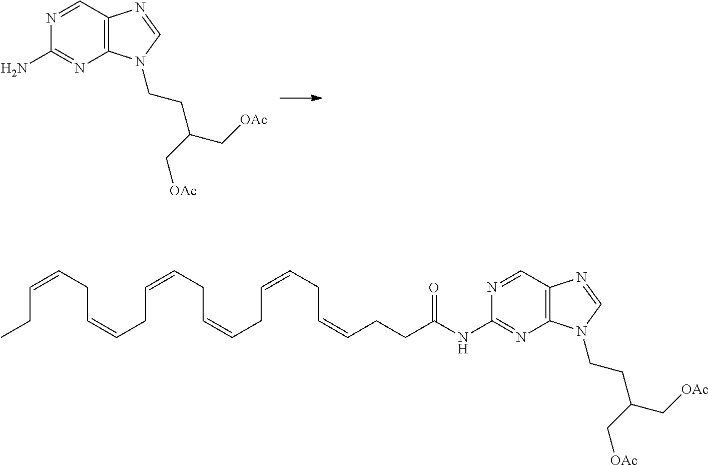 [SMILES: CC(=O)OCC(CCn1cnc2cnc(N)nc21)COC(C)=O ; CC/C=C\C/C=C\C/C=C\C/C=C\C/C=C\C/C=C\CCC(=O)Nc1ncc2ncn(CCC(COC(C)=O)COC(C)=O)c2n1]

The same experimental procedure detailed in the preparation of (4Z,7Z,10Z,13Z,16Z,19Z)—N-(1-((2R,5S)-2-(hydroxymethyl)-1,3-oxathiolan-5-yl)-2-oxo-1,2-dihydropyrimidin-4-yl)docosa-4,7,10,13,16,19-hexaenamide (example 40) was used. Famciclovir was used as the appropriate starting material. Purification by silica gel chromatography (95% CH₂Cl₂, 5% MeOH) afforded 2-(2-(2-((4Z,7Z,10Z,13Z,16Z,19Z)-docosa-4,7,10,13,16,19-hexaenamido)-9H-purin-9-yl)ethyl)propane-1,3-diyl diacetate. MS calculated for C₃₆H₄₉N₅O₅: 631.37. found: 632 [M+H]⁺.

### Example 43

### Preparation of (4Z,7Z,10Z,13Z,16Z,19Z)—N-(9-(4-hydroxy-3-(hydroxymethyl)butyl)-9H-purin-2-yl)docosa-4,7,10,13,16,19-hexaenamide (V-11)

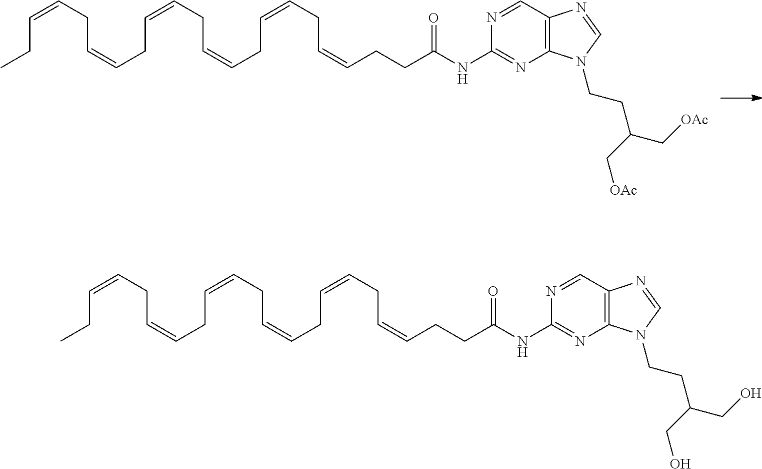 [SMILES: CC/C=C\C/C=C\C/C=C\C/C=C\C/C=C\C/C=C\CCC(=O)Nc1ncc2ncn(CCC(COC(C)=O)COC(C)=O)c2n1 ; CC/C=C\C/C=C\C/C=C\C/C=C\C/C=C\C/C=C\CCC(=O)Nc1ncc2ncn(CCC(CO)CO)c2n1]

2-(2-(2-((4Z,7Z,10Z,13Z,16Z,19Z)-Docosa-4,7,10,13,16,19-hexaenamido)-9H-purin-9-yl)ethyl)propane-1,3-diyl diacetate (500 mg, 0.79 mmol) was taken up in 5 mL of 1:1 THF/H₂O containing K₂CO₃ (0.4 mmol). The resulting reaction mixture was stirred at room temperature for 3 h. It was then extracted with EtOAc. The combined organic layers were washed with brine, dried (Na₂SO₄) and concentrated under reduced pressure. Purification by silica gel chromatography (95% CH₂Cl₂, 5% MeOH) afforded 120 mg of (4Z,7Z,10Z,13Z,16Z,19Z)—N-(9-(4-hydroxy-3-(hydroxymethyl)butyl)-9H-purin-2-yl)docosa-4,7,10,13,16,19-hexaenamide. MS calculated for C₃₂H₄₅N₅O₃: 547.35. found: 548 [M+H]⁺.

### Example 44

### Preparation of 4-(2-β1-cyclopropyl-2-oxo-1H-imidazo[4,5-c]pyridin-3(2H)-yl)methyl)-1H-benzo[d]imidazol-1-yl)butyl (2-((4Z,7Z,10Z,13Z,16Z,19Z)-docosa-4,7,10,13,16,19-hexaenamido)ethyl)carbamate (VI-1)

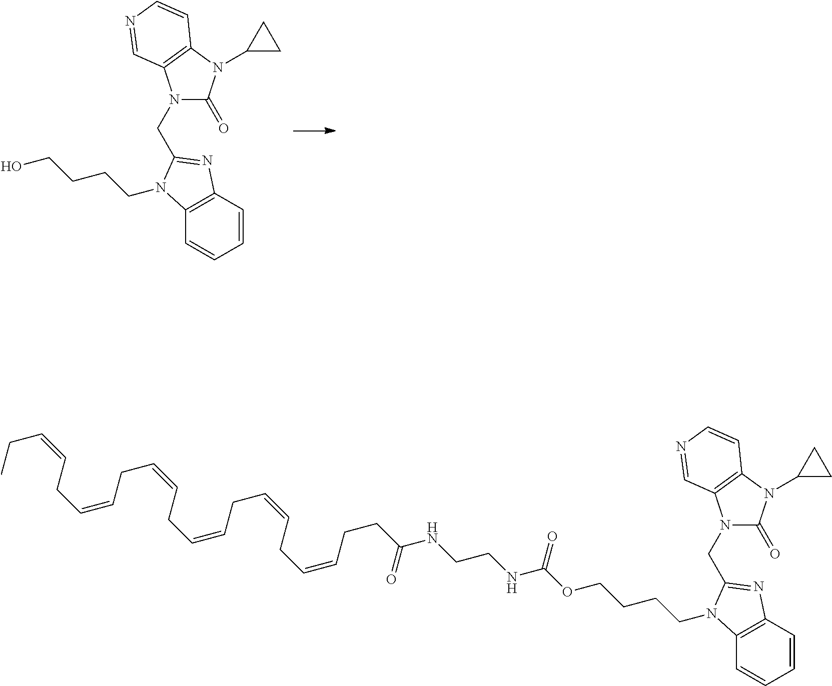 [SMILES: O=c1n(Cc2nc3ccccc3n2CCCCO)c2cnccc2n1C1CC1 ; CC/C=C\C/C=C\C/C=C\C/C=C\C/C=C\C/C=C\CCC(=O)NCCNC(=O)OCCCCn1c(Cn2c(=O)n(C3CC3)c3ccncc32)nc2ccccc21]

The same experimental procedure detailed in the preparation of ((2S,5R)-5-(6-oxo-3H-purin-9(6H)-yl)tetrahydrofuran-2-yl)methyl (2-((4Z,7Z,10Z,13Z,16Z,19Z)-docosa-4,7,10,13,16,19-hexaenamido)ethyl)carbamate (example 29) was used. 1-Cyclopropyl-3-((1-(4-hydroxybutyl)-1H-benzo[d]imidazol-2-yl)methyl)-1H-imidazo[4,5-c]pyridin-2(3H)-one was used as the appropriate starting material. This compound, in turn, could be prepared using the procedures outlined in Provencal et al Org. Process Research & Development 2004, p. 903-908.

MS calculated for C₄₆H₅₉N₇O₄: 773.46. found: 774 [M+H]⁺.

### Example 45

### Preparation of (3R,3aS,6aR)-hexahydrofuro[2,3-b]furan-3-yl((2S,3R)-4-(4-((4Z,7Z,10Z,13Z,16Z,19Z)-docosa-4,7,10,13,16,19-hexaenamido)-N-isobutylphenylsulfonamido)-3-hydroxy-1-phenylbutan-2-yl)carbamate (VII-1)

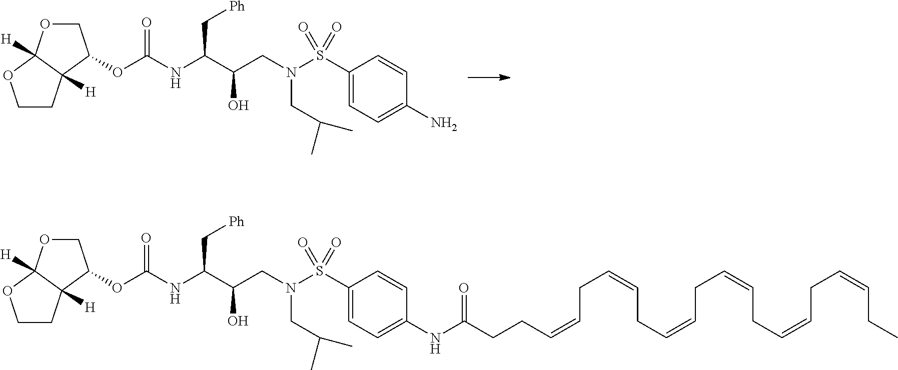 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1 ; CC/C=C\C/C=C\C/C=C\C/C=C\C/C=C\C/C=C\CCC(=O)Nc1ccc(S(=O)(=O)N(CC(C)C)C[C@@H](O)[C@H](Cc2ccccc2)NC(=O)O[C@H]2CO[C@H]3OCC[C@H]32)cc1]

A solution containing (4Z,7Z,10Z,13Z,16Z,19Z)-docosa-4,7,10,13,16,19-hexaenoic acid (0.3 g, 0.91 mmol) in CH₂Cl₂ (20 mL) was cooled to 0° C. Oxalyl chloride (0.3 mL) was then added with cooling in an ice-bath, followed by a few drops of DMF. The resulting reaction mixture was stirred at room temperature for 2 h and then concentrated under reduced pressure to afford 0.31 g of (4Z,7Z,10Z,13Z,16Z,19Z)-docosa-4,7,10,13,16,19-hexaenoyl chloride (100%), which was used in next step without further purification.

A mixture containing darunavir (0.5 g, 0.91 mmol), DIEA (0.17 g, 1.36 mmol) and (4Z,7Z,10Z,13Z,16Z,19Z)-docosa-4,7,10,13,16,19-hexaenoyl chloride (0.31 g, 0.91 mmol) in CH₂Cl₂ (20 mL) was stirred at room temperature for 2 h. The resulting reaction mixture was diluted with CH₂Cl₂ (60 mL) and washed with saturated aq. NH₄Cl (3×20 mL) and brine (3×20 mL). The organic layer was dried over anhydrous Na₂SO₄ and concentrated under reduced pressure. The resulting residue was purified by silica gel chromatography (EtOAc/pentanes) to afford 0.4 g of (3R,3aS,6aR)-hexahydrofuro[2,3-b]furan-3-yl((2S,3R)-4-(4-((4Z,7Z,10Z,13Z,16Z,19Z)-docosa-4,7,10,13,16,19-hexaenamido)-N-isobutylphenylsulfonamido)-3-hydroxy-1-phenylbutan-2-yl)carbamate (51%).

MS calculated for C₄₉H₆₇N₃O₈S: 858.1. Found: 859.2 [M+H]⁺.

¹H NMR (400 MHz, CDCl₃) δ 0.80-0.91 (m, 9H), 1.52-1.60 (m, 1H), 1.74-1.78 (m, 2H), 2.38-2.45 (m, 4H), 2.70-3.10 (m, 17H), 3.51 (s, 1H), 3.59-3.65 (m, 2H), 3.76-3.90 (m, 4H), 4.83-4.86 (s, 1H), 4.91-4.95 (s, 1H), 5.23-5.43 (m, 12H), 5.57-5.58 (m, 1H), 7.13-7.23 (m, 5H), 7.59-7.66 (m, 4H).

### Example 46

### Preparation of (13Z,16Z,19Z,22Z,25Z)-methyl 4-(((2R,5S)-5-(4-amino-2-oxopyrimidin-1(2H)-yl)-1,3-oxathiolan-2-yl)methoxy)-4,9-dioxo-3,5,8-triaza-4-phosphaoctacosa-13,16,19,22,25-pentaen-1-oate (II-71)

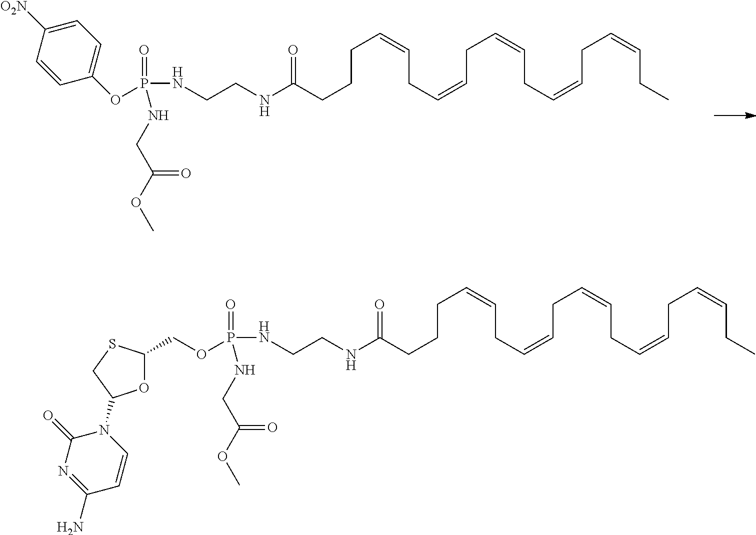

The same experimental procedure detailed in the preparation of ((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (example 9) was used. Glycine methyl ester was used as the appropriate starting material to prepare the intermediate (13Z,16Z,19Z,22Z,25Z)-methyl 4-(4-nitrophenoxy)-4,9-dioxo-3,5,8-triaza-4-phosphaoctacosa-13,16,19,22,25-pentaen-1-oate. The final product was purified by silica gel chromatography. MS calculated for C₃₃H₅₁N₆O₇PS: 706.33. Found: 707 [M+H]⁺.

### Example 47

### Preparation of ((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-1)

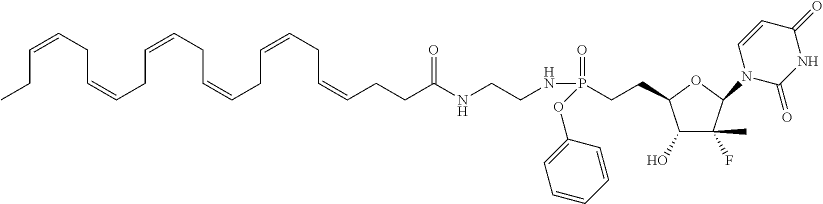 [SMILES: CC/C=C\C/C=C\C/C=C\C/C=C\C/C=C\C/C=C\CCC(=O)NCCNP(=O)(CC[C@H]1O[C@@H](n2ccc(=O)[nH]c2=O)[C@](C)(F)[C@@H]1O)Oc1ccccc1]

((2R,3R,4R,5R)-5-(2,4-Dioxo-3,4-dihydropyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate was prepared using the same procedures detailed in example 9. MS calculated for C₄₀H₅₄FN₄O₈P: 768.37. found: 769 [M+H]⁺.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising an antiviral agent and a fatty acid directly or indirectly covalently linked, selected from omega-3 fatty acids, fatty acids metabolized in vivo into omega-3 fatty acids, or lipoic acid, with the proviso that the molecular conjugate is not 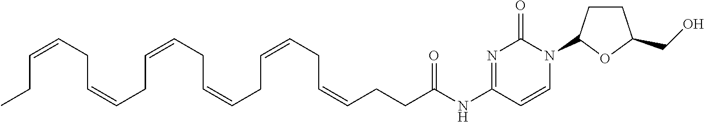
(4Z,7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-4,7,10,13,16,19-hexaenamide, 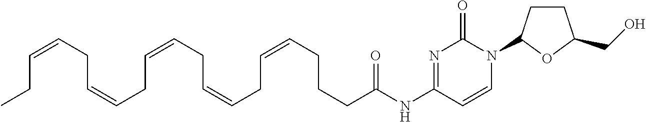
(5Z,8Z,11Z,14Z,17Z)—N-(1-((2S,5S)-5-(hydroxymethyl)tetrahydrofuran-2-yl)-2-oxo-1,2-dihydropyrimidin-4-yl)icosa-5,8,11,14,17-pentaenamide, 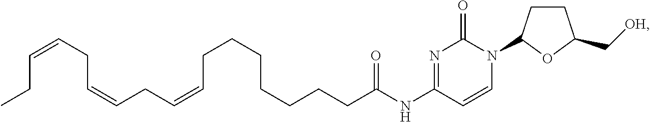
(9Z,12Z,15Z)—N-(1-((2S,5S)-5-(hydroxymethyl)tetrahydrofuran-2-yl)-2-oxo-1,2-dihydropyrimidin-4-yl)octadeca-9,12,15-trienamide, 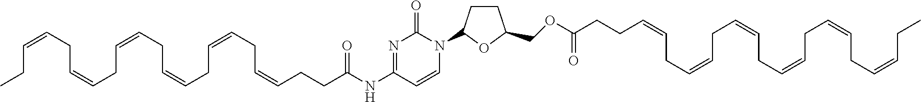
(4Z,7Z,10Z,13Z,16Z,19Z)-((2S,5S)-5-(4-((4Z,7Z,10Z,13Z,16Z,19Z)-docosa-4,7,10,13,16,19-hexaenamido)-2-oxopyrimidin-1(2H)-yl)tetrahydrofuran-2-yl)methyl docosa-4,7,10,13,16,19-hexaenoate, 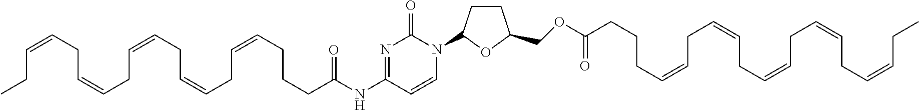
(5Z,8Z,11Z,14Z,17Z)-((2S,5S)-5-(4-((5Z,8Z,11Z,14Z,17Z)-icosa-5,8,11,14,17-pentaenamido)-2-oxopyrimidin-1(2H)-yl)tetrahydrofuran-2-yl)methyl icosa-5,8,11,14,17-pentaenoate, 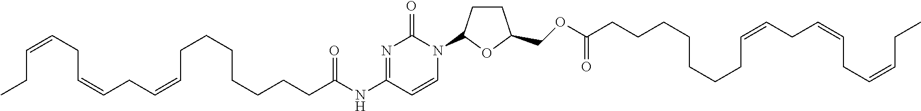
(9Z,12Z,15Z)-((2S,5S)-5-(4-((9Z,12Z,15Z)-octadeca-9,12,15-trienamido)-2-oxopyrimidin-1(2H)-yl)tetrahydrofuran-2-yl)methyl octadeca-9,12,15-trienoate, 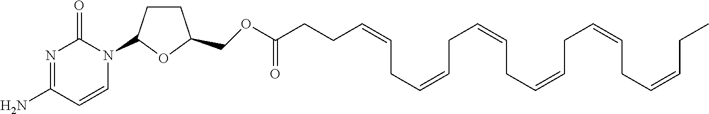
(4Z,7Z,10Z,13Z,16Z,19Z)-((2S,5S)-5-(4-amino-2-oxopyrimidin-1(2H)-yl)tetrahydrofuran-2-yl)methyl docosa-4,7,10,13,16,19-hexaenoate, 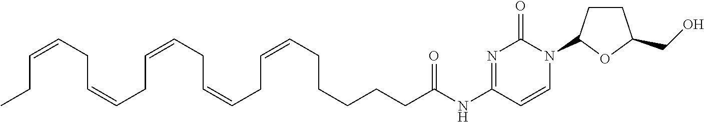
(7Z,10Z,13Z,16Z,19Z)—N-(1-((2S,5S)-5-(hydroxymethyl)tetrahydrofuran-2-yl)-2-oxo-1,2-dihydropyrimidin-4-yl)docosa-7,10,13,16,19-pentaenamide, 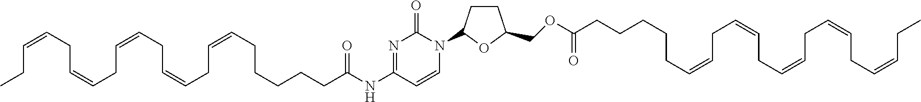
(7Z,10Z,13Z,16Z,19Z)-((2S,5S)-5-(4-((7Z,10Z,13Z,16Z,19Z)-docosa-7,10,13,16,19-pentaenamido)-2-oxopyrimidin-1(2H)-yl)tetrahydrofuran-2-yl)methyl docosa-7,10,13,16,19-pentaenoate, 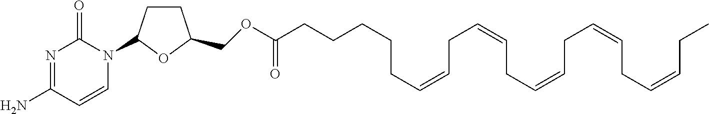
(7Z,10Z,13Z,16Z,19Z)-((2S,5S)-5-(4-amino-2-oxopyrimidin-1(2H)-yl)tetrahydrofuran-2-yl)methyl docosa-7,10,13,16,19-pentaenoate, 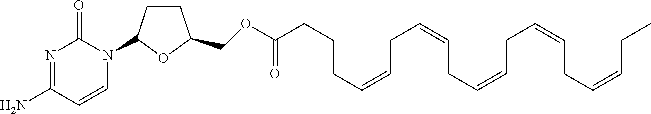
(5Z,8Z,11Z,14Z,17Z)-((2S,5S)-5-(4-amino-2-oxopyrimidin-1(2H)-yl)tetrahydrofuran-2-yl)methyl icosa-5,8,11,14,17-pentaenoate, 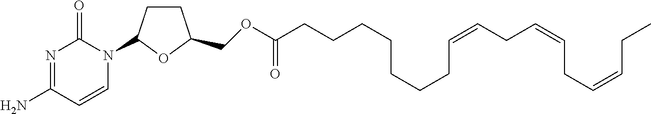
(9Z,12Z,15Z)-((2S,5S)-5-(4-amino-2-oxopyrimidin-1(2H)-yl)tetrahydrofuran-2-yl)methyl octadeca-9,12,15-trienoate, or 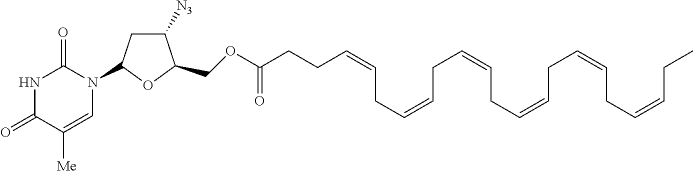
(4Z,7Z,10Z,13Z,16Z,19Z)-((2S,3S,5S)-3-azido-5-(5-methyl-2,4-dioxo-3,4-dihydropyrimidin-1 (2H)-yl)tetrahydrofuran-2-yl)methyl docosa-4,7,10,13,16,19-hexaenoate;

2. A compound of the Formula I: 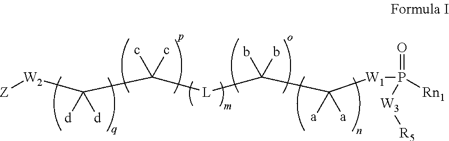
or a pharmaceutically acceptable salt, hydrates, solvate, prodrug, enantiomer, or a stereoisomer thereof;
wherein
Rₙ₁ is a nucleoside antiviral agent;
W₁ and W₂ are each independently null, O, S, NH, NR, or W₁ and W₂ can be taken together can form an imidazolidine or piperazine group, with the proviso that W₁ and W₂ can not be O simultaneously;
W₃ is each independently O or NR.
each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;
each n, o, p, and q is independently 0, 1 or 2;
each L is independently null, —O—, —S—, —S(O)—, —S(O)₂—, —S—S—, —(C₁-C₆alkyl)-, —(C₃-C₆cycloalkyl)-, a heterocycle, a heteroaryl, 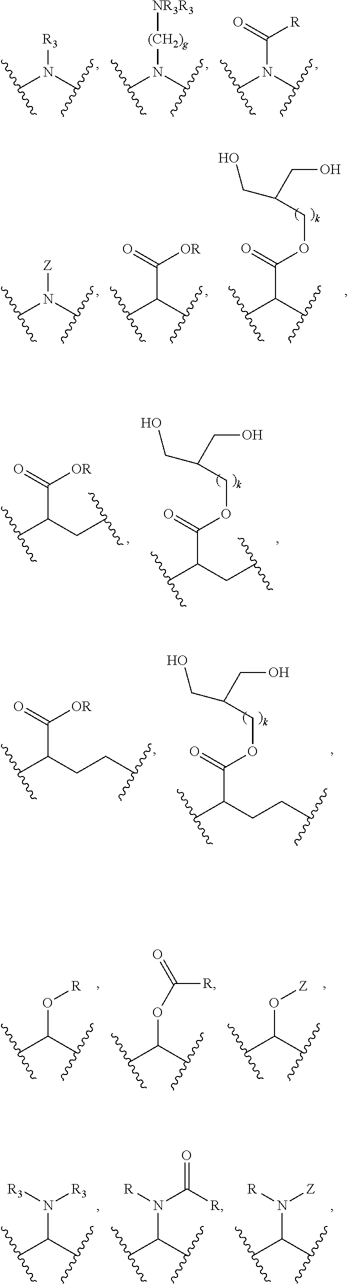 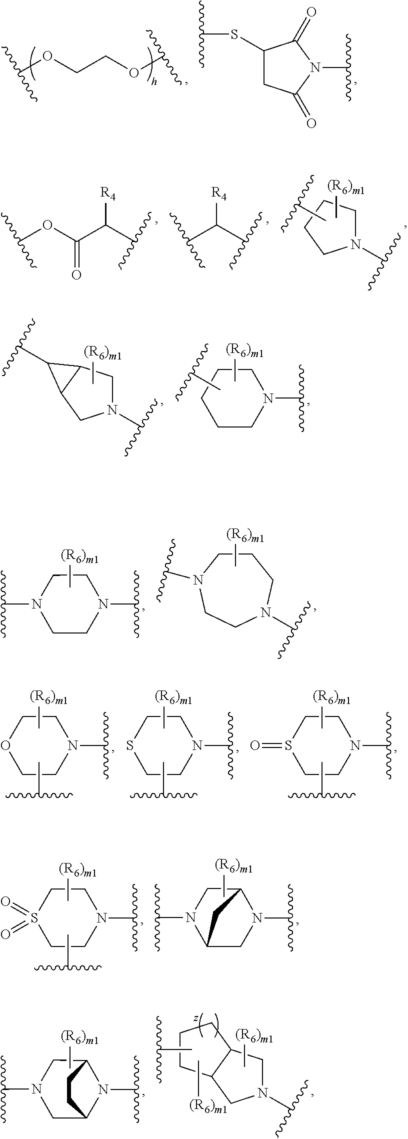 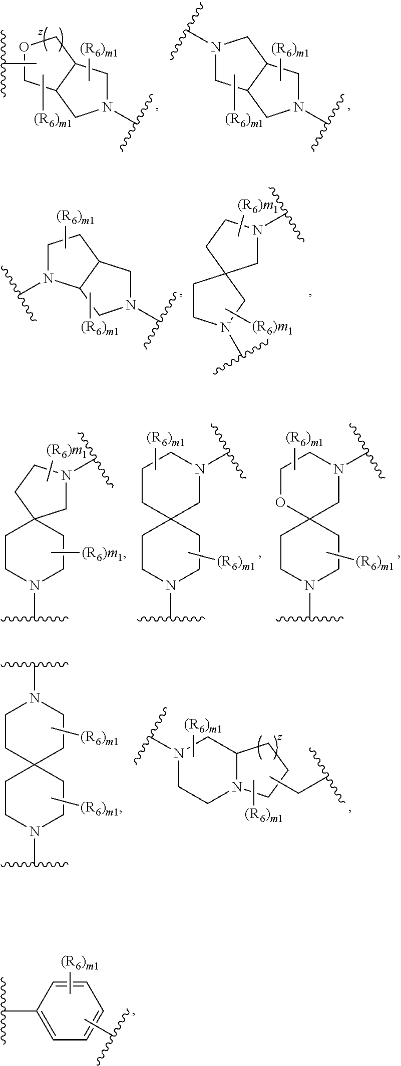
wherein the representation of L is not limited directionally left to right as is depicted, rather either the left side or the right side of L can be bound to the W₁ side of the compound of Formula I;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each R₅ is independently H, aryl, heteroaryl, heterocyclic, straight or branched C₁-C₁₀ alkyl which can be optionally substituted with one or two groups selected from halogen, e, OH, NH₂, CO₂R, CONH₂, CONR₂, phenyl, C₆H₄OH, imidazole or arginine;
each Z is independently —H, 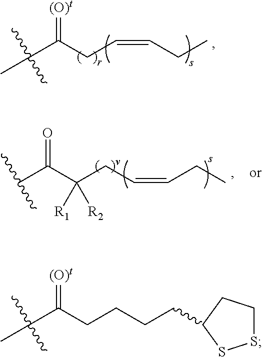
with the proviso that there is at least one 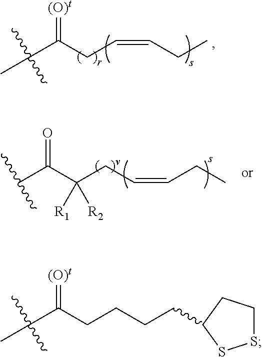
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 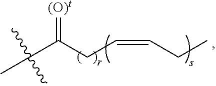
then t must be 0; and
when m, n, o, p, and q are each 0, and W₁ and W₂ are each null, then Z must not be

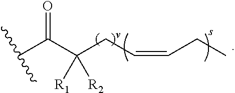

3. A compound of the Formula II: 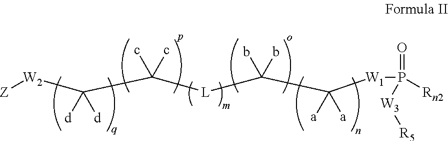
or a pharmaceutically acceptable salt, hydrate, solvate, prodrug, enantiomer, or a stereoisomer thereof;
wherein Rₙ₂ is independently 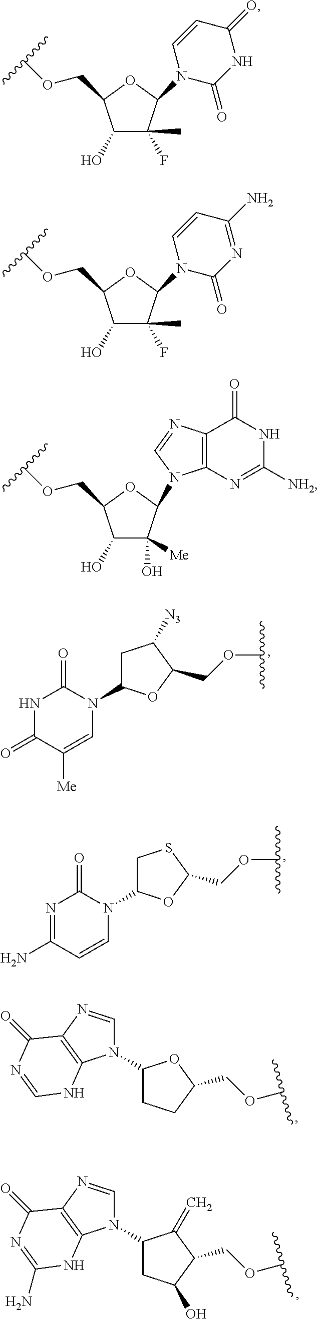 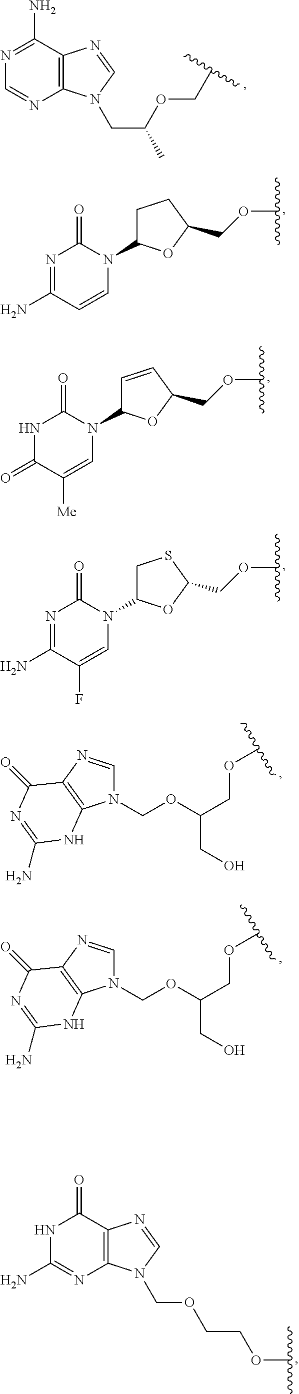 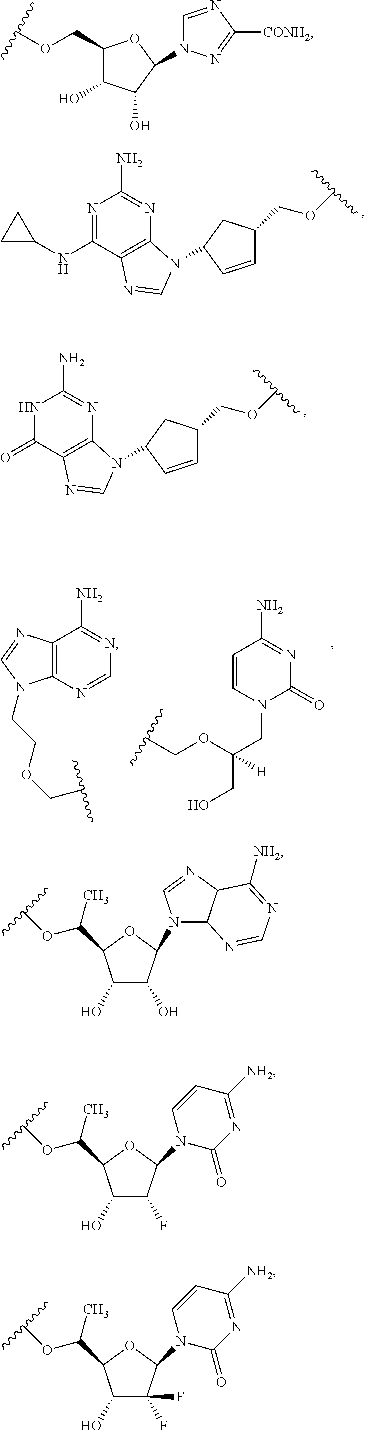 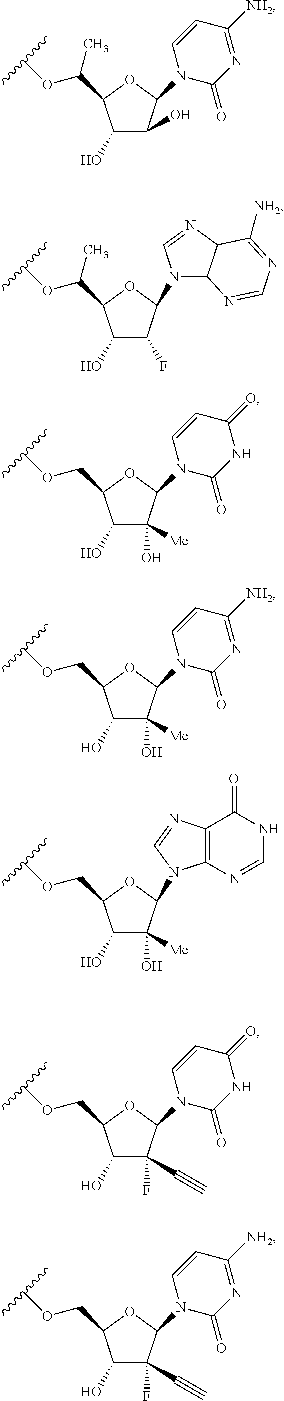 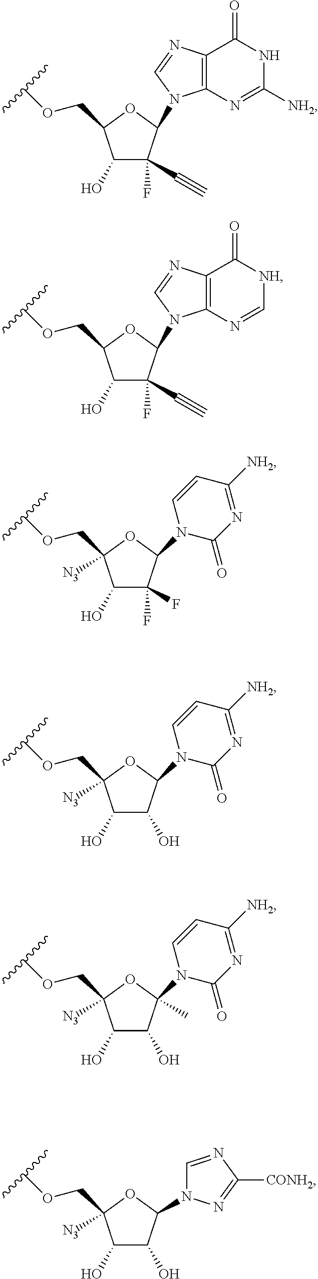 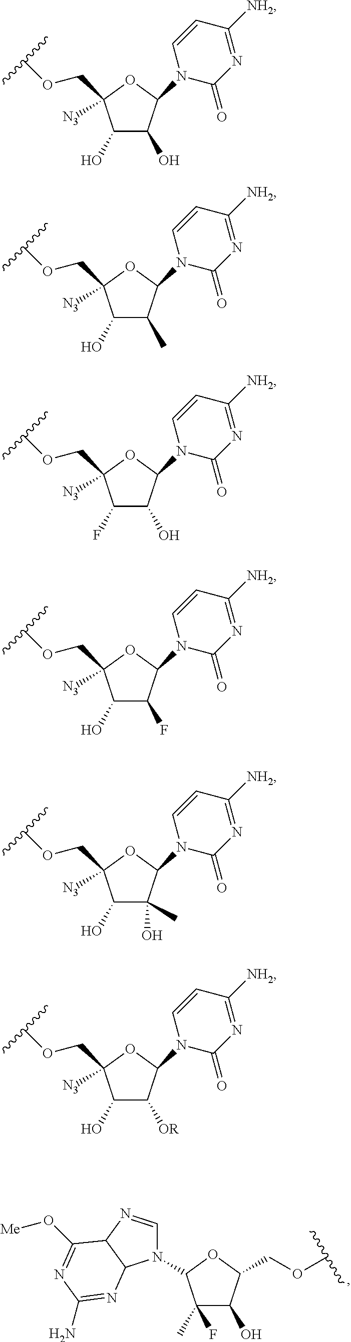 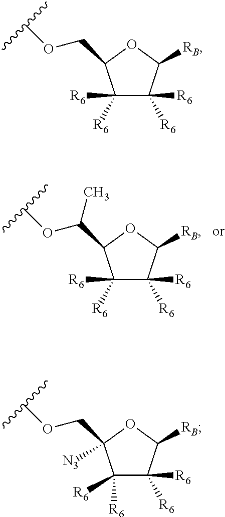
W₁ and W₂ are each independently null, O, S, NH, NR, or W₁ and W₂ can be taken together can form an imidazolidine or piperazine group, with the proviso that W₁ and W₂ can not be O simultaneously;
W₃ is each independently O or NR.
each a, b, c and d is independently —H, -D, —CH₃, —OCH₃, —OCH₂CH₃, —C(O)OR, or —O—Z, or benzyl, or two of a, b, c, and d can be taken together, along with the single carbon to which they are bound, to form a cycloalkyl or heterocycle;
each n, o, p, and q is independently 0, 1 or 2;
each L is independently null, —O—, —S—, —S(O)—, —S(O)₂—, —S—S—, —(C₁-C₆alkyl)-, —(C₃-C₆cycloalkyl)-, a heterocycle, a heteroaryl, 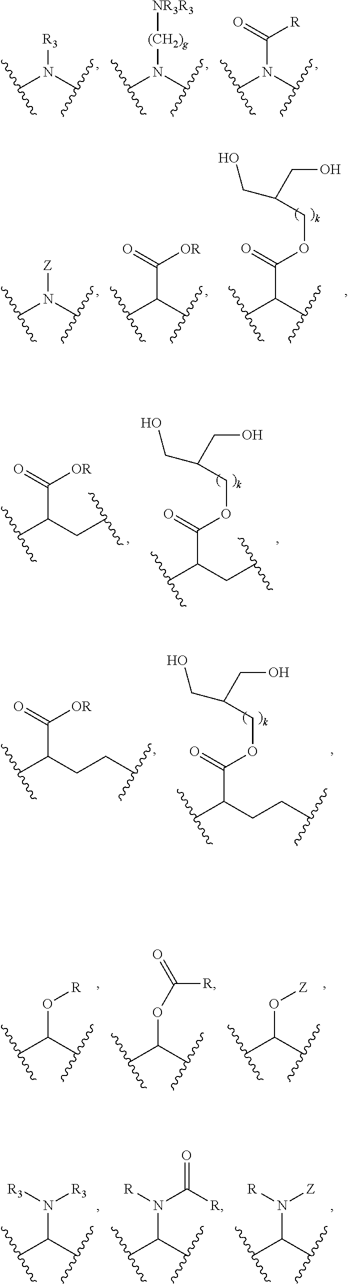 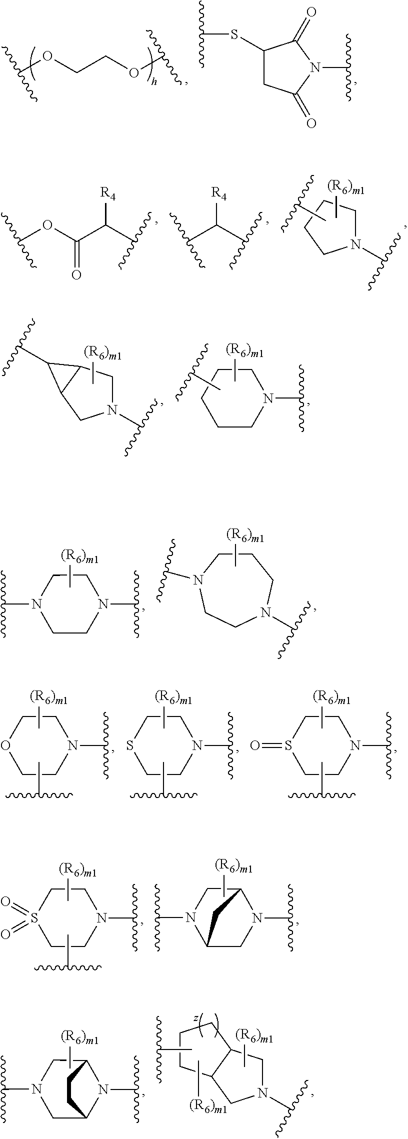 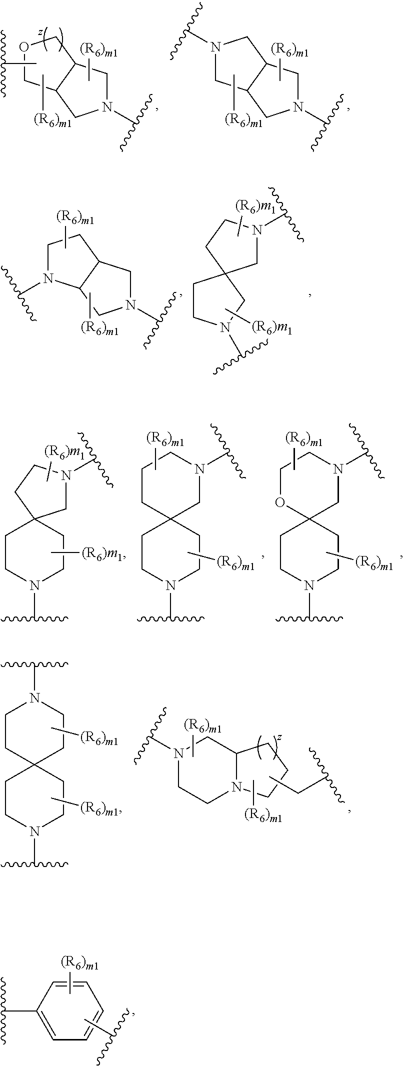
wherein the representation of L is not limited directionally left to right as is depicted, rather either the left side or the right side of L can be bound to the W₁ side of the compound of Formula II;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
R_{B} is independently 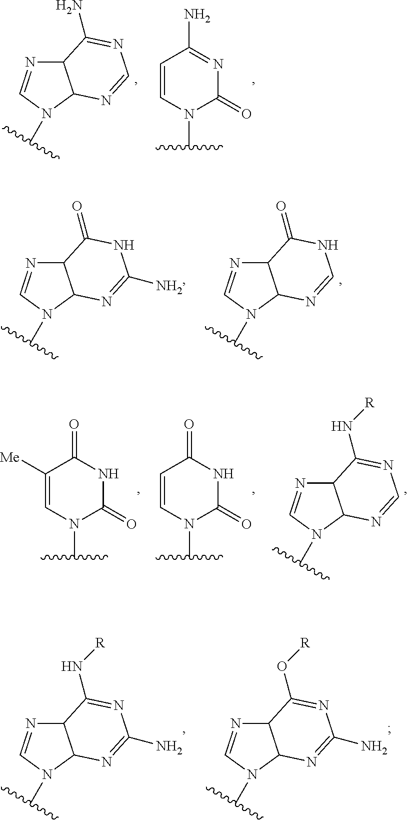
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each R₅ is independently H, aryl, heteroaryl, heterocyclic, straight or branched C₁-C₁₀ alkyl which can be optionally substituted with one or two groups selected from halogen, e, OH, NH₂, CO₂R, CONH₂, CONR₂, phenyl, C₆H₄OH, imidazole or arginine;
each Z is independently —H, 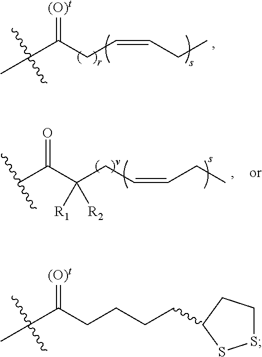
with the proviso that there is at least one 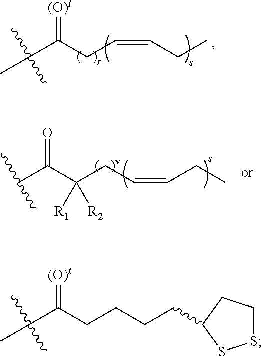
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 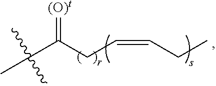
then t must be 0; and
when m, n, o, p, and q are each 0, and W₁ and W₂ are each null, then Z must not be

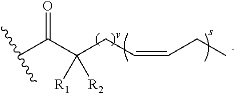

4-9. (canceled)

10. The compound of claim 3, wherein Rₙ₂ is selected from the group consisting

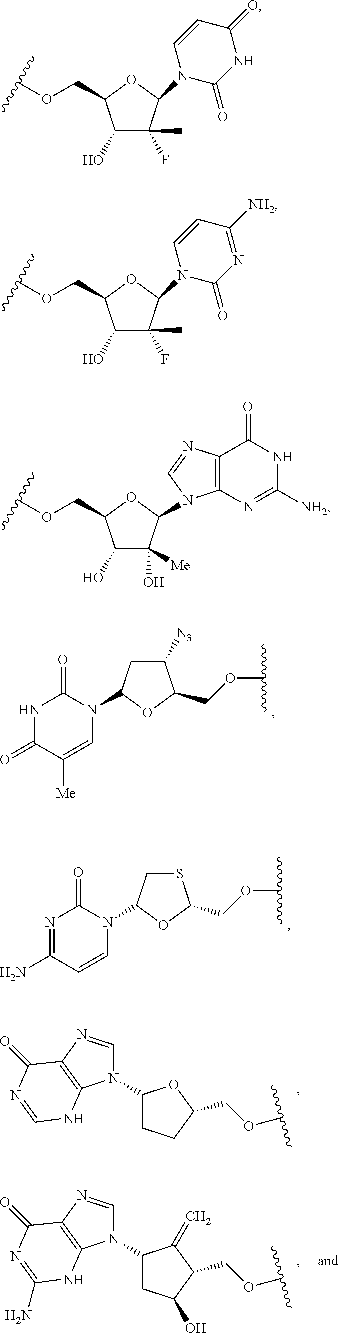

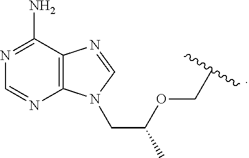 [SMILES: *CO[C@H](C)Cn1cnc2c(N)ncnc21]

11-13. (canceled)

14. The compound of claim 10 wherein the compound is selected from the group consisting of 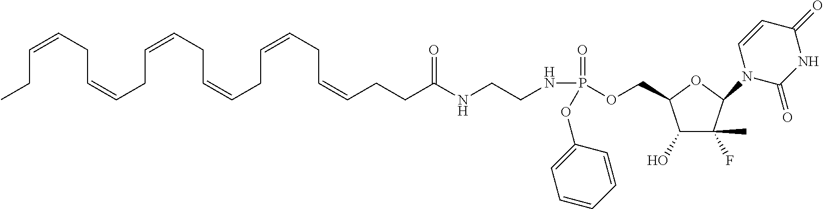 [SMILES: CC/C=C\C/C=C\C/C=C\C/C=C\C/C=C\C/C=C\CCC(=O)NCCNP(=O)(OC[C@H]1O[C@@H](n2ccc(=O)[nH]c2=O)[C@](C)(F)[C@@H]1O)Oc1ccccc1]
((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-1) 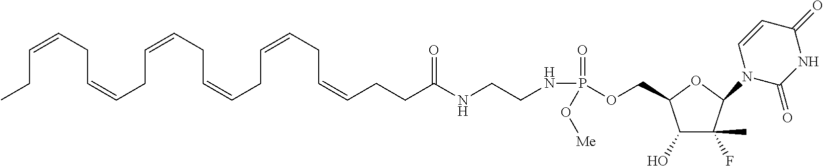 [SMILES: CC/C=C\C/C=C\C/C=C\C/C=C\C/C=C\C/C=C\CCC(=O)NCCNP(=O)(OC)OC[C@H]1O[C@@H](n2ccc(=O)[nH]c2=O)[C@](C)(F)[C@@H]1O]
((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-2) 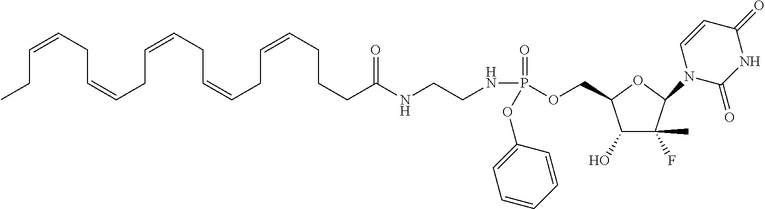 [SMILES: CC/C=C\C/C=C\C/C=C\C/C=C\C/C=C\CCCC(=O)NCCNP(=O)(OC[C@H]1O[C@@H](n2ccc(=O)[nH]c2=O)[C@](C)(F)[C@@H]1O)Oc1ccccc1]
((2R,3R,4R,5R)-5-(2,4-dioxo-3,4-dihydropyrimidin-1(2H)-yl)-4-fluoro-3-hydroxy-4-methyltetrahydrofuran-2-yl)methyl phenyl (2-((5Z,8Z,11Z,14Z,17Z)-icosa-5,8,11,14,17-pentaenamido)ethyl)phosphoramidate (II-3) 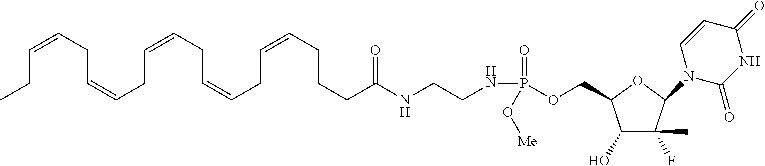 [SMILES: CC/C=C\C/C=C\C/C=C\C/C=C\C/C=C\CCCC(=O)NCCNP(=O)(OC)OC[C@H]1O[C@@H](n2ccc(=O)[nH]c2=O)[C@](C)(F)[C@@H]1O]
((2R,3R,4R,5R)-5-(2,4-dioxo-3,4-dihydropyrimidin-1(2H)-yl)-4-fluoro-3-hydroxy-4-methyltetrahydrofuran-2-yl)methyl methyl (2-((5Z,8Z,11Z,14Z,17Z)-icosa-5,8,11,14,17-pentaenamido)ethyl)phosphoramidate (II-4) 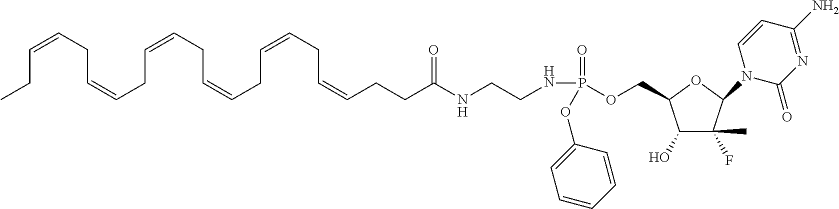 [SMILES: CC/C=C\C/C=C\C/C=C\C/C=C\C/C=C\C/C=C\CCC(=O)NCCNP(=O)(OC[C@H]1O[C@@H](n2ccc(N)nc2=O)[C@](C)(F)[C@@H]1O)Oc1ccccc1]
((2R,3R,4R,5R)-5-(4-amino-2-oxopyrimidin-1(2H)-yl)-4-fluoro-3-hydroxy-4-methyltetrahydrofuran-2-yl)methyl phenyl (2-((4Z,7Z,10Z,13Z,16Z,19Z)-docosa-4,7,10,13,16,19-hexaenamido)ethyl)phosphoramidate (II-17); and 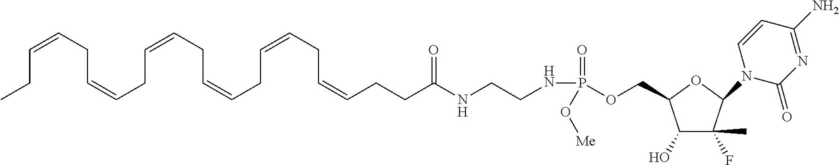 [SMILES: CC/C=C\C/C=C\C/C=C\C/C=C\C/C=C\C/C=C\CCC(=O)NCCNP(=O)(OC)OC[C@H]1O[C@@H](n2ccc(N)nc2=O)[C@](C)(F)[C@@H]1O]
((2R,3R,4R,5R)-5-(4-amino-2-oxopyrimidin-1(2H)-yl)-4-fluoro-3-hydroxy-4-methyltetrahydrofuran-2-yl)methyl methyl (2-((4Z,7Z,10Z,13Z,16Z,19Z)-docosa-4,7,10,13,16,19-hexaenamido)ethyl)phosphoramidate (II-18).

15. The compound of claim 10 wherein the compound is selected from the group consisting of 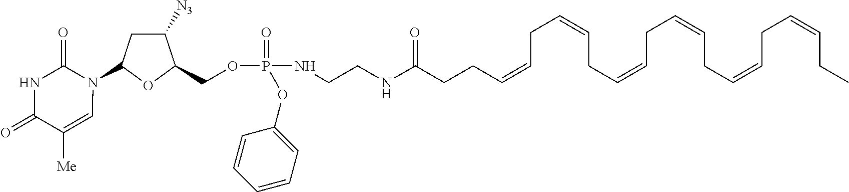
((2S,3S,5S)-3-azido-5-(5-methyl-2,4-dioxo-3,4-dihydropyrimidin-1(2H)-yl)tetrahydrofuran-2-yl)methyl phenyl (2-((4Z,7Z,10Z,13Z,16Z,19Z)-docosa-4,7,10,13,16,19-hexaenamido)ethyl)phosphoramidate (II-25) 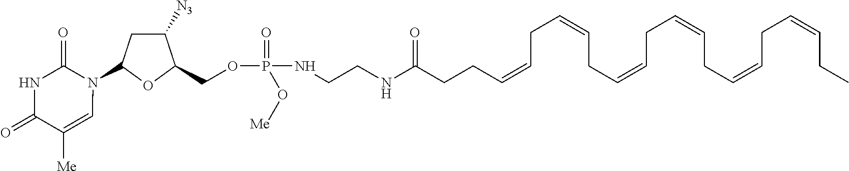
((2S,3S,5S)-3-azido-5-(5-methyl-2,4-dioxo-3,4-dihydropyrimidin-1(2H)-yl)tetrahydrofuran-2-yl)methyl methyl (2-((4Z,7Z,10Z,13Z,16Z,19Z)-docosa-4,7,10,13,16,19-hexaenamido)ethyl)phosphoramidate (II-26) 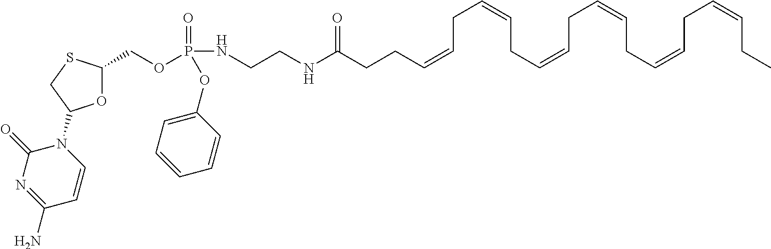
((2R,5S)-5-(4-amino-2-oxopyrimidin-1(2H)-yl)-1,3-oxathiolan-2-yl)methyl phenyl (2-((4Z,7Z,10Z,13Z,16Z,19Z)-docosa-4,7,10,13,16,19-hexaenamido)ethyl)phosphoramidate (II-67) 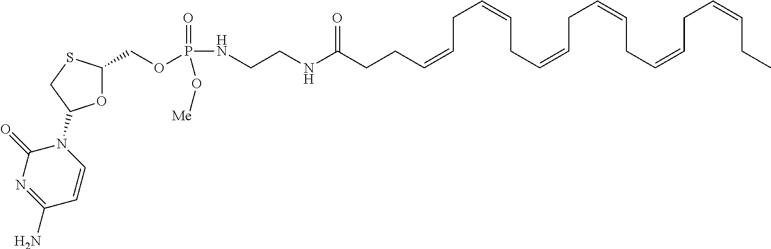
((2R,5S)-5-(4-amino-2-oxopyrimidin-1(2H)-yl)-1,3-oxathiolan-2-yl)methyl methyl (2-((4Z,7Z,10Z,13Z,16Z,19Z)-docosa-4,7,10,13,16,19-hexaenamido)ethyl)phosphoramidate (II-68) 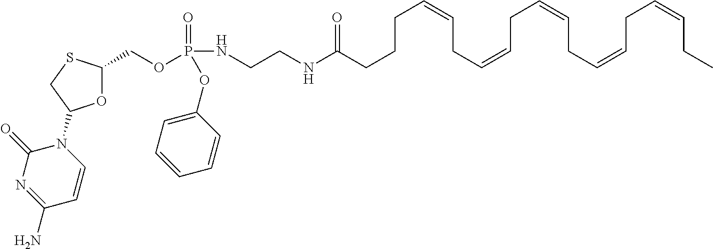
((2R,5S)-5-(4-amino-2-oxopyrimidin-1(2H)-yl)-1,3-oxathiolan-2-yl)methyl phenyl (2-((5Z,8Z,11Z,14Z,17Z)-icosa-5,8,11,14,17-pentaenamido)ethyl)phosphoramidate (II-69) 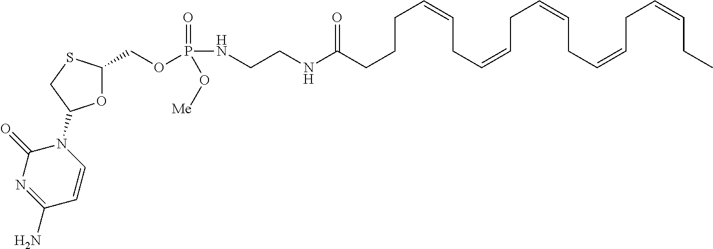
((2R,5S)-5-(4-amino-2-oxopyrimidin-1(2H)-yl)-1,3-oxathiolan-2-yl)methyl methyl (2-((5Z,8Z,11Z,14Z,17Z)-icosa-5,8,11,14,17-pentaenamido)ethyl)phosphoramidate (II-70)

16. The compound of claim 10, wherein the compound is selected from the group consisting of 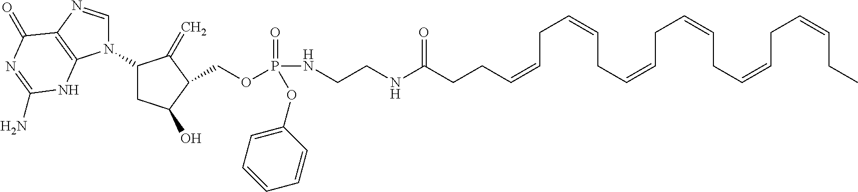
((1R,3S,5S)-3-(2-amino-6-oxo-3H-purin-9(6H)-yl)-5-hydroxy-2-methylenecyclopentyl)methyl phenyl (2-((4Z,7Z,10Z,13Z,16Z,19Z)-docosa-4,7,10,13,16,19-hexaenamido)ethyl)phosphoramidate (II-45) and 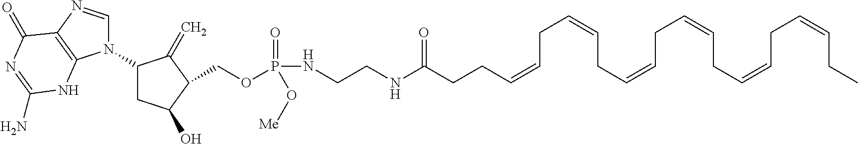
((1R,3S,5S)-3-(2-amino-6-oxo-3H-purin-9(6H)-yl)-5-hydroxy-2-methylenecyclopentyl)methyl methyl (2-((4Z,7Z,10Z,13Z,16Z,19Z)-docosa-4,7,10,13,16,19-hexaenamido)ethyl)phosphoramidate (II-46)

17. The compound of claim 10 wherein the compound is selected from the group consisting of 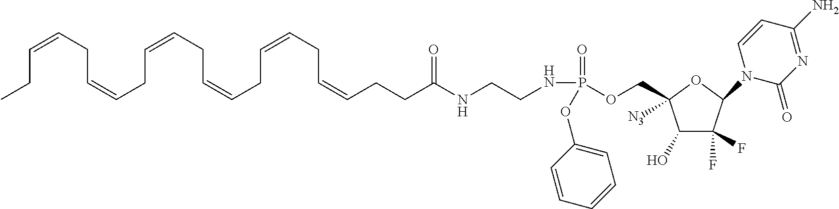
((2R,3R,5R)-5-(4-amino-2-oxopyrimidin-1(2H)-yl)-2-azido-4,4-difluoro-3-hydroxytetrahydrofuran-2-yl)methyl phenyl (2-((4Z,7Z,10Z,13Z,16Z,19Z)-docosa-4,7,10,13,16,19-hexaenamido)ethyl)phosphoramidate (II-59) 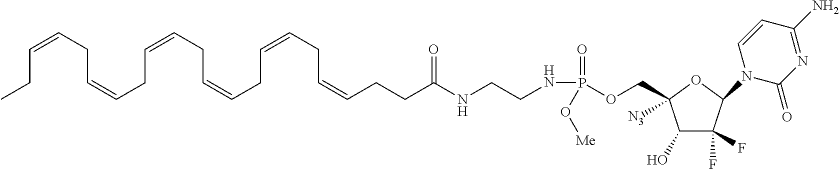
((2R,3R,5R)-5-(4-amino-2-oxopyrimidin-1(2H)-yl)-2-azido-4,4-difluoro-3-hydroxytetrahydrofuran-2-yl)methyl methyl (2-((4Z,7Z,10Z,13Z,16Z,19Z)-docosa-4,7,10,13,16,19-hexaenamido)ethyl)phosphoramidate (II-60) 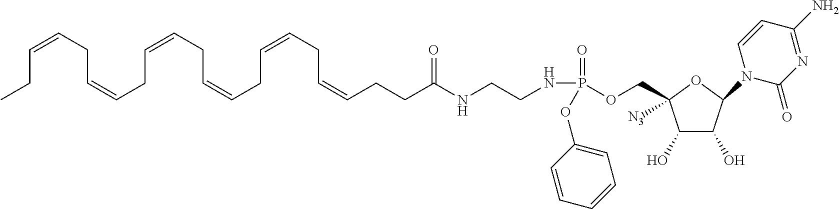 [SMILES: CC/C=C\C/C=C\C/C=C\C/C=C\C/C=C\C/C=C\CCC(=O)NCCNP(=O)(OC[C@@]1(N=[N+]=[N-])O[C@@H](n2ccc(N)nc2=O)[C@H](O)[C@@H]1O)Oc1ccccc1]
((2R,3S,4R,5R)-5-(4-amino-2-oxopyrimidin-1(2H)-yl)-2-azido-3,4-dihydroxytetrahydrofuran-2-yl)methyl phenyl (2-((4Z,7Z,10Z,13Z,16Z,19Z)-docosa-4,7,10,13,16,19-hexaenamido)ethyl)phosphoramidate (II-61); and 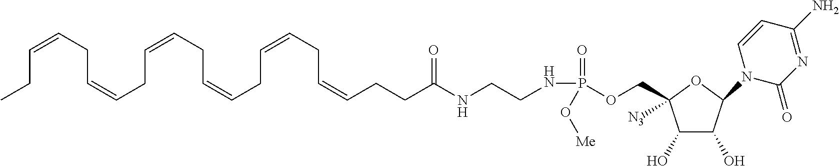 [SMILES: CC/C=C\C/C=C\C/C=C\C/C=C\C/C=C\C/C=C\CCC(=O)NCCNP(=O)(OC)OC[C@@]1(N=[N+]=[N-])O[C@@H](n2ccc(N)nc2=O)[C@H](O)[C@@H]1O]
((2R,3S,4R,5R)-5-(4-amino-2-oxopyrimidin-1(2H)-yl)-2-azido-3,4-dihydroxytetrahydrofuran-2-yl)methyl methyl (2-((4Z,7Z,10Z,13Z,16Z,19Z)-docosa-4,7,10,13,16,19-hexaenamido)ethyl)phosphoramidate (II-62)

18-20. (canceled)

21. A pharmaceutical composition comprising the conjugate of claim 1 and a pharmaceutically acceptable carrier.

22. A pharmaceutical composition comprising the conjugate of claim 2 and a pharmaceutically acceptable carrier.

23. A pharmaceutical composition comprising the compound of claim 3 and a pharmaceutically acceptable carrier.

24-29. (canceled)

30. A method of treating or preventing a viral infection, the method comprising administering to a patient in need thereof an effective amount of a molecular conjugate of claim 1.

31. A method of treating or preventing a viral infection, the method comprising administering to a patient in need thereof an effective amount of the compound of claim 2.

32. A method of treating or preventing a viral infection, the method comprising administering to a patient in need thereof an effective amount of the compound of claim 3.

33-38. (canceled)

39. A method of treating or preventing a viral infection, the method comprising administering to a patient in need thereof an effective amount of the compound of claim 14.

40. The method of claim 39, wherein the viral infection is hepatitis C.

41. A method of treating or preventing a viral infection, the method comprising administering to a patient in need thereof an effective amount of the compound of claim 15.

42. The method of claim 41, wherein the viral infection is HIV.

43. A method of treating or preventing a viral infection, the method comprising administering to a patient in need thereof an effective amount of the compound of claim 16,

44. The method of claim 43, wherein the viral infection is hepatitis B.

45-55. (canceled)
